(19) United States
(12) Patent Application Publication
(10) Pub. No.: US 2004/0071700 A1
(43) Pub. Date: Apr. 15, 2004

(54) Obesity linked genes

(75) Inventors: Jaeseob Kim, Yousong-Gu Taejon (KR); Ron Galant, Madison, WI (US)

Correspondence Address: David A. Casimir, MEDLEN & CARROLL, LLP, Suite 350, 101 Howard Street, San Francisco, CA, 94105, US

(73) Assignee: Life Sciences Development Corp., Madison, WI

(21) Appl. No.: 10/267,502

(22) Filed: Oct. 9, 2002

(51) Int. Cl. A61K 39/395; G01N 33/53; G01N 33/567

(52) U.S. Cl. 424/145.1; 435/7.2

(57) ABSTRACT

The present invention relates to newly identified nucleic acids, their encoded proteins, and to the use of such nucleic acids and proteins. The invention also relates the correlation between the expression of genes and fat cell size and number. The invention also relates to modifying the activity of a protein that affects the number and/or size of fat cells by regulating the expression of the nucleic acids, homologs, or active variants or their encoded proteins.

### FIELD OF THE INVENTION

The present invention relates to newly identified nucleic acids, their encoded proteins, and to the use of such nucleic acids and proteins. The invention also relates the correlation between the expression of genes and fat cell size and number. The invention also relates to modifying the activity of a protein that affects the number and/or size of fat cells by regulating the expression of the nucleic acids, homologs, or active variants or their encoded proteins.

### BACKGROUND OF THE INVENTION

Obesity is a chronic, stigmatized, and costly condition characterized by excess adipose (fat) tissue caused by increased storage of fat molecules (Bray and Tartaglia, Nature, 404:672 (2000); Flier, Nature, 409:292 (2001); Lazar, Genes Dev. 1:16 (2002)). It is rarely curable and is increasing in prevalence in most of the world (Bray and Tartaglia, 2000). Obesity is also often associated with insulin-resistant diabetes, and the health-related consequences of excessive fat can principally be attributed to this connection (Friedman, Nature 415:268 (2002)). There exists strong evidence of a genetic contribution to obesity, both in human studies and a number of animal models (Robinson et al., Annu Rev Genet. 34:687 (2000); Spiegelman and Flier, Cell 104:531 (2001)). Genes that have demonstrated effects on the number and/or size of fat cells include those that encode enyzmes such as glycerol 3-phosphate dehydrogenase and hormone-sensitive lipase; transcriptional regulators such as sterol regulatory element-binding proteins (SREBPs) and peroxisome proliferator-activated receptor-gamma (PPAR-gamma), and; secreted signaling proteins such as leptin (Robinson et al., 2000). Thus, a substantial number of genes play critical roles in regulating the number and/or size of fat cells and hence in obesity.

It would thus be advantageous to identify nucleic acids and their encoded proteins that modify the number and/or size of fat cells, thereby providing drug screening targets and therapeutic compositions for preventing or treating obesity and/or diabetes. Furthermore, genes that influence the number and/or size of fat cells might vary in sequence among humans, and some variants may increase the predisposition for developing obesity and/or diabetes. It would also be advantageous to provide a method for detecting an individual's predisposition to obesity and/or diabetes in order to prevent or treat this condition.

### SUMMARY OF THE INVENTION

The present invention relates to newly identified nucleic acids, their encoded proteins, and to the use of such nucleic acids and proteins. The invention also relates the correlation between the expression of genes and fat cell size and number. The invention also relates to modifying the activity of a protein that affects the number and/or size of fat cells by regulating the expression of the nucleic acids, homologs, or active variants or their encoded proteins.

Thus, in some embodiments, the present invention provides an isolated and purified nucleic acid comprising a sequence encoding a protein selected from the group consisting of SEQ ID NOs: 218-434. In some embodiments, the nucleic acid sequence is operably linked to a heterologous promoter. In some embodiments, the nucleic acid sequence is contained within a vector. In some further embodiments, the vector is within a host cell. In some preferred embodiments, the host cell is a adipocyte or a cell that capable of differentiating into an adipocyte (e.g., preadipocytes, fibroblasts, etc.).

In other embodiments, the present invention provides an isolated and purified nucleic acid sequence that hybridizes under conditions of low stringency to a nucleic acid selected from the group consisting of SEQ ID NOs: 1-217. In some embodiments, the nucleic acid sequence encodes a protein. In other embodiments, the present invention provides a vector comprising the nucleic acid sequence. In still other embodiments, the vector is within a host cell. In some embodiments, the host cell is located in an organism selected from the group consisting of a plant and an animal.

In yet other embodiments the present invention provides a protein encoded by a nucleic acid selected from the group consisting of SEQ ID NOs: 1-217 and variants thereof that are at least 80% identical to SEQ ID NOs: 1-217 and wherein the protein has at least one activity of a protein encoded by SEQ ID NOs: 218-434. In some embodiments, the activity is increasing or decreasing the number and/or size of fat cells. In some embodiments, the protein is at least 90% identical to SEQ ID NOs: 218-434. In other embodiments, the protein is at least 95% identical to SEQ ID NOs: 218-434.

In still further embodiments, the present invention provides a method for producing variants of genes comprising: providing a nucleic acid sequence selected from the group consisting of SEQ ID NOs: 1-217; mutagenizing the nucleic acid sequence; and screening the variant for activity.

In other embodiments, the present invention provides a composition comprising a nucleic acid that inhibits the binding of at least a portion of a nucleic acid selected from the group consisting of SEQ ID NOs: 1-217 to their complementary sequences. In yet other embodiments, the present invention provides a nucleic acid sequence comprising at least fifteen (e.g., 15-1000) nucleotides capable of hybridizing under stringent conditions to the isolated nucleotide sequence selected from the group consisting of SEQ ID NOs: 1-217.

The present invention also provides a method for detection of a nucleic acid encoding a protein of SEQ ID NOs: 218-434 in a biological sample suspected of containing a nucleic acid encoding SEQ ID NOs: 218-434. The method includes hybridizing the nucleic acid sequence selected from the group consisting of SEQ ID NOs: 1-217 and variants thereof that are at least 80% identical to SEQ ID NOs: 1-217 to the nucleic acid of the biological sample to produce a hybridization complex. In some embodiments, the method further includes the step of detecting the hybridization complex, wherein the presence of the hybridization complex indicates the presence of a nucleic acid encoding the protein in the biological sample. In some embodiments, prior to hybridization, the nucleic acid of the biological sample is amplified.

The present invention also provides a method for identifying compounds that influence fat cell number or size, comprising the steps of providing: a cell that expresses a gene selected from the group consisting of SEQ ID Nos: 1-217, and an agent; exposing the cell to the agent; and identifying fat cell number or size relative to cells not exposed to the agent. The present invention is not limited by the nature of the cell. The cell may reside in vitro (e.g., in tissue culture, on a cell array, etc.), ex vivo (e.g., in an isolated tissue), or in vivo (e.g., in an organism). The present invention is also not limited by the nature of the agent. Agents include antibodies, antisense molecules, small molecule drugs, peptides, and the like. In some preferred embodiments, the agent is part of a compound library. The present invention is not limited by the method in which fat cell size or number are identified. Identification can be direct (e.g., cell counting, lipid quantitation, etc.) or indirect (e.g., body weight measurements, detection of differentiation markers or metabolism markers, etc.).

In some embodiments, agents identified in the methods above are tested in studies to demonstrate the safety and/or efficacy of the agents (e.g., regulatory trials such as Food and Drug Administration trials). In some embodiments, the agents are sold for the purpose of treating or preventing conditions related to dysregulation of fat cell size or number (e.g., obesity, diabetes, wasting diseases). In some embodiments, the agents are labeled with instructions for use with other labels (e.g., labeling required by a government agency such as the Food and Drug Administration). In some embodiments, the agents are marketed (e.g., advertised) for use in treating or preventing diseases or conditions associated with dysregulation of fat cell size or number.

The present invention also provides methods for identifying compounds that influence fat cell number or size, whereby an agent is first tested for a biological activity prior to treating a cell or organism with the agent to identify changes in fat cell number or size. In some embodiments, an expression vector comprising a gene selected from the group consisting of SEQ ID Nos: 1-217 is exposed to an agent and a change in expression of the gene relative to the expression of the gene in an expression vector not exposed to the agent is determined. In alternative embodiments, a polypeptide selected from the group consisting of SEQ ID Nos: 218-434 is exposed to an agent and binding of the agent to the polypeptide (or the ability of the agent to prevent the polypeptide from binding a known binding partner) or a change in an activity of the polypeptide is detected.

The present invention also provides methods for regulating fat cell size or number, comprising the steps of providing a subject containing fat cells and an agent that changes the expression of a gene selected from the group consisting of SEQ ID NOs: 1-217; and treating the subject with the agent under conditions such that fat cell size or number in the subject is altered. In some embodiments, the agent is an expression vector that expresses the gene.

The present invention also provides methods for regulating fat cell size or number, comprising the steps of providing a subject containing fat cells and an agent that changes that the in vivo activity of a polypeptide selected from the group consisting of SEQ ID NOs: 218-434; and treating the subject with the agent under conditions such that fat cell size or number in the subject is altered. In some embodiments, the agent is a polypeptide selected from the group consisting of SEQ ID NOs. 218-434. In other embodiments, the agent is an antibody, peptide, or small molecule drug that binds to the polypeptide.

In yet other embodiments, the present invention provides a kit for determining if a subject is at risk of developing obesity, diabetes, or other diseases or conditions associated with fat cell size or number, comprising: at least one reagent that specifically detects expression of SEQ ID NOs. 1-217 or a specific allele of SEQ ID NOs. 1-217; and instructions for determining that the subject is at increased risk of developing obesity, diabetes, or other diseases or conditions associated with fat cell size or number.

### DESCRIPTION OF THE FIGURES

FIG. 1 shows the sequence of SEQ ID NOs: 1-434 found in Table 2.

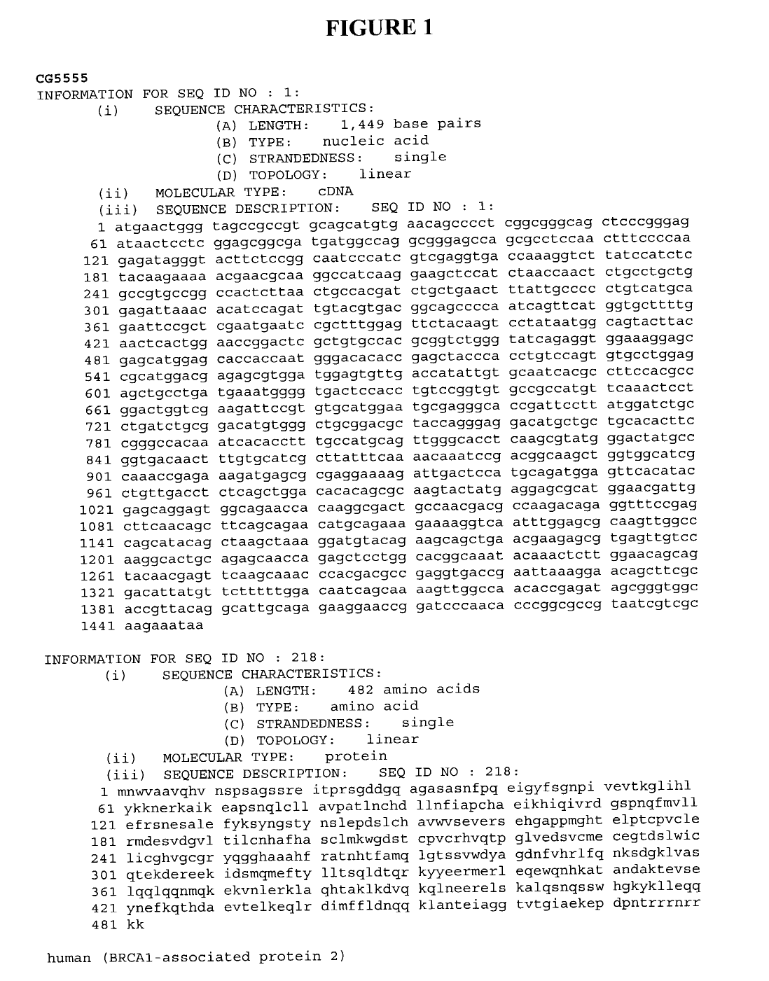

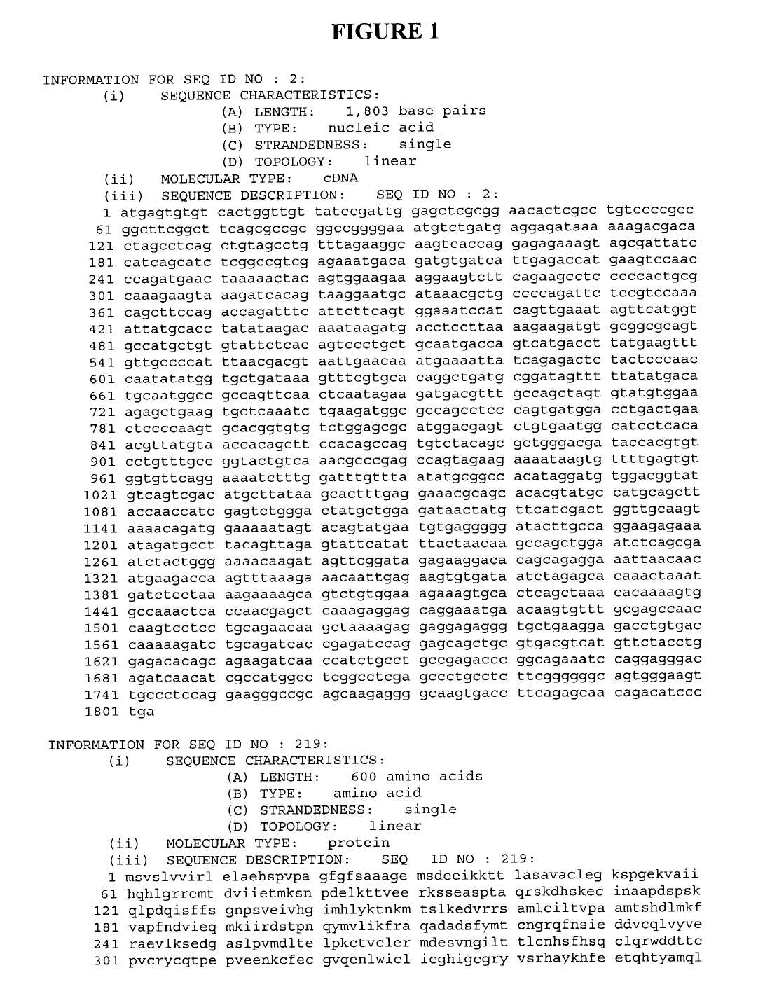

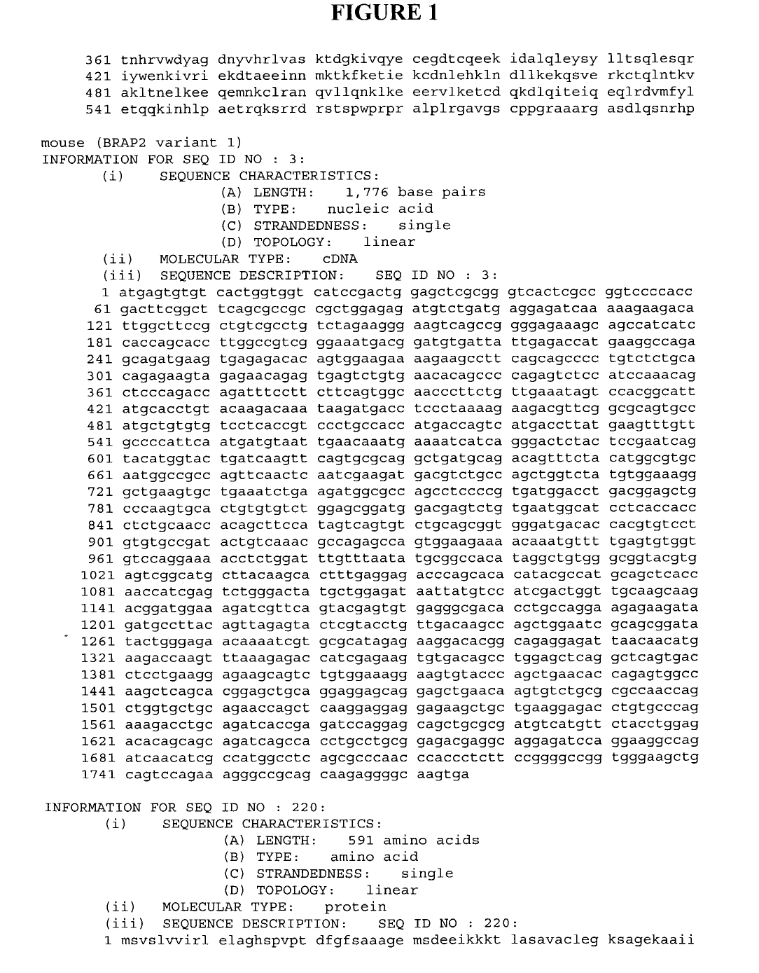

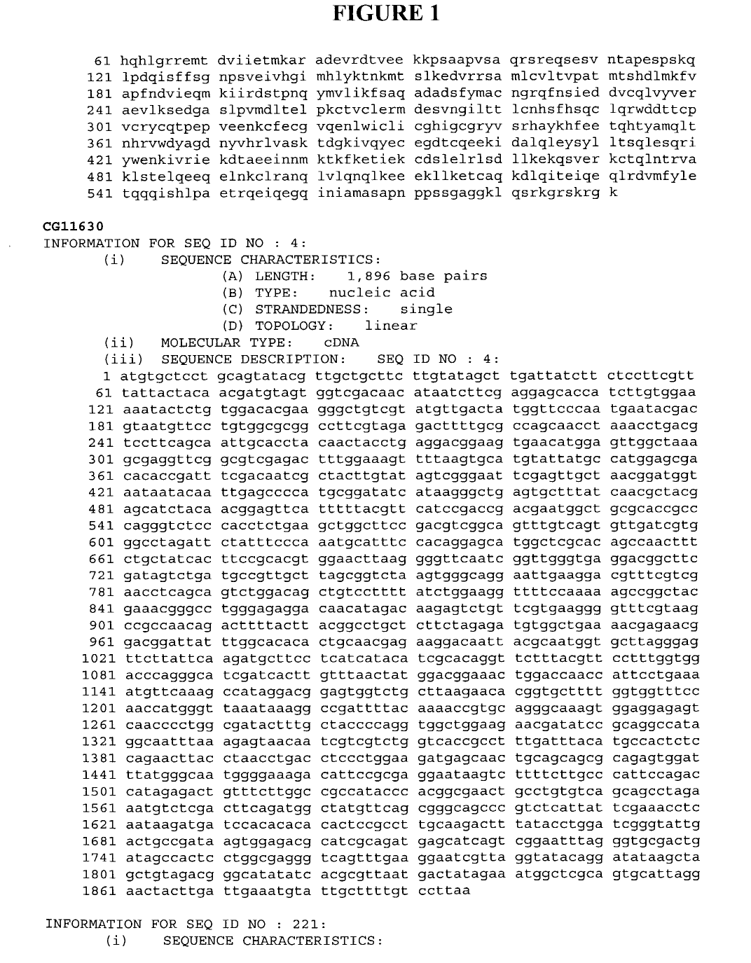

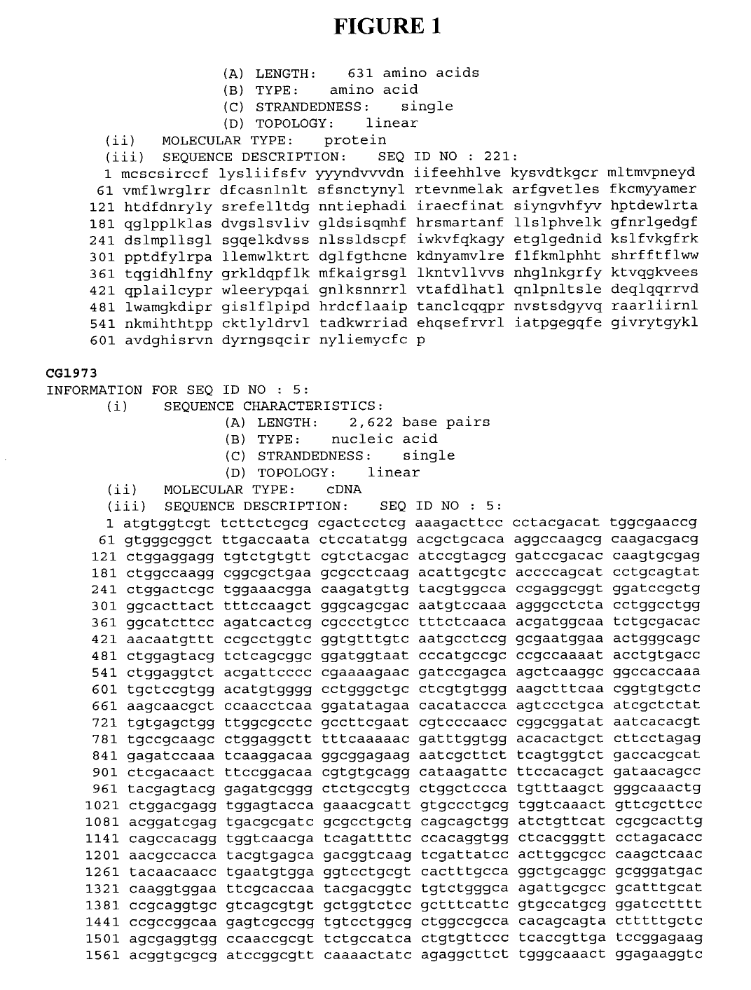

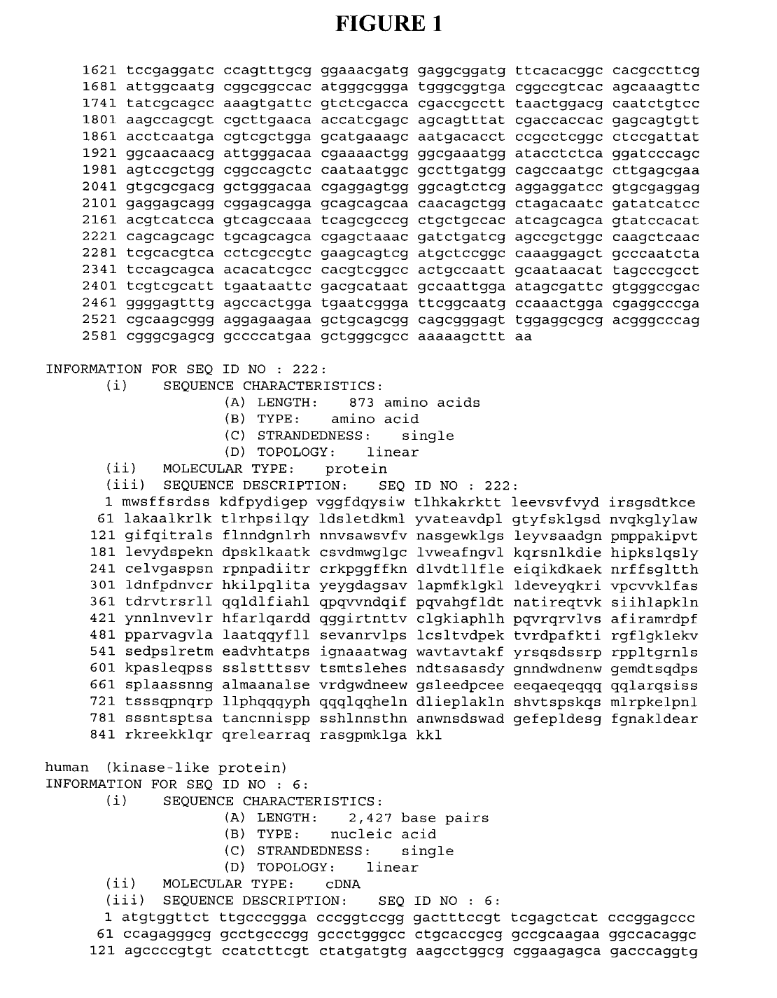

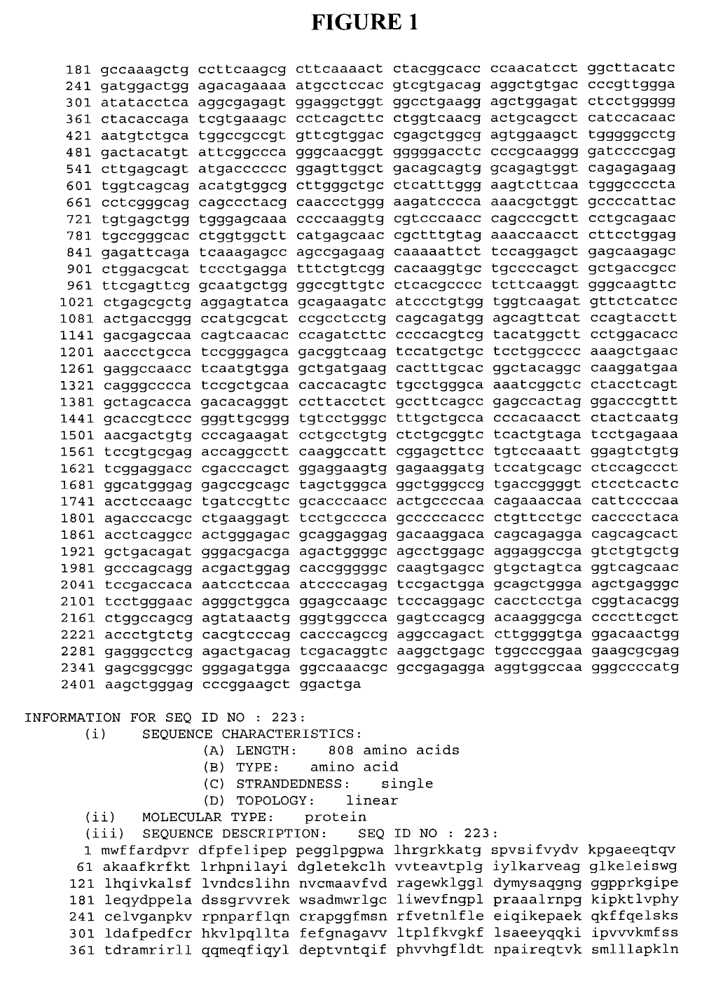

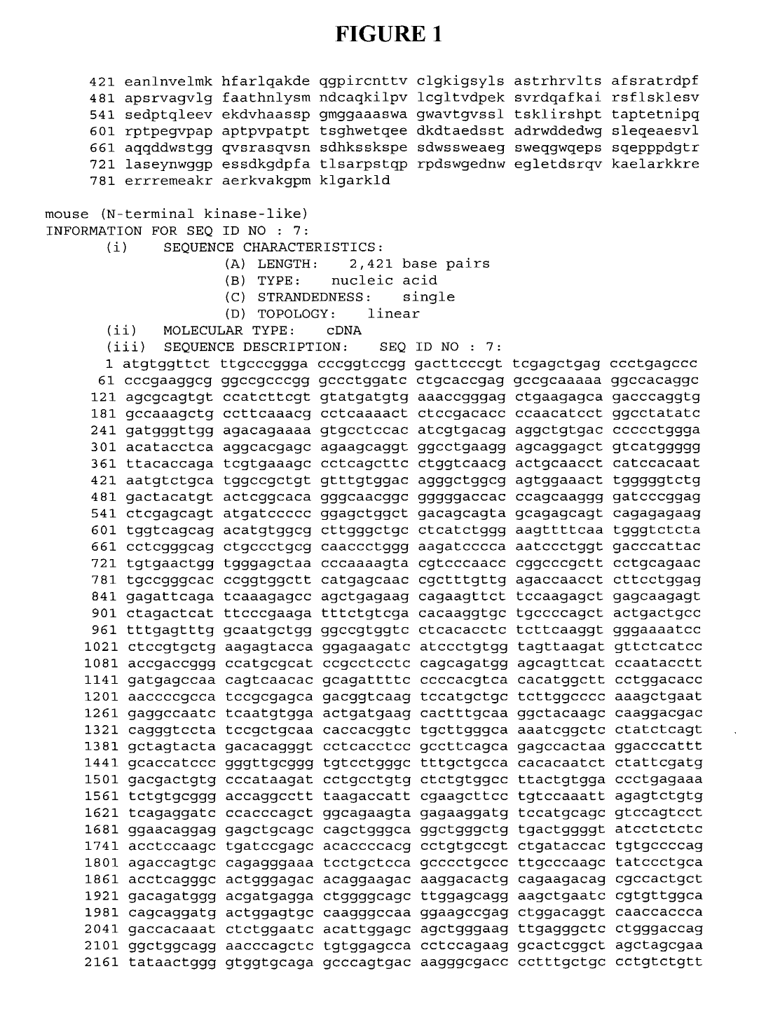

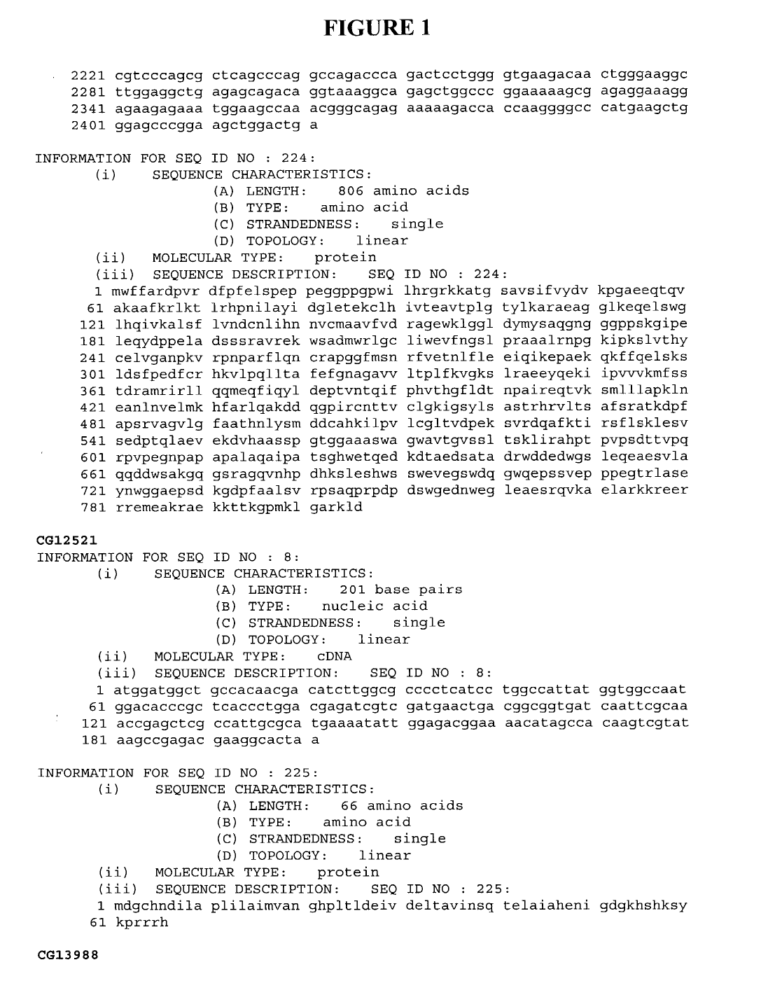

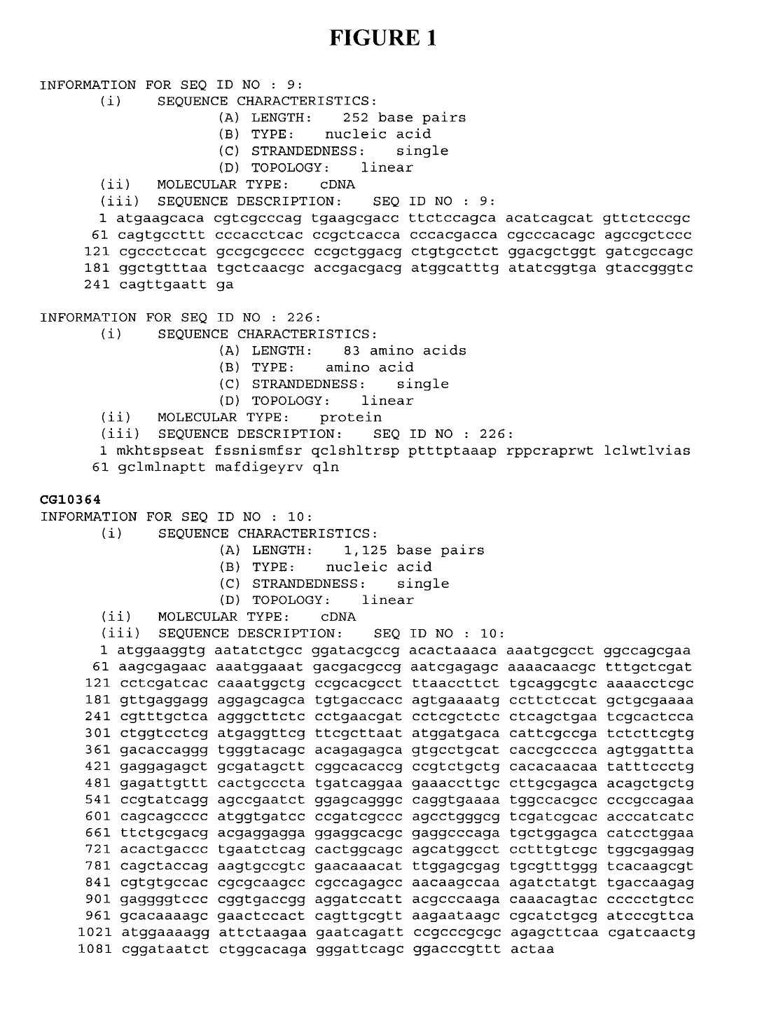

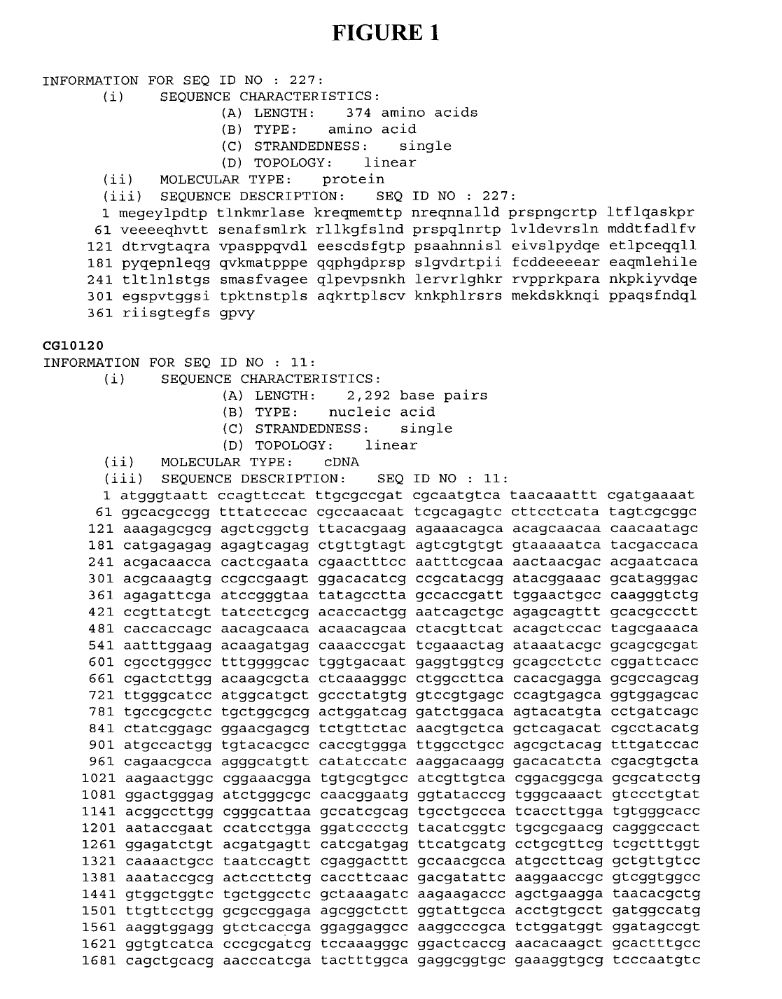

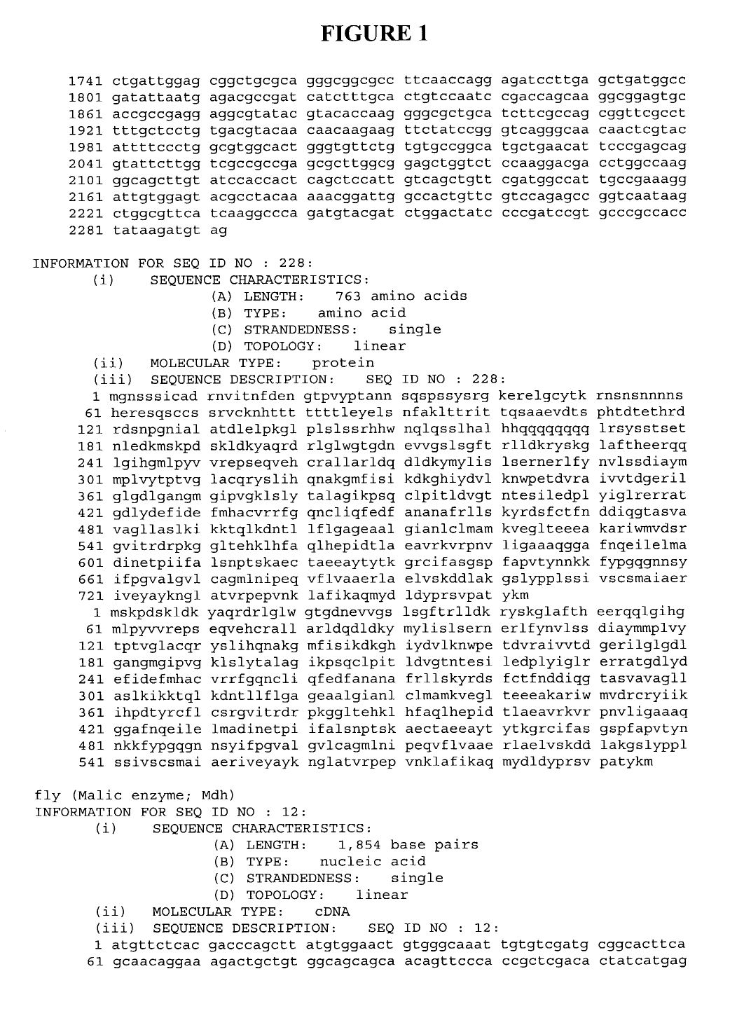

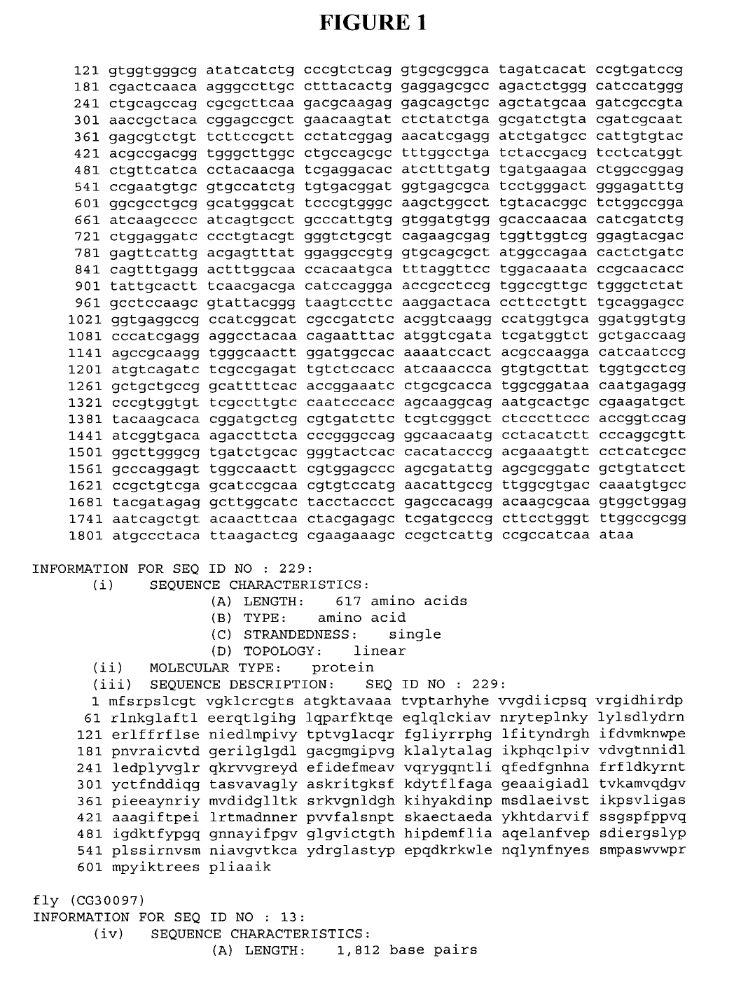

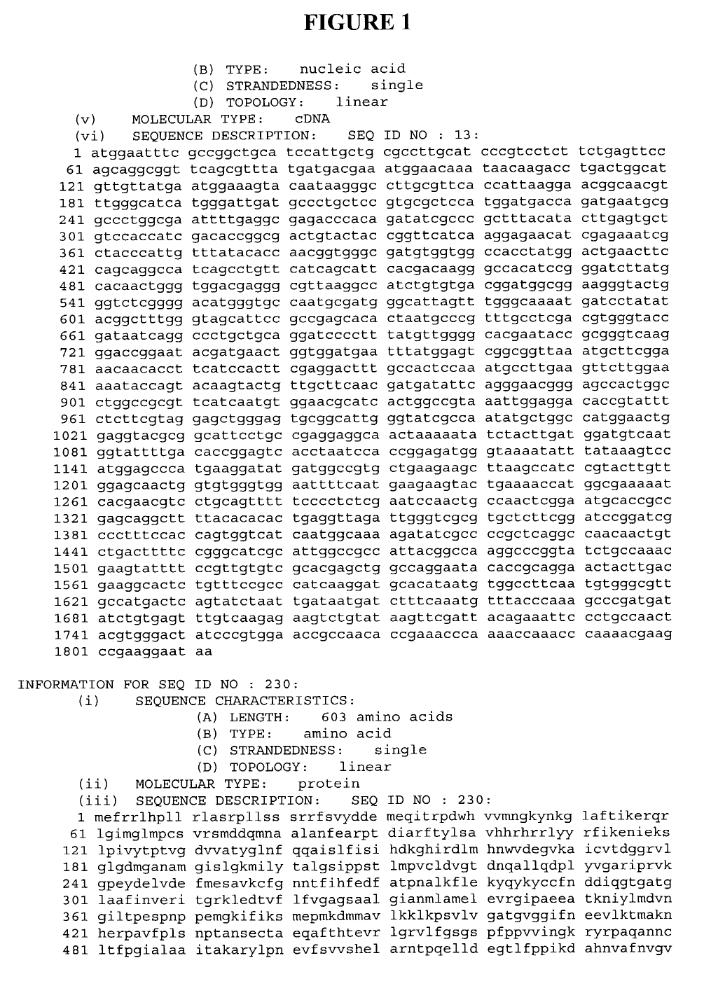

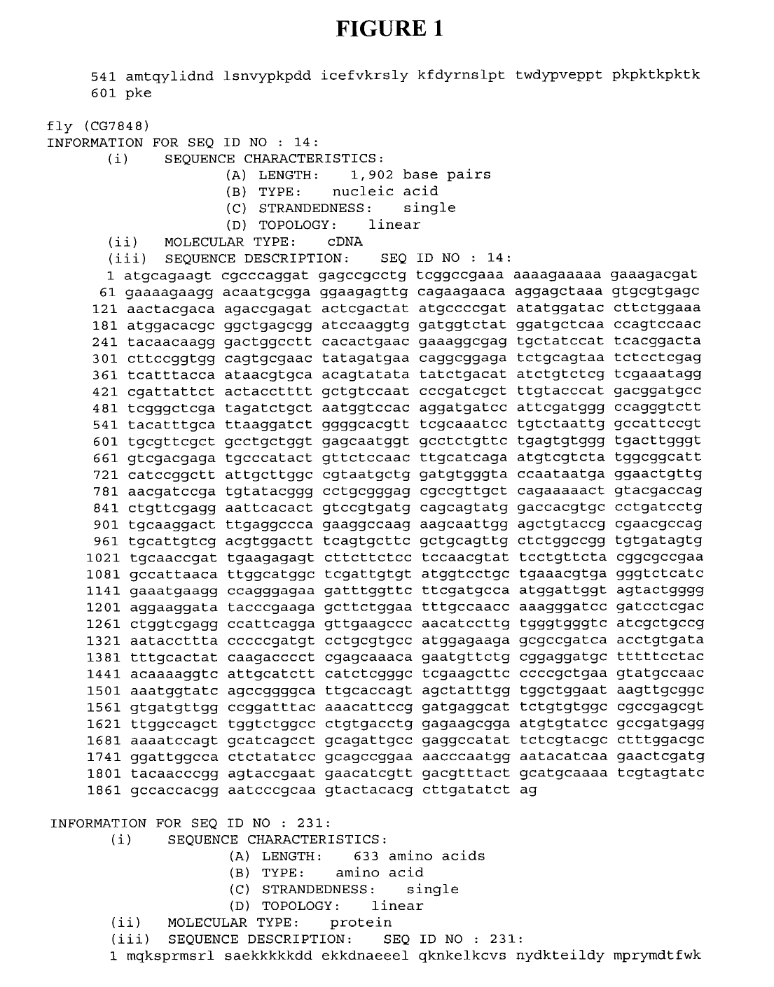

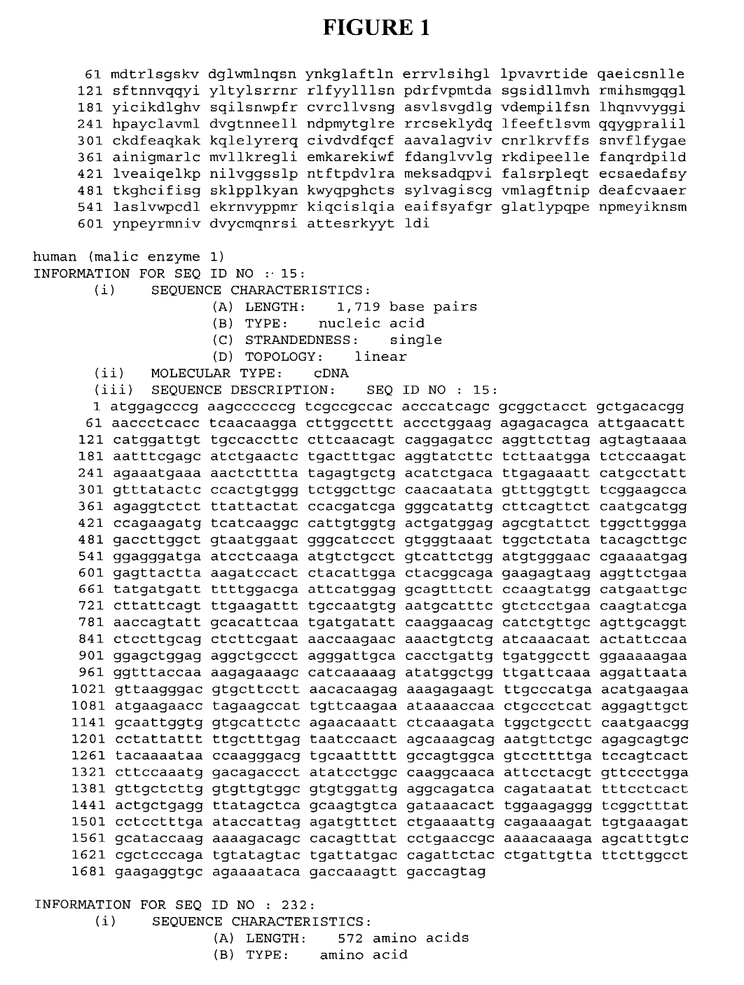

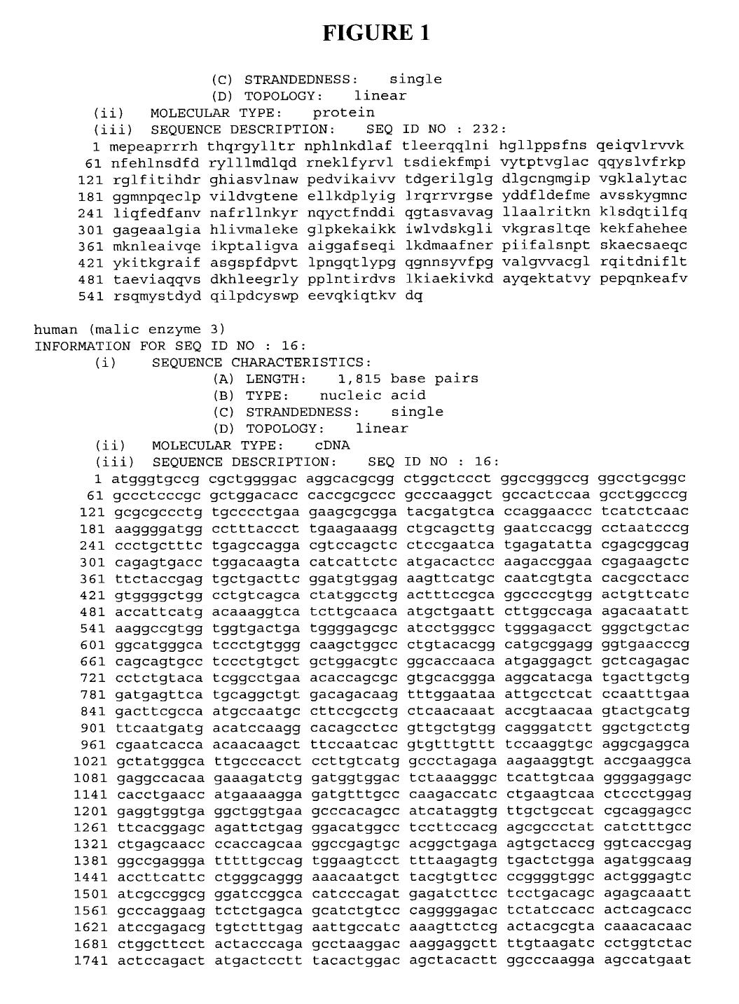

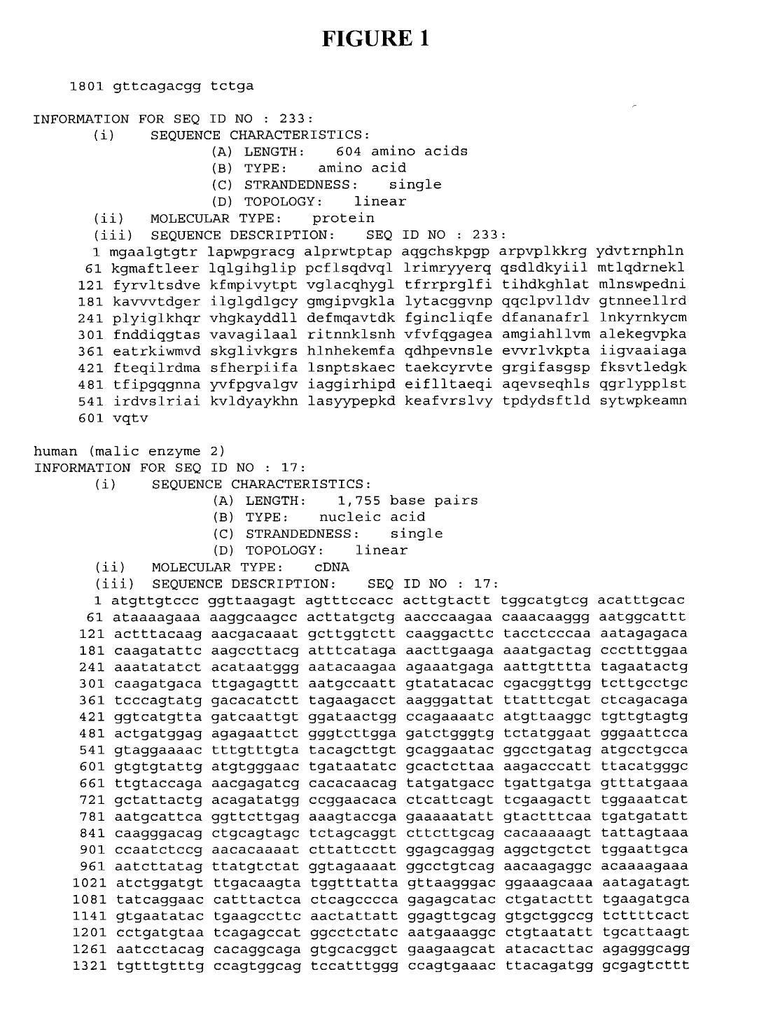

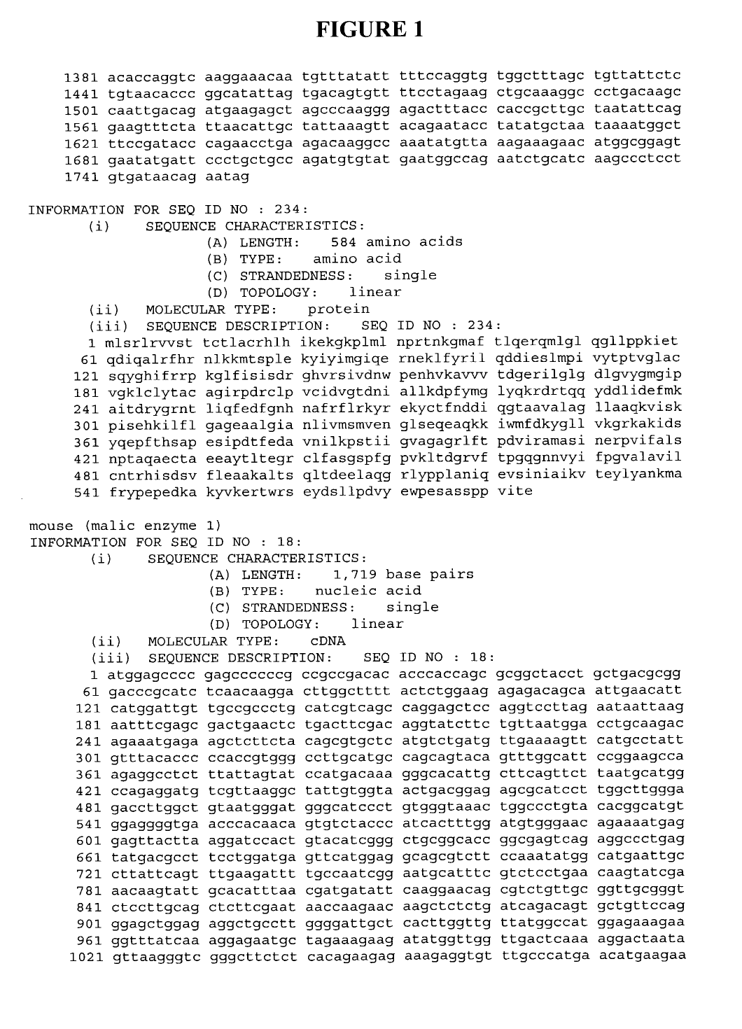

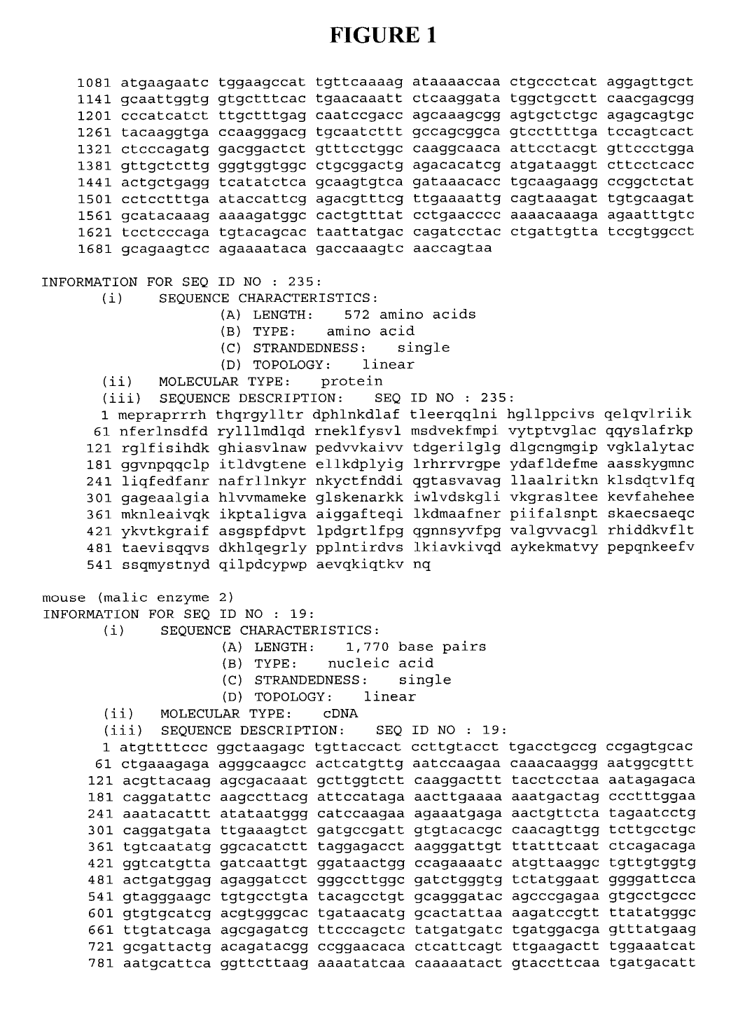

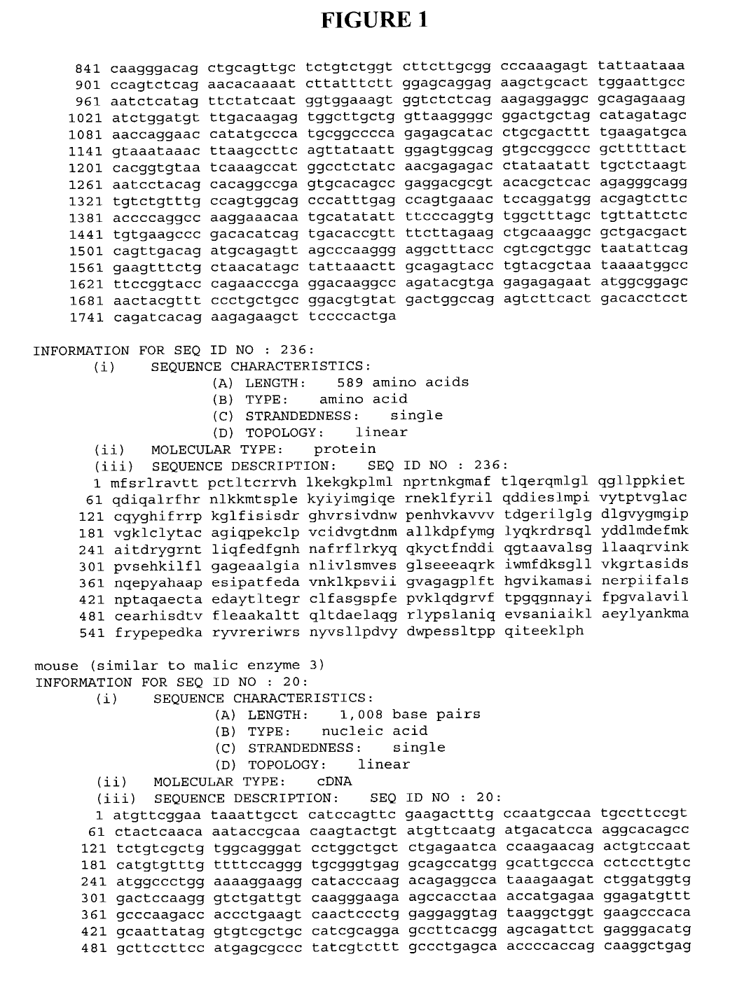

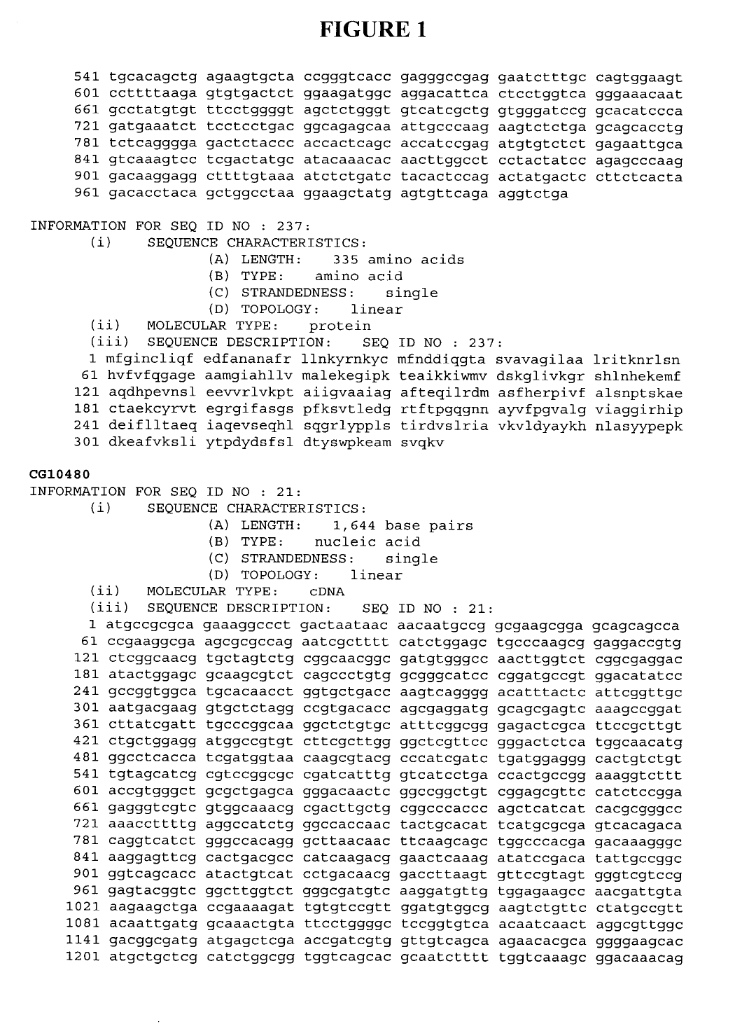

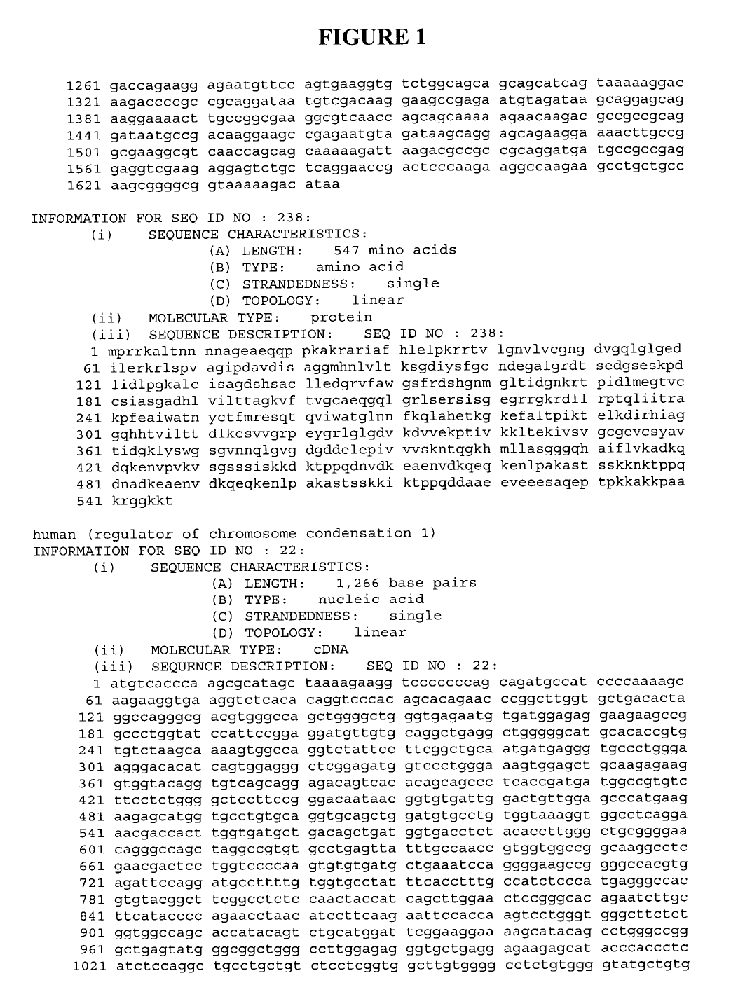

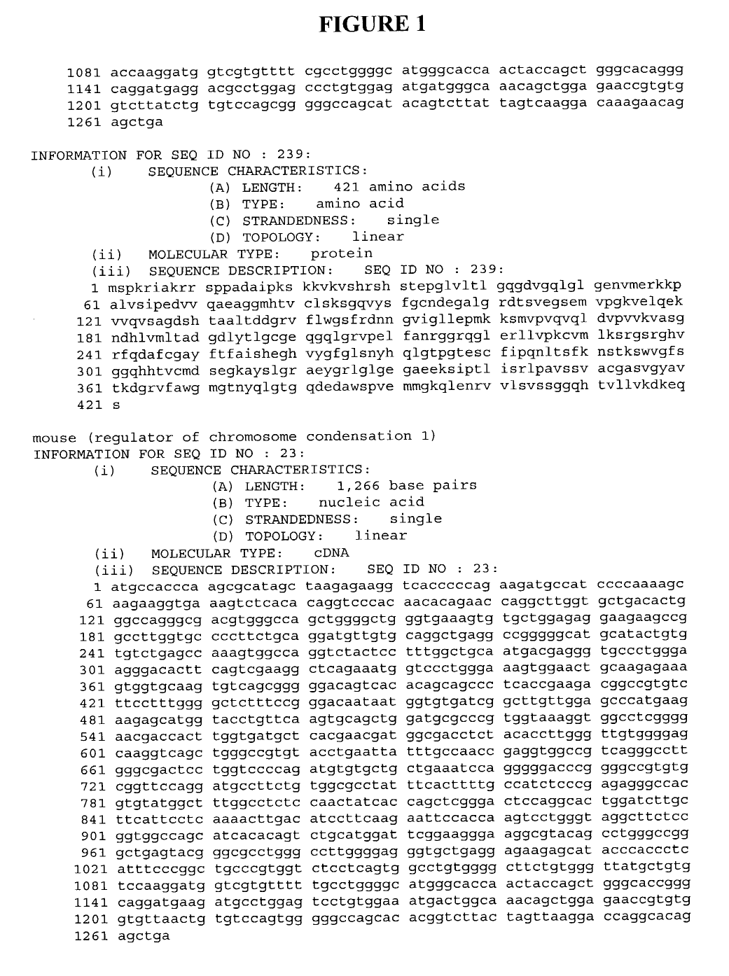

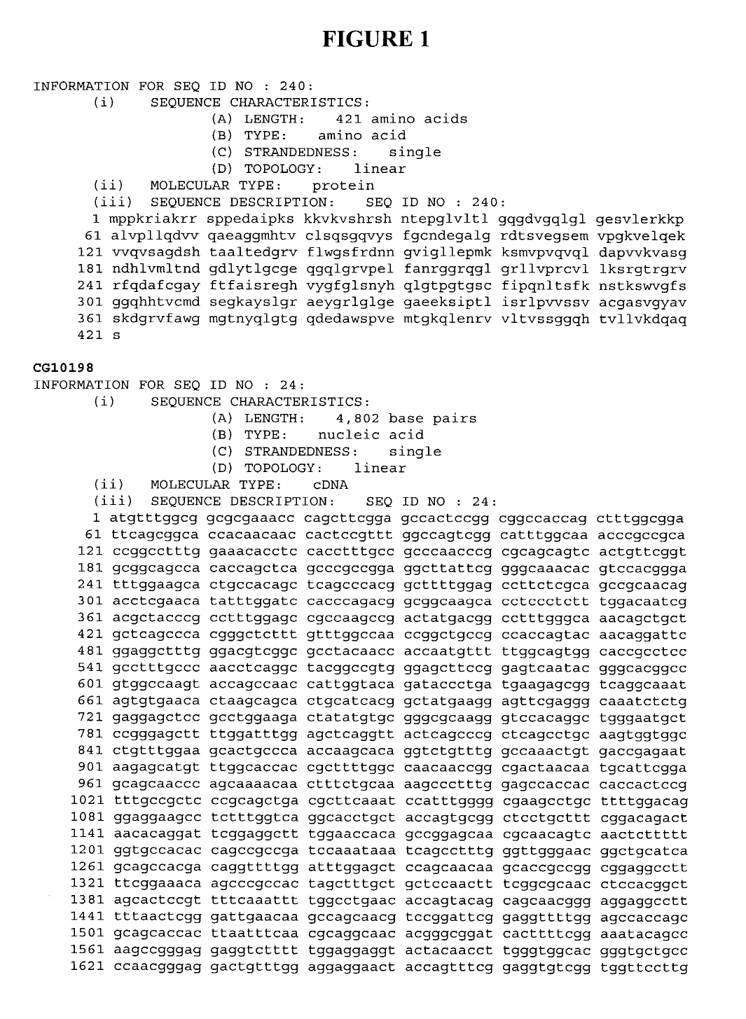

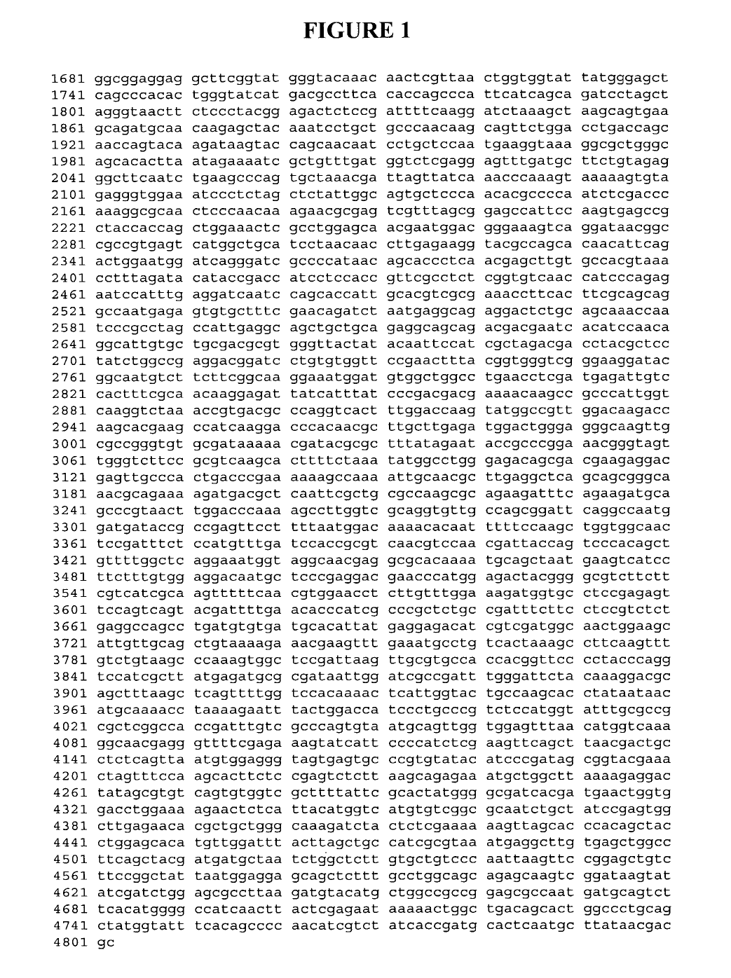

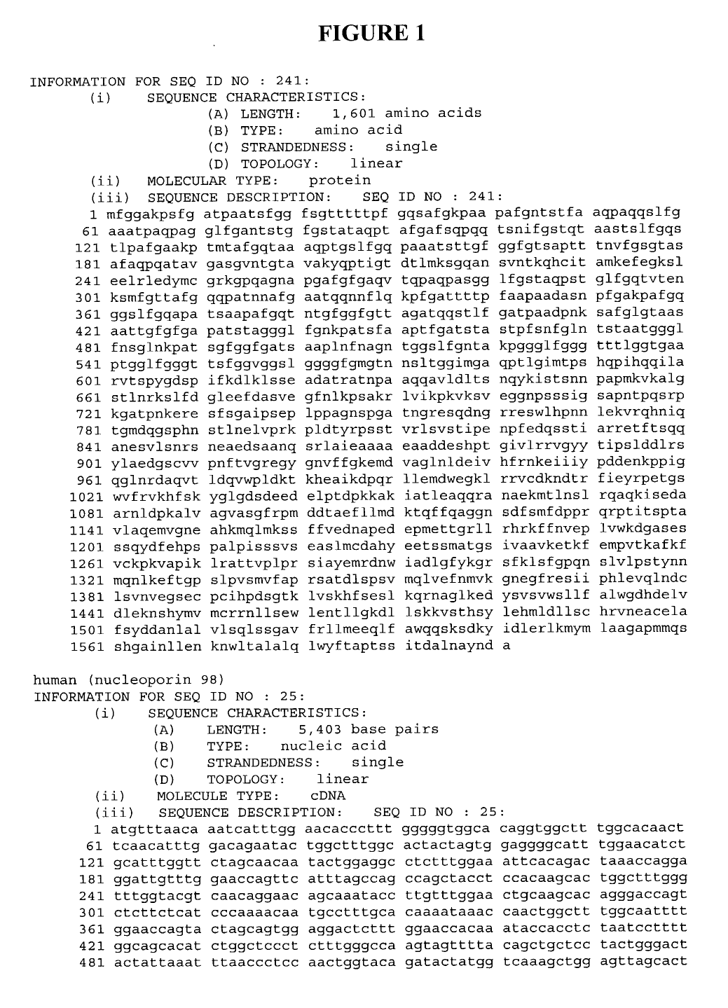

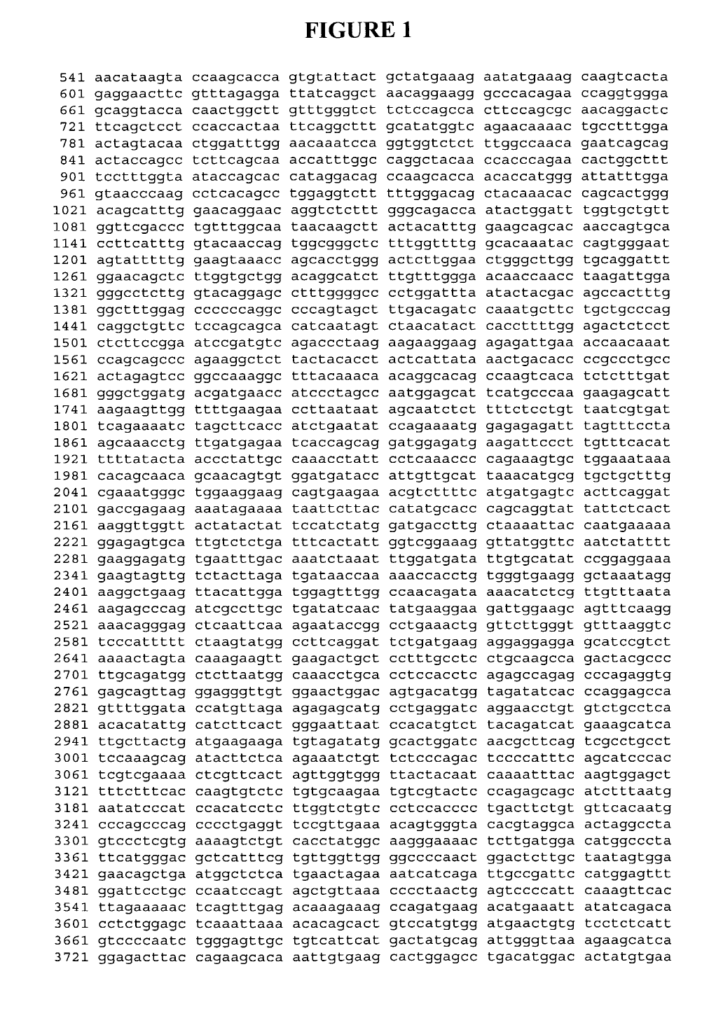

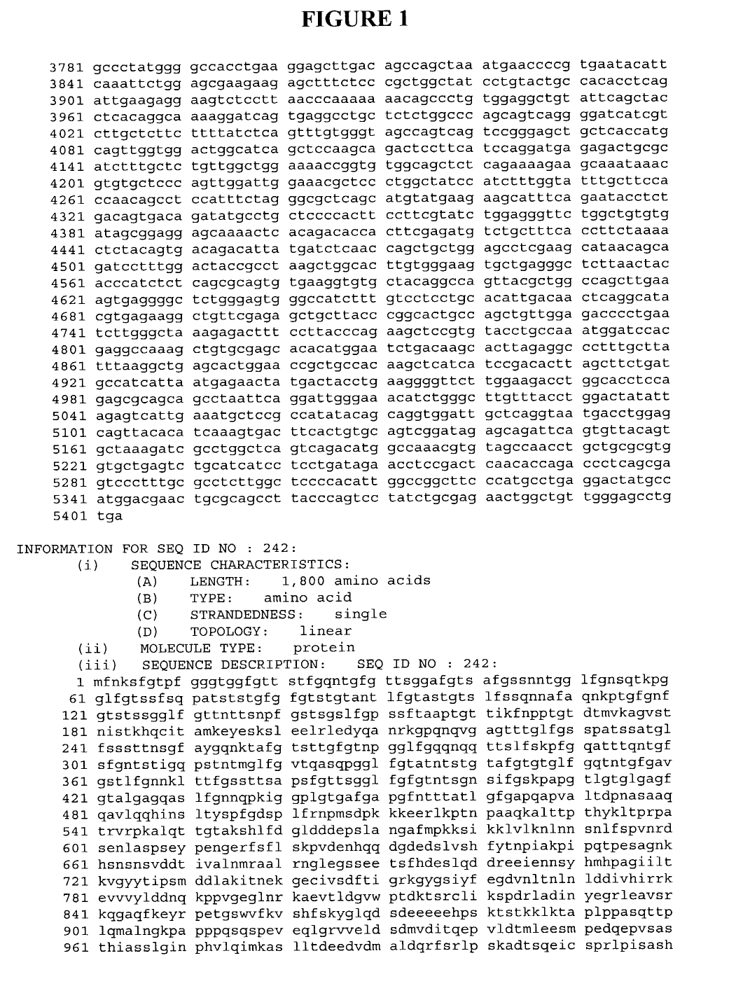

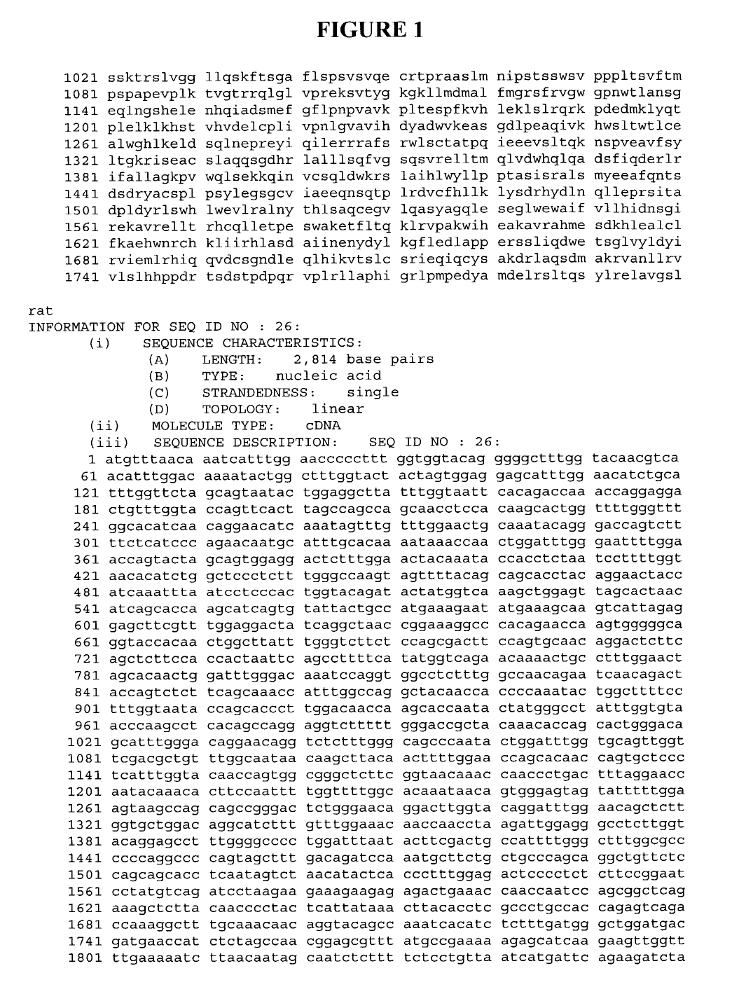

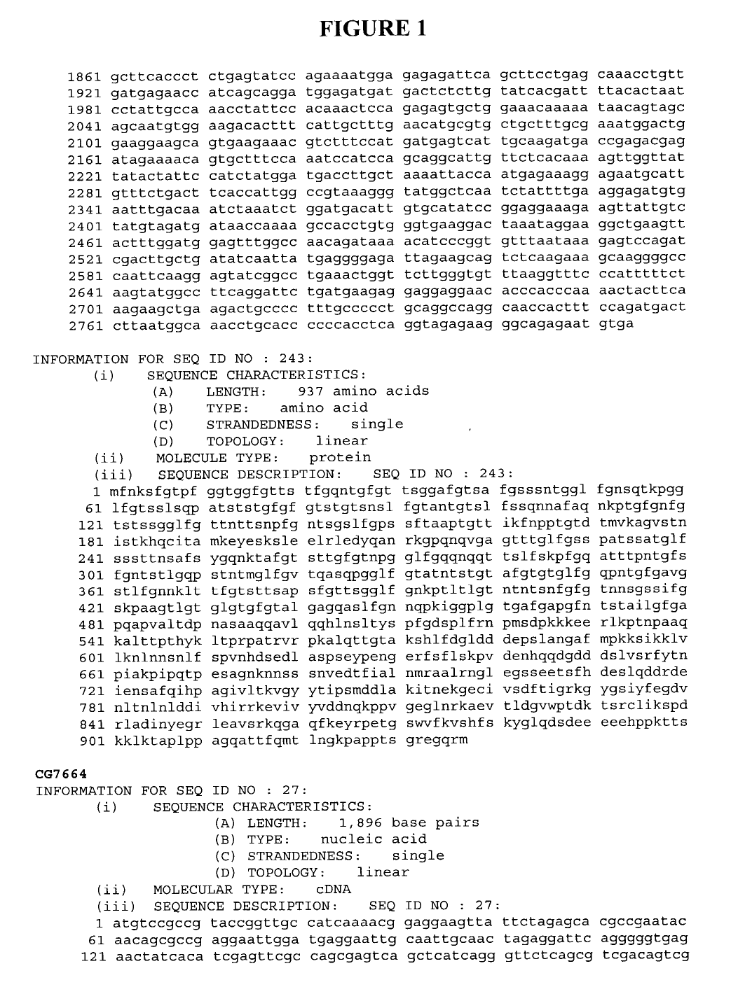

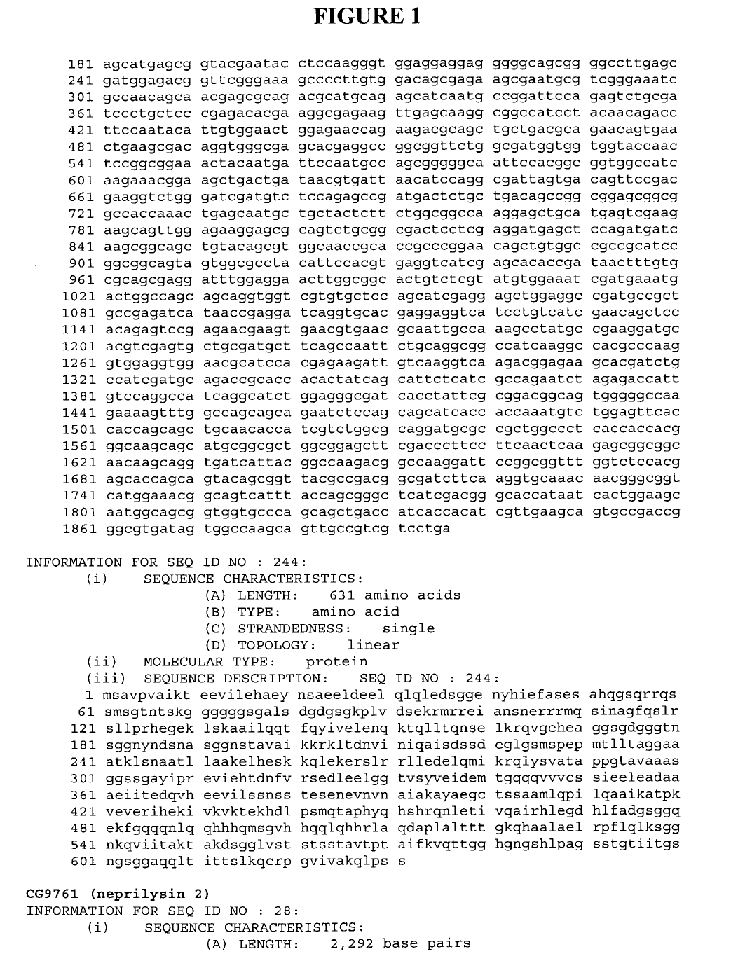

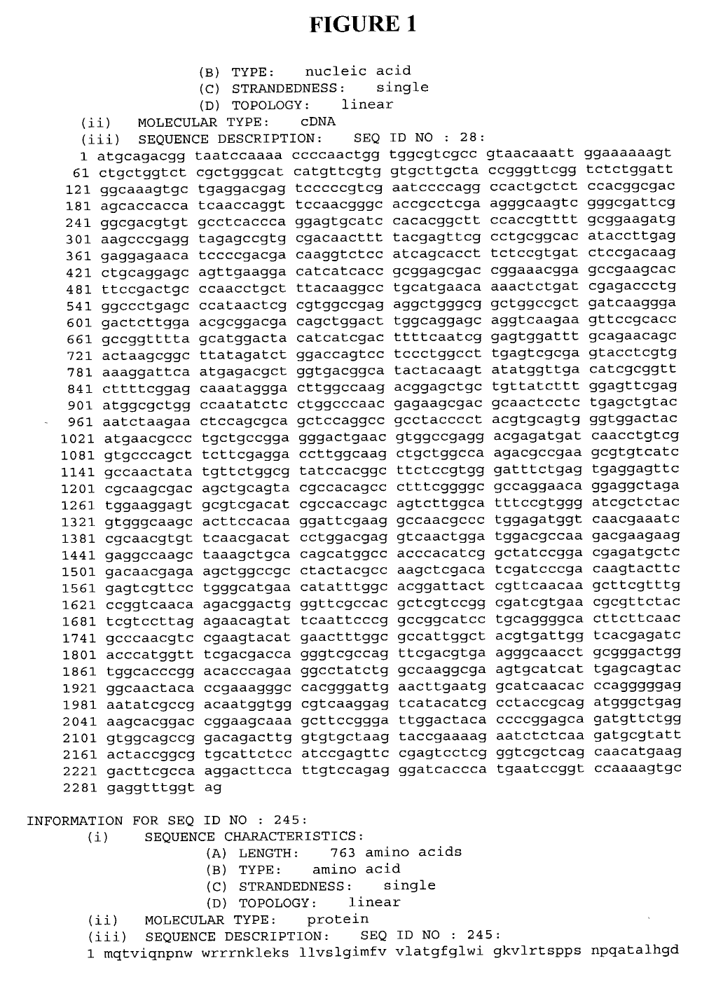

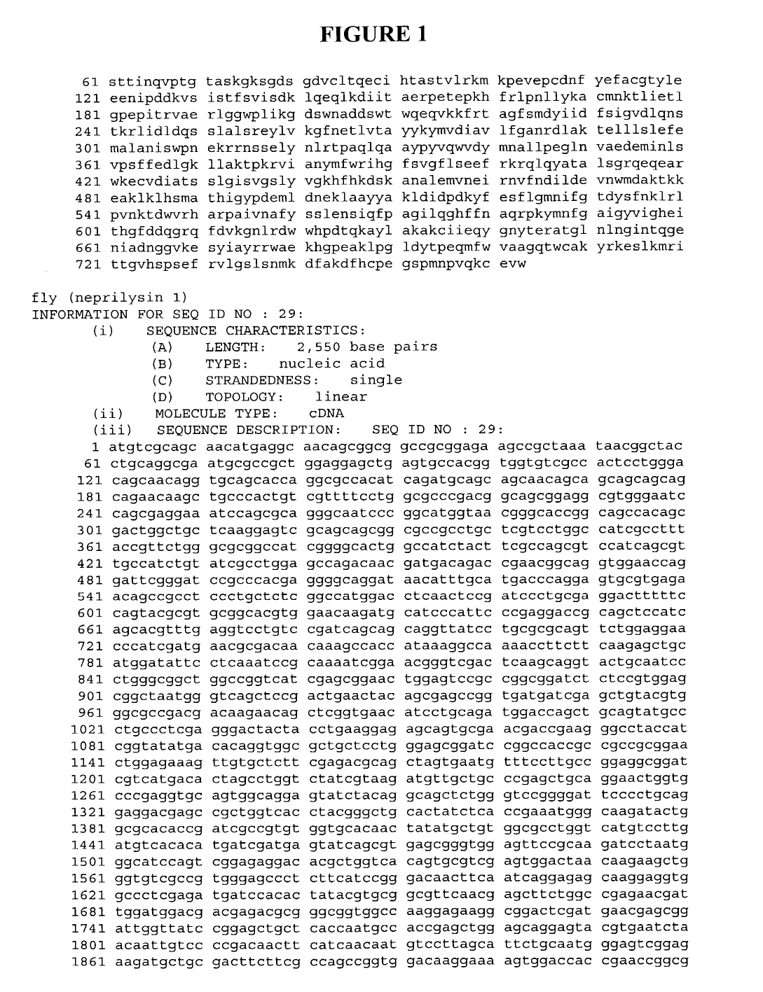

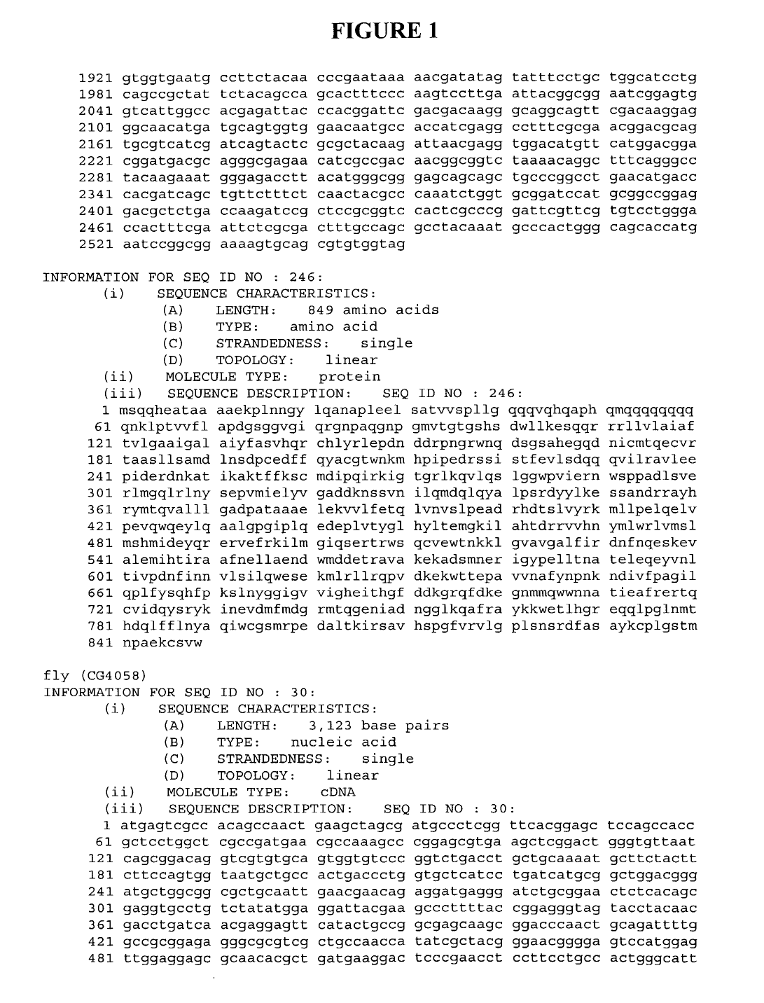

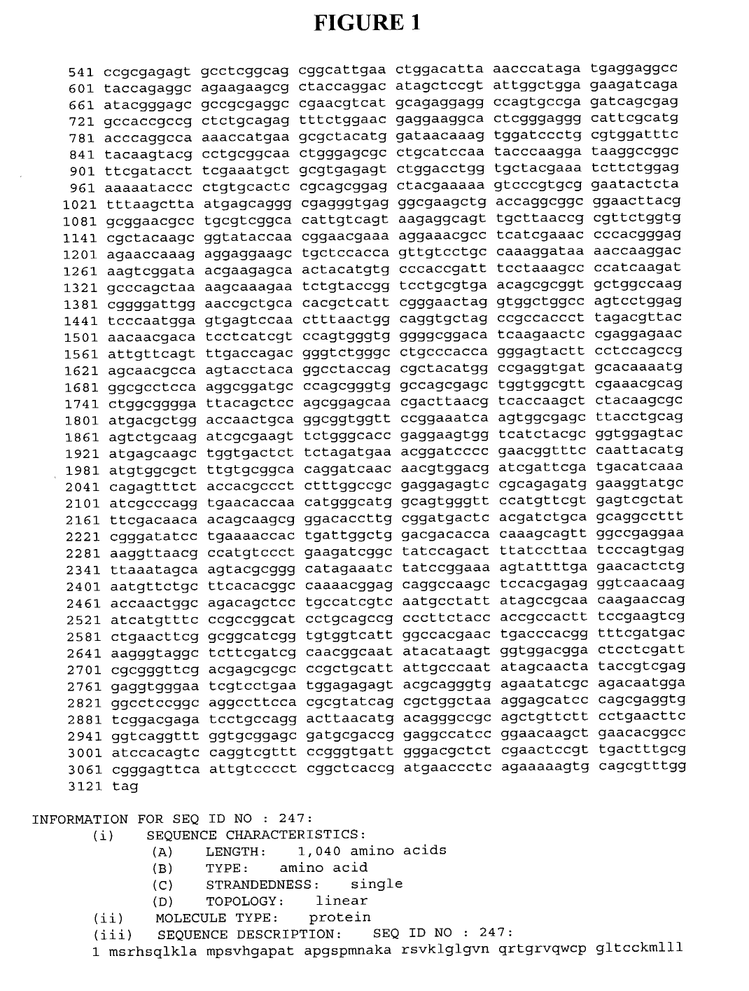

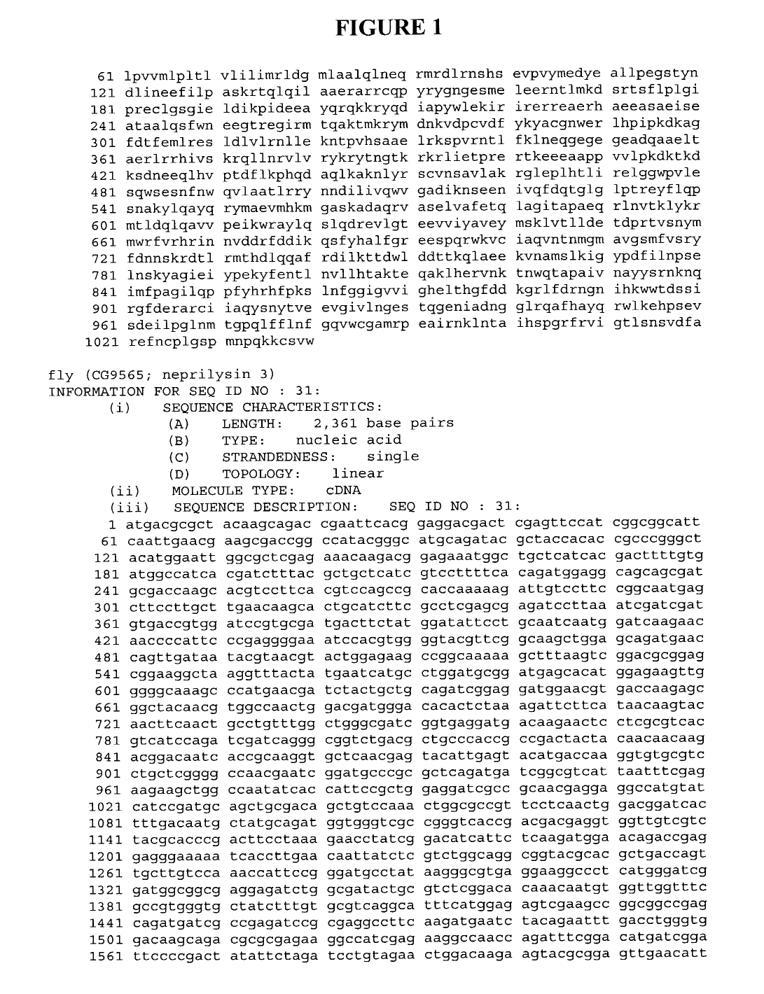

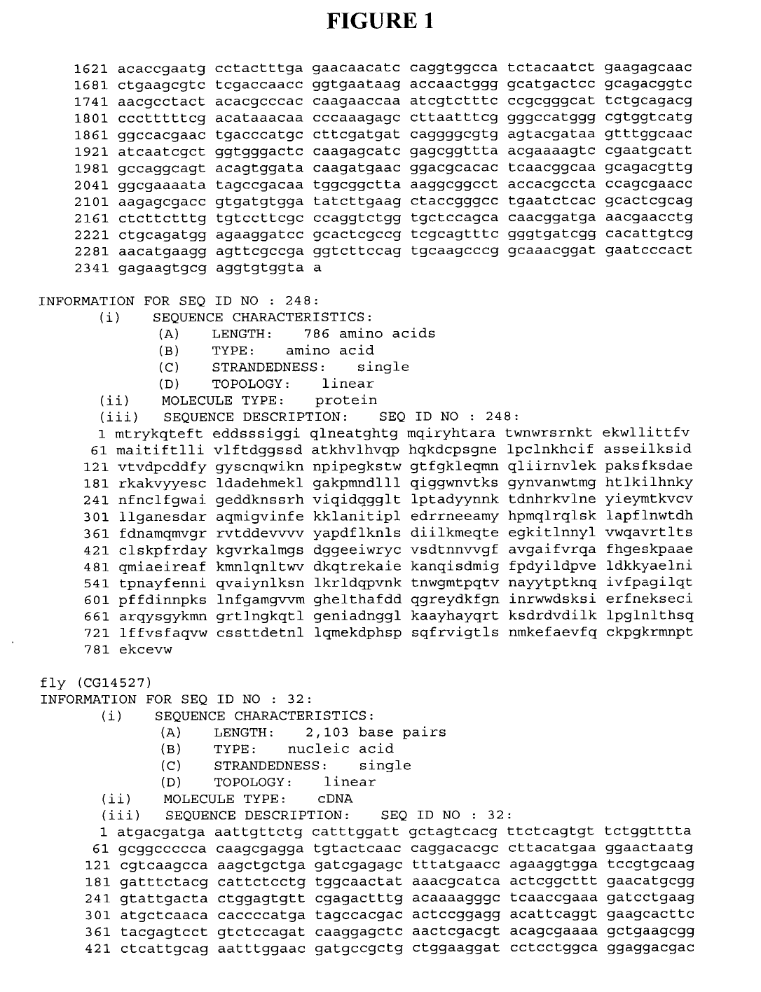

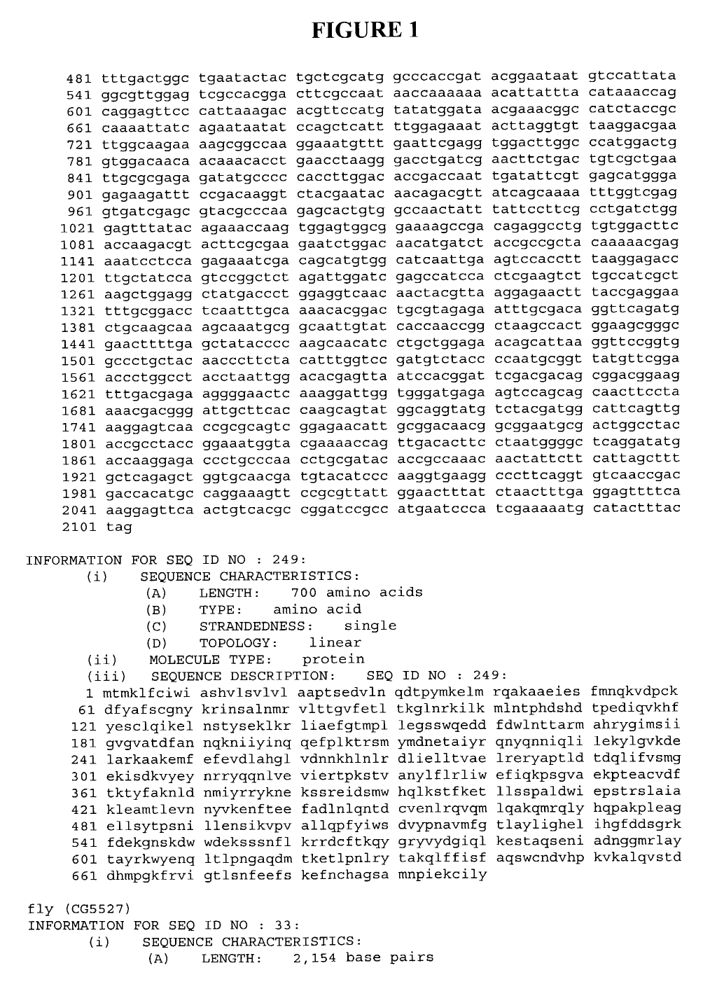

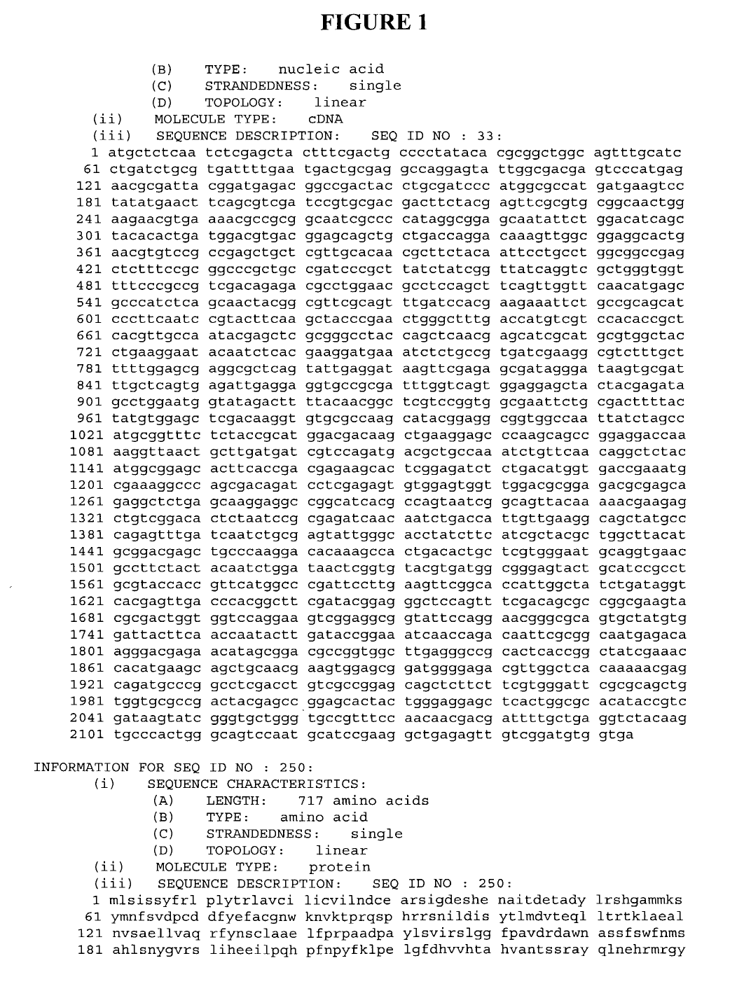

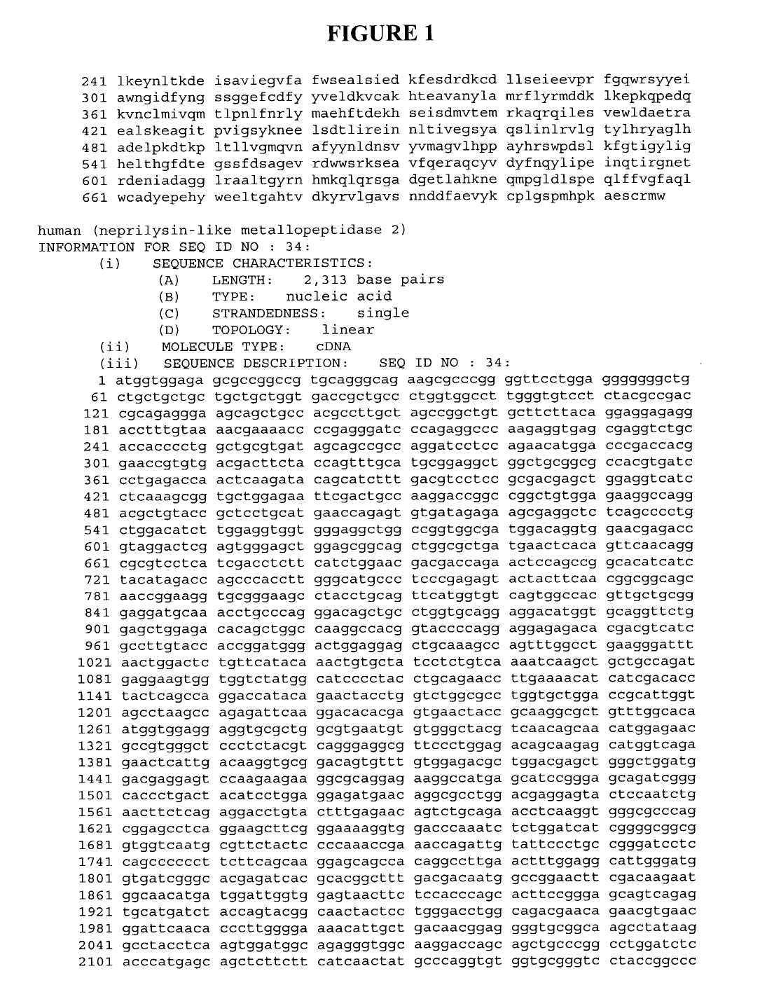

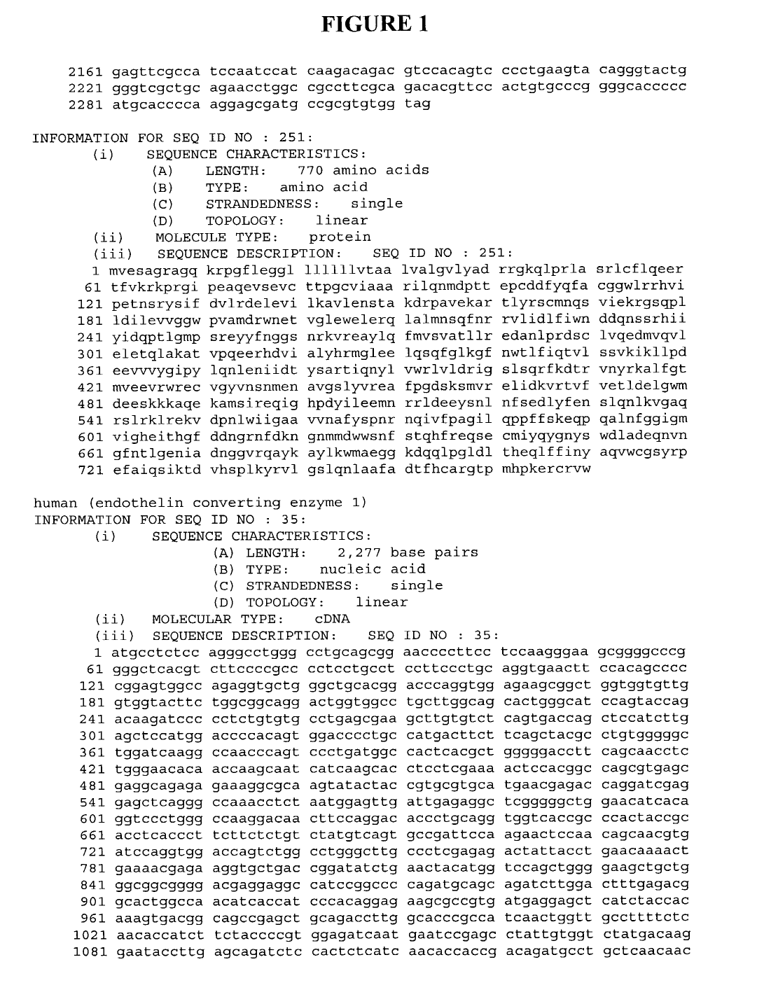

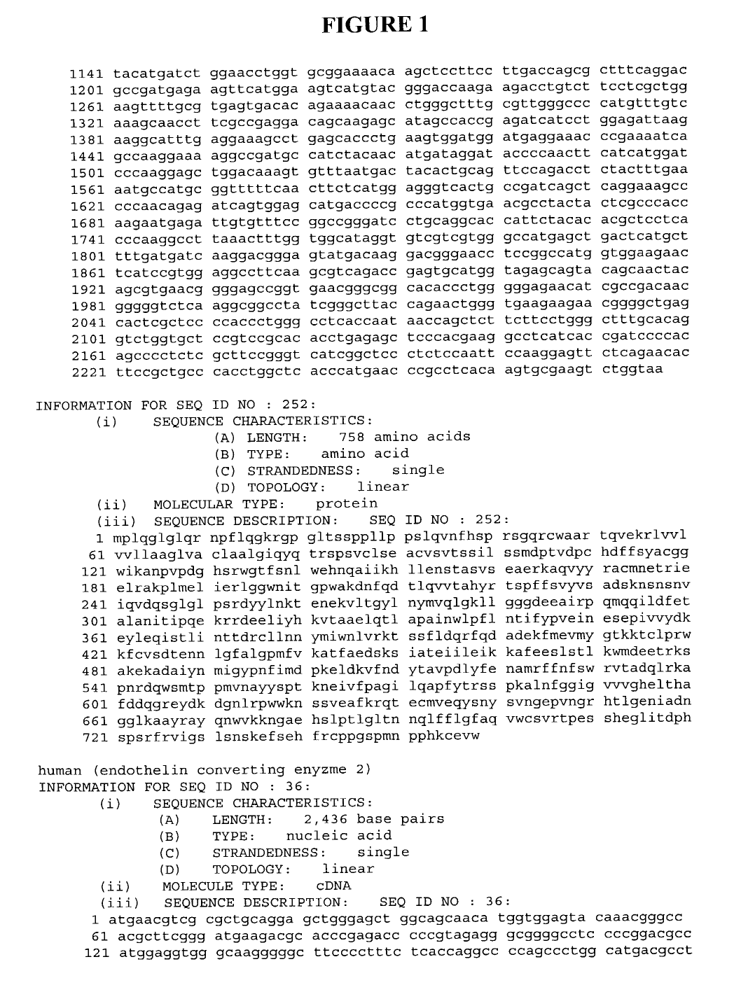

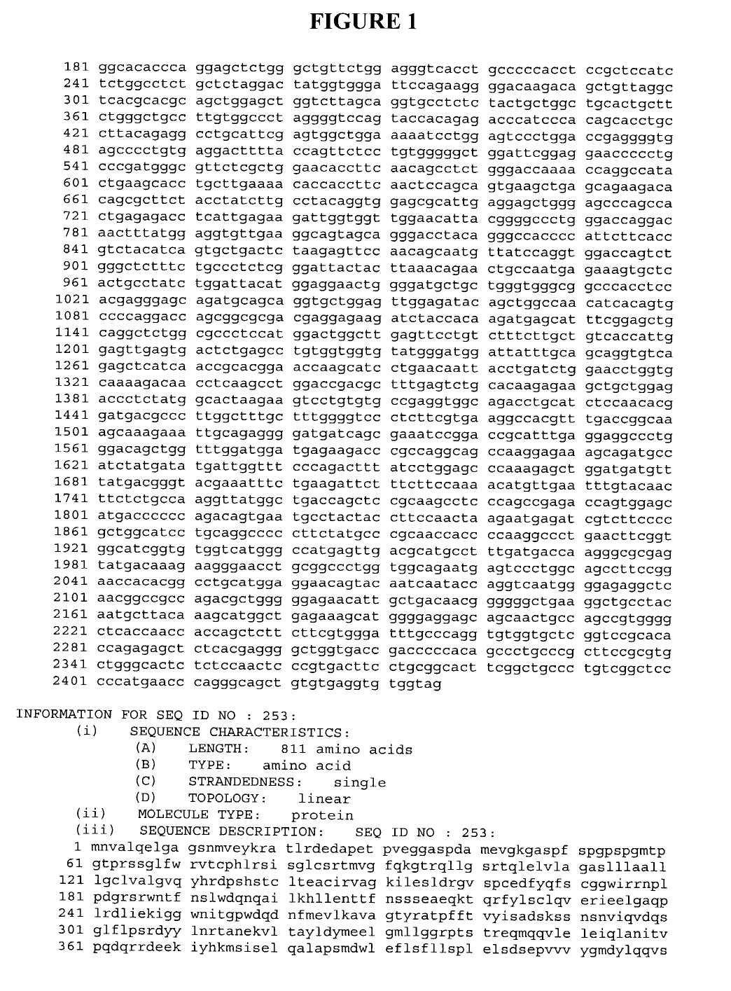

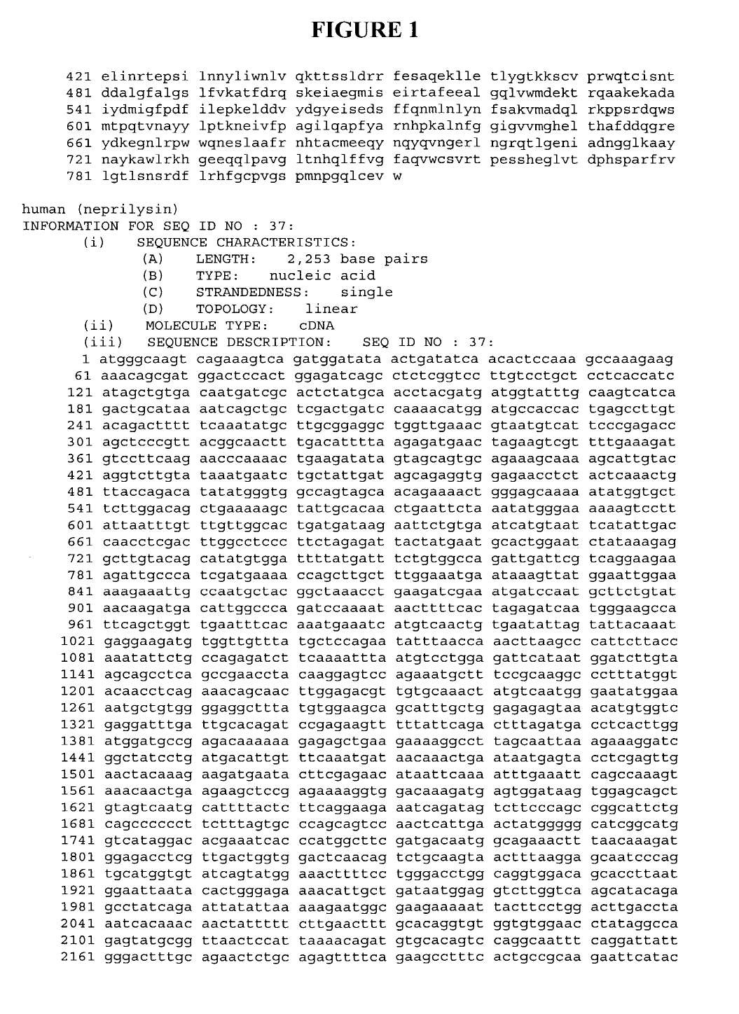

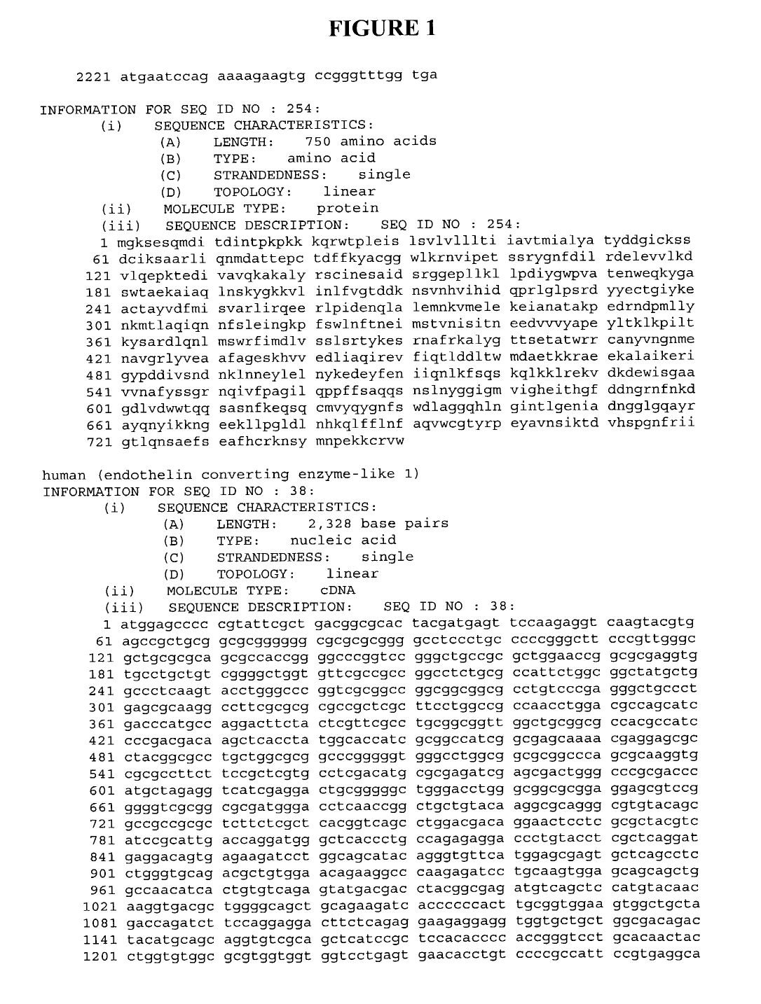

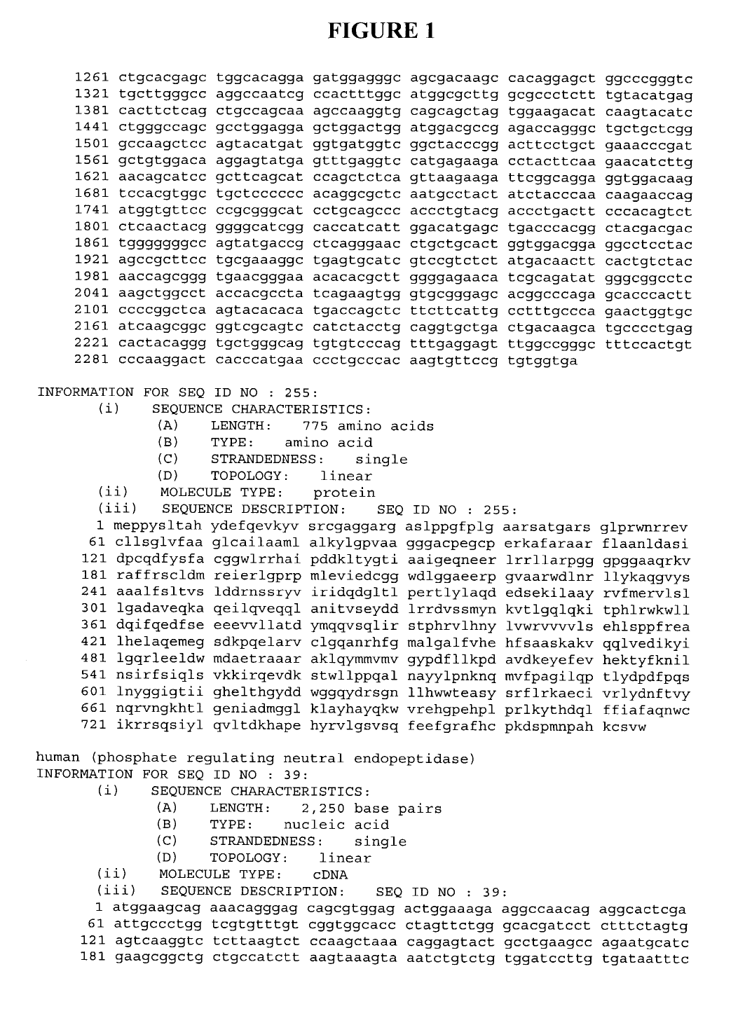

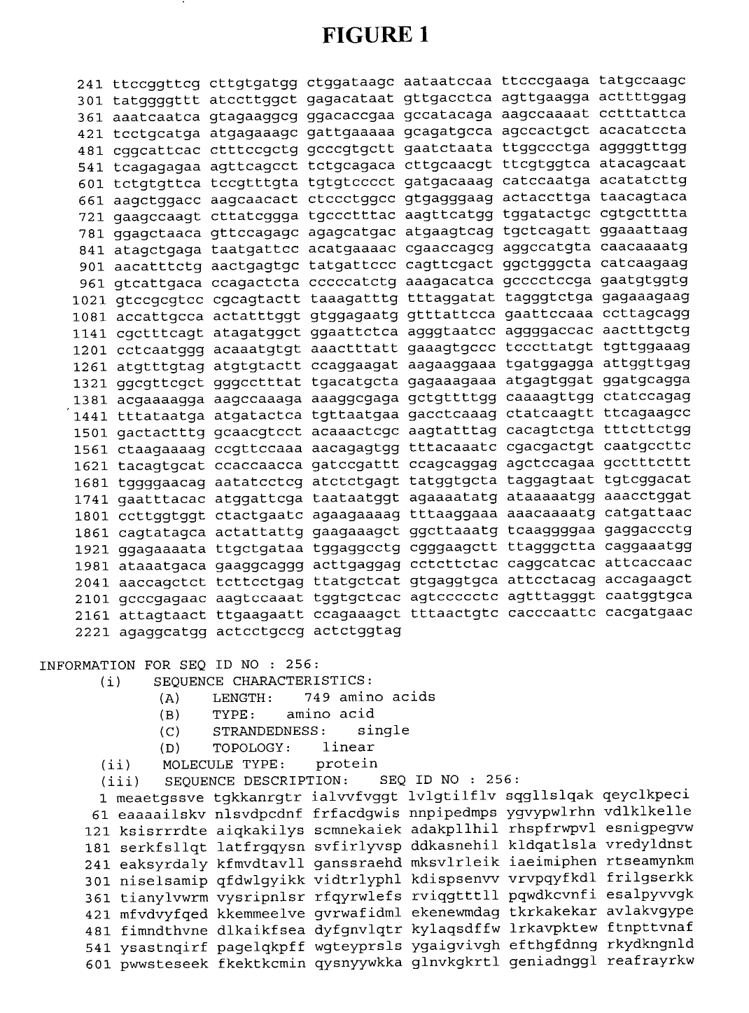

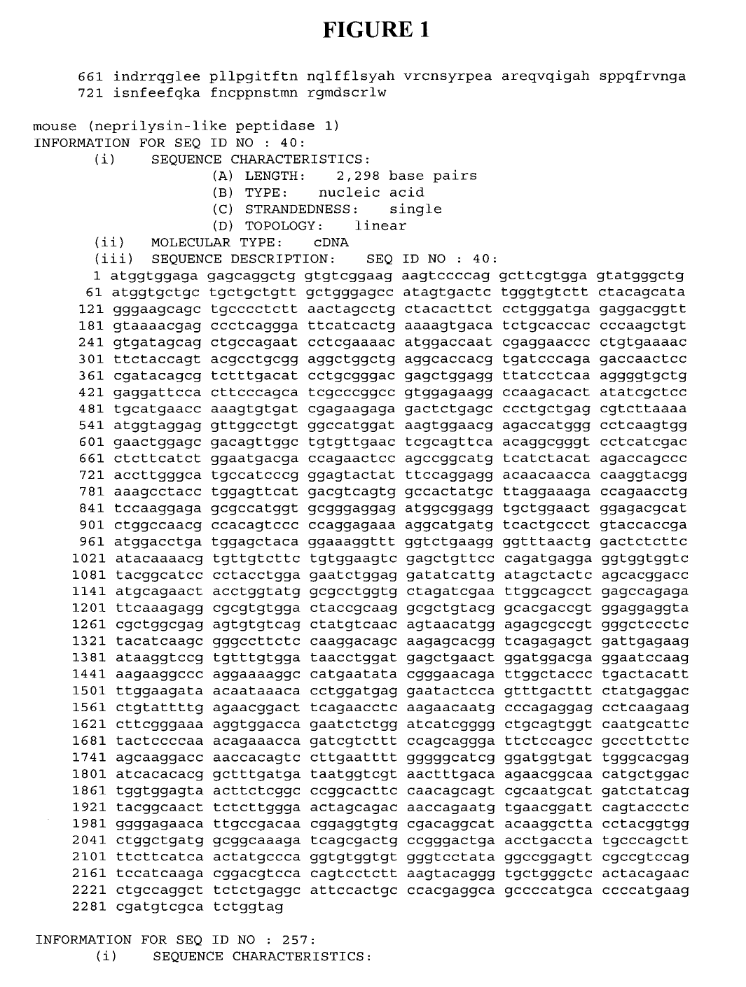

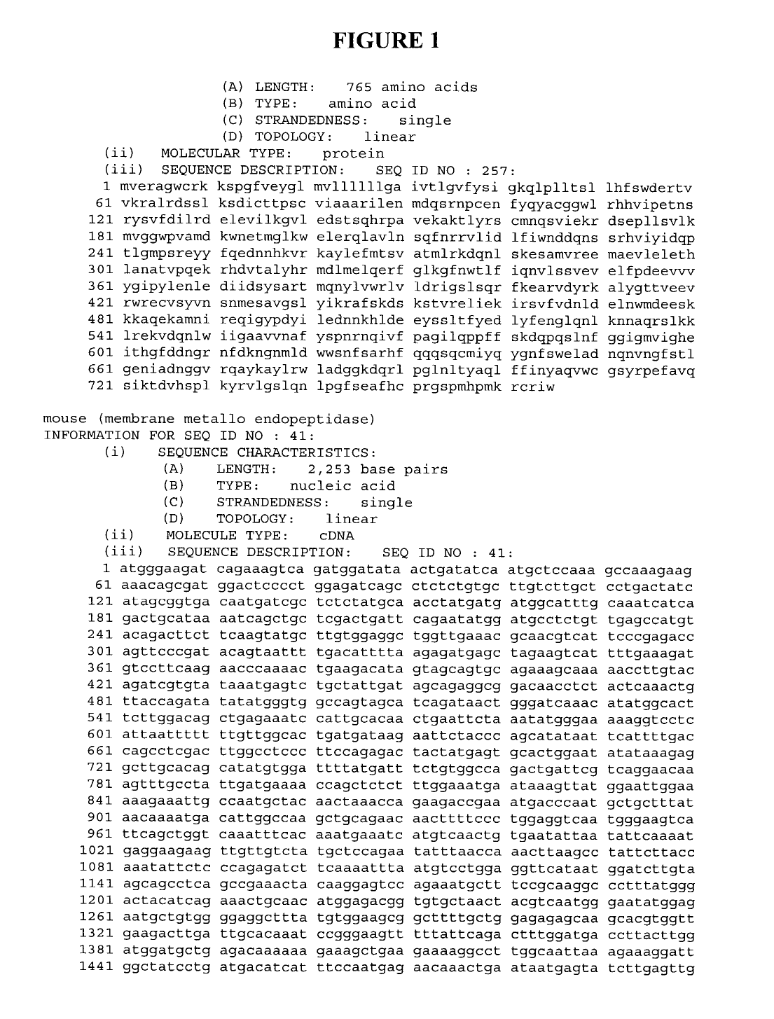

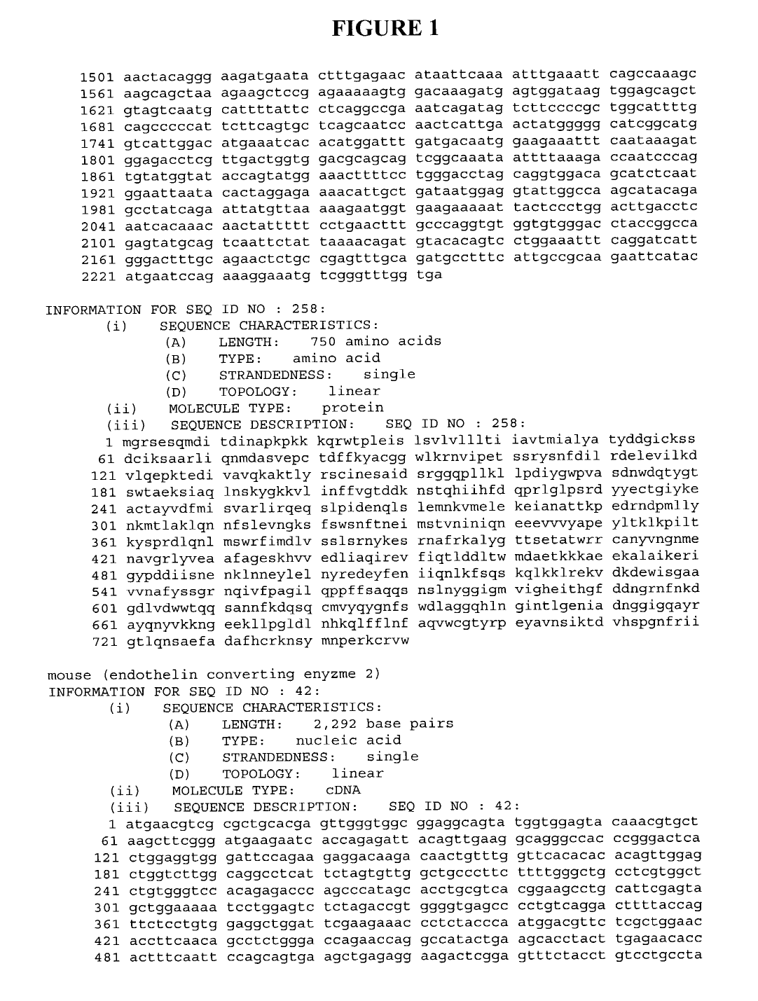

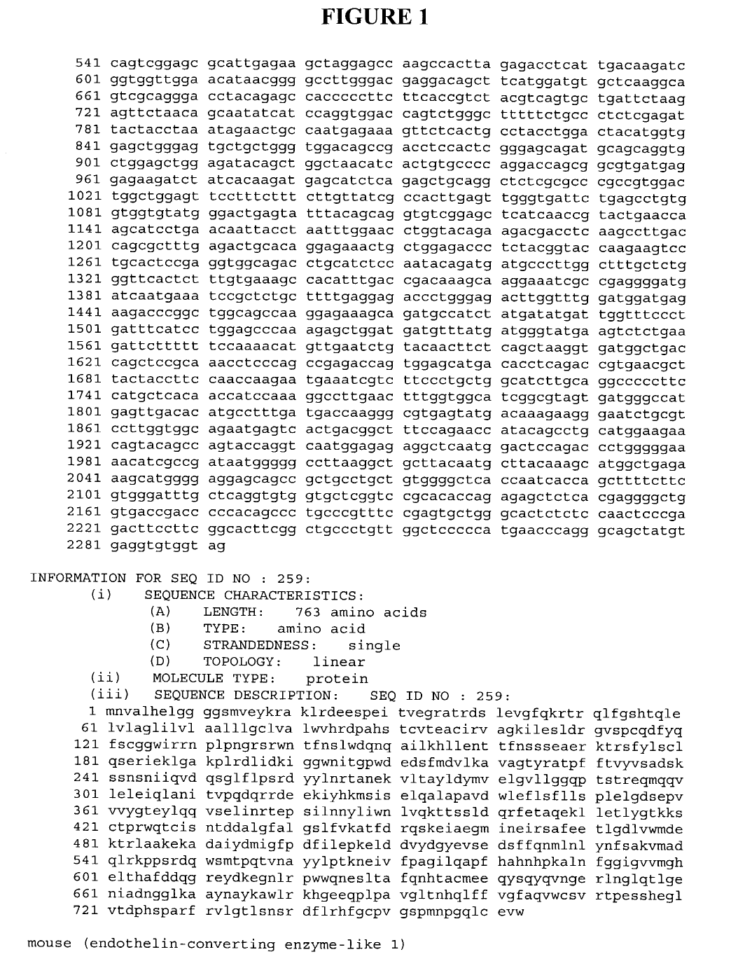

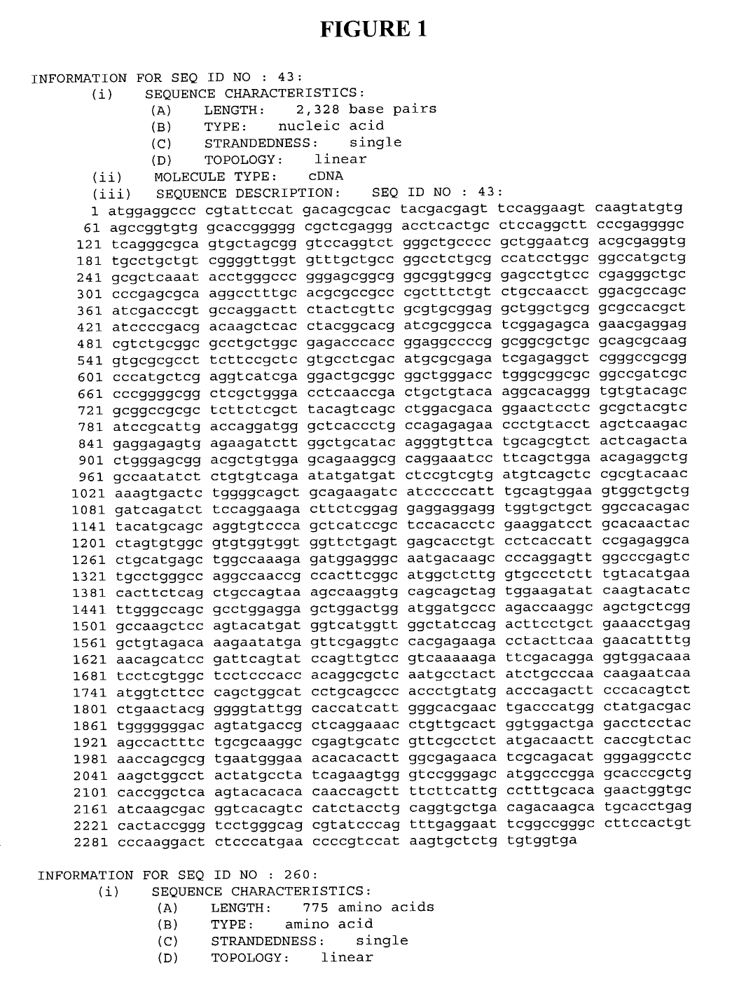

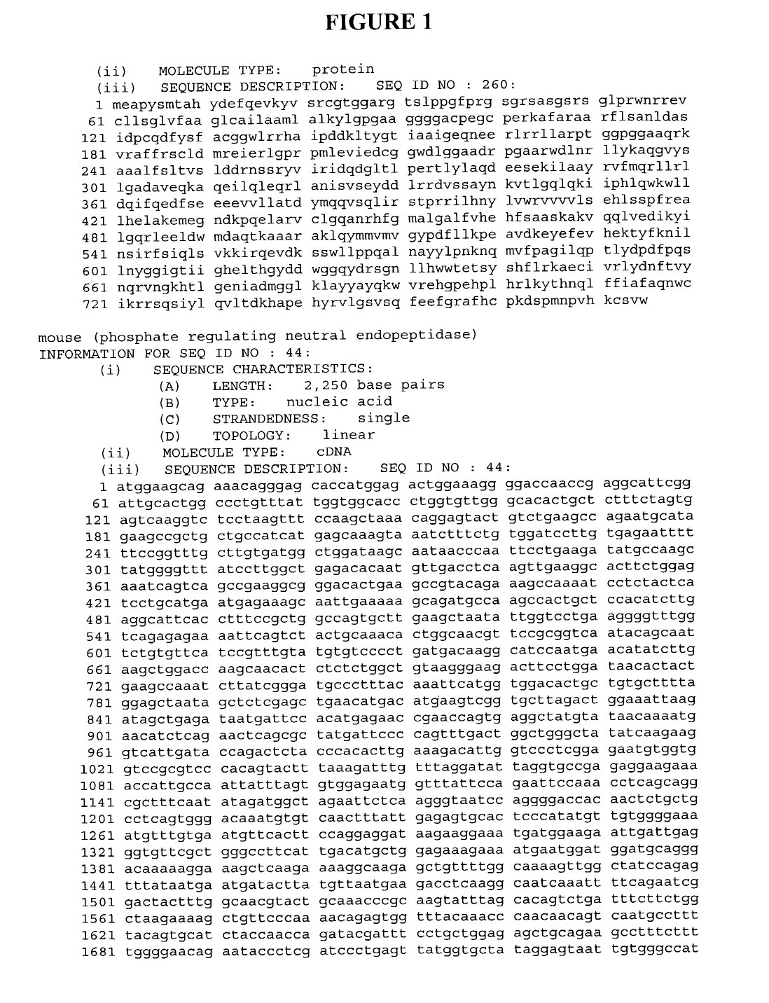

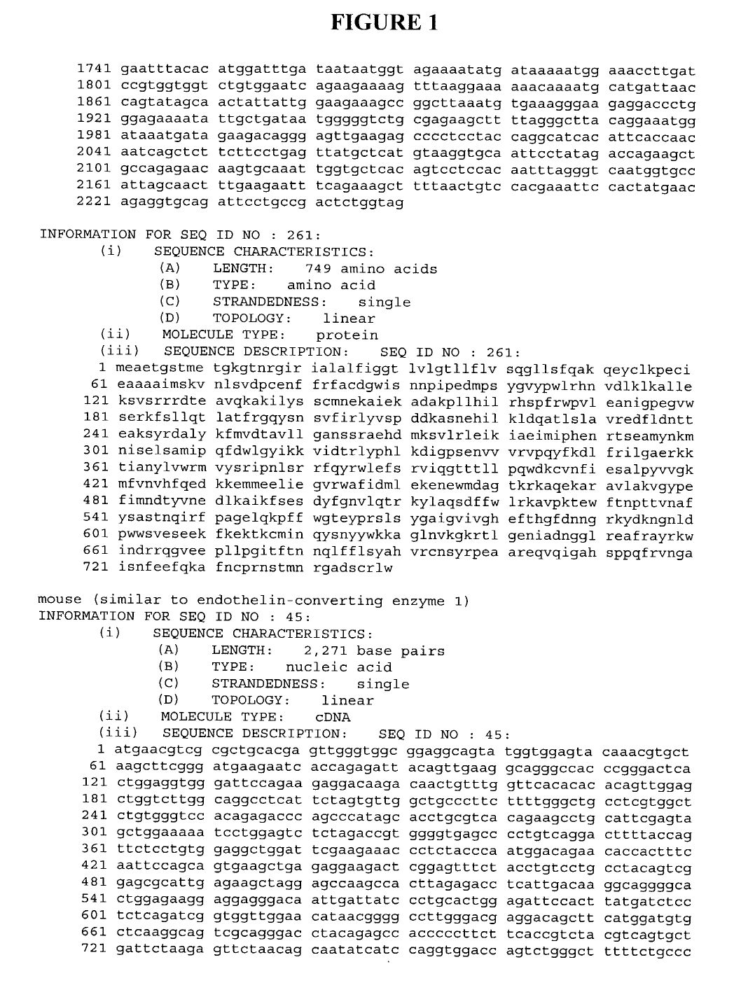

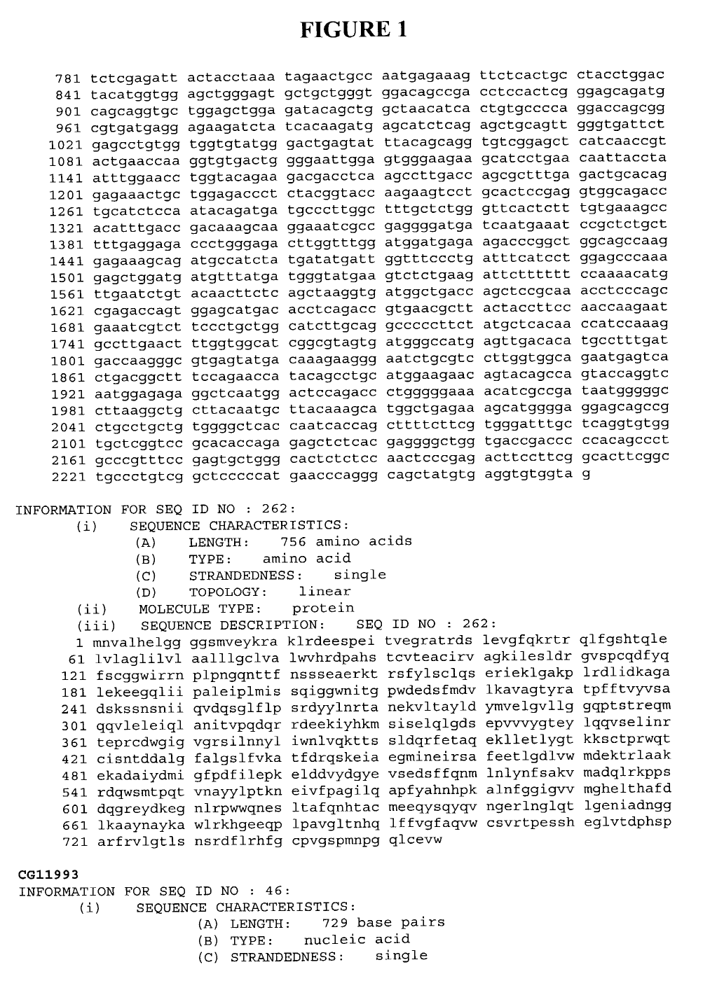

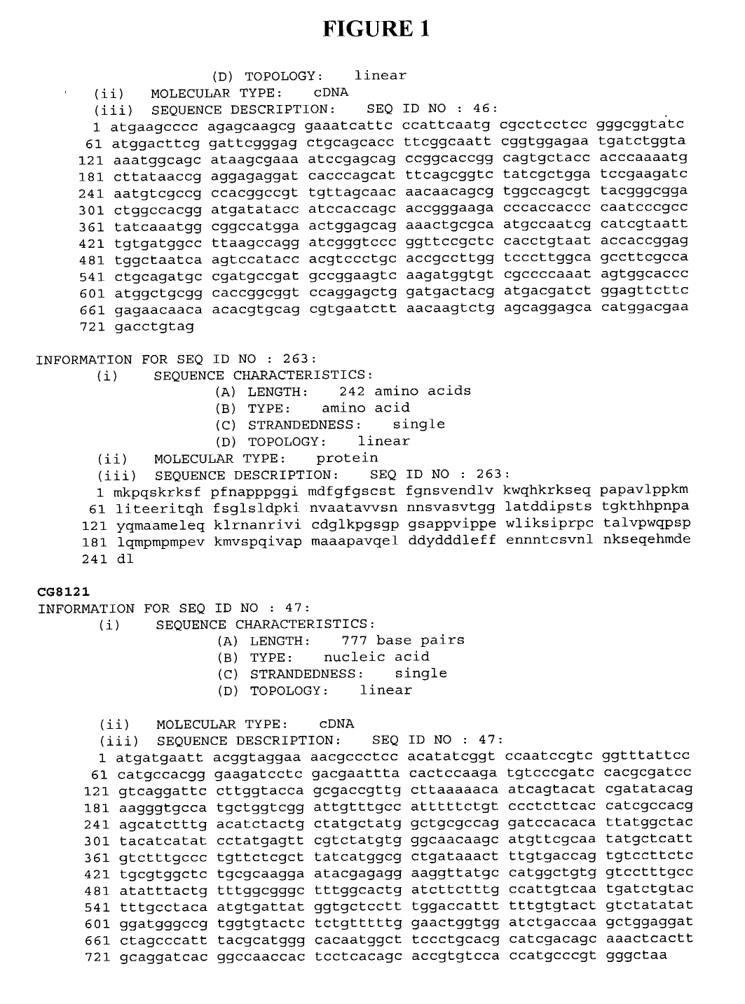

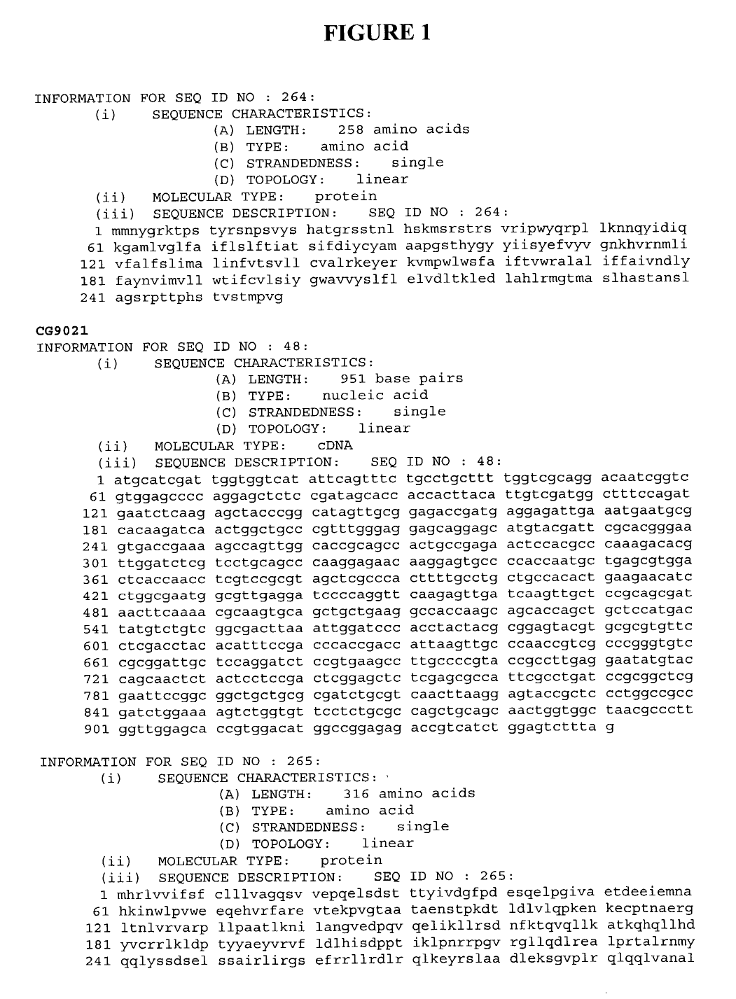

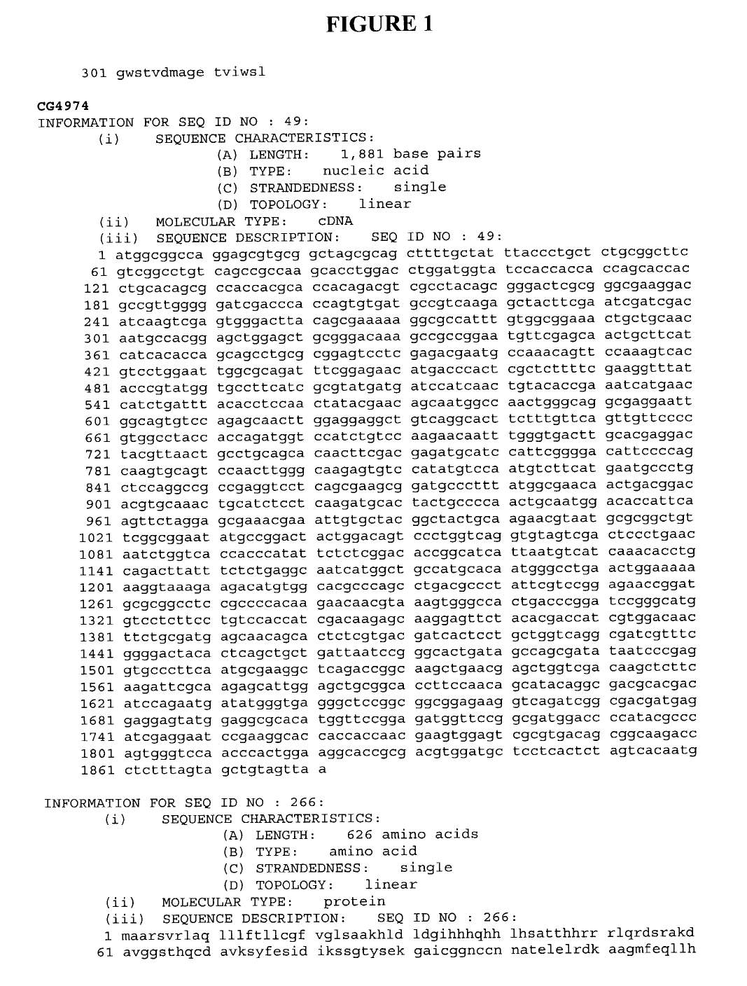

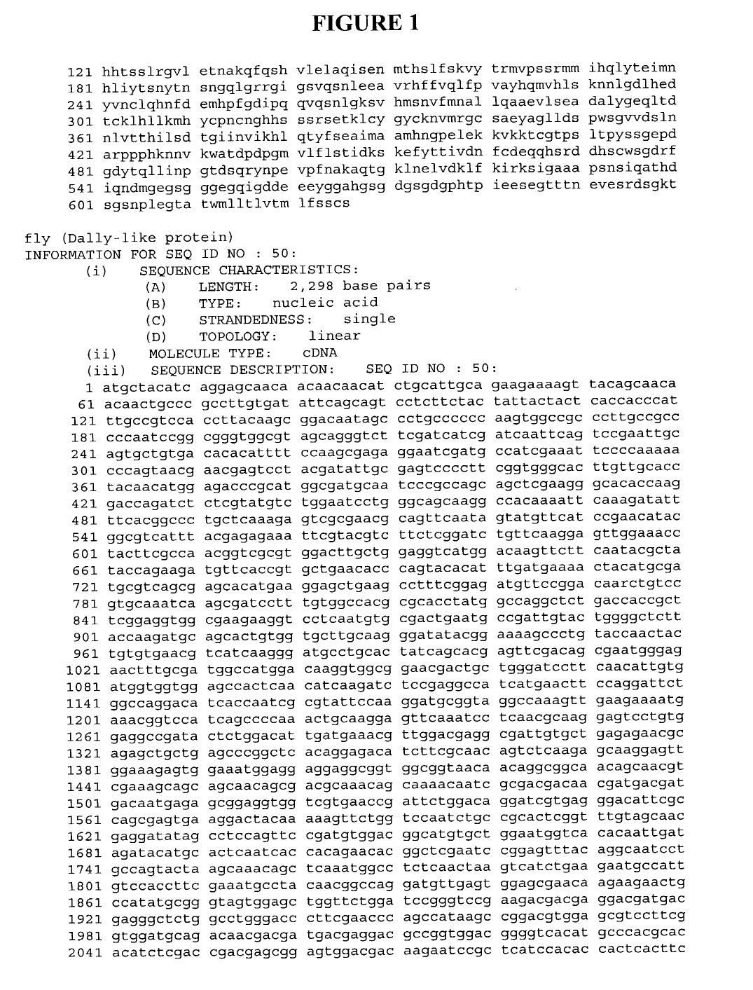

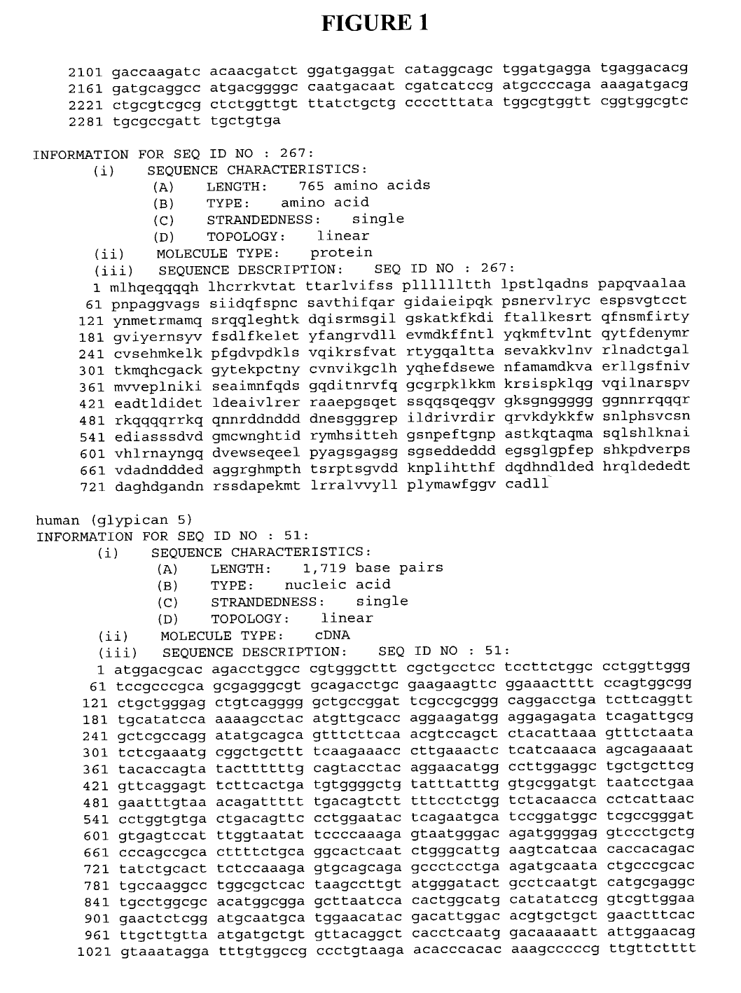

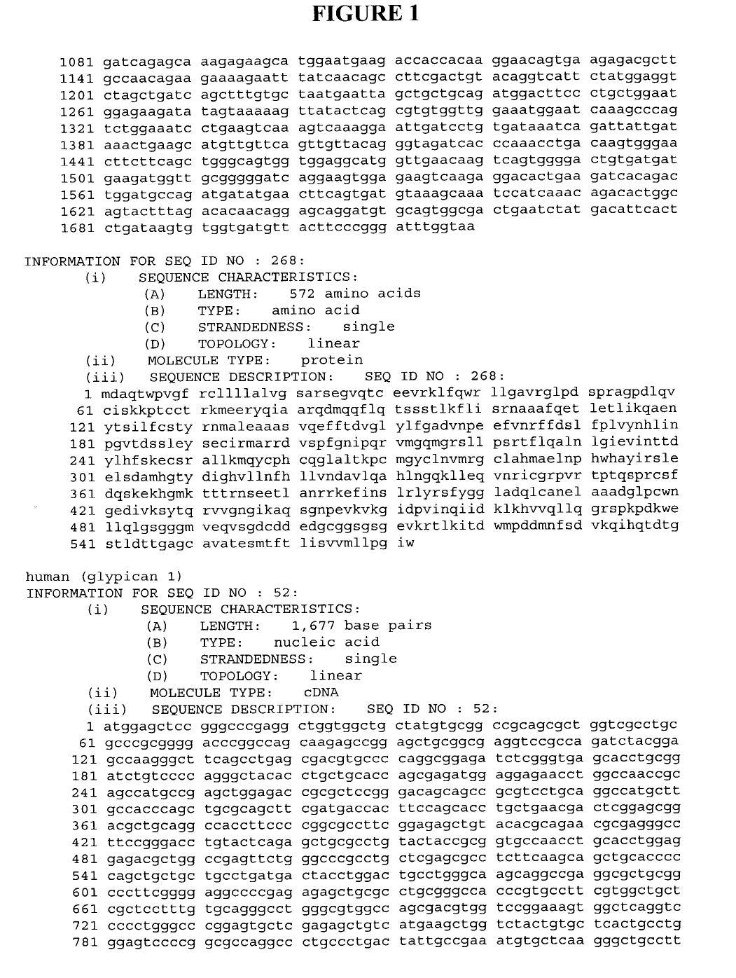

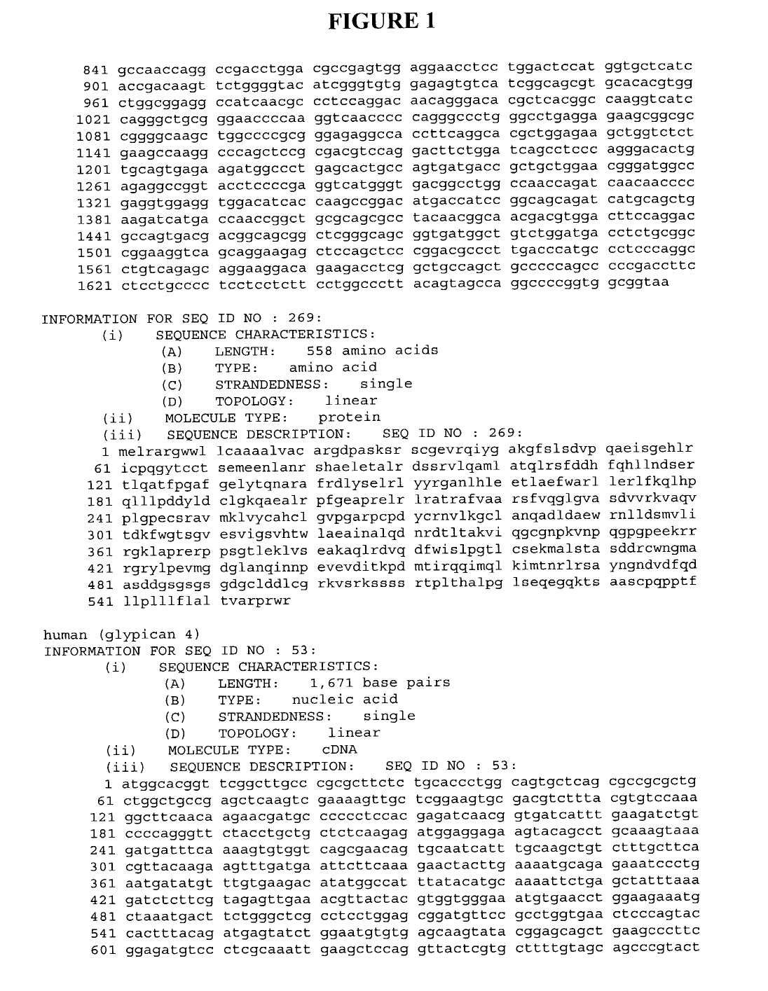

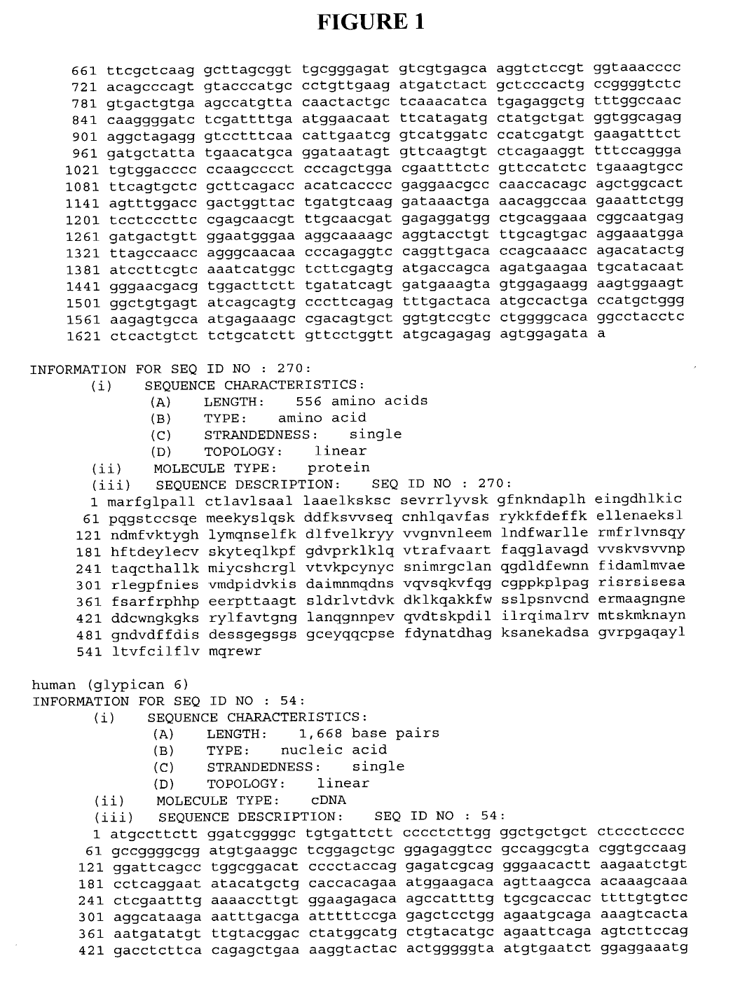

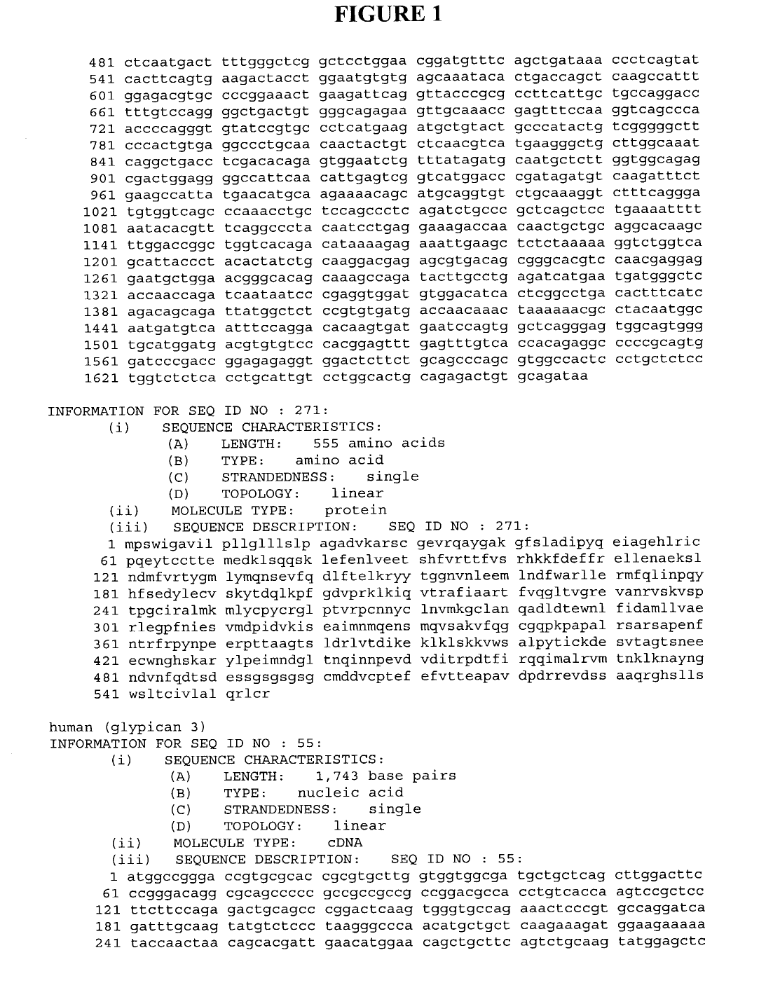

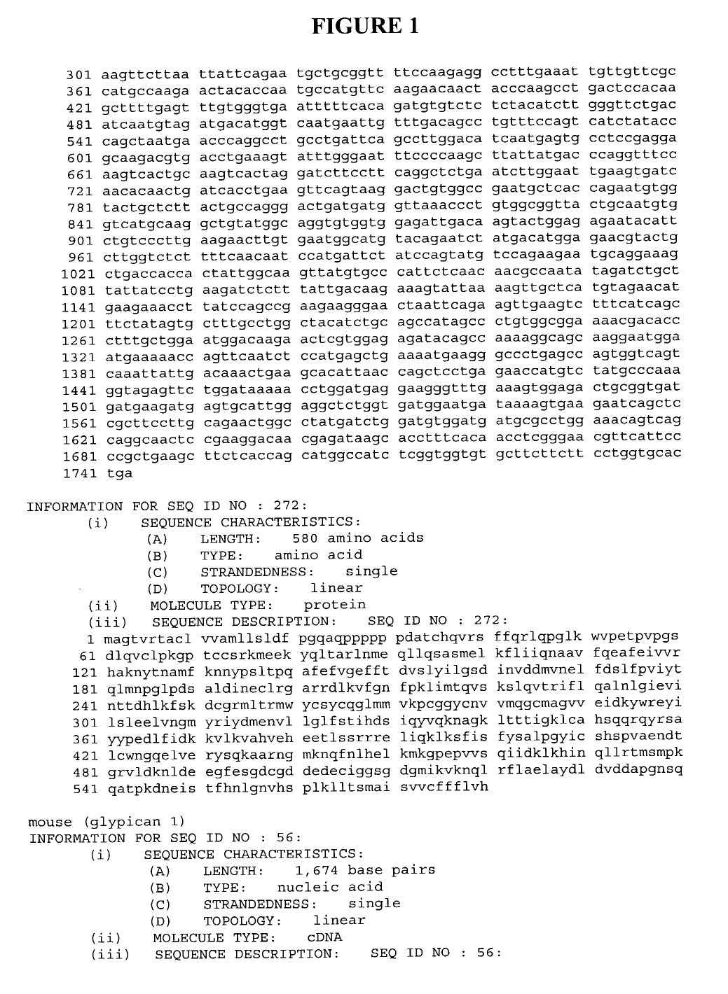

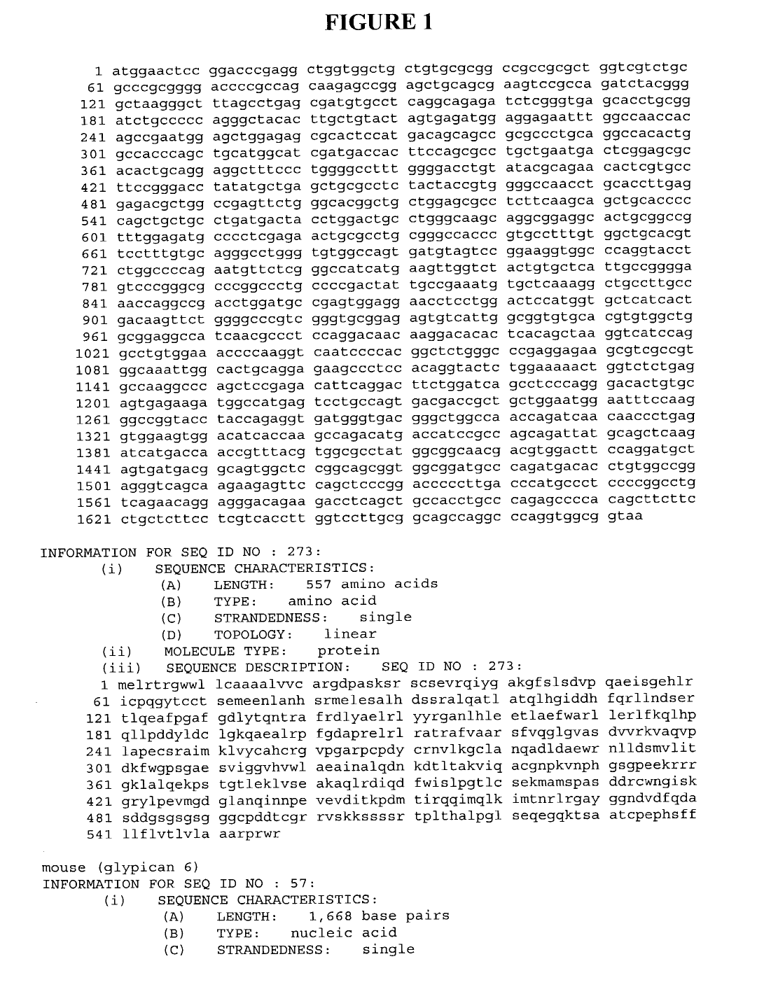

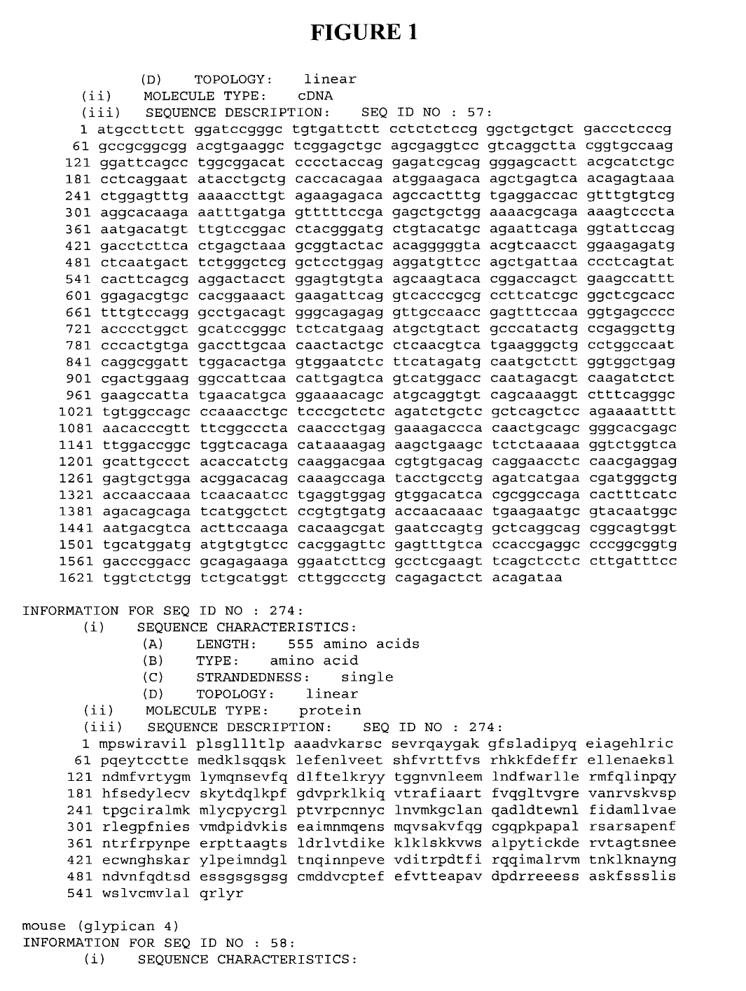

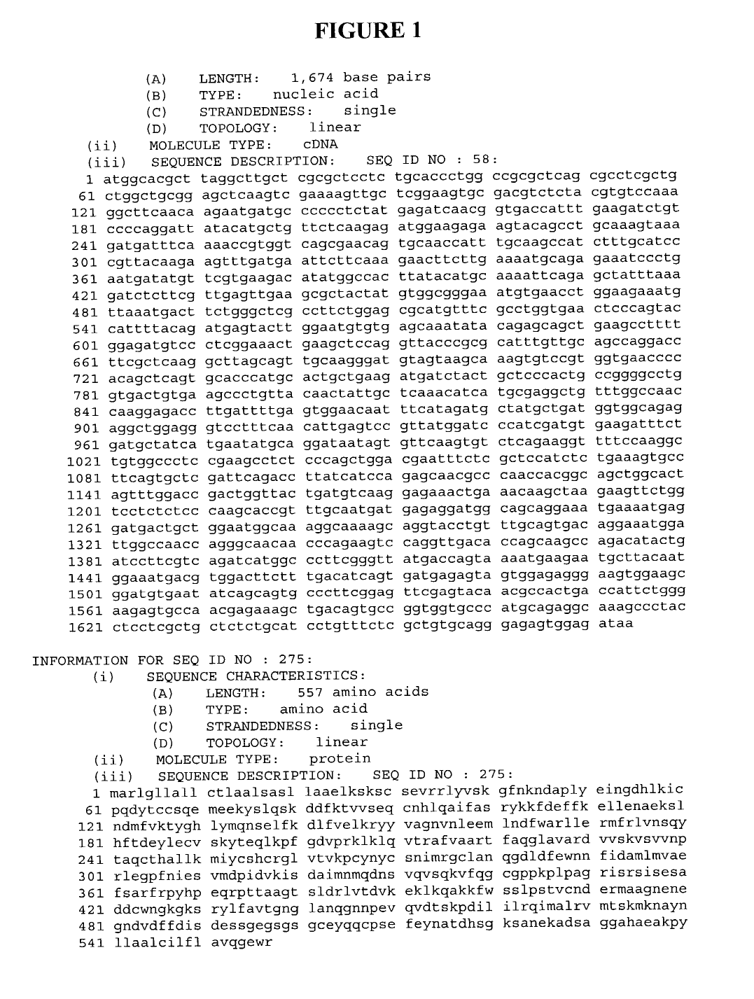

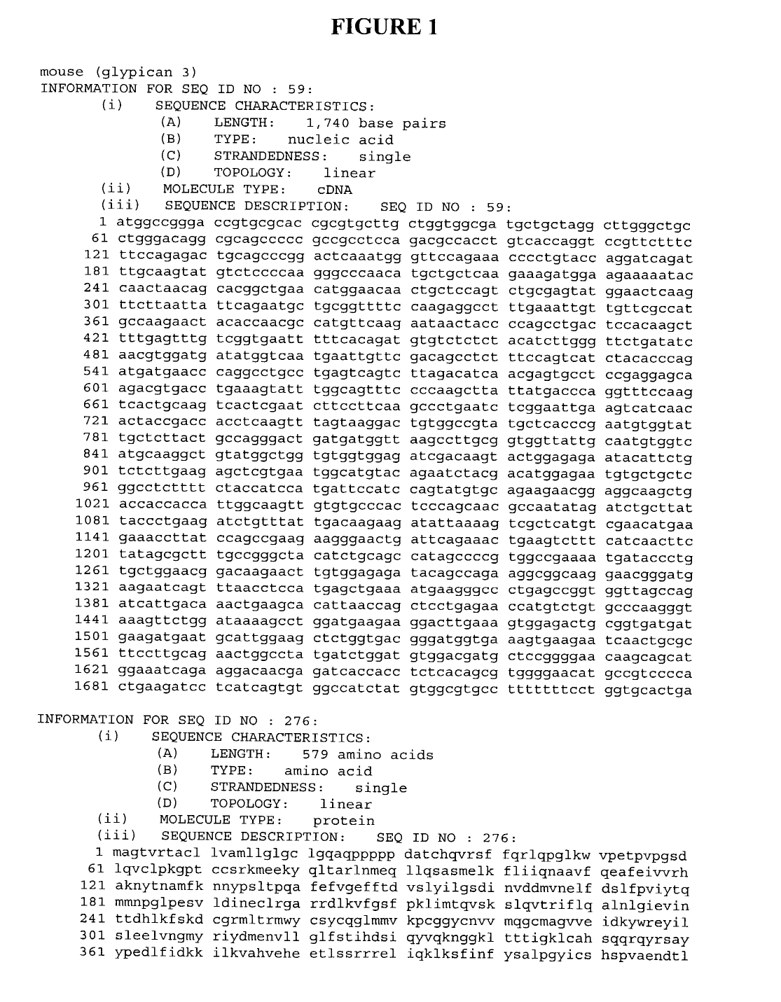

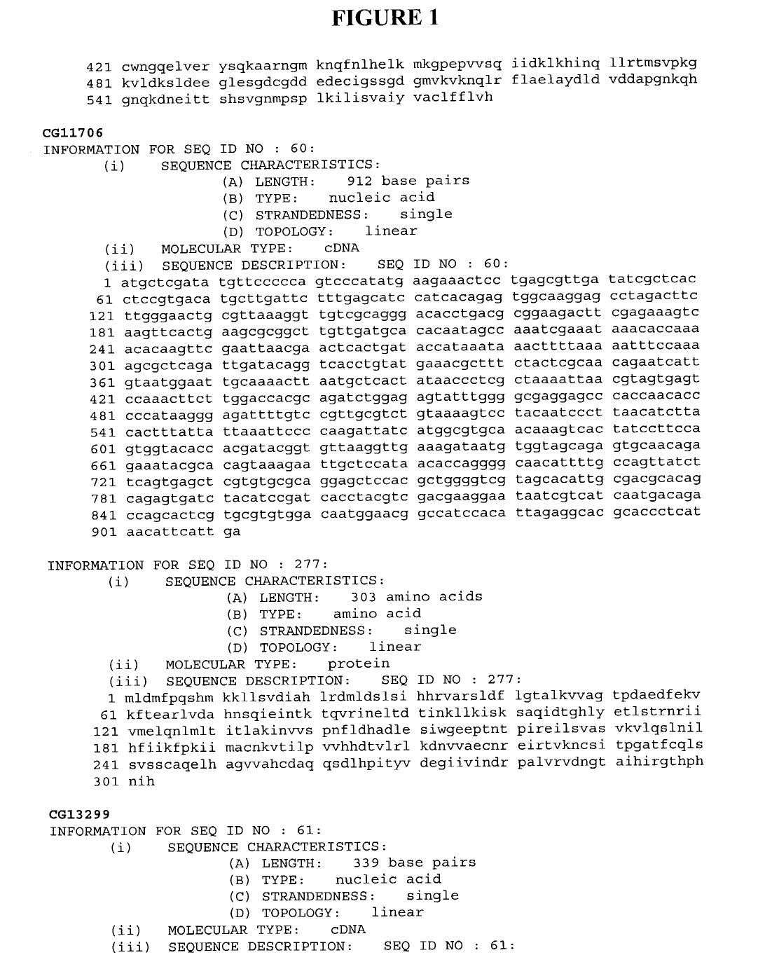

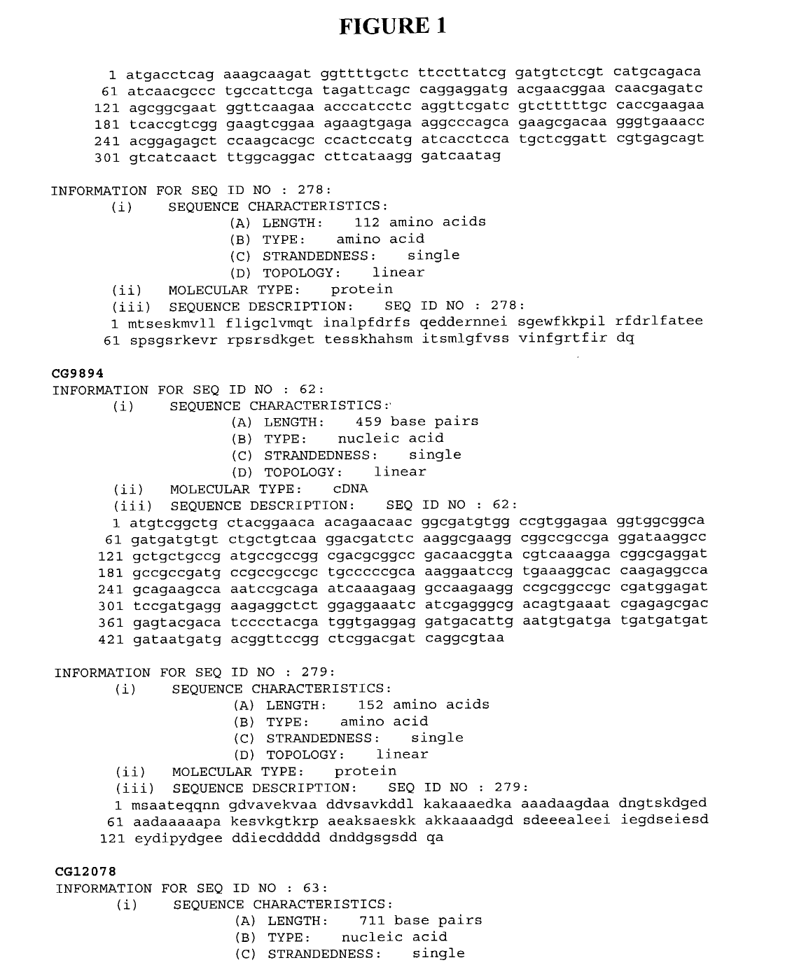

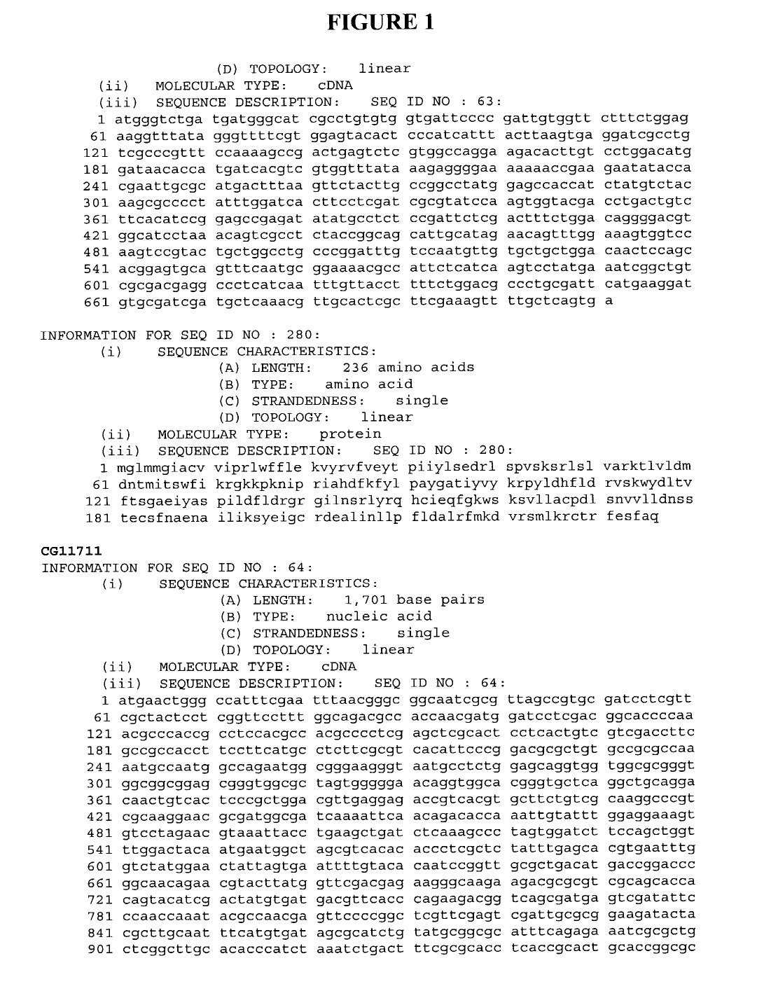

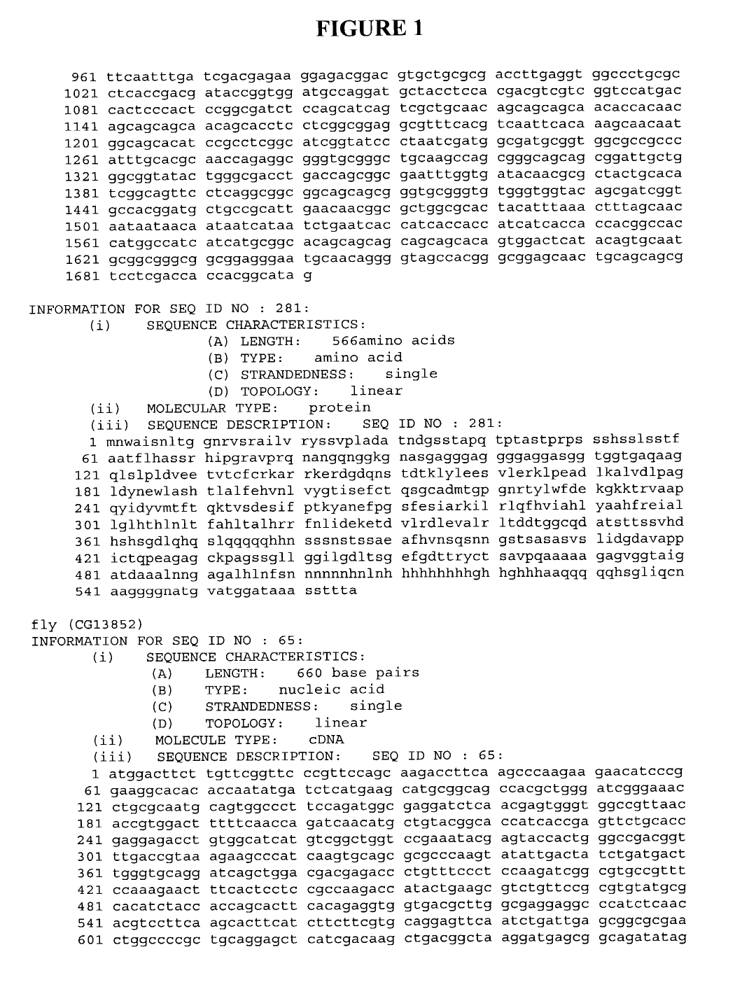

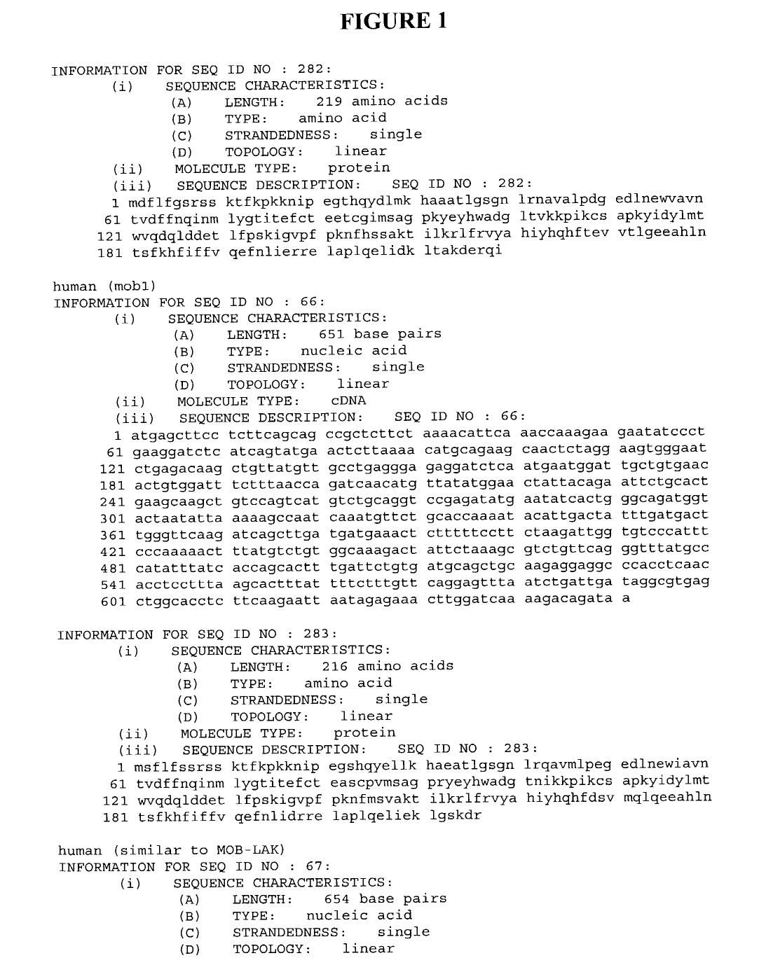

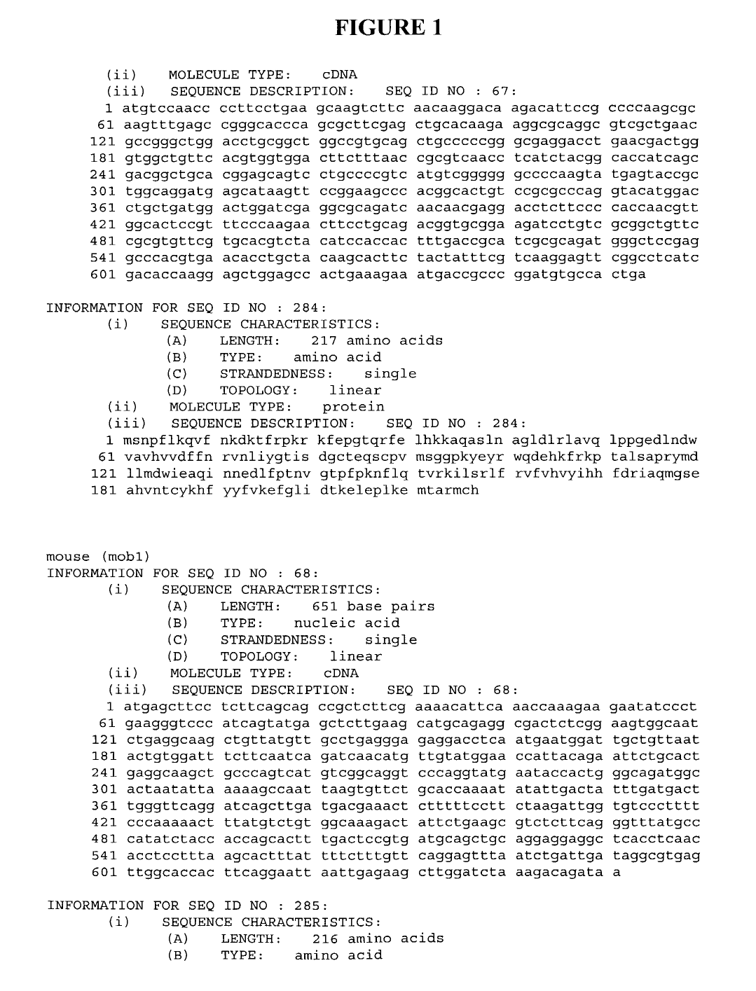

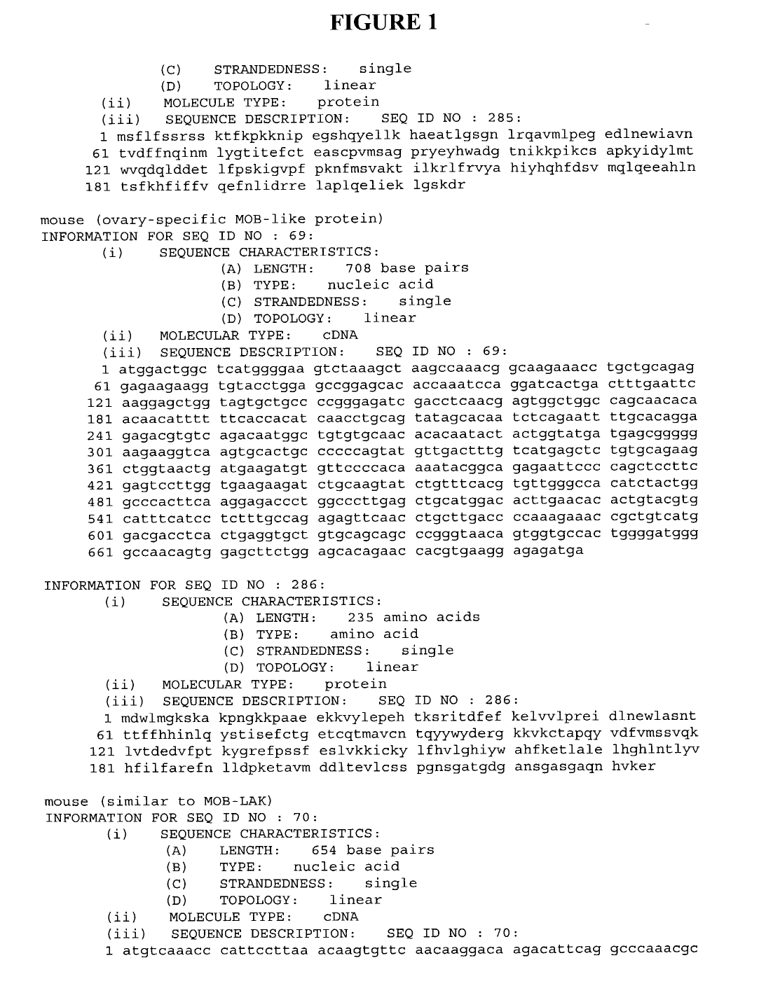

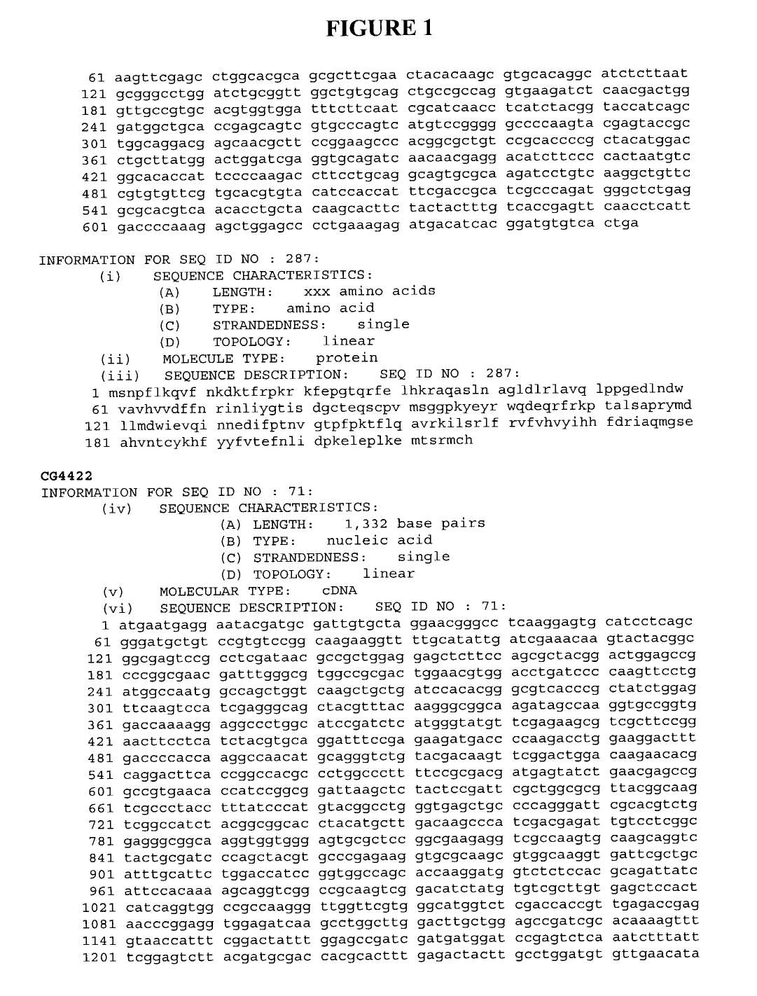

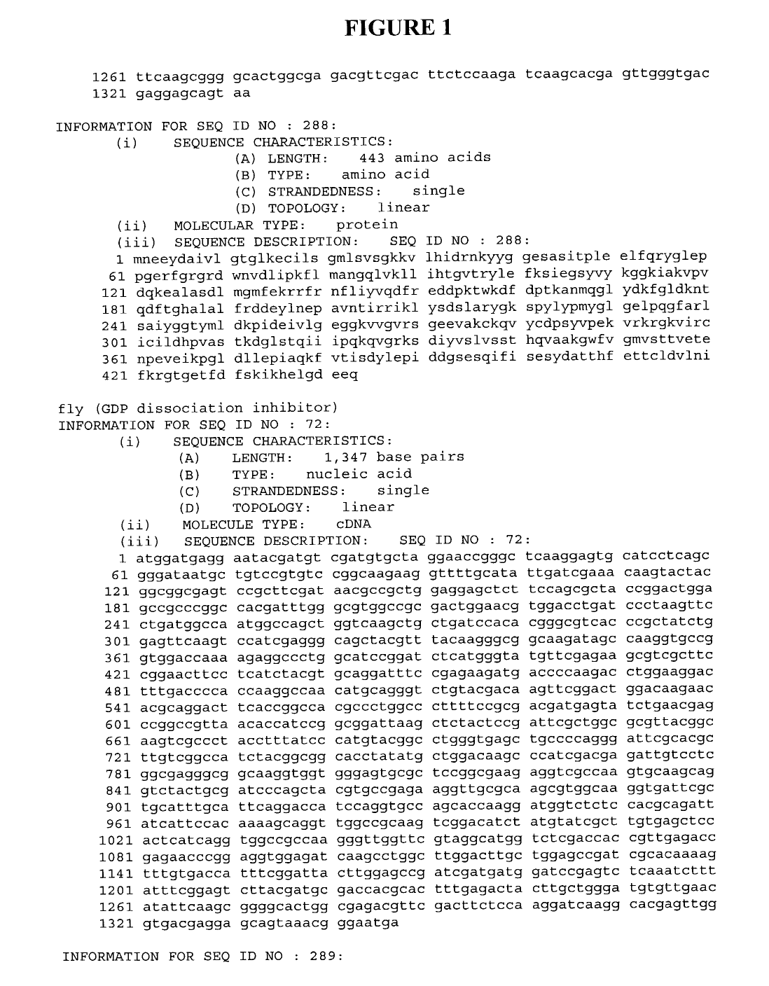

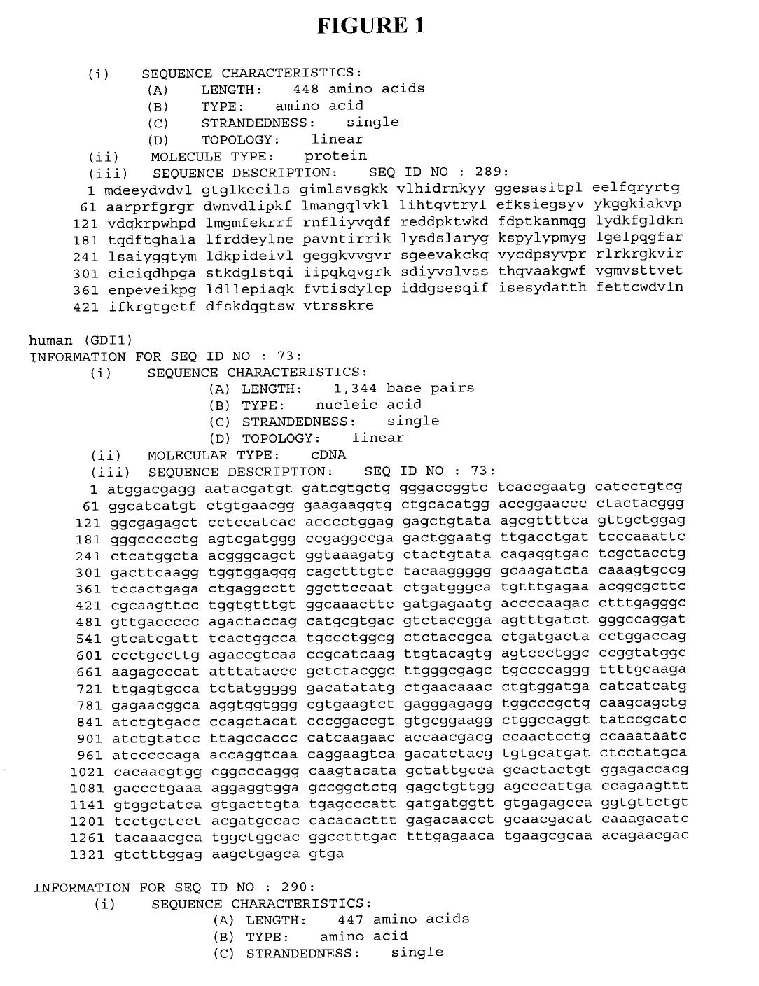

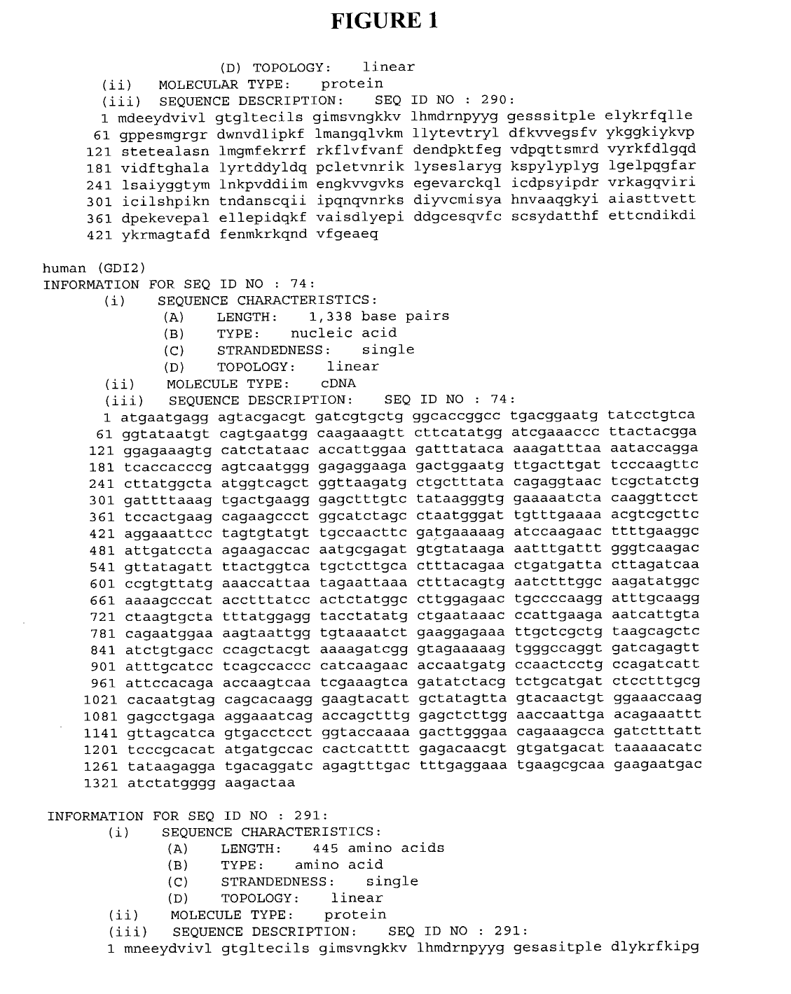

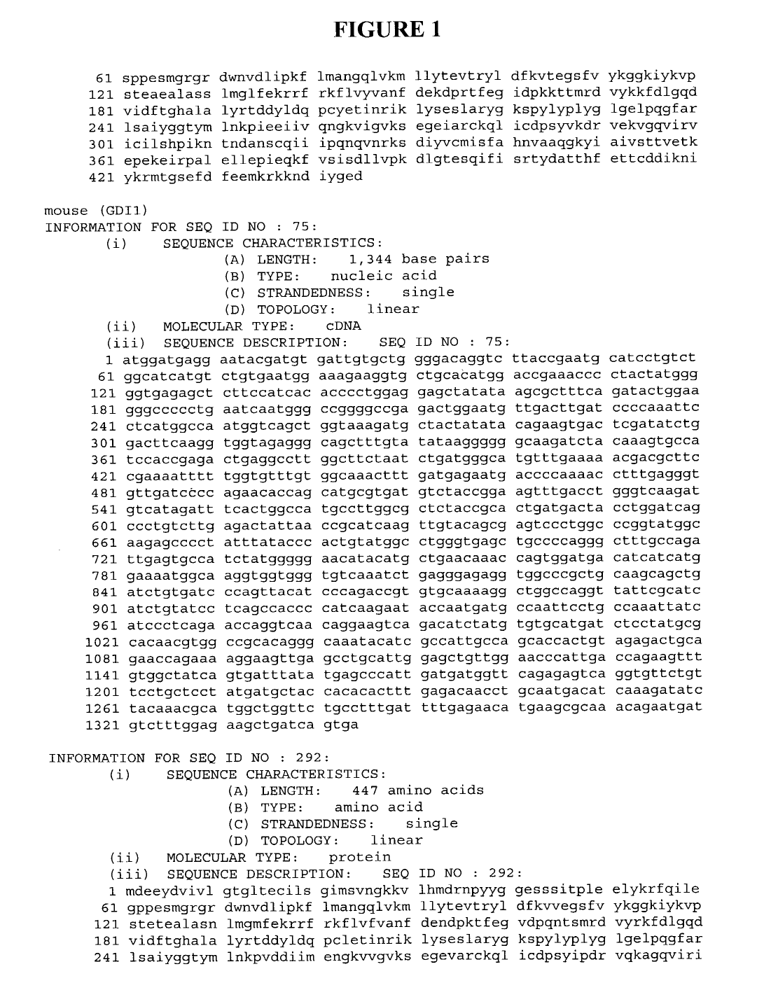

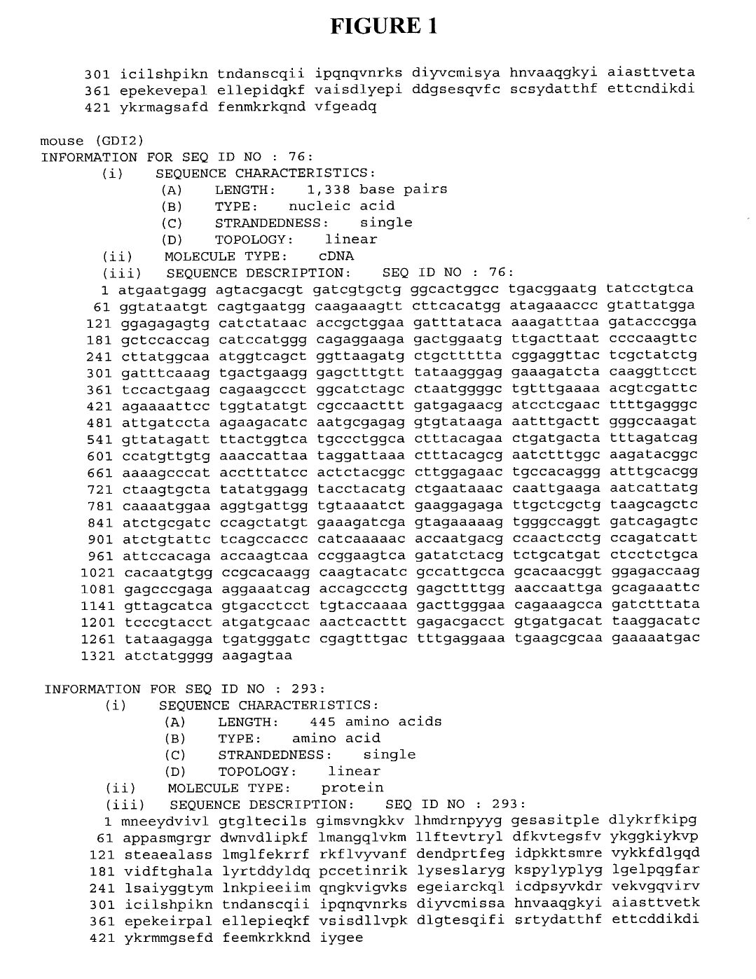

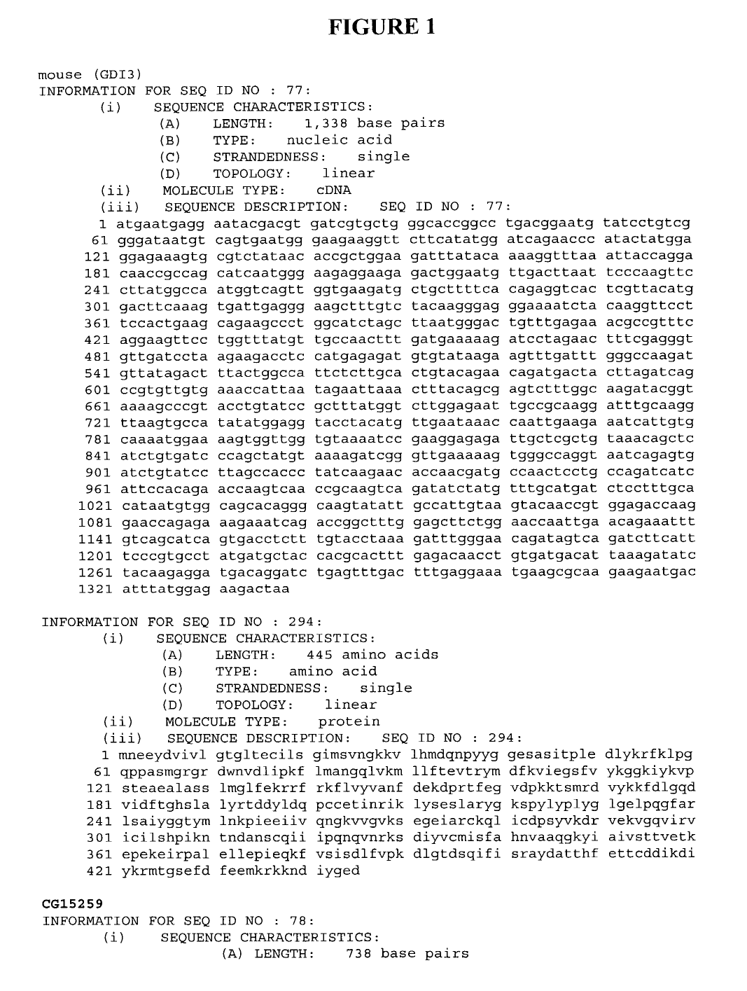

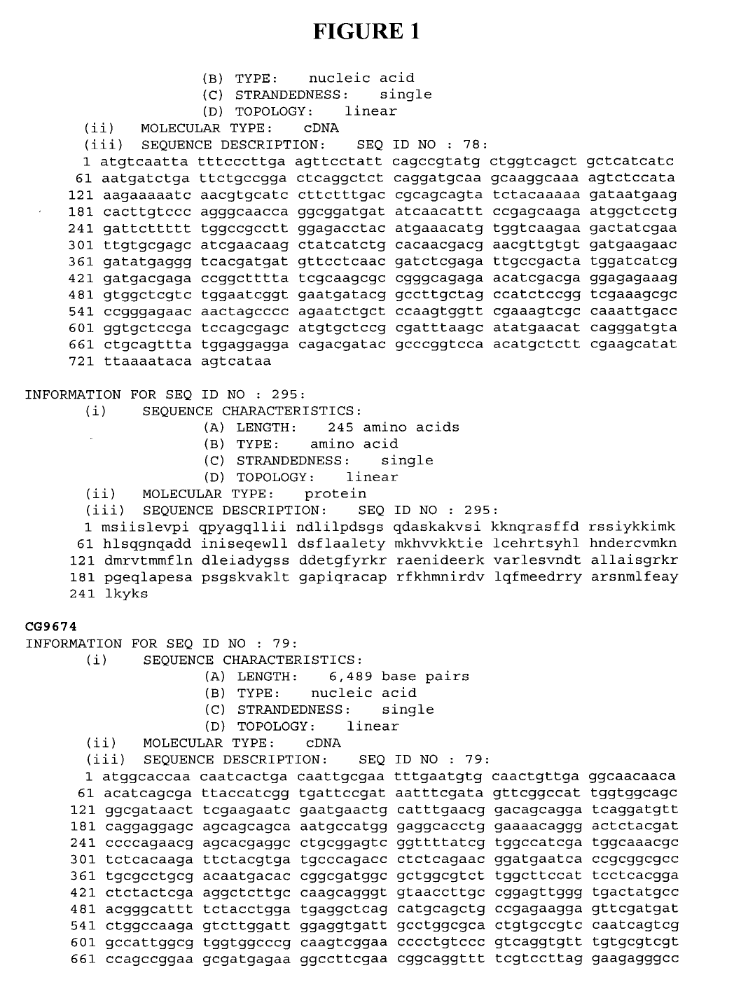

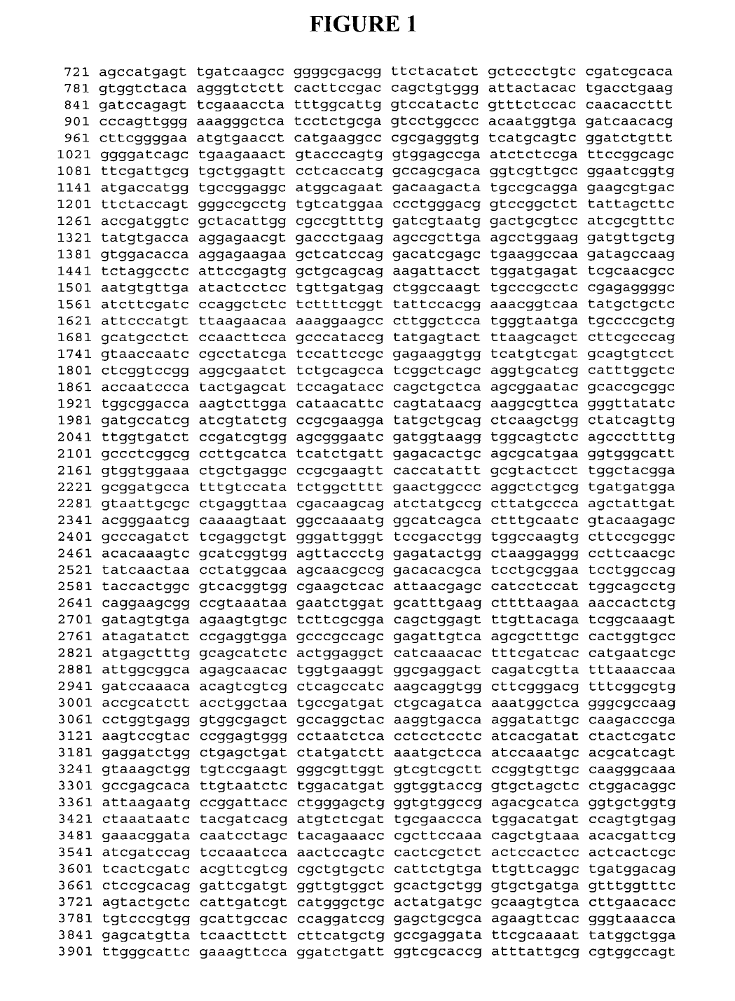

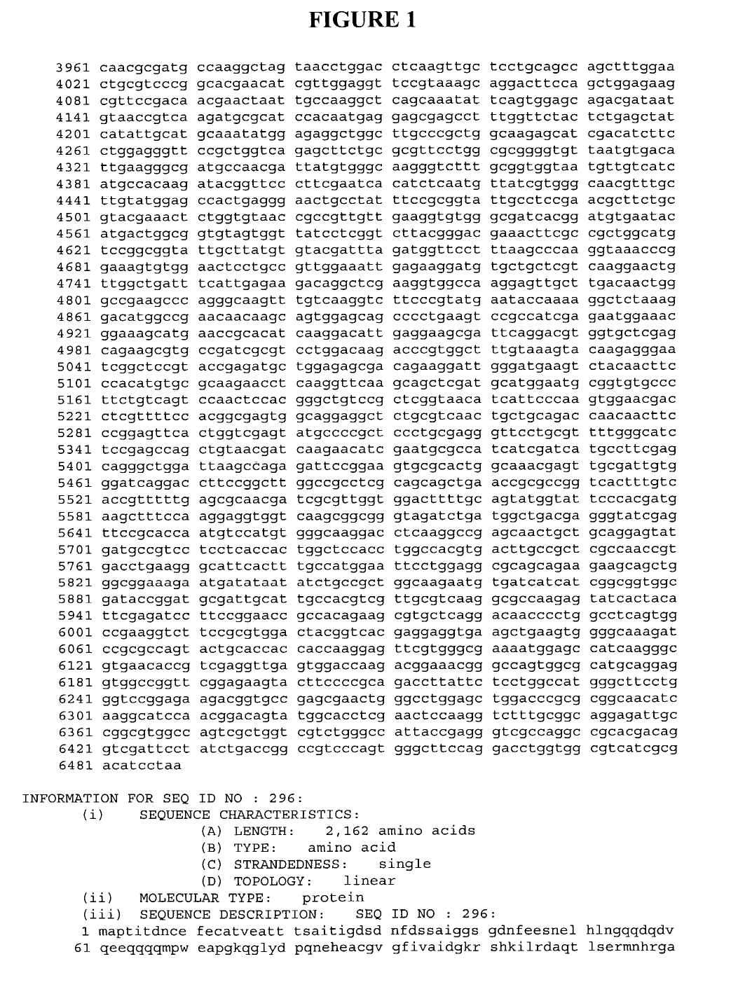

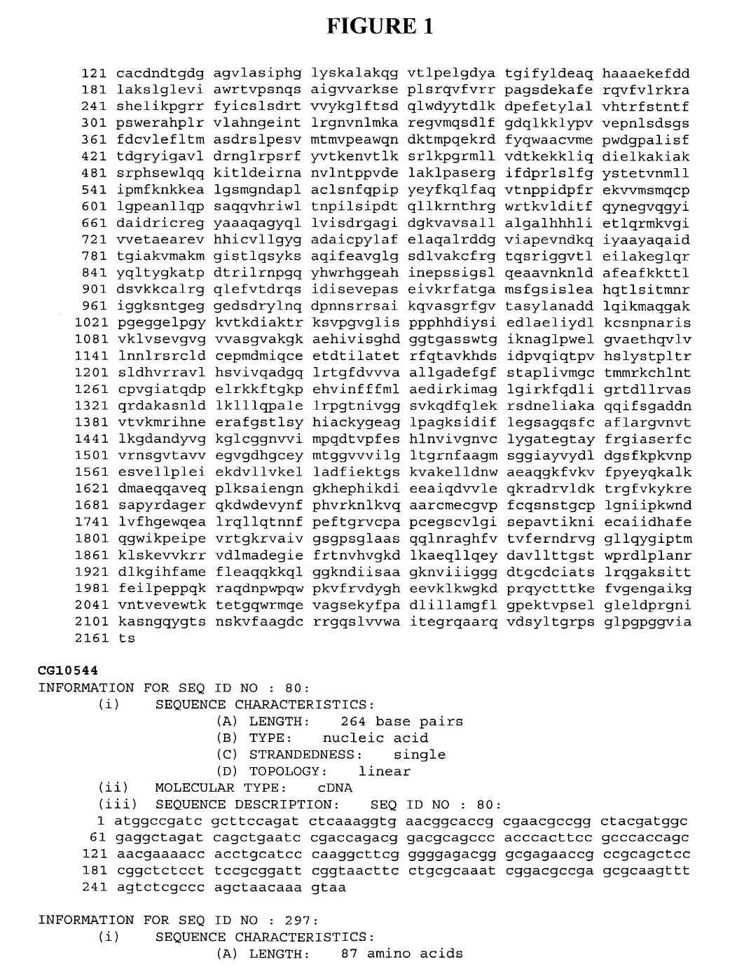

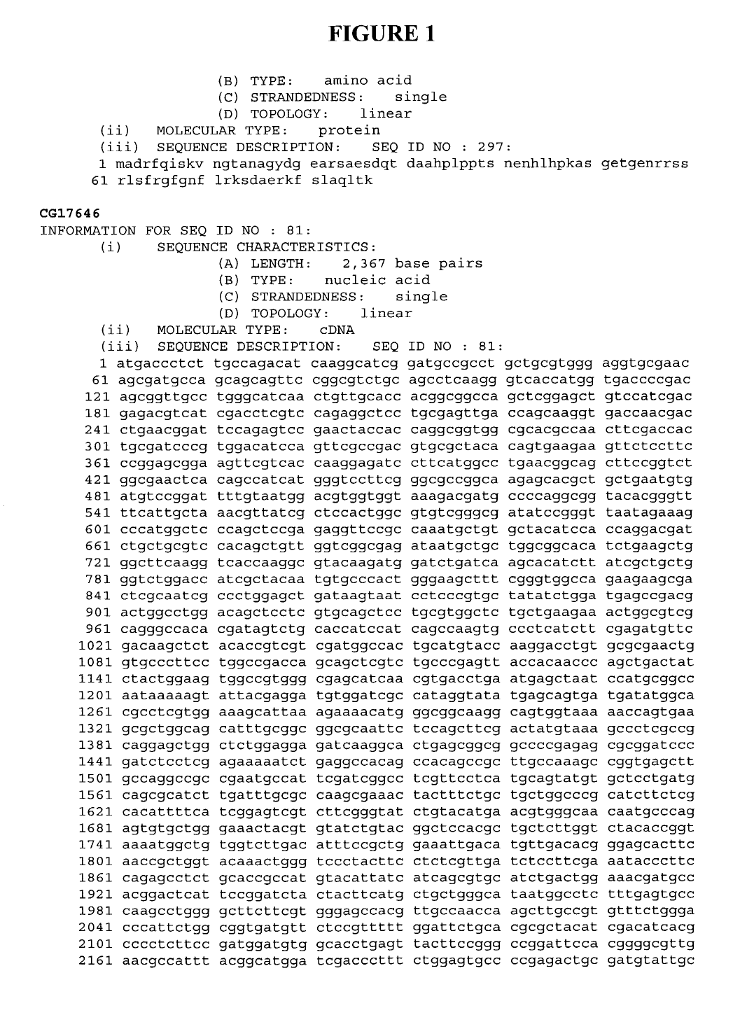

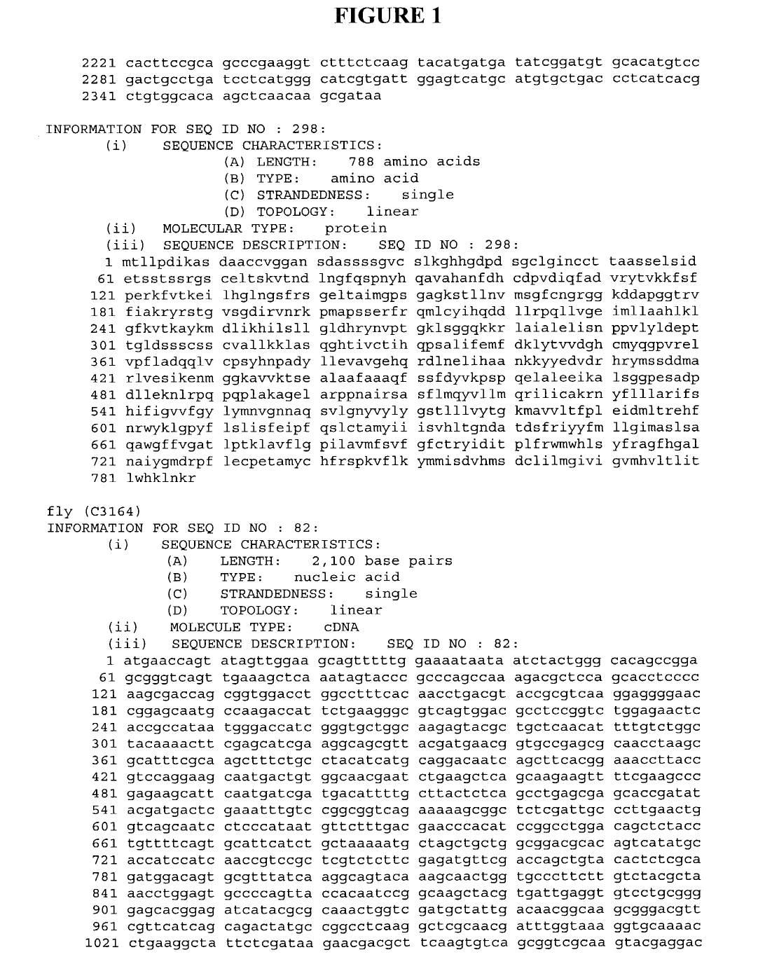

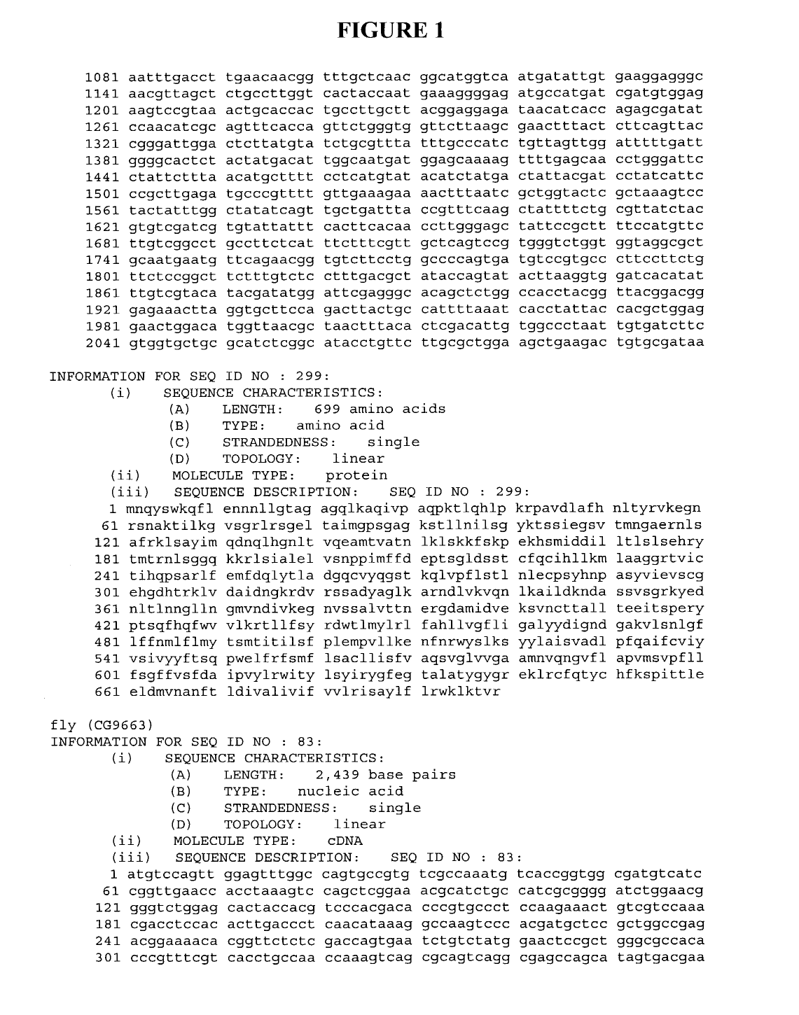

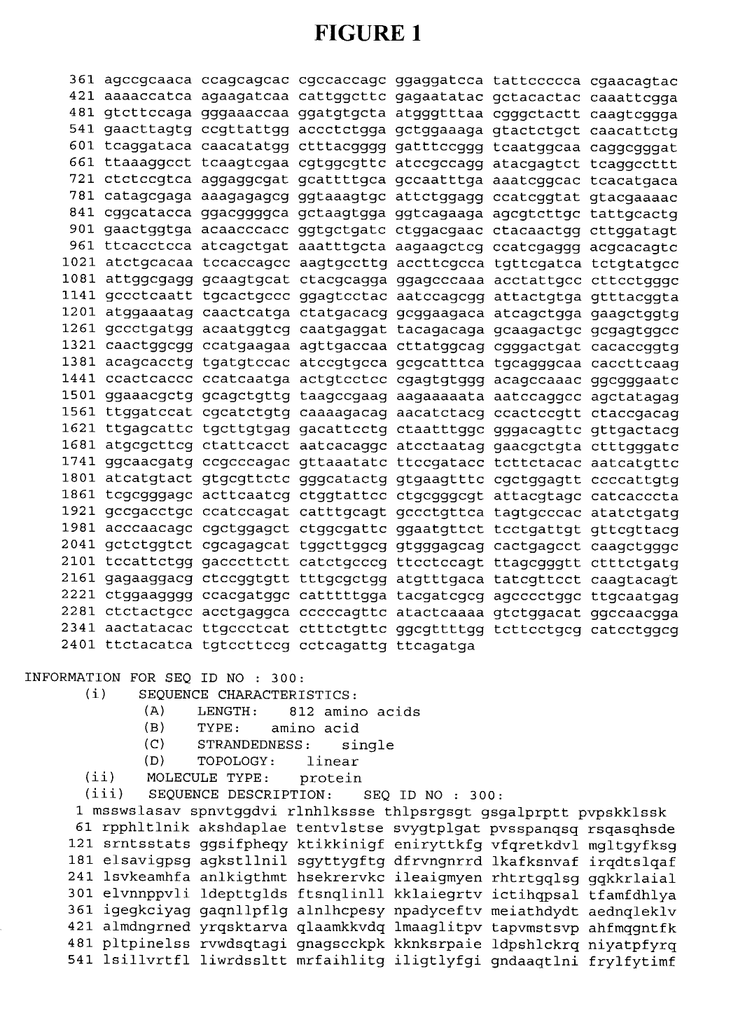

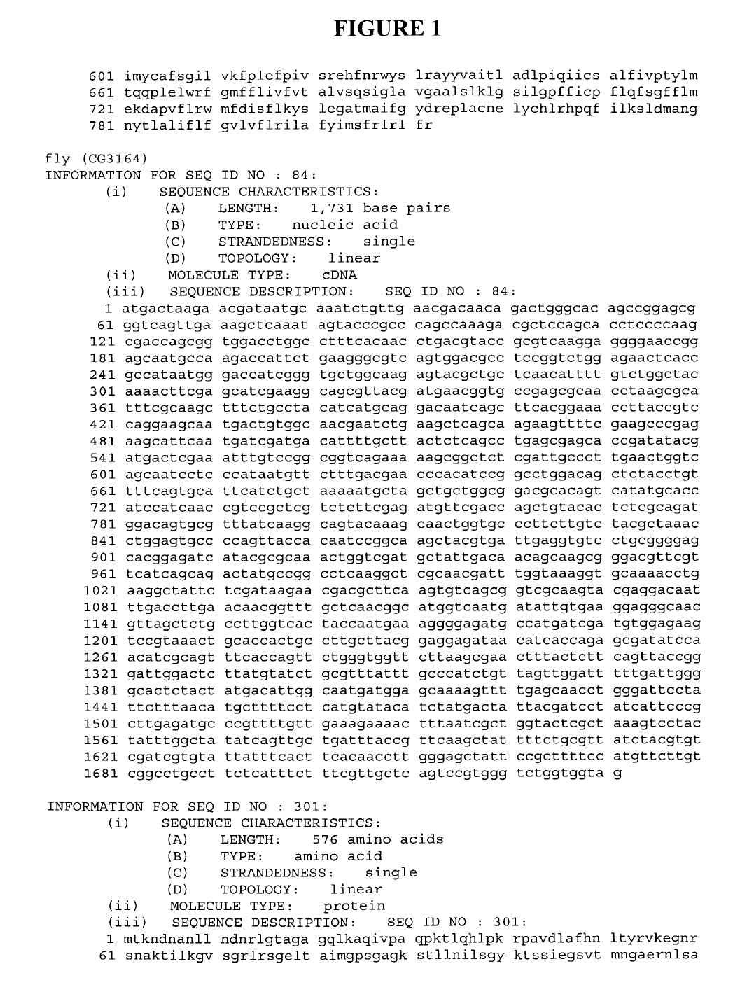

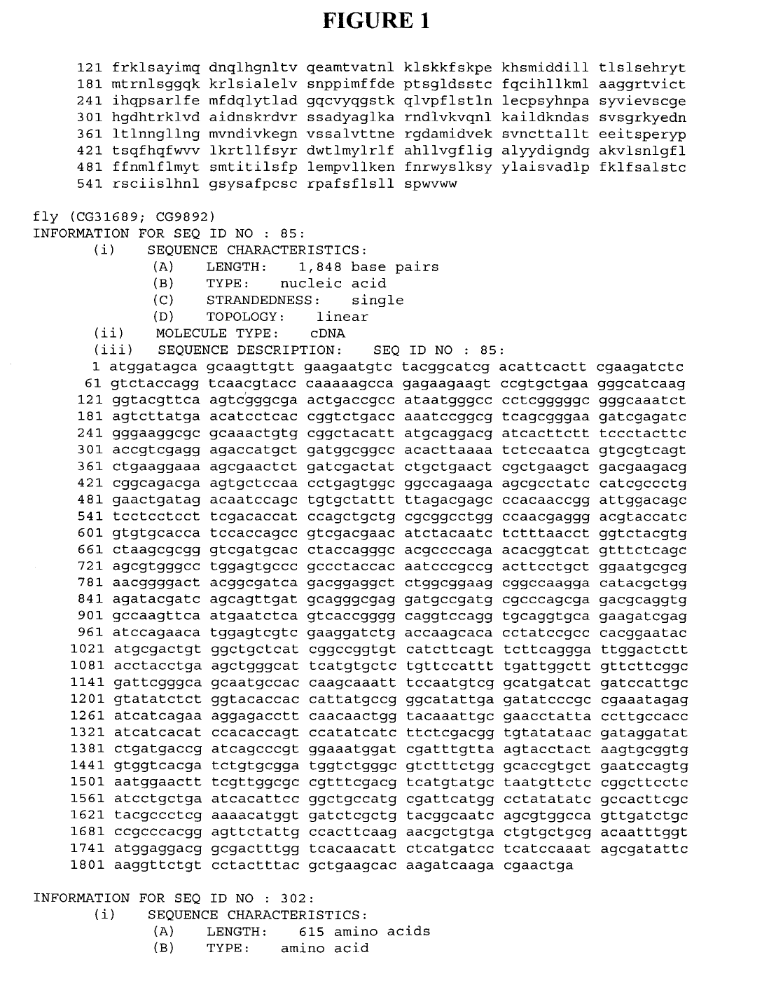

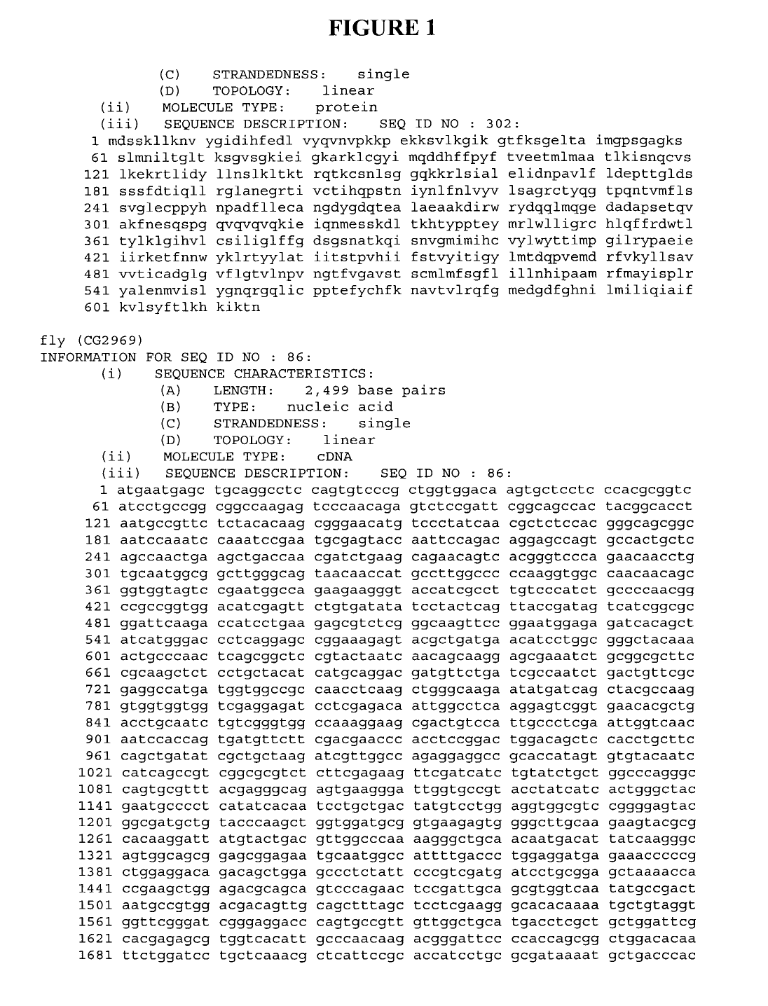

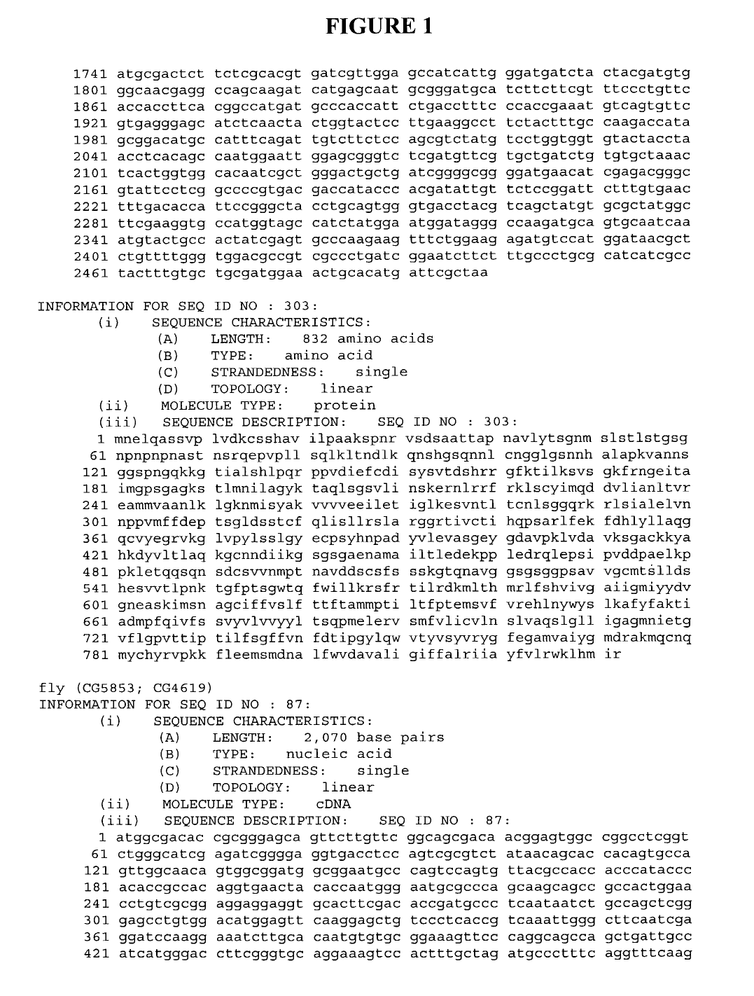

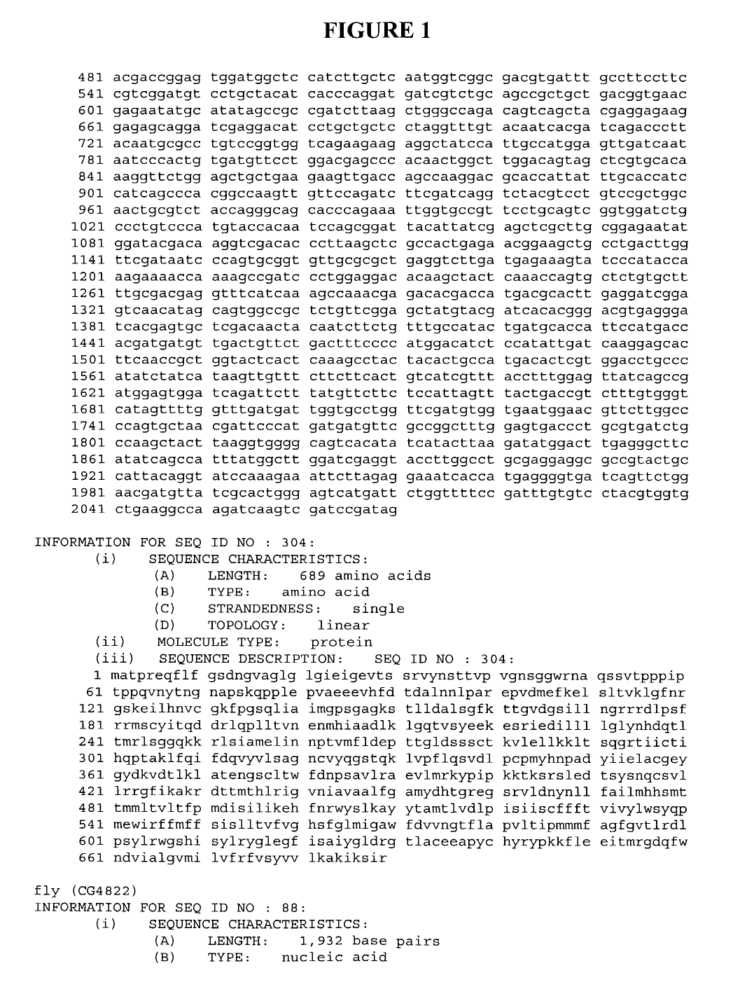

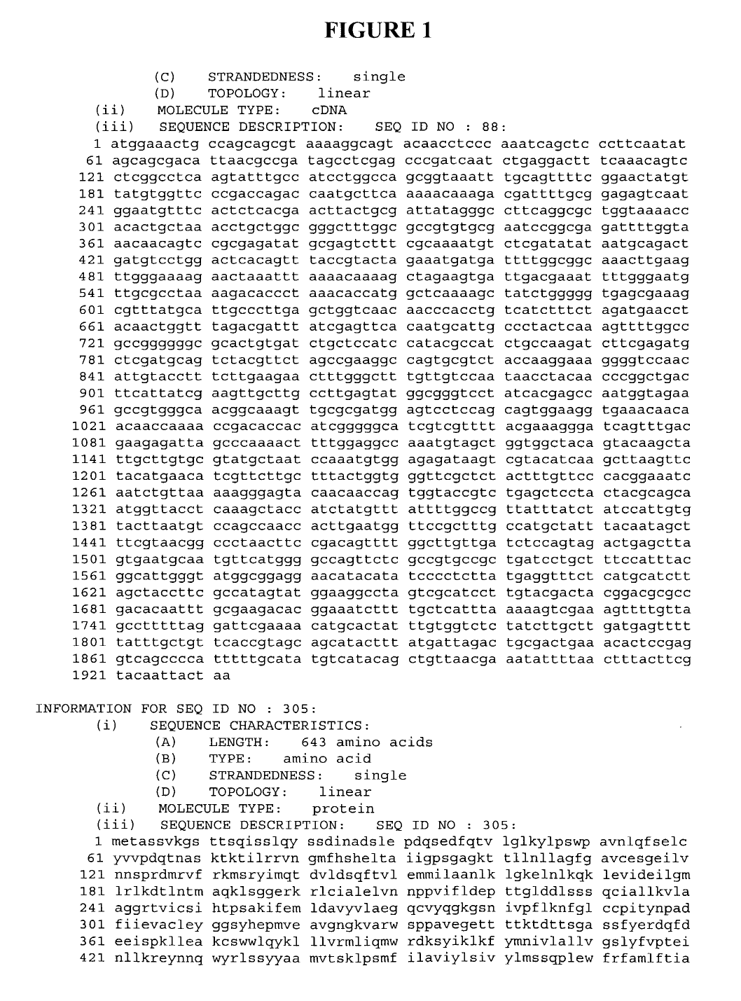

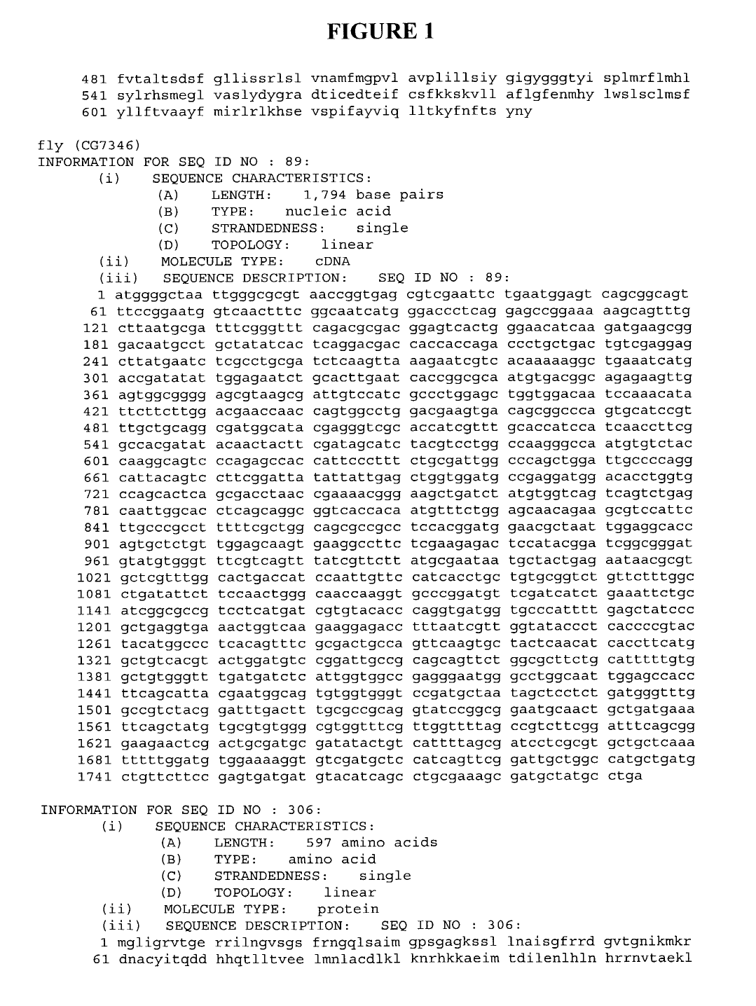

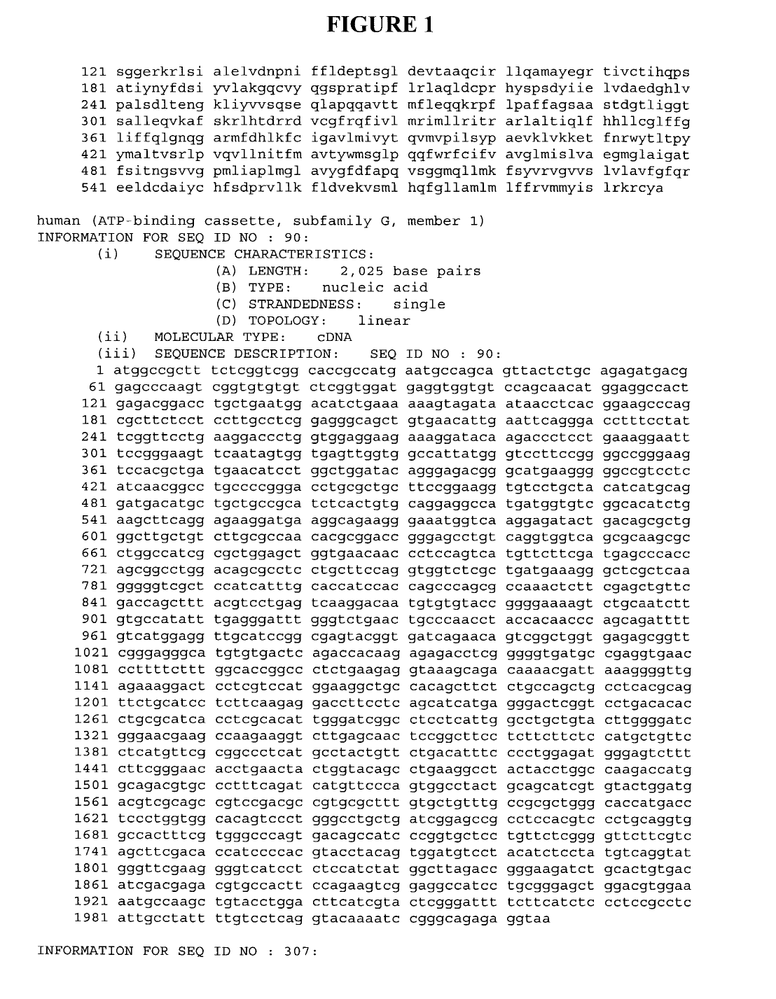

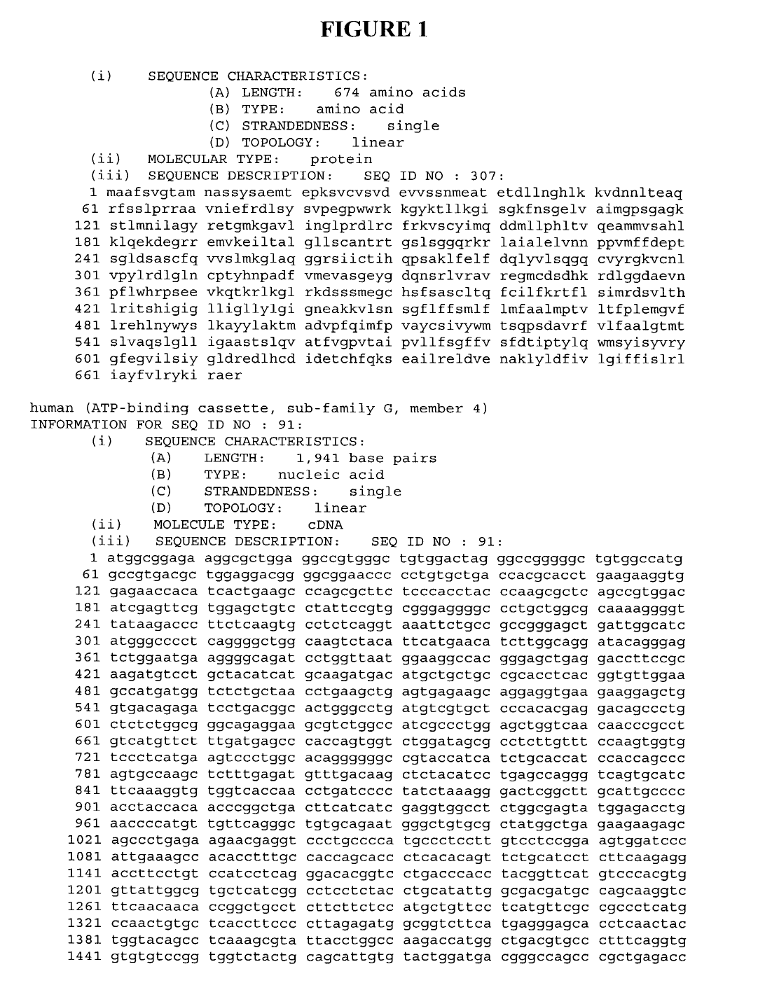

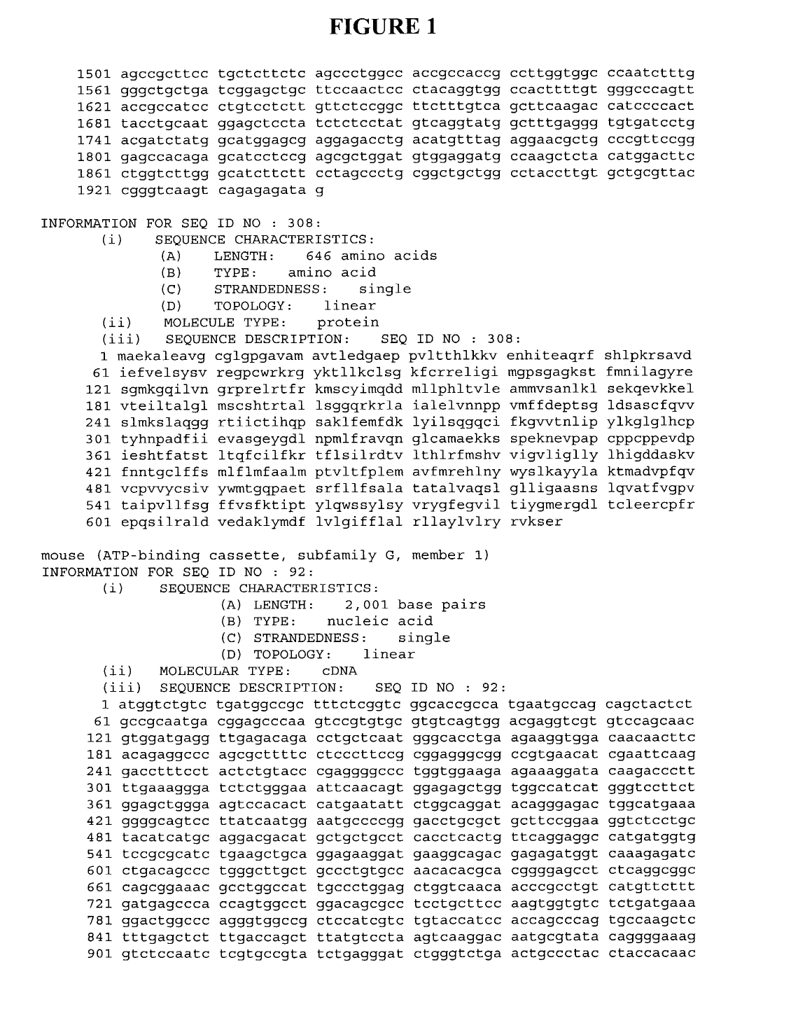

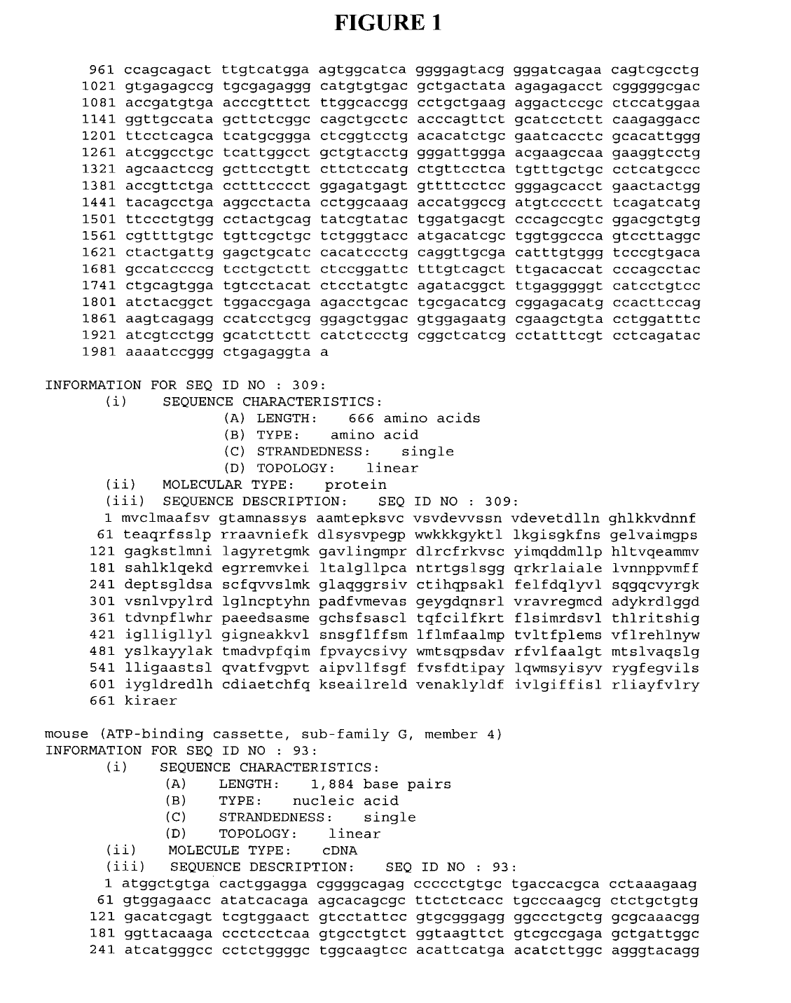

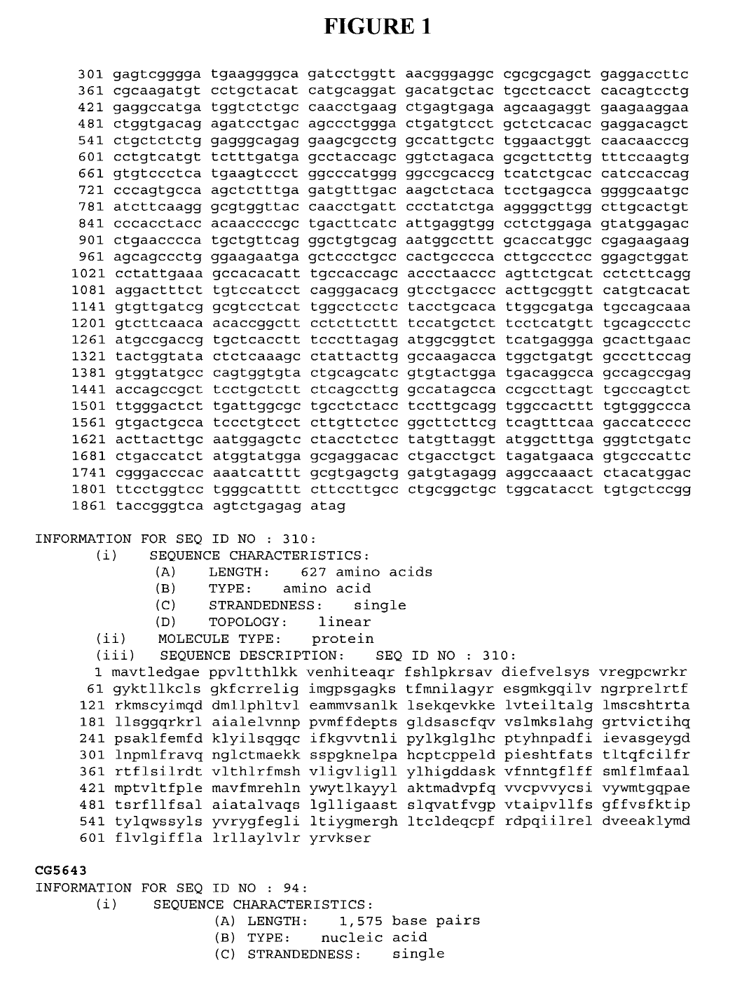

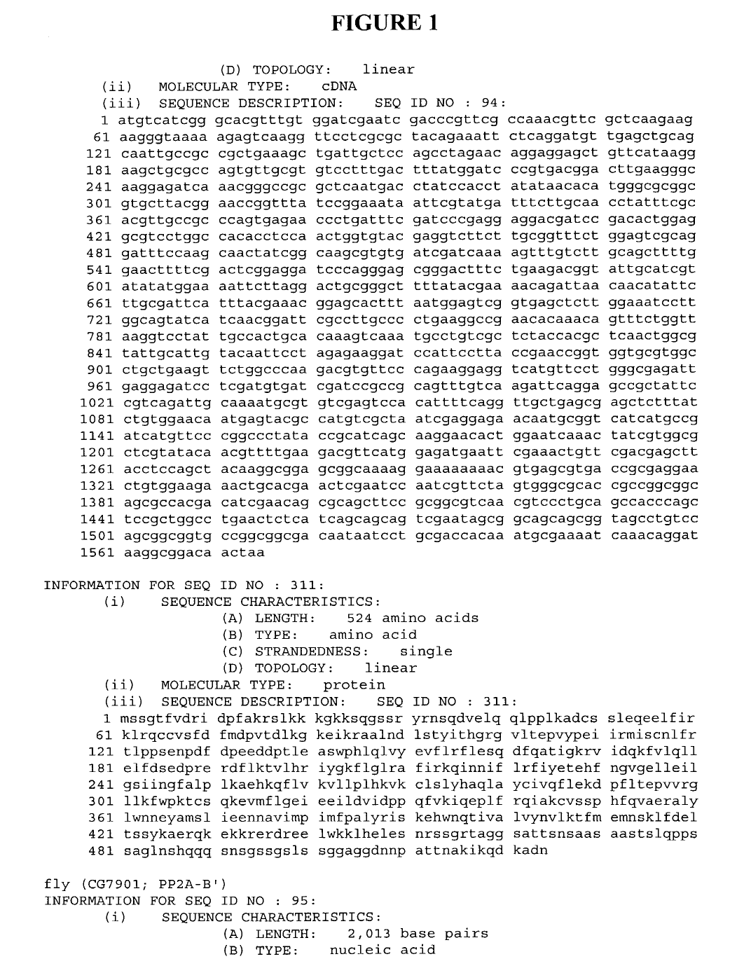

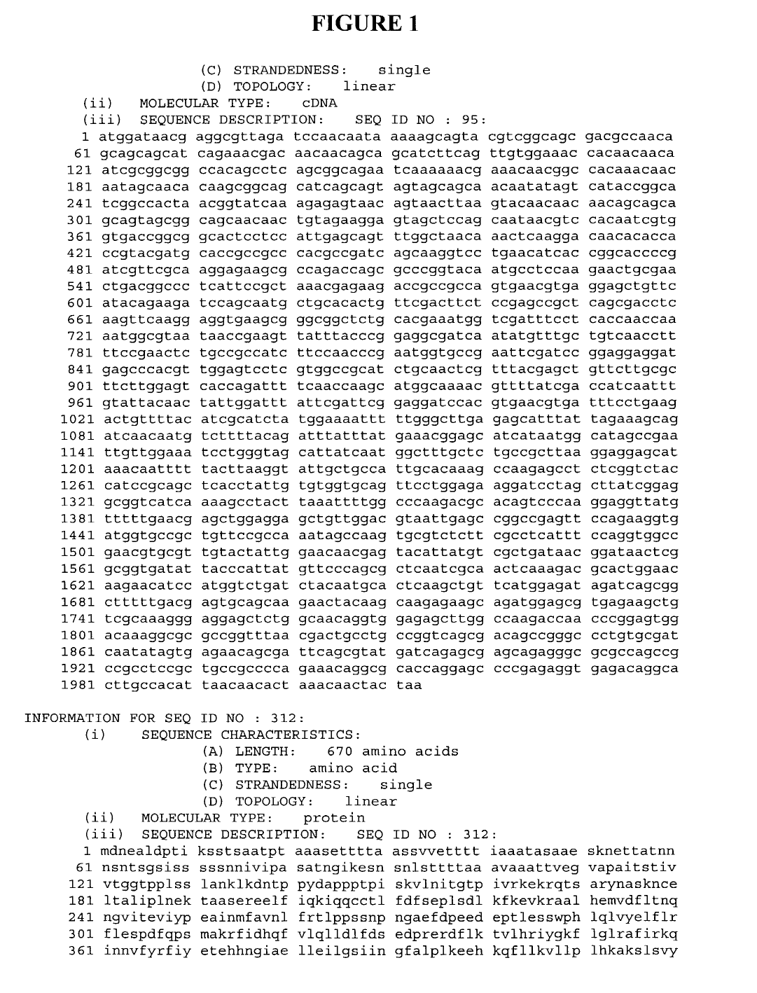

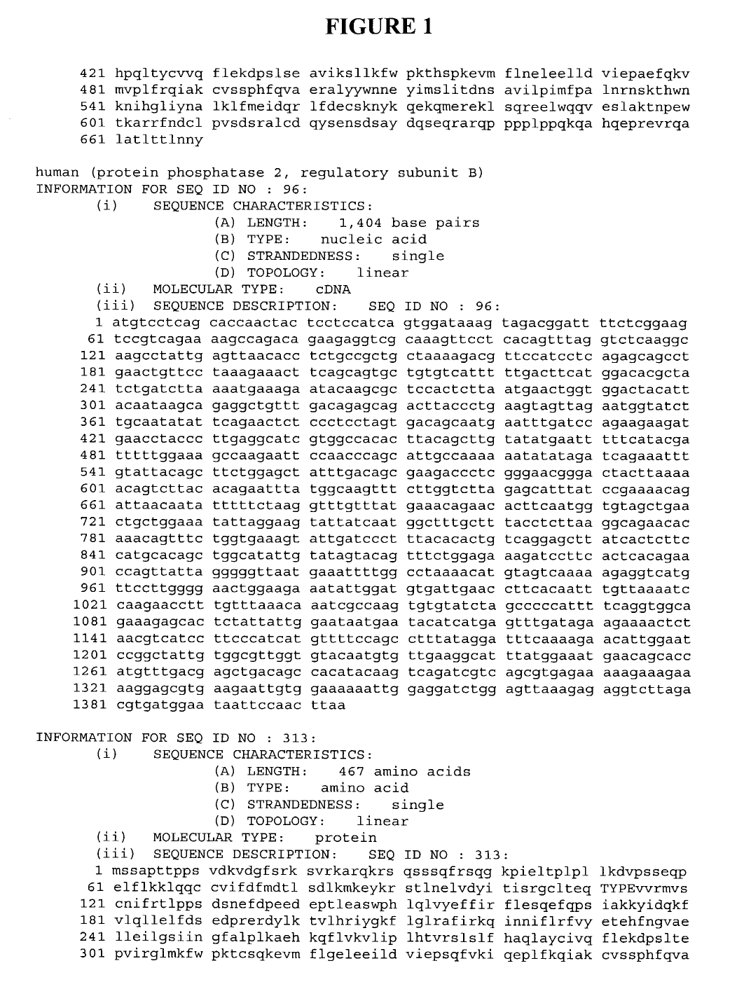

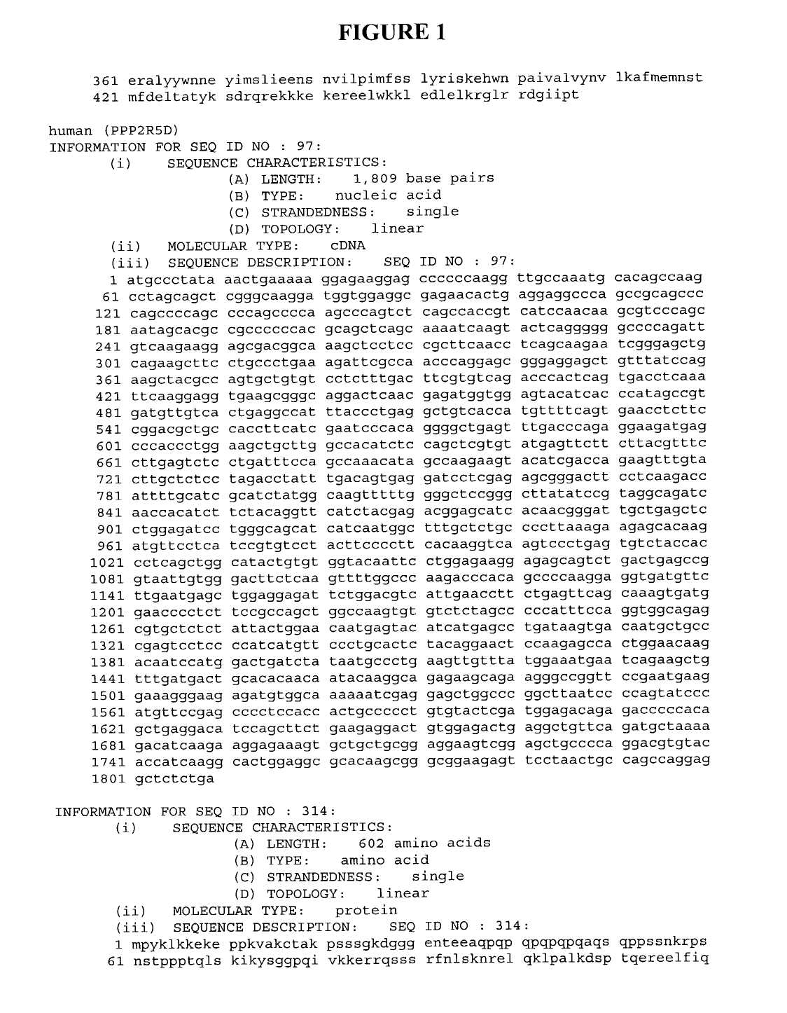

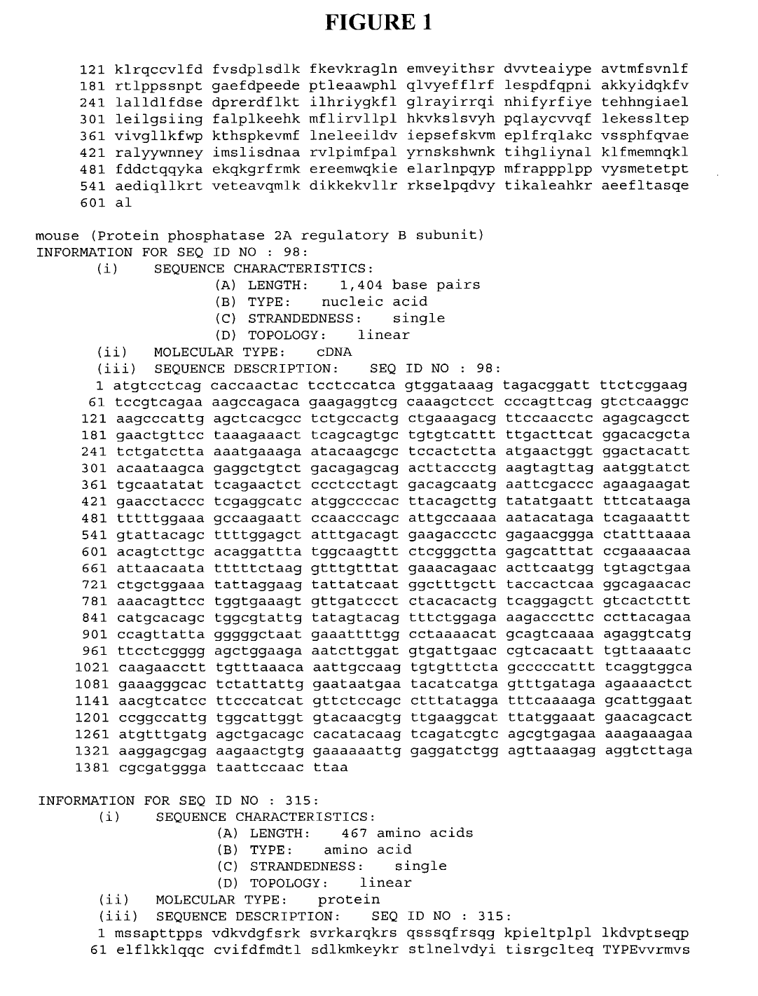

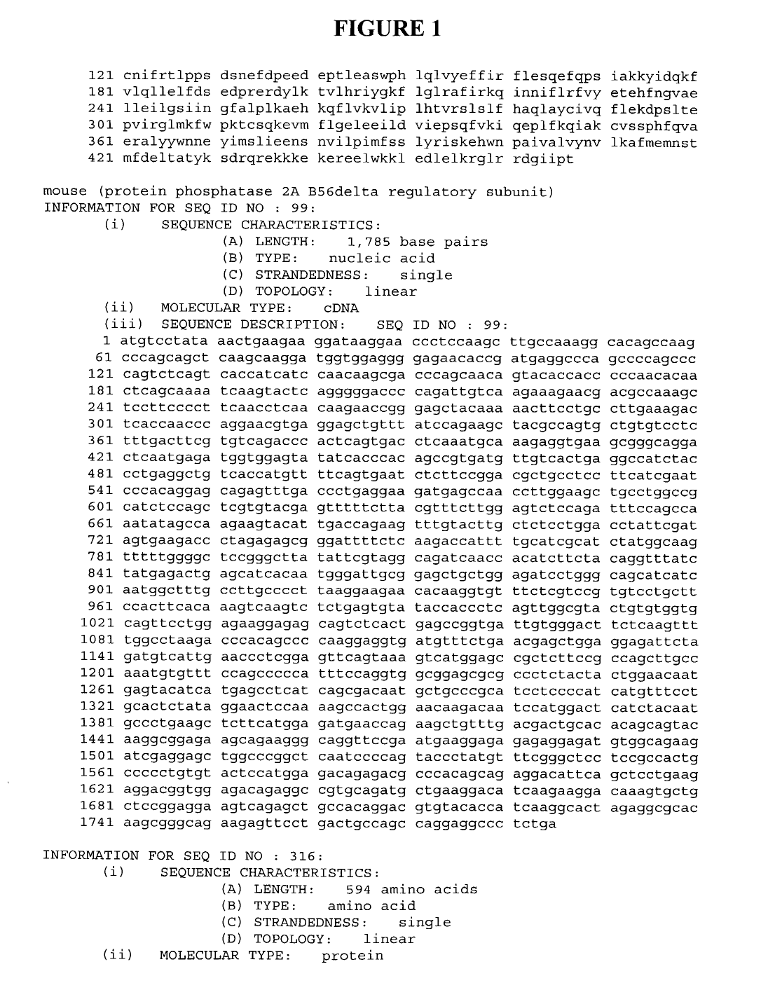

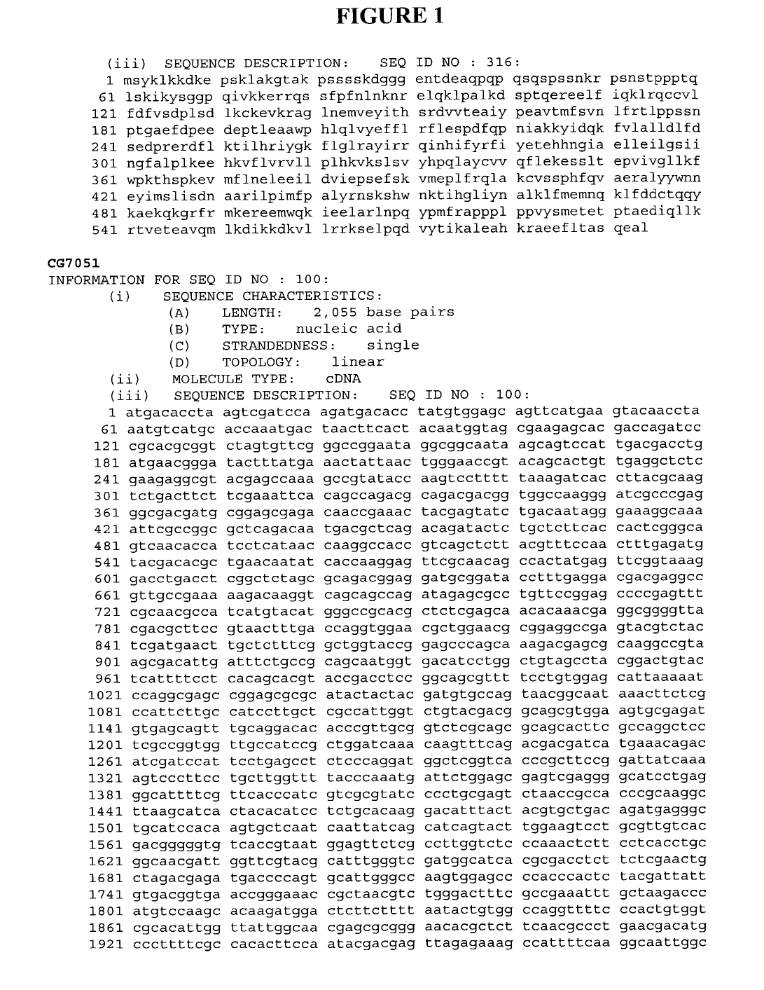

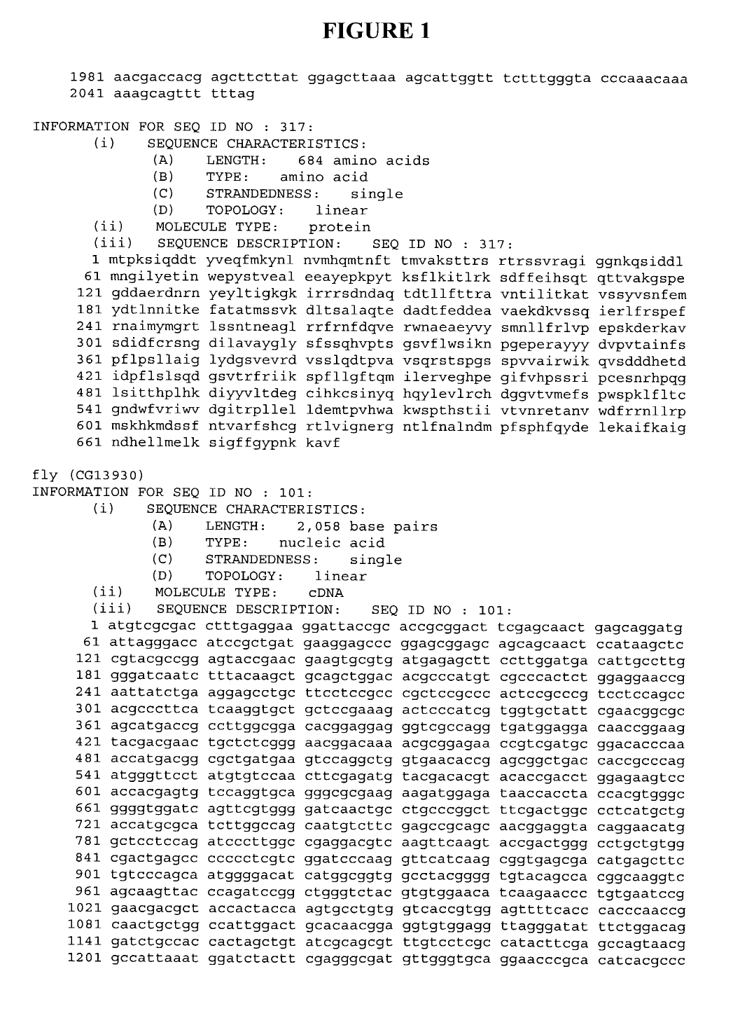

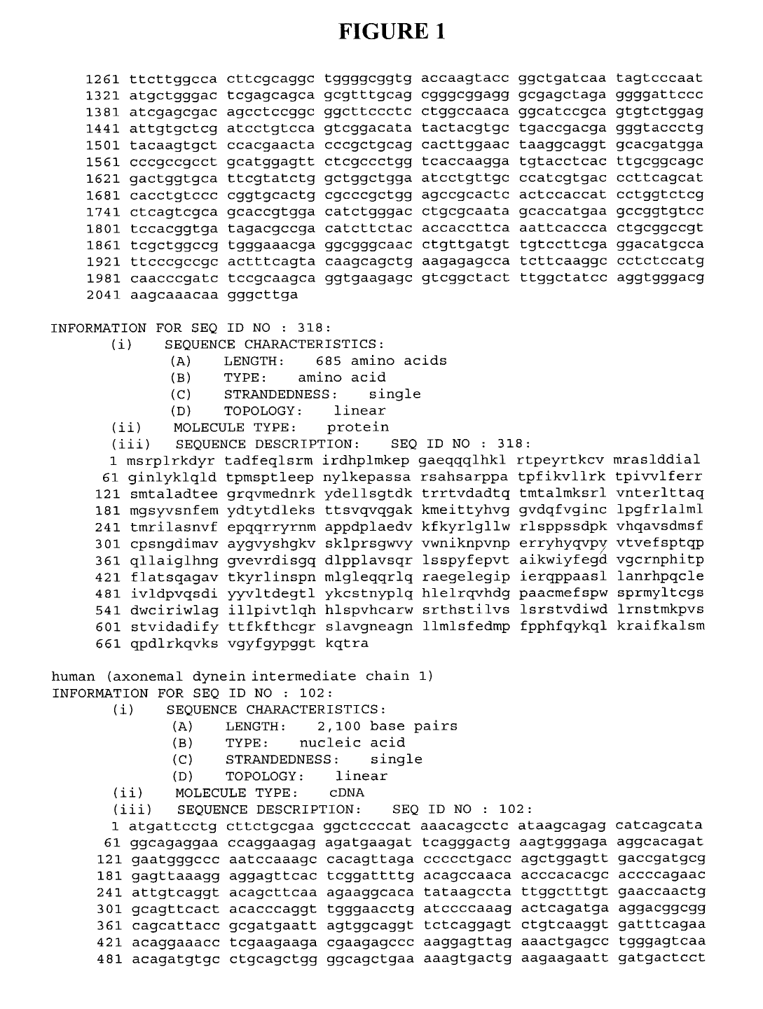

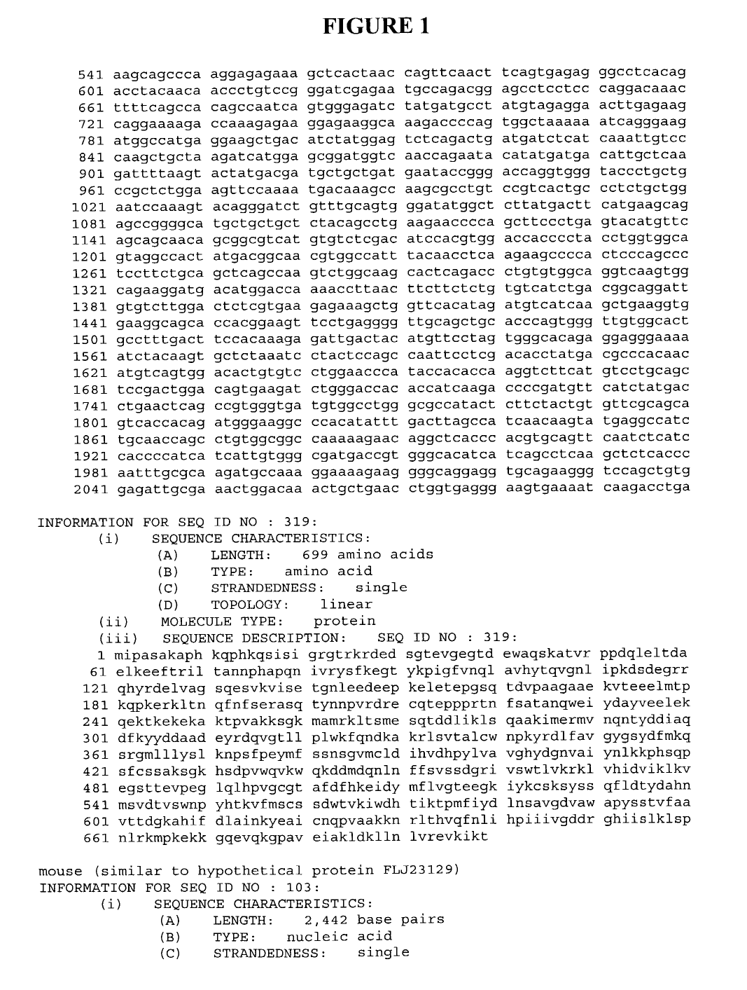

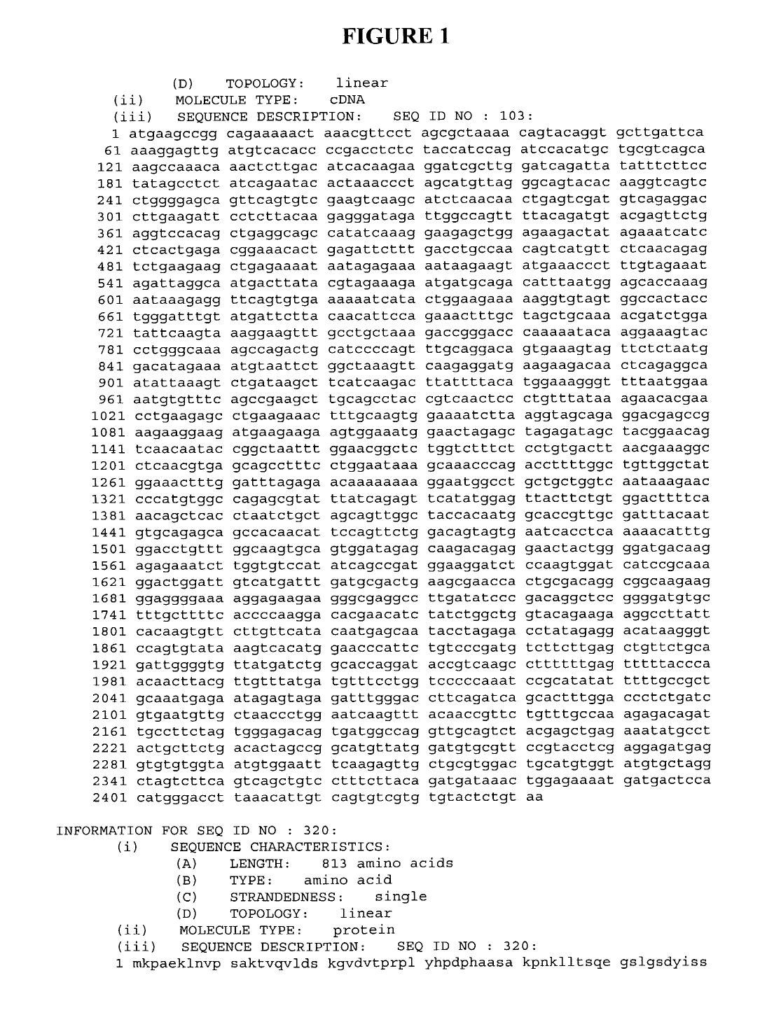

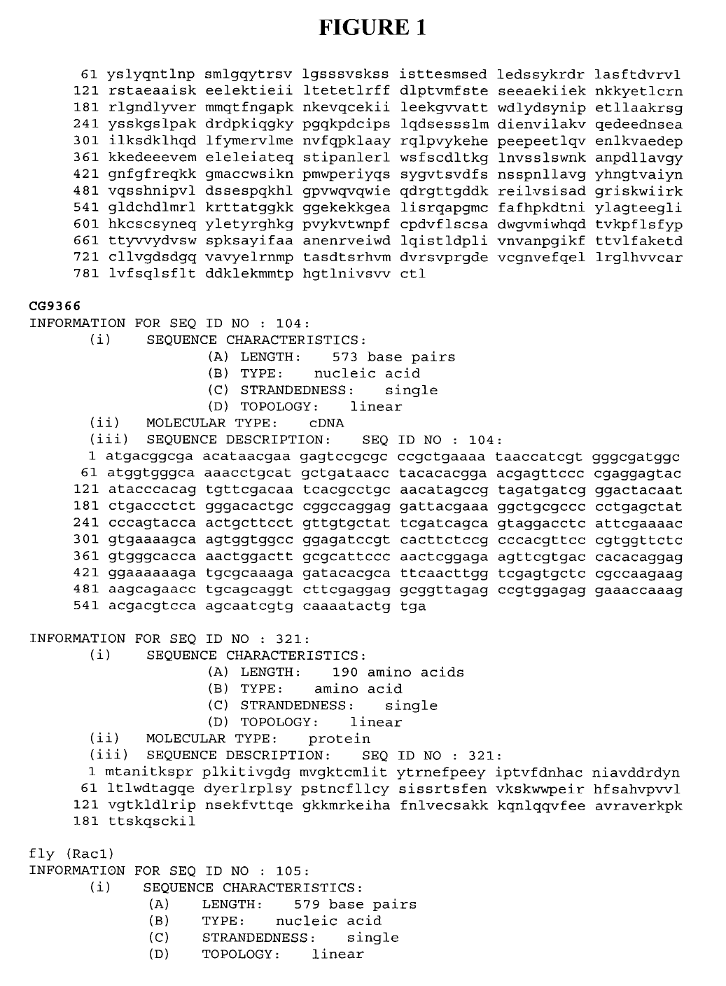

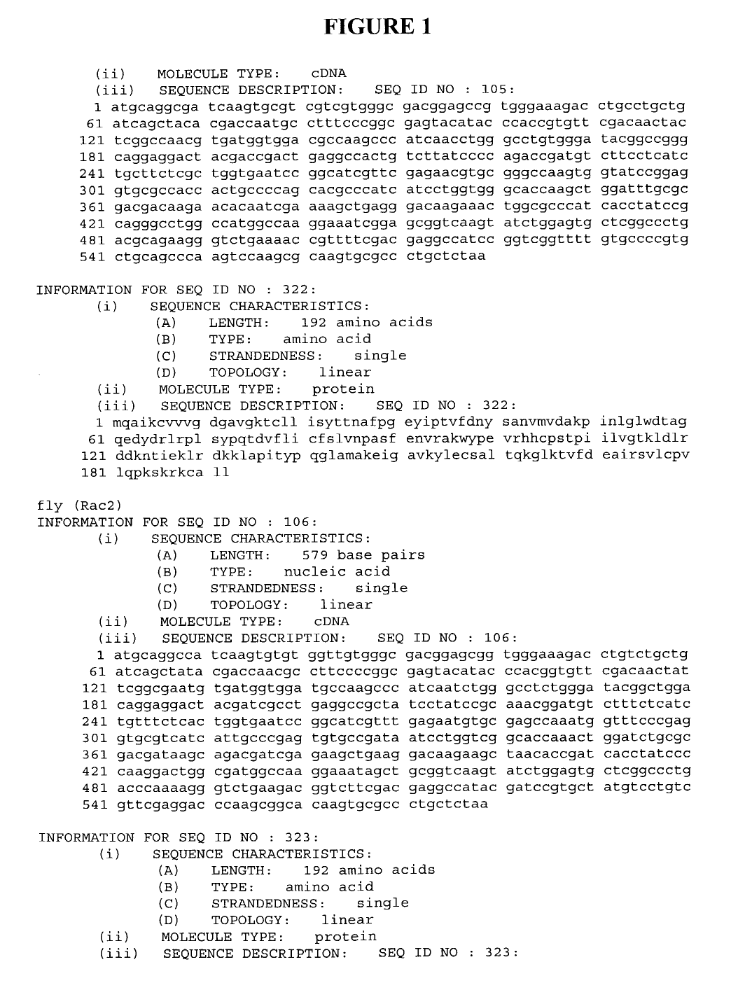

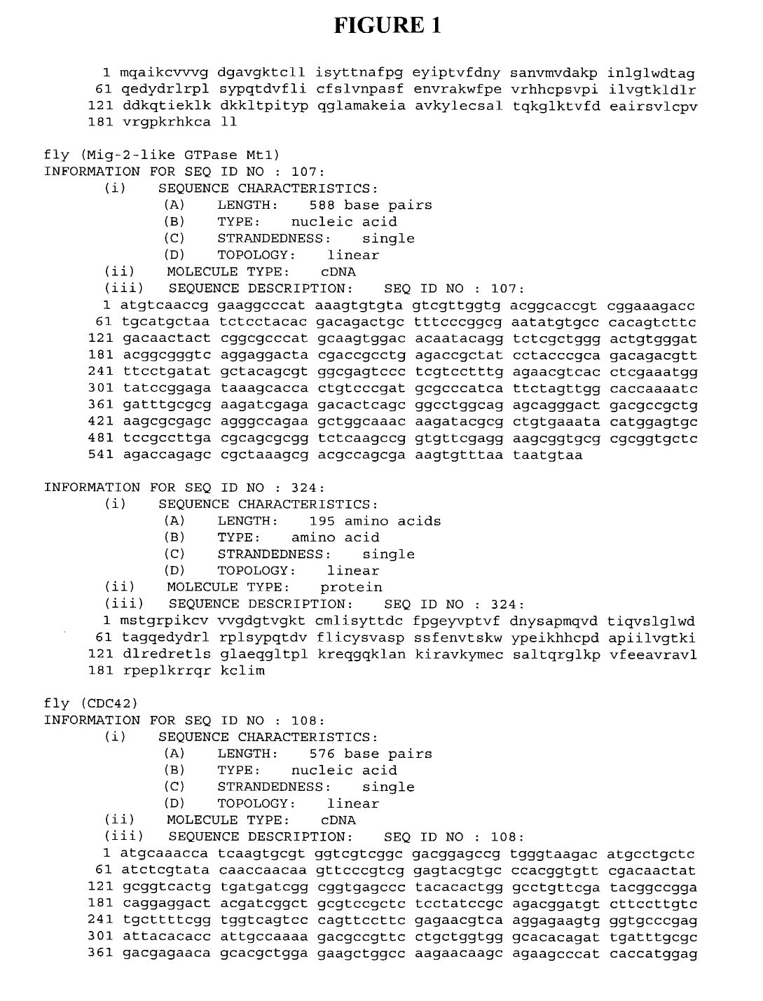

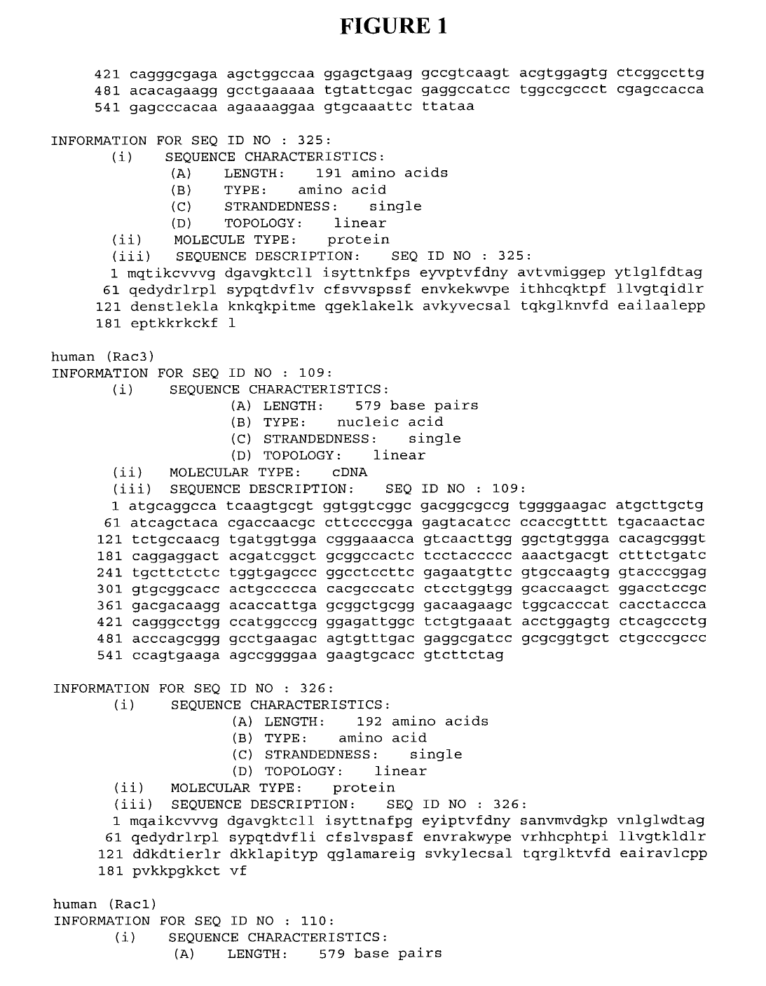

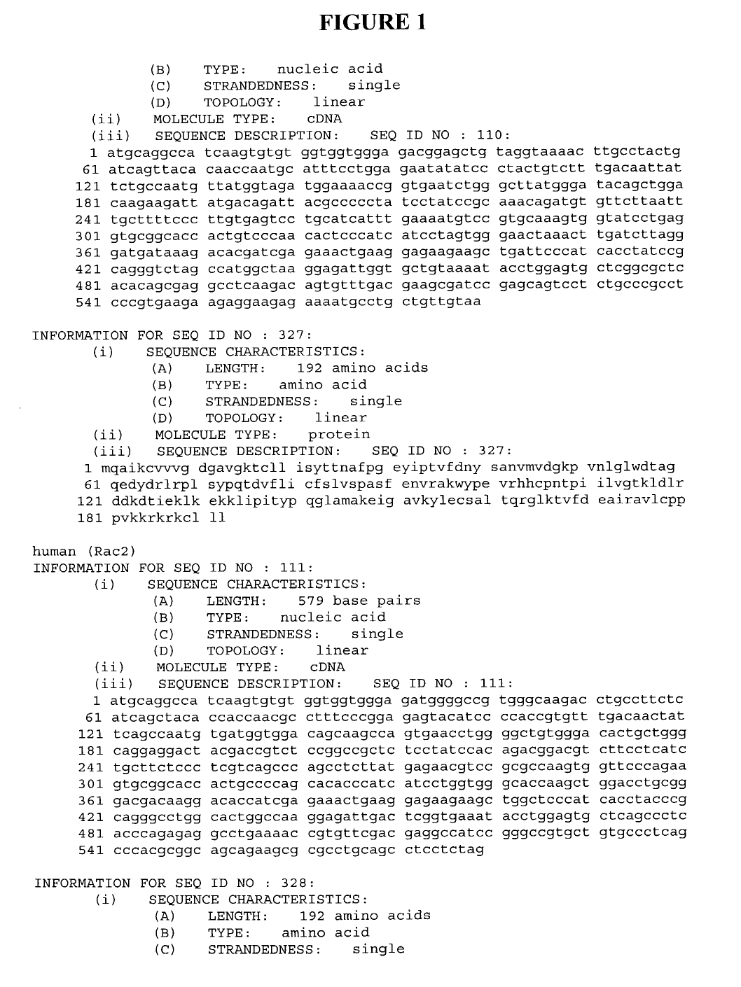

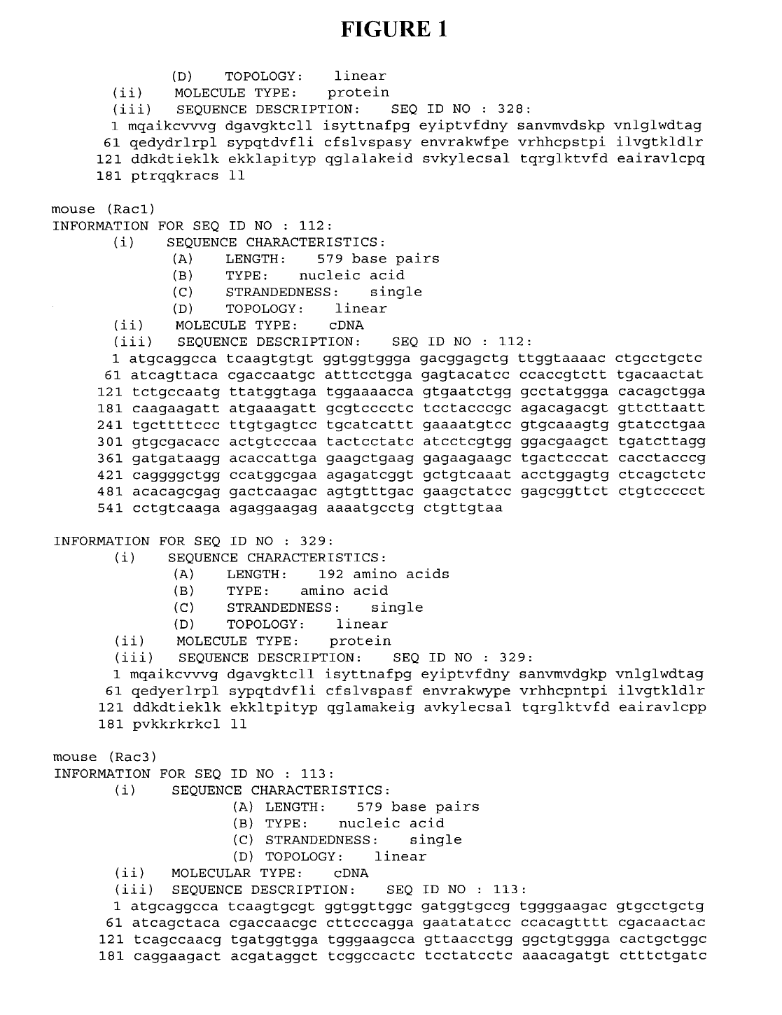

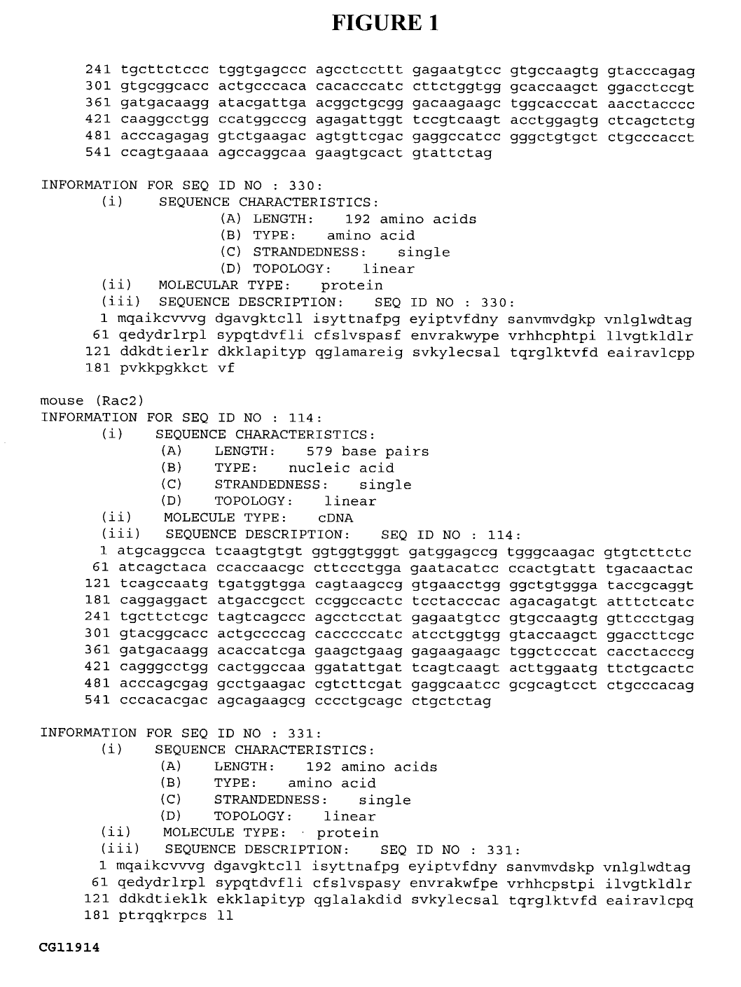

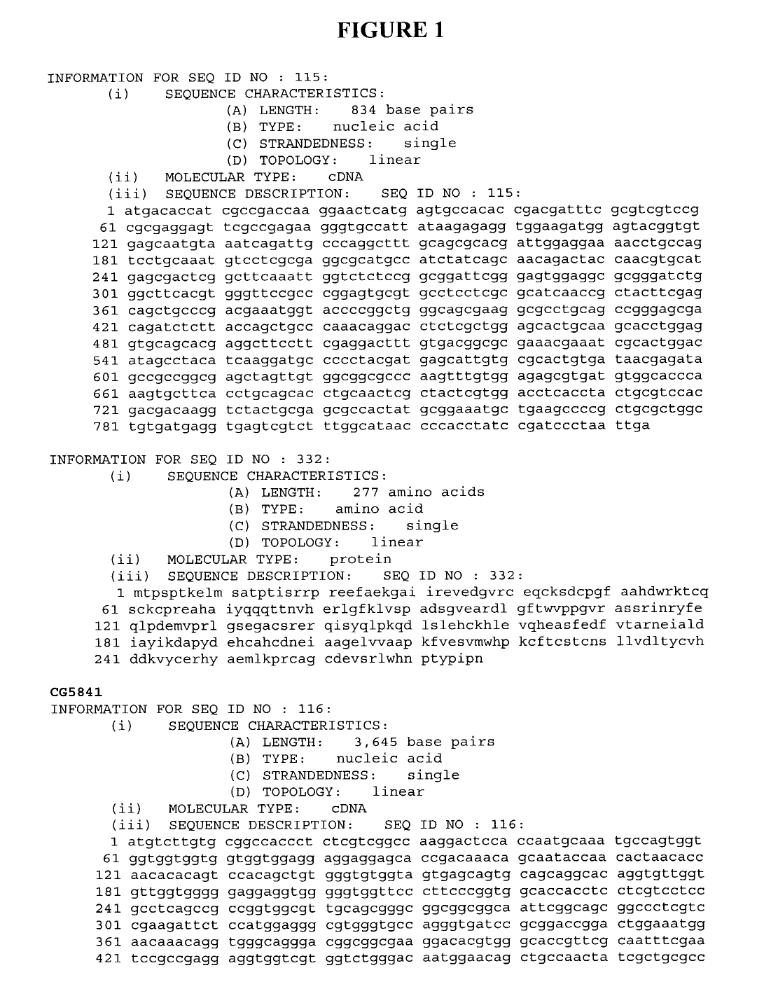

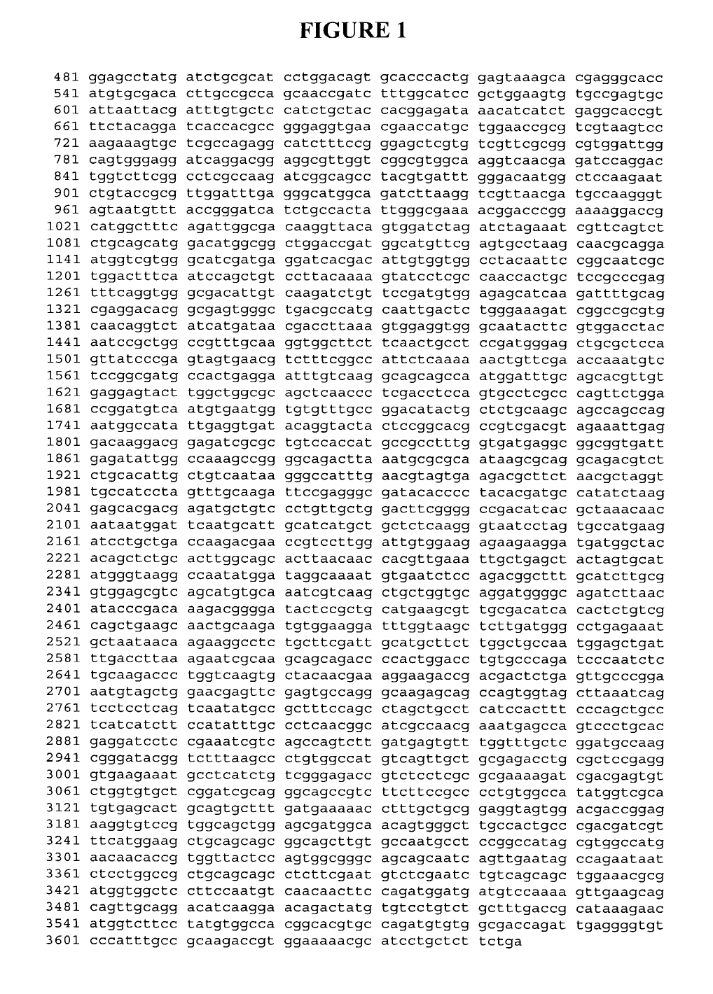

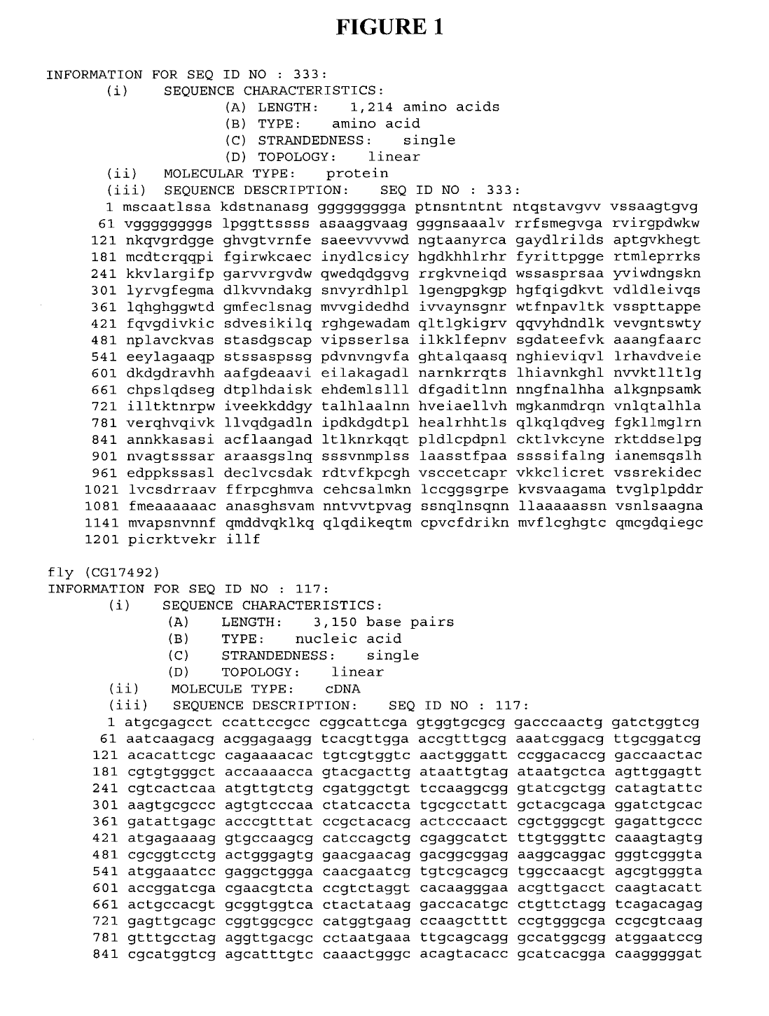

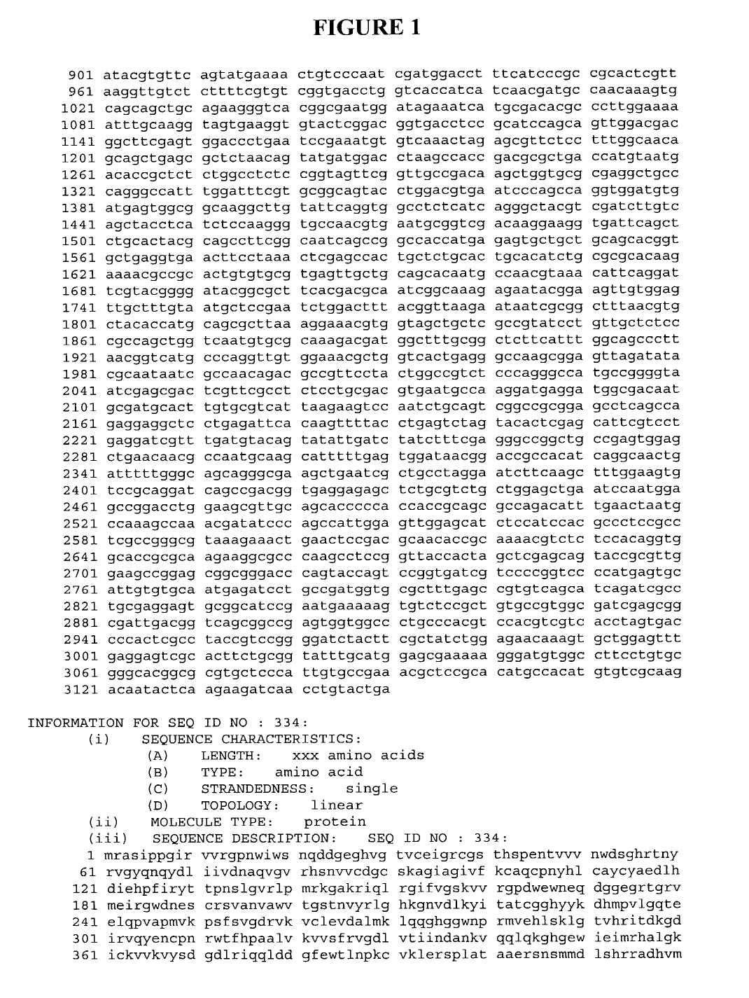

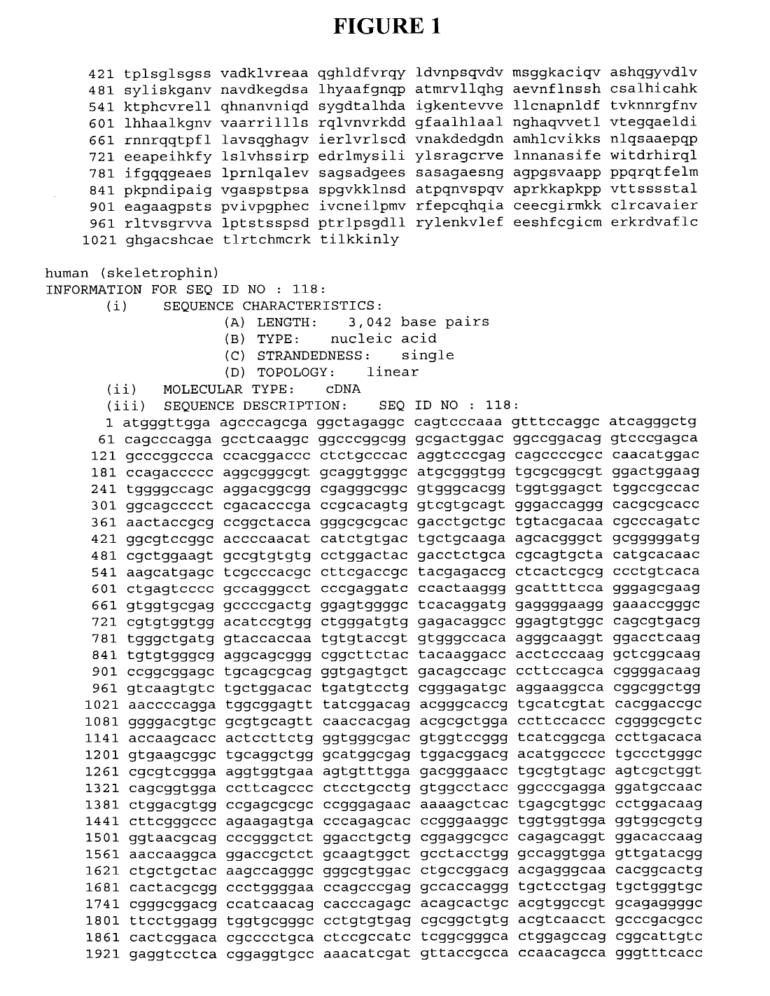

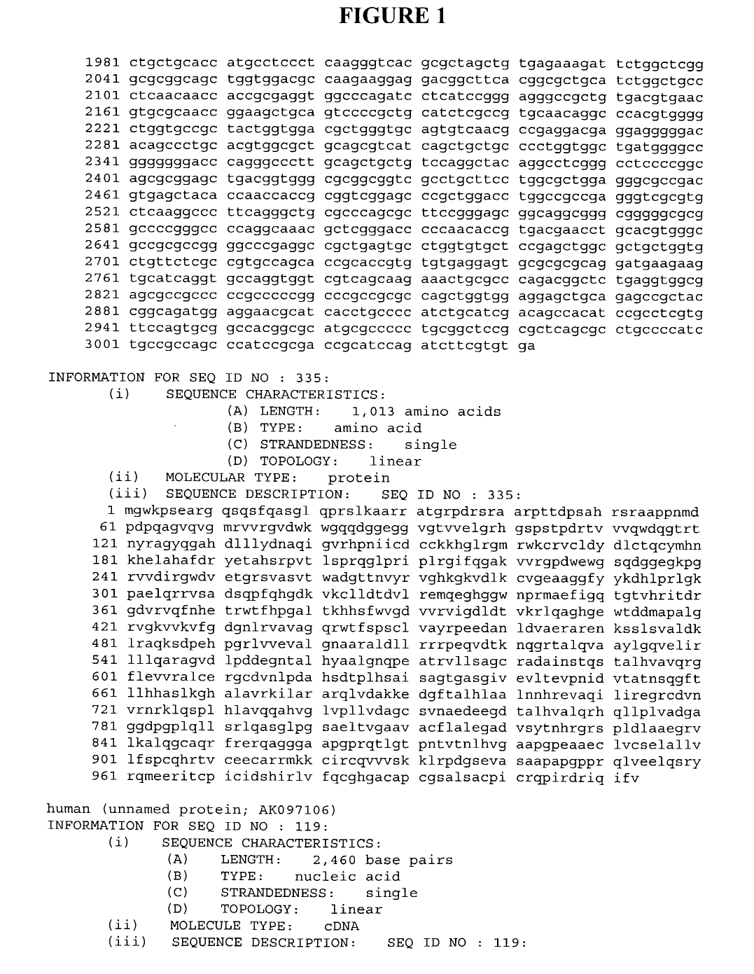

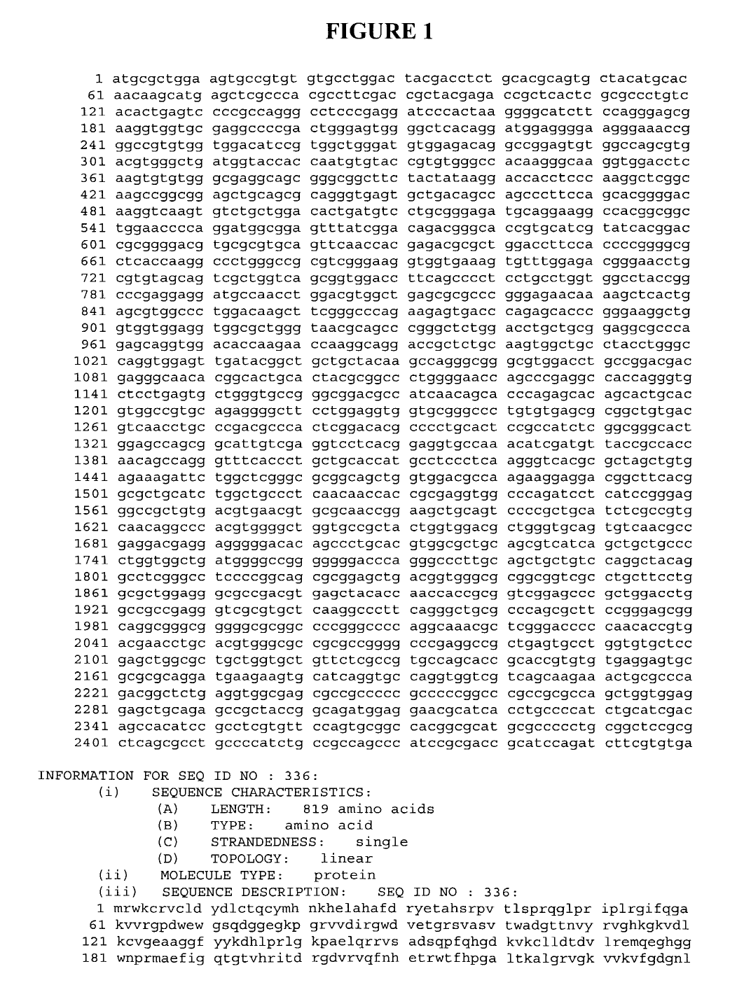

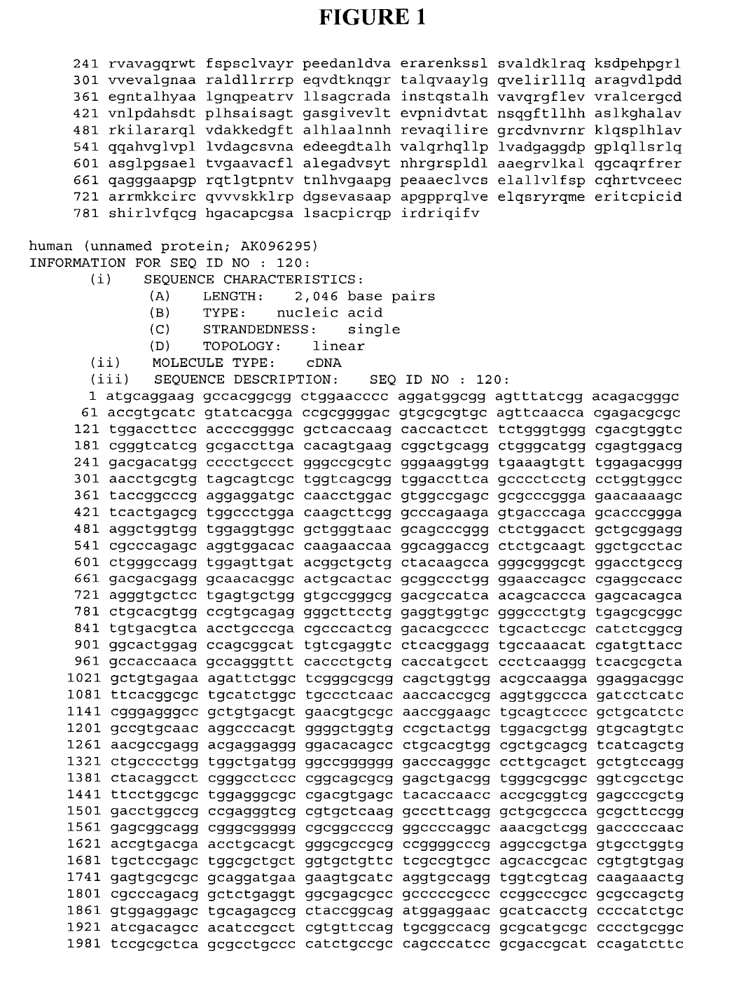

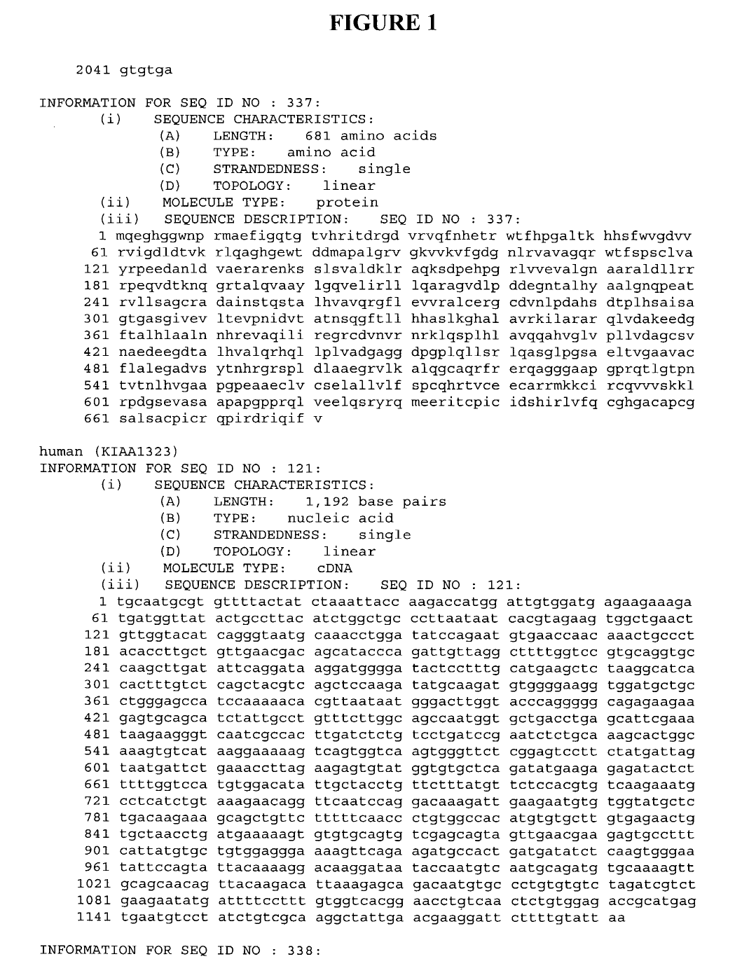

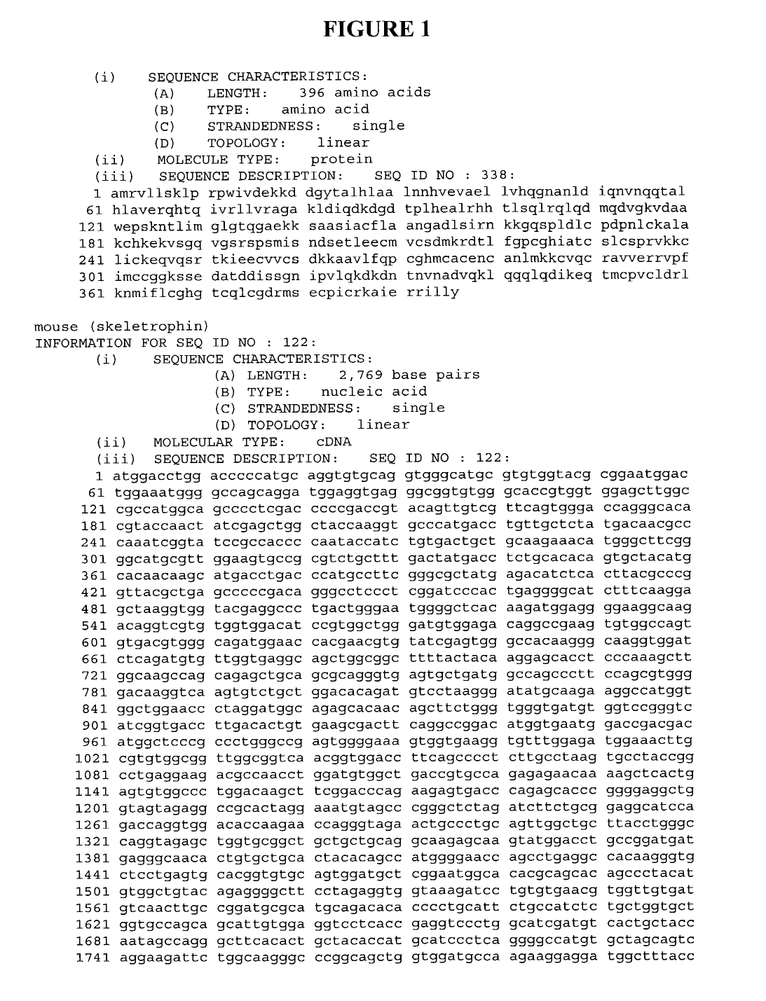

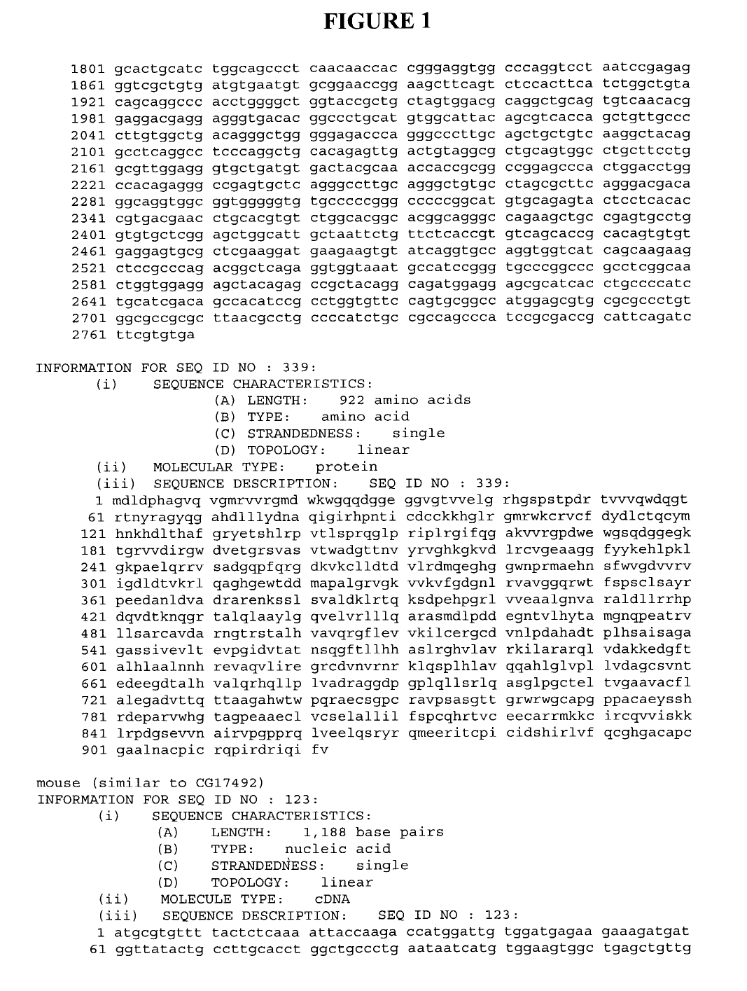

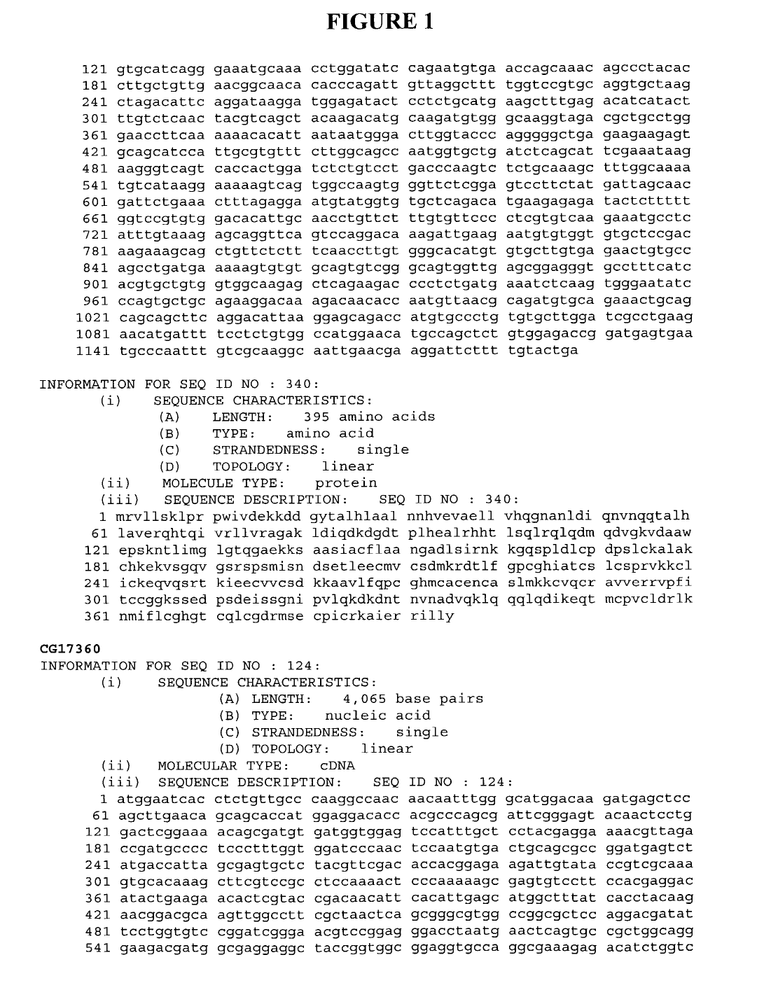

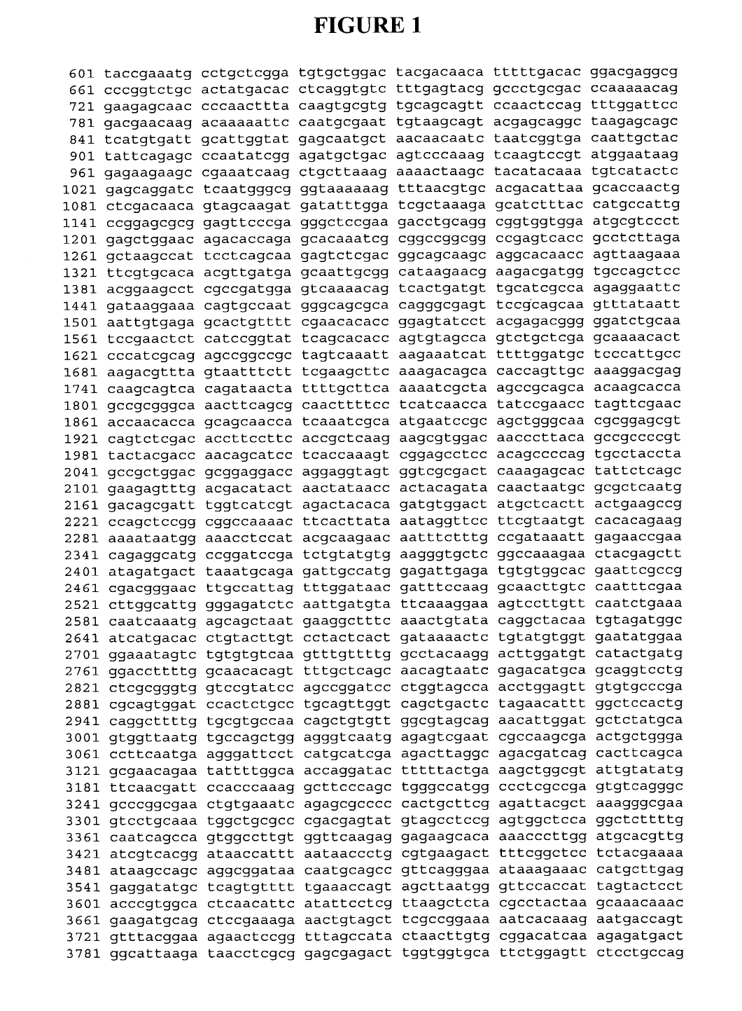

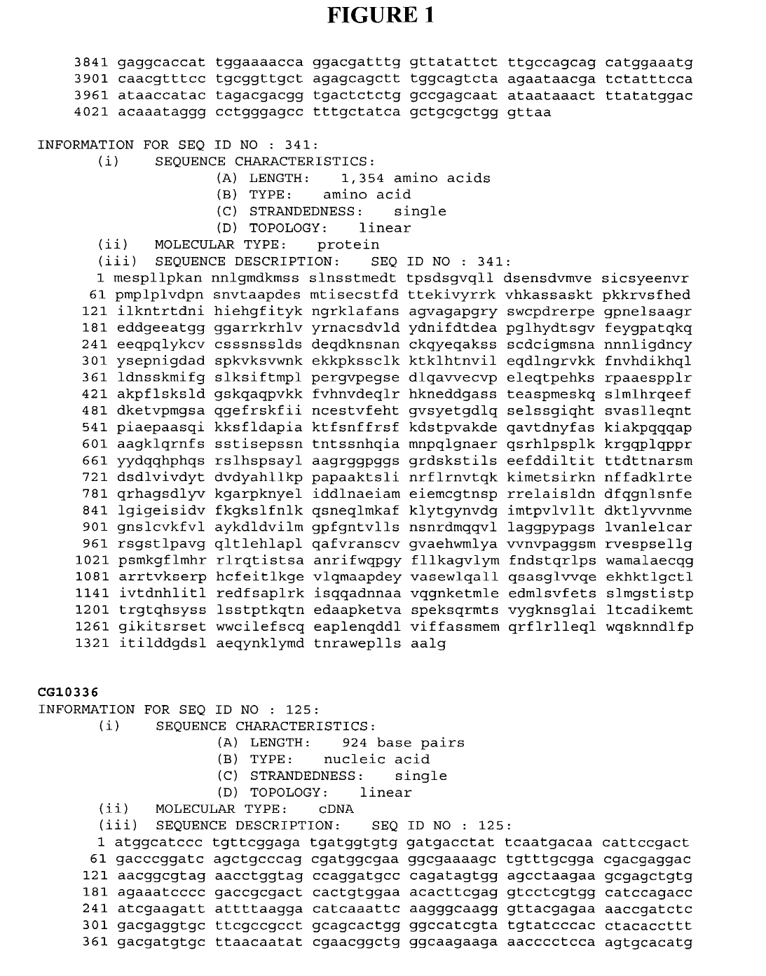

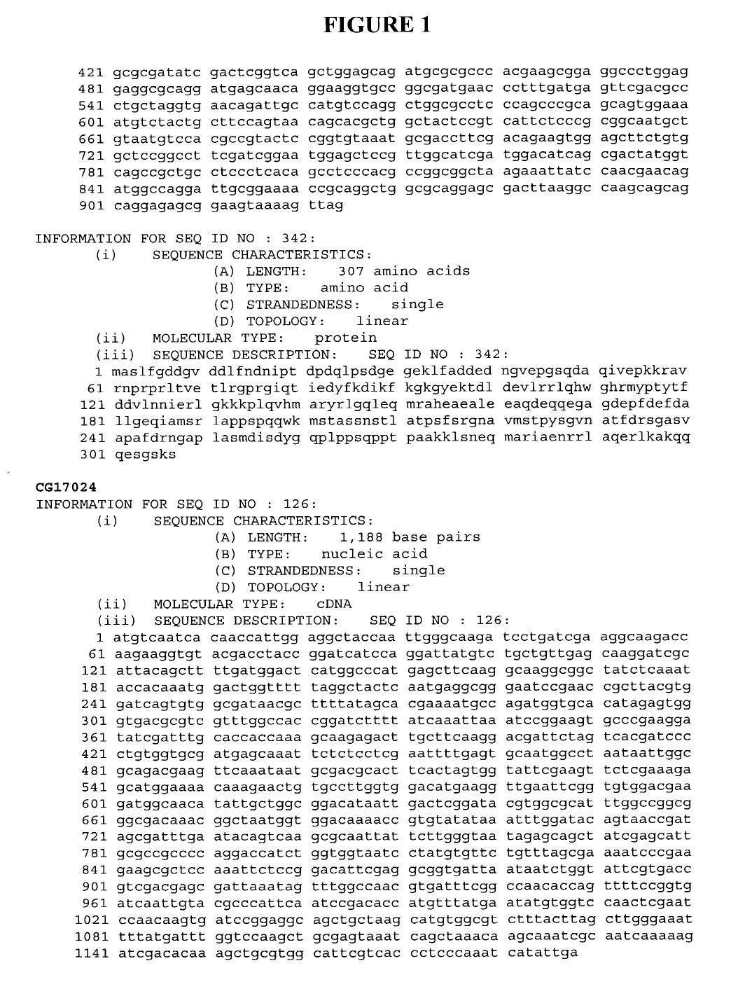

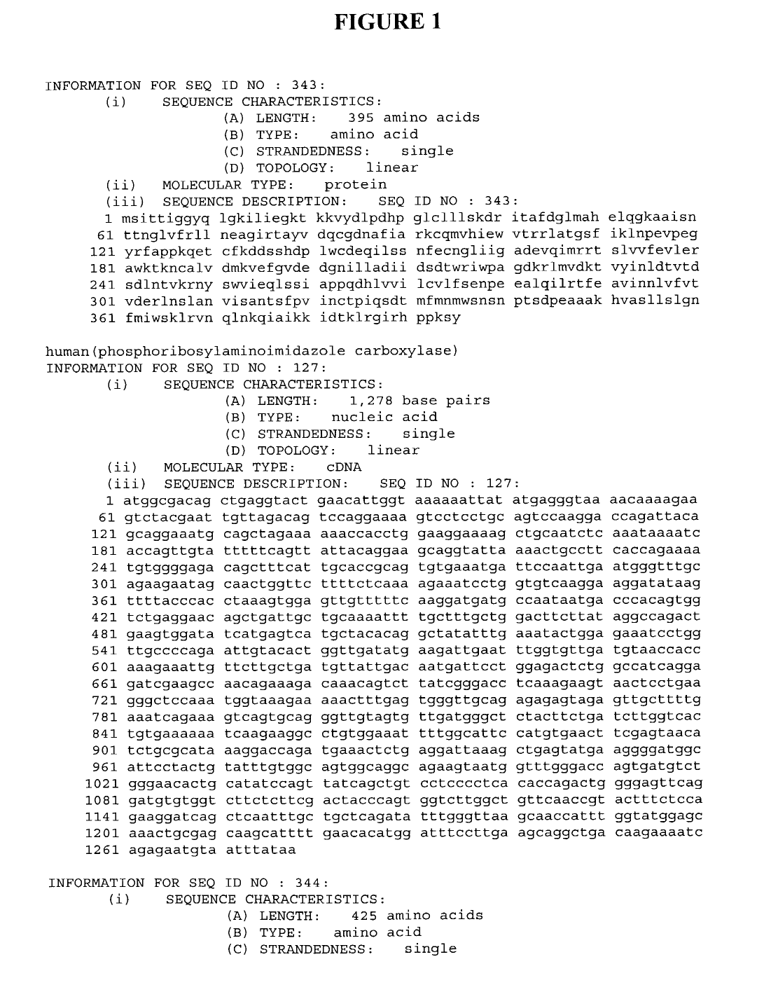

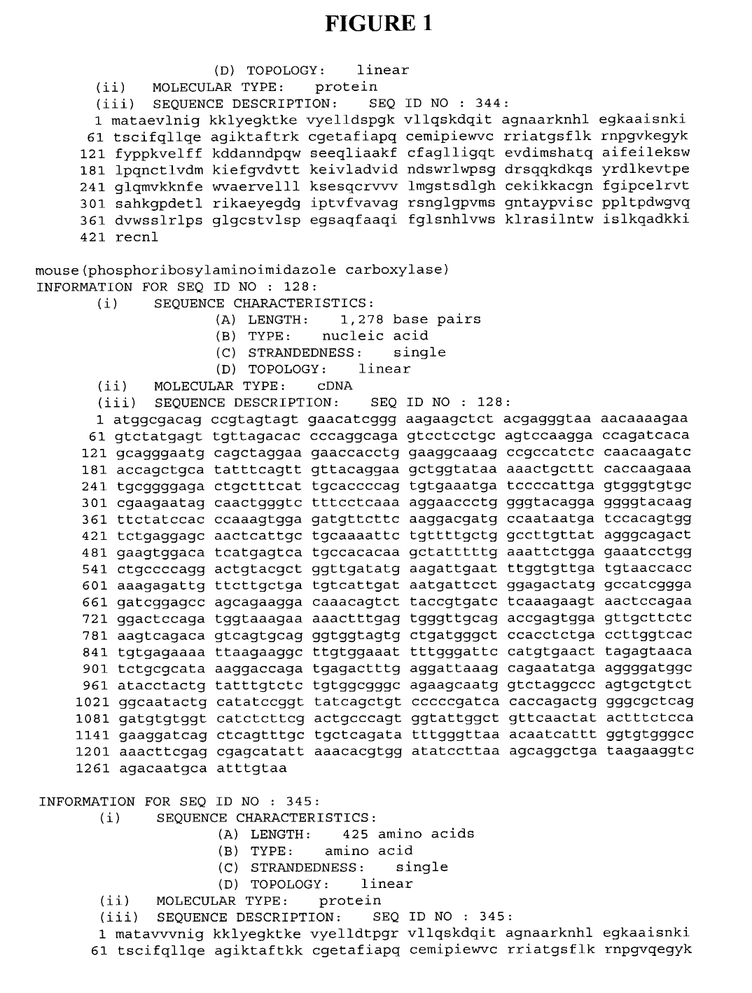

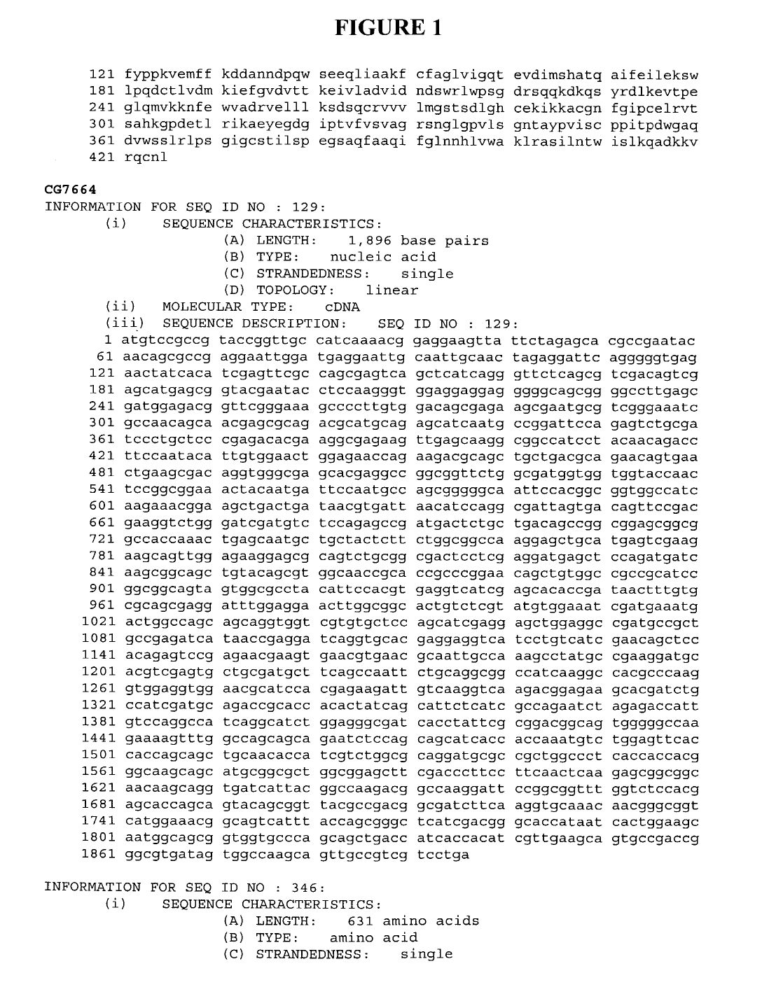

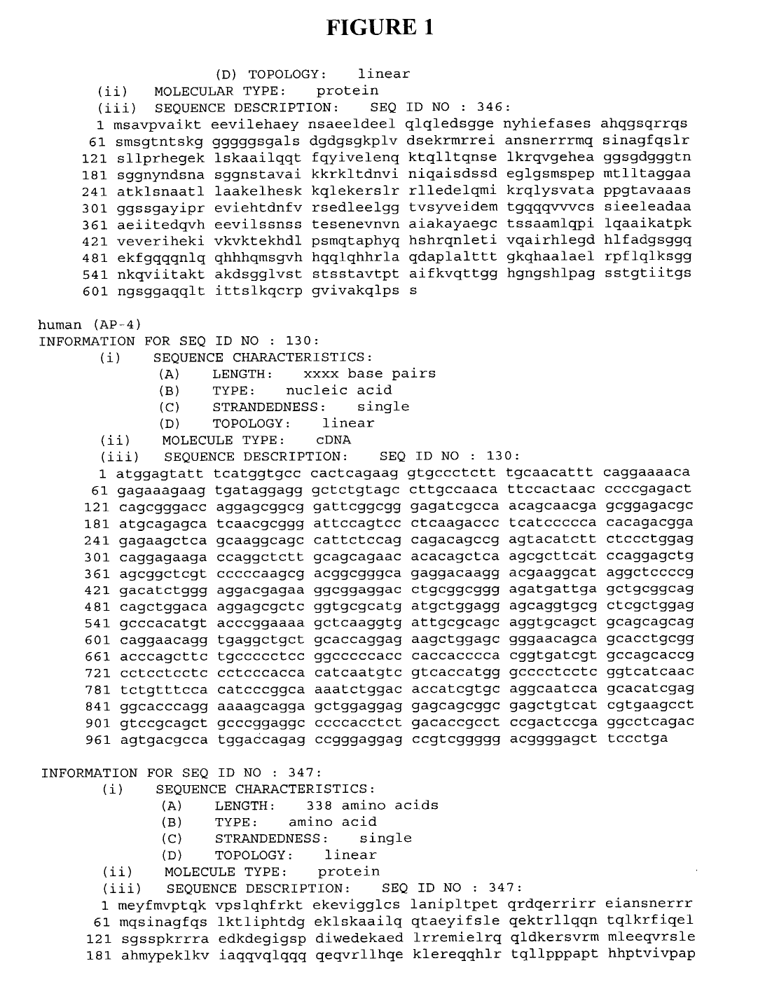

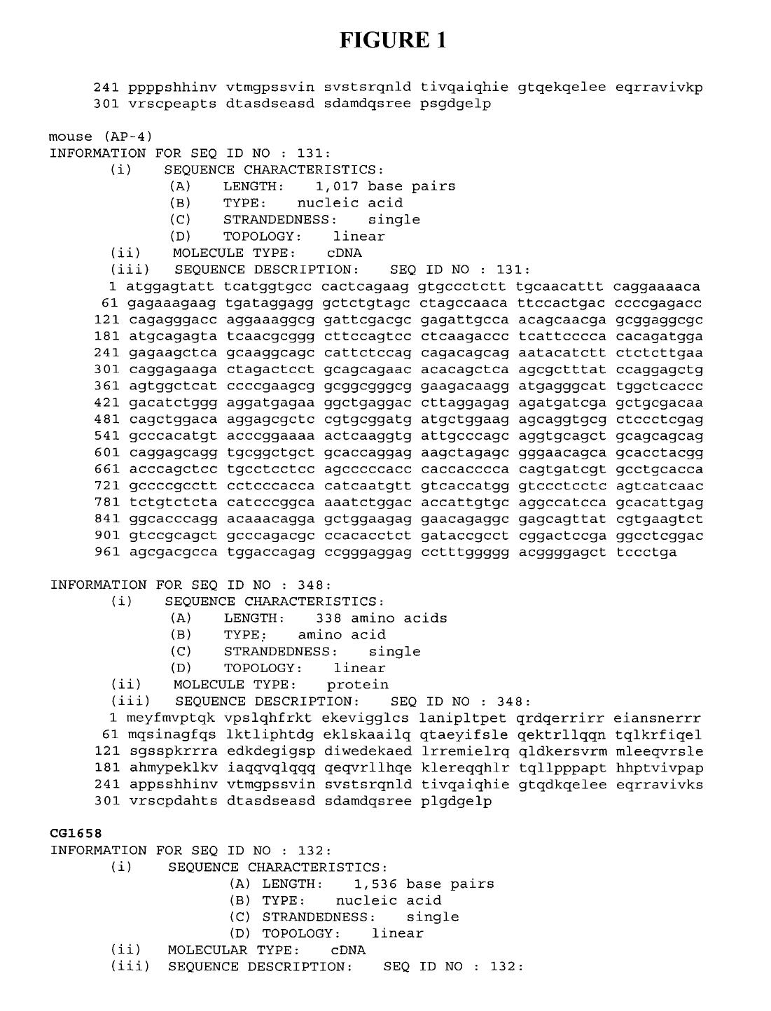

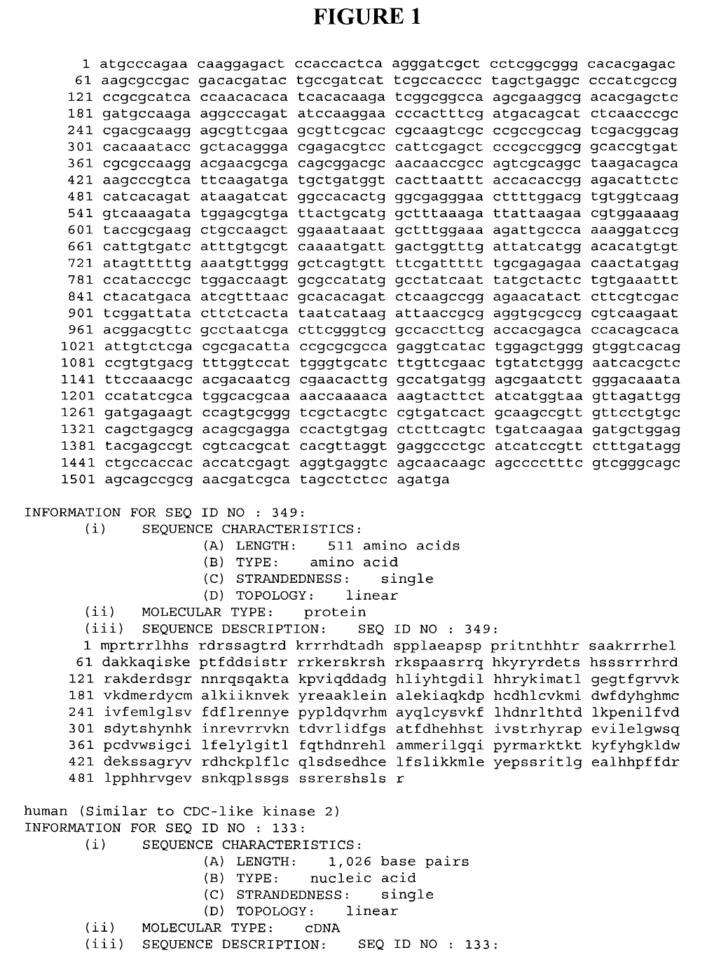

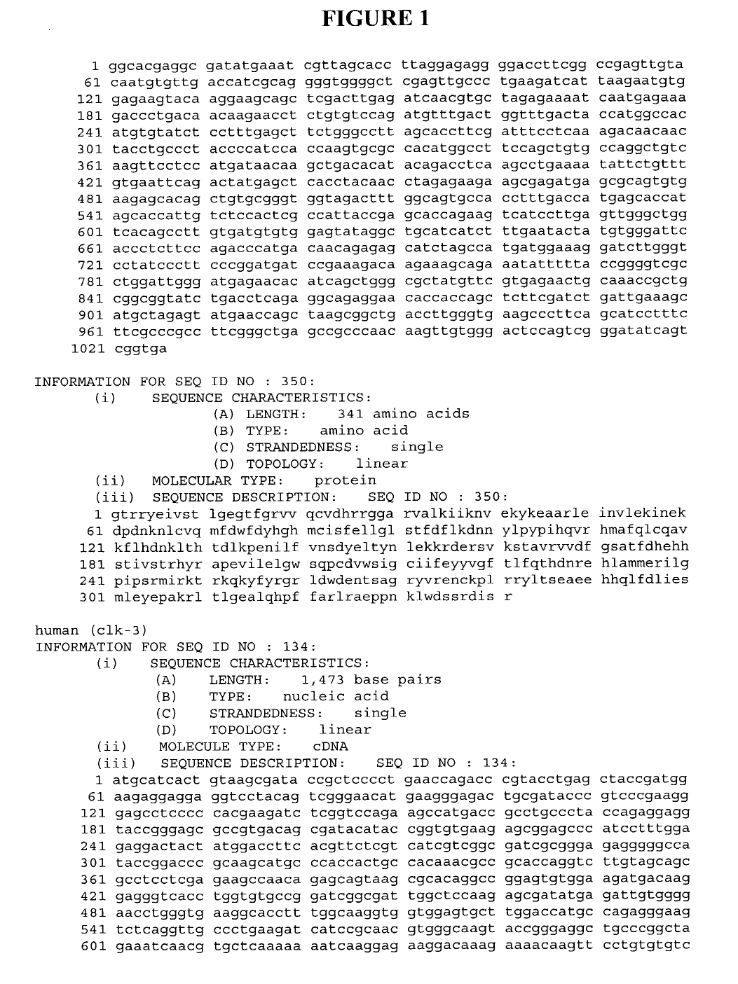

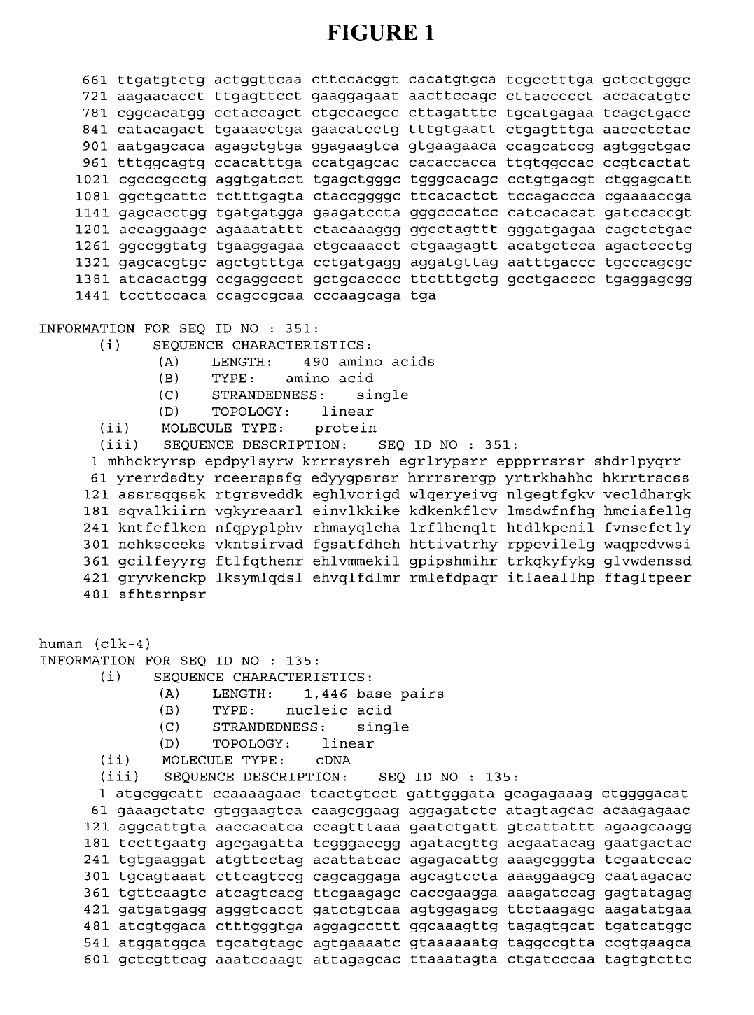

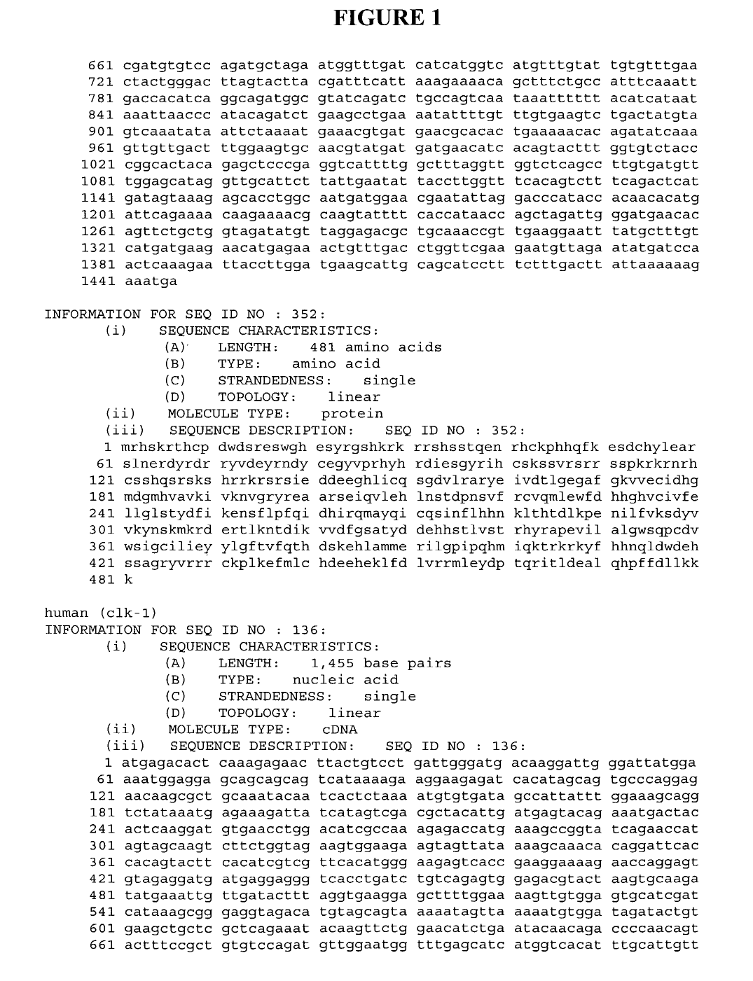

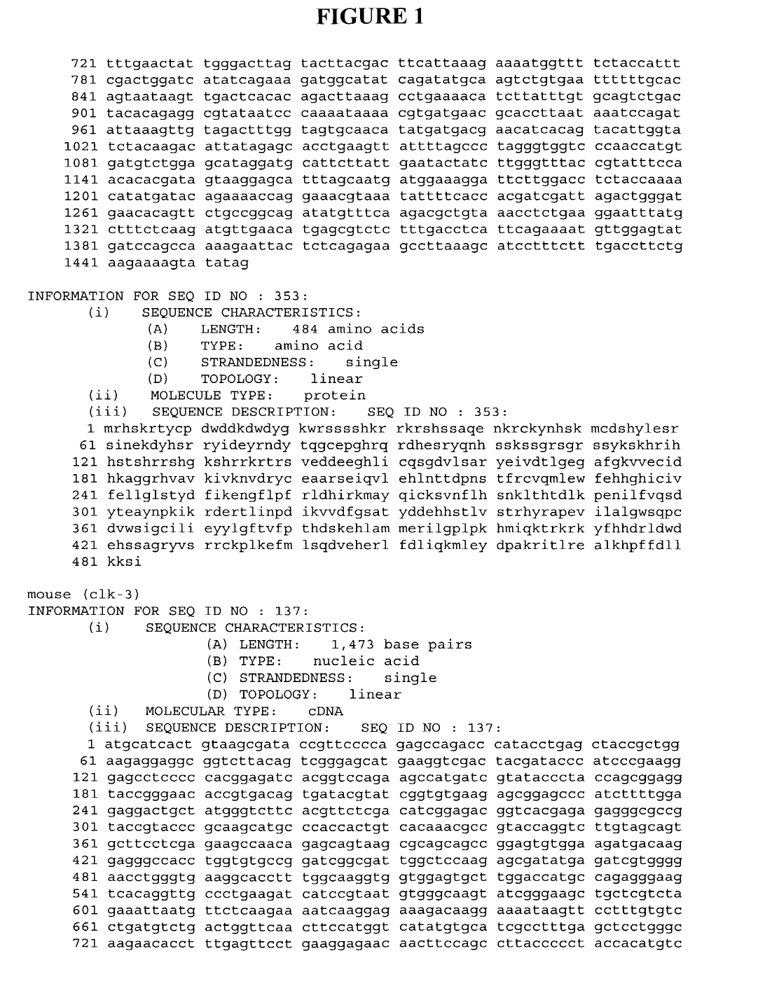

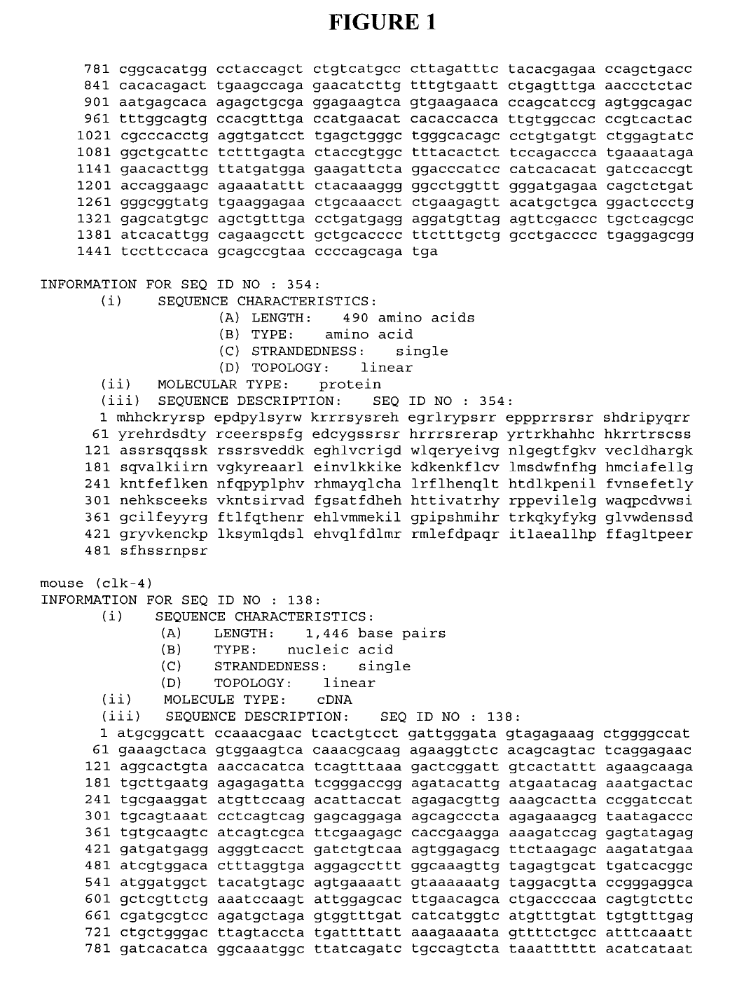

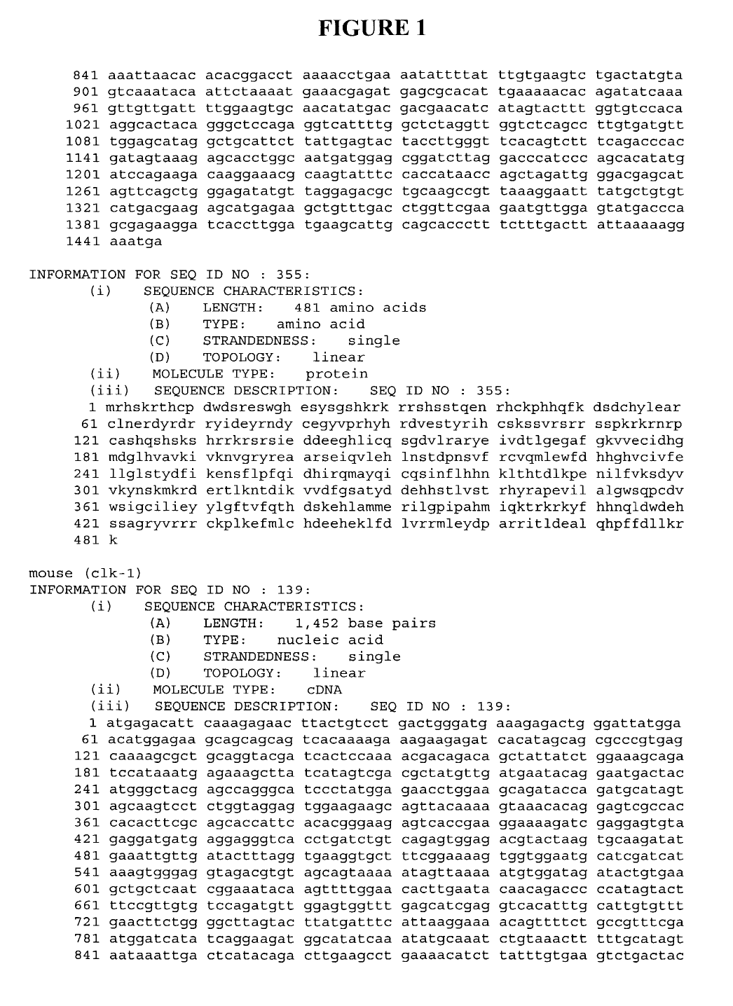

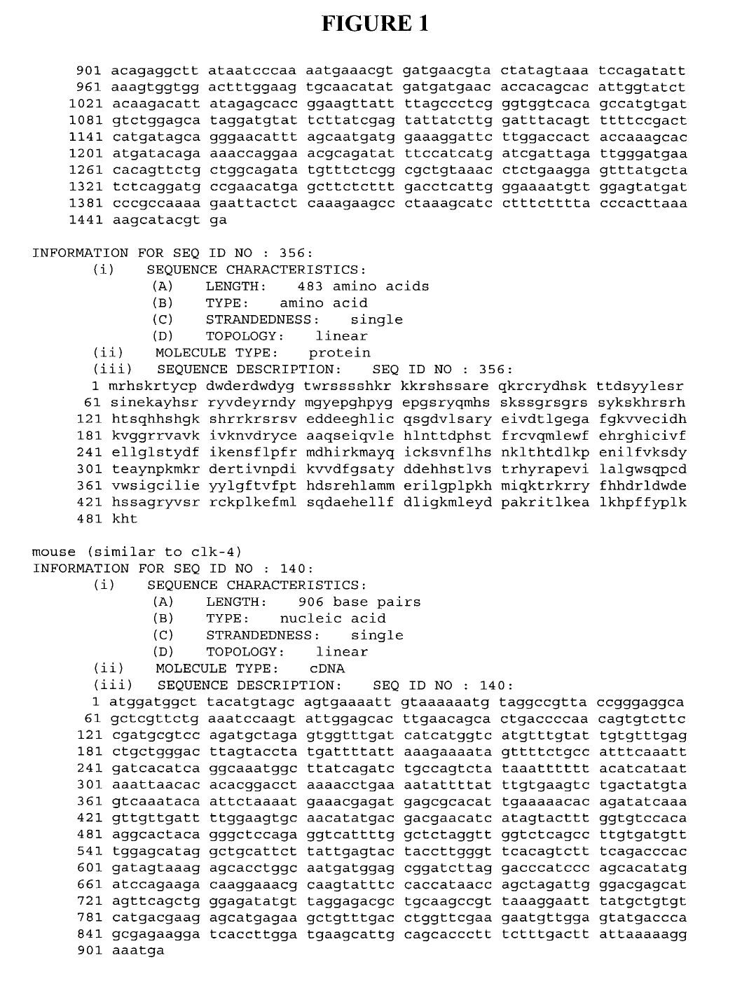

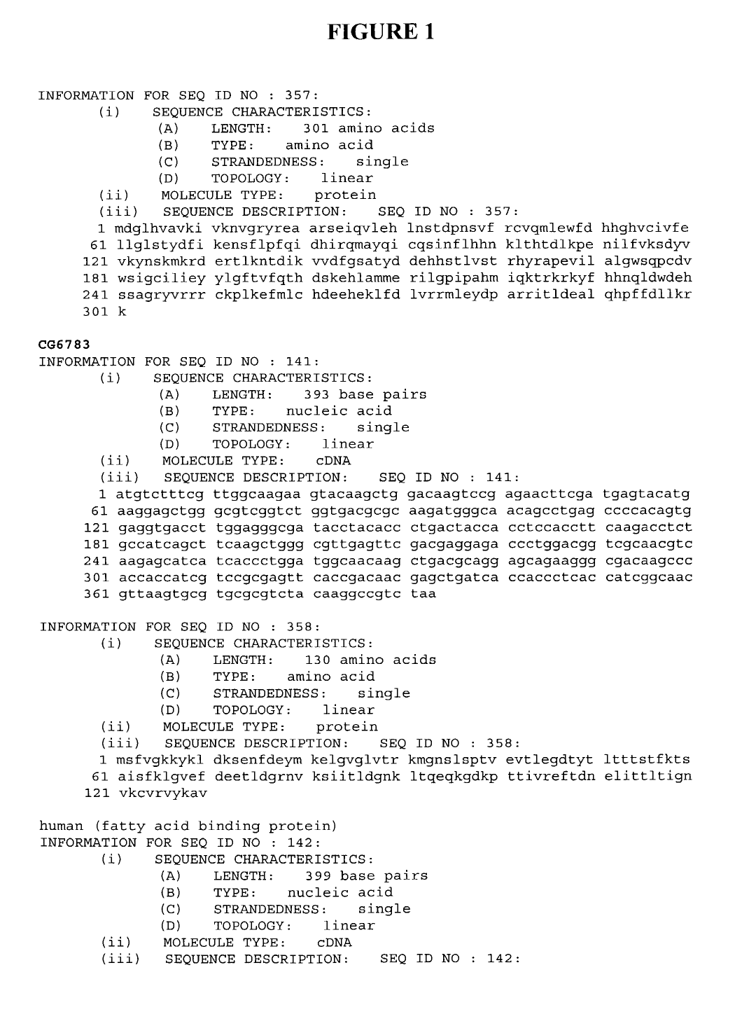

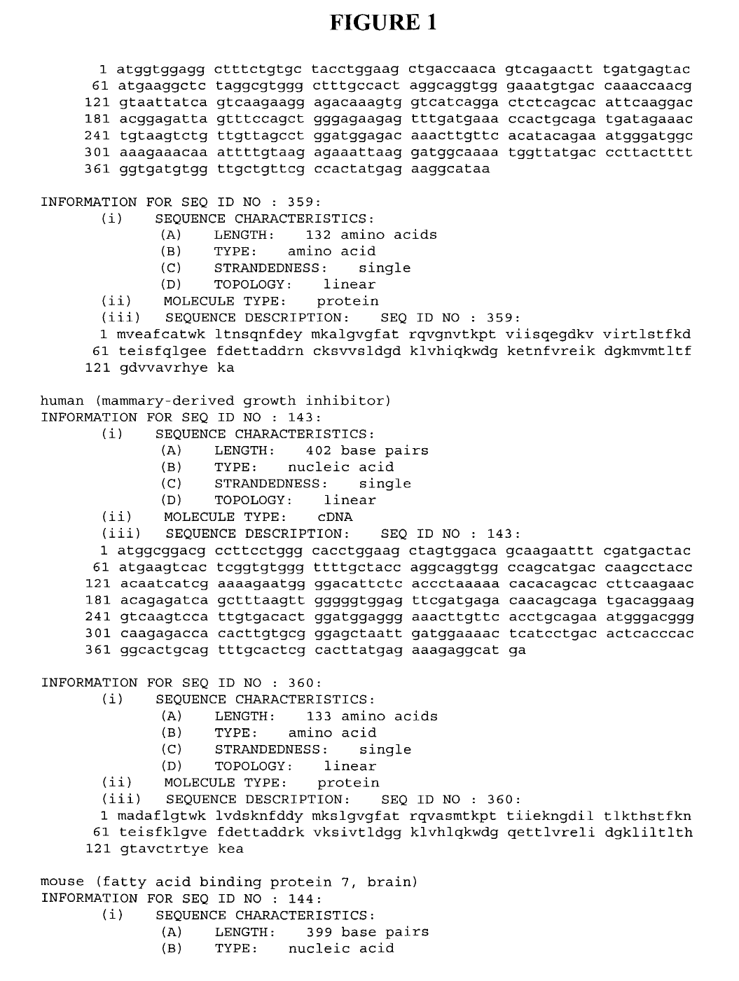

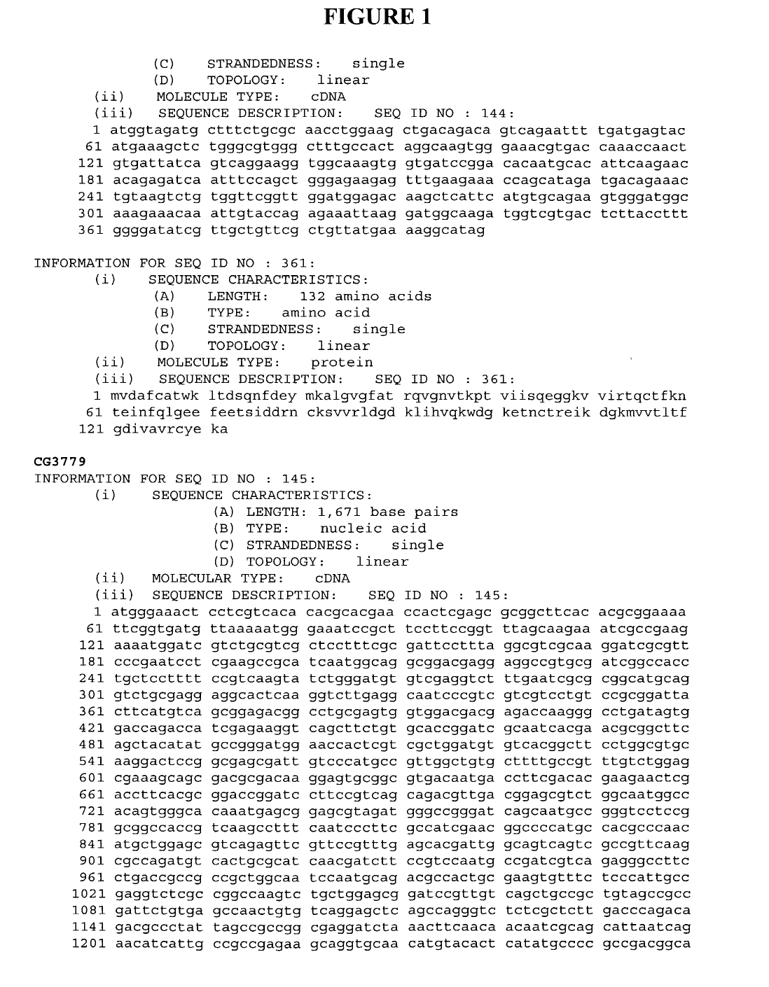

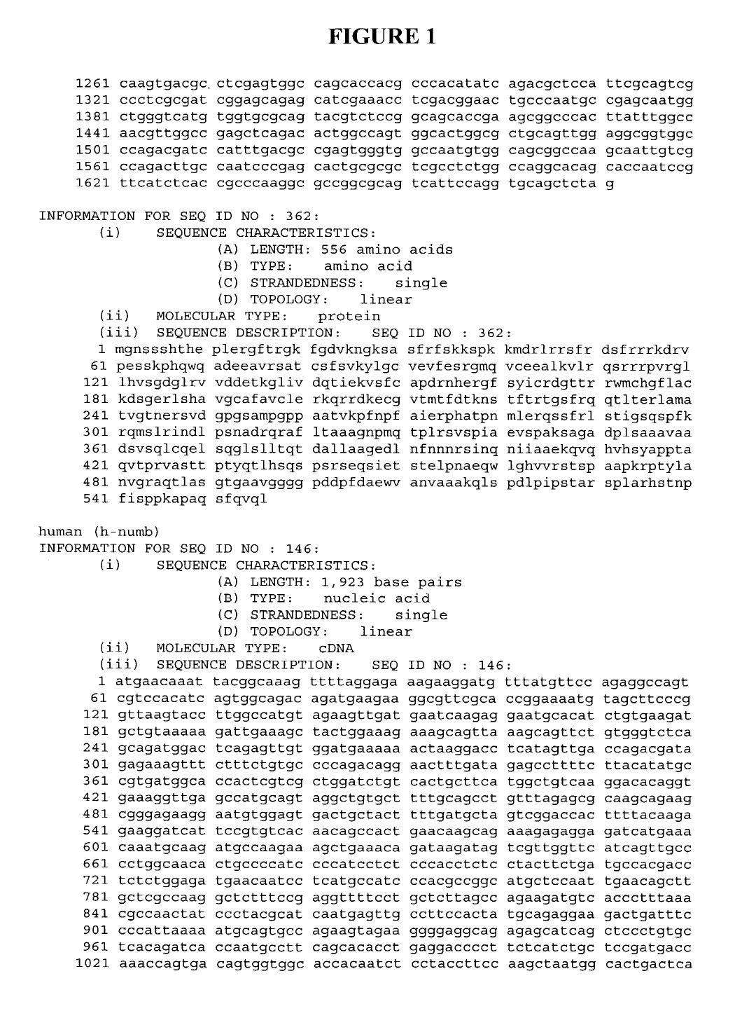

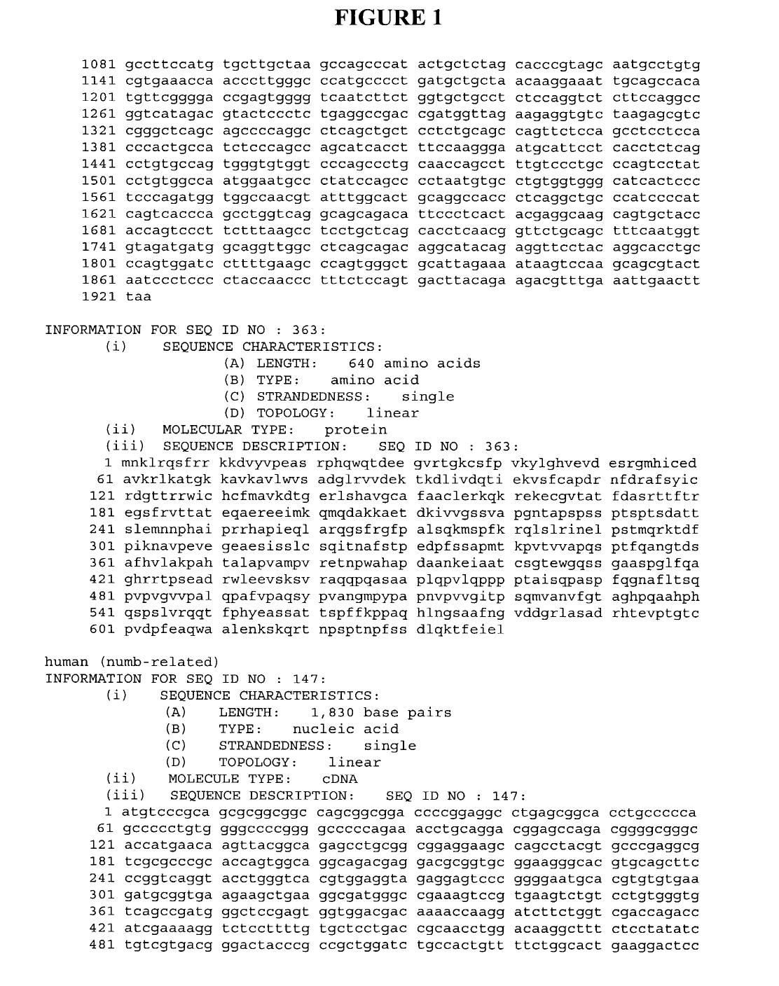

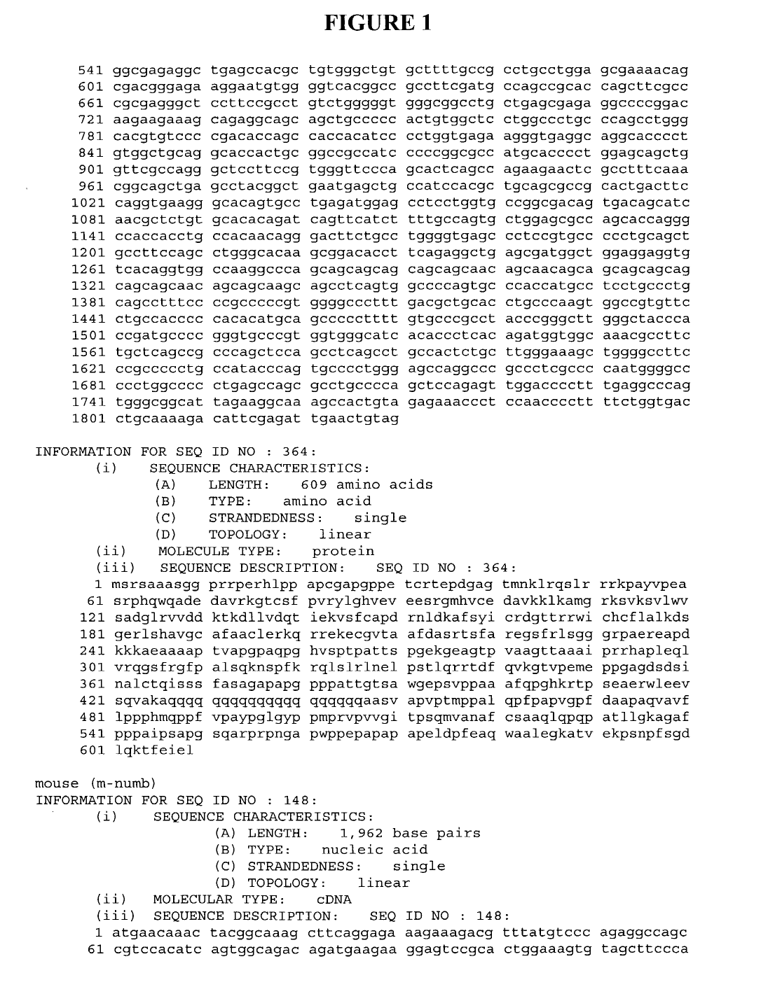

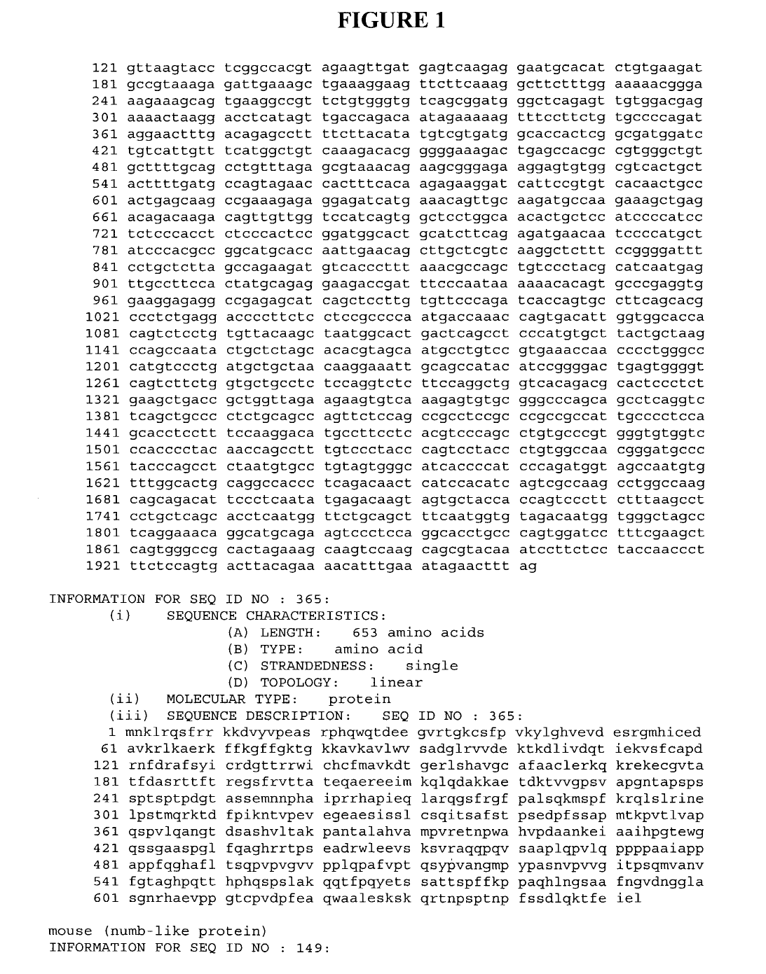

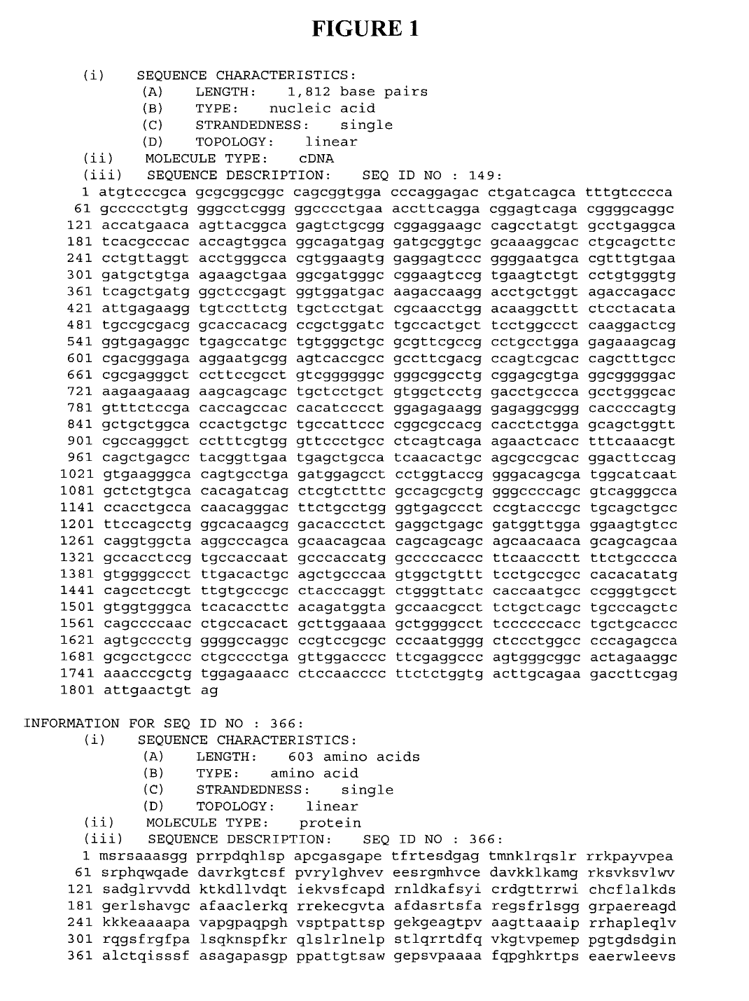

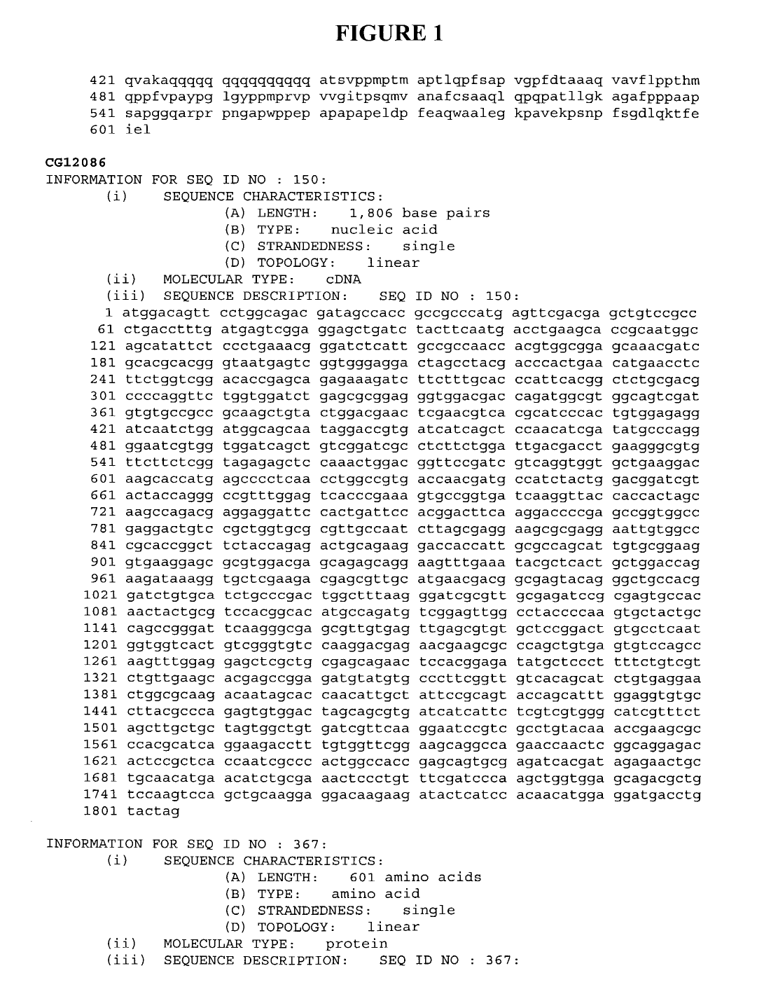

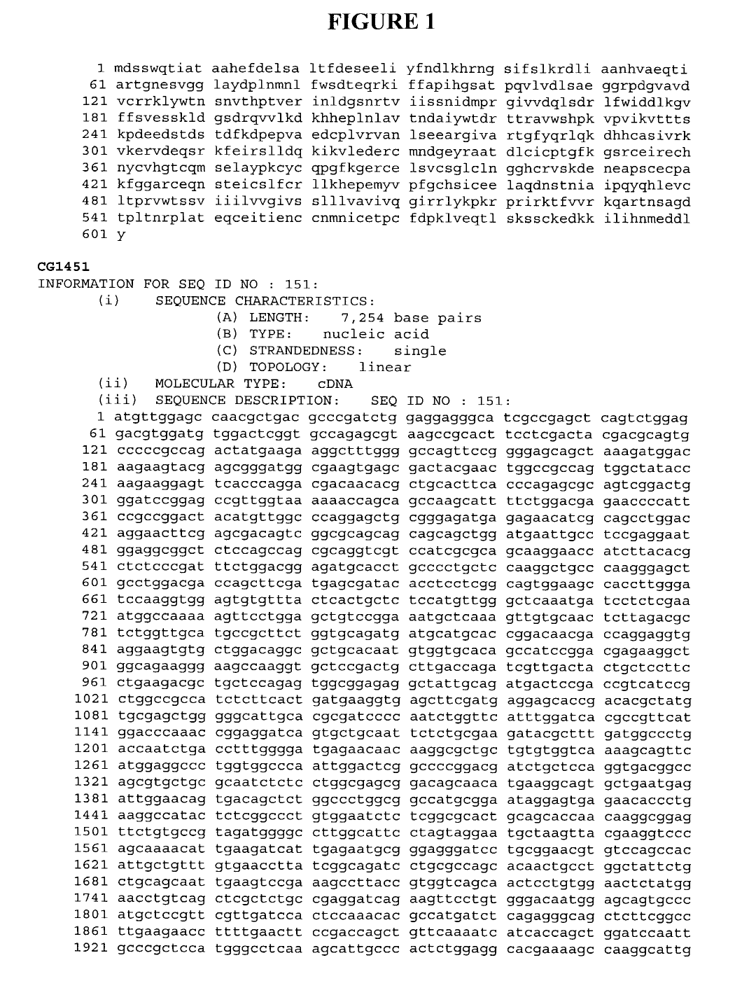

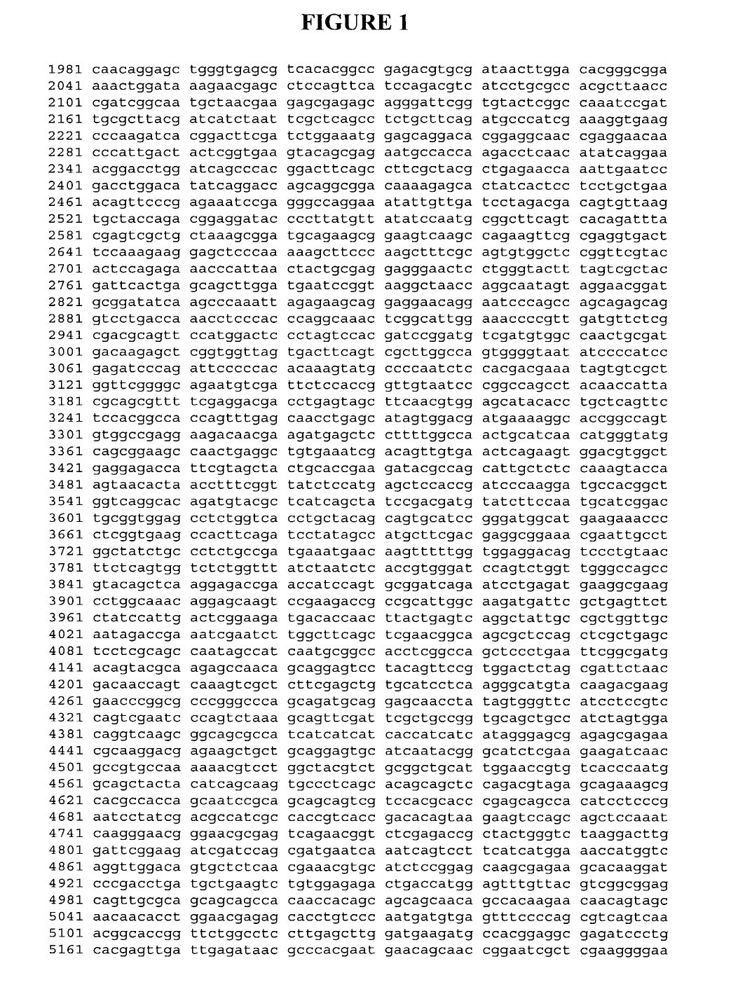

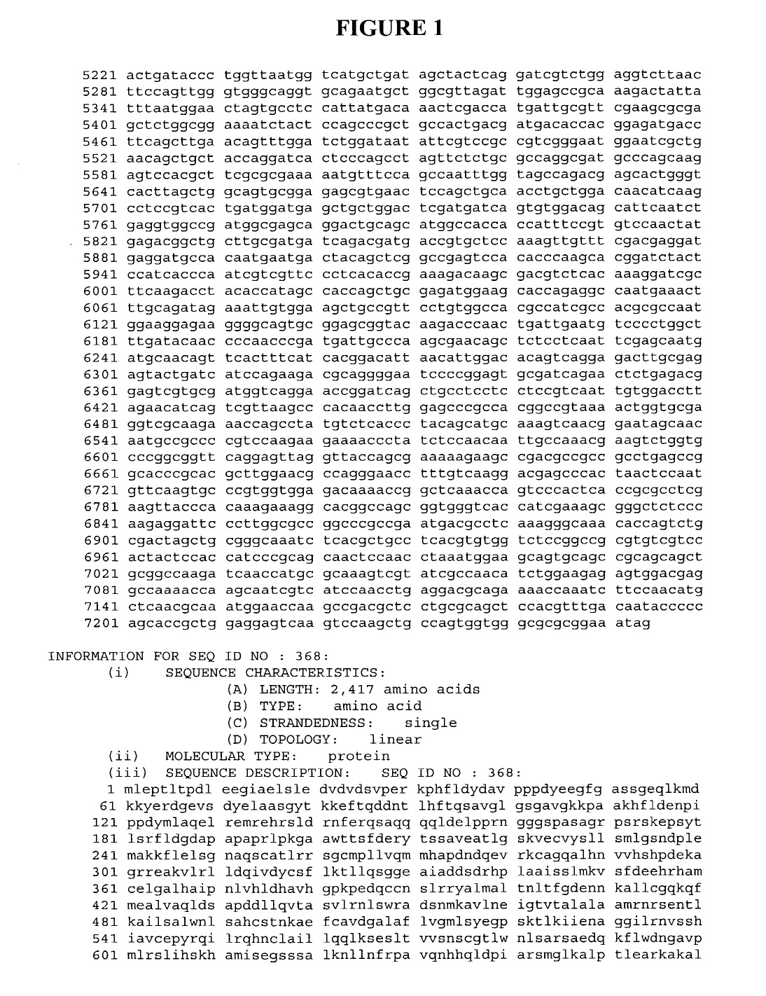

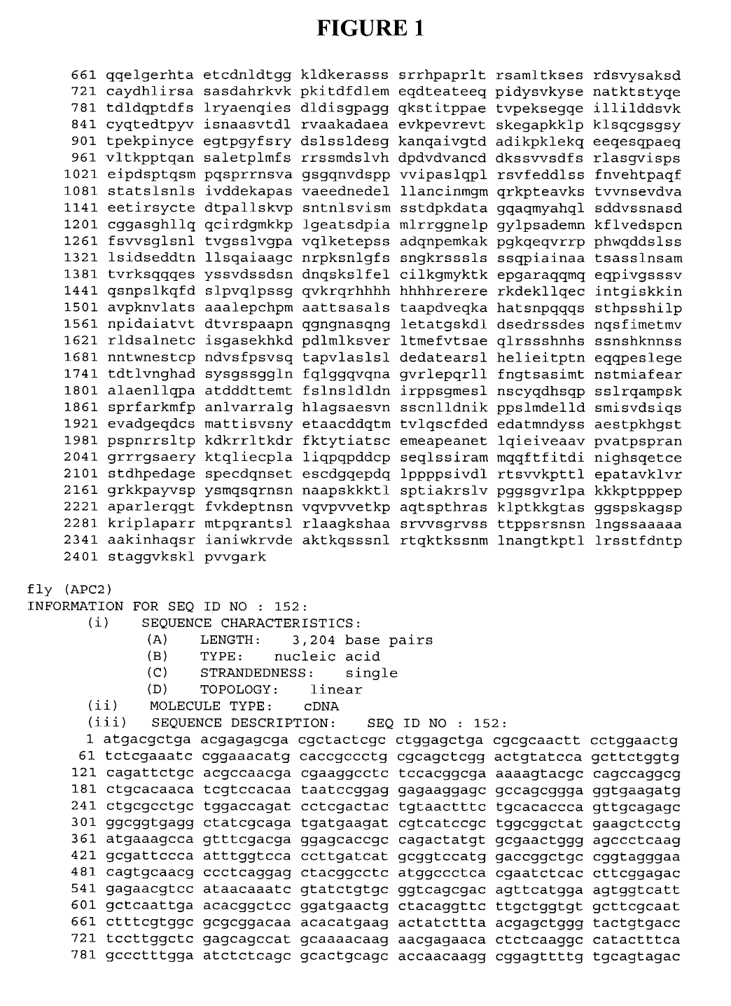

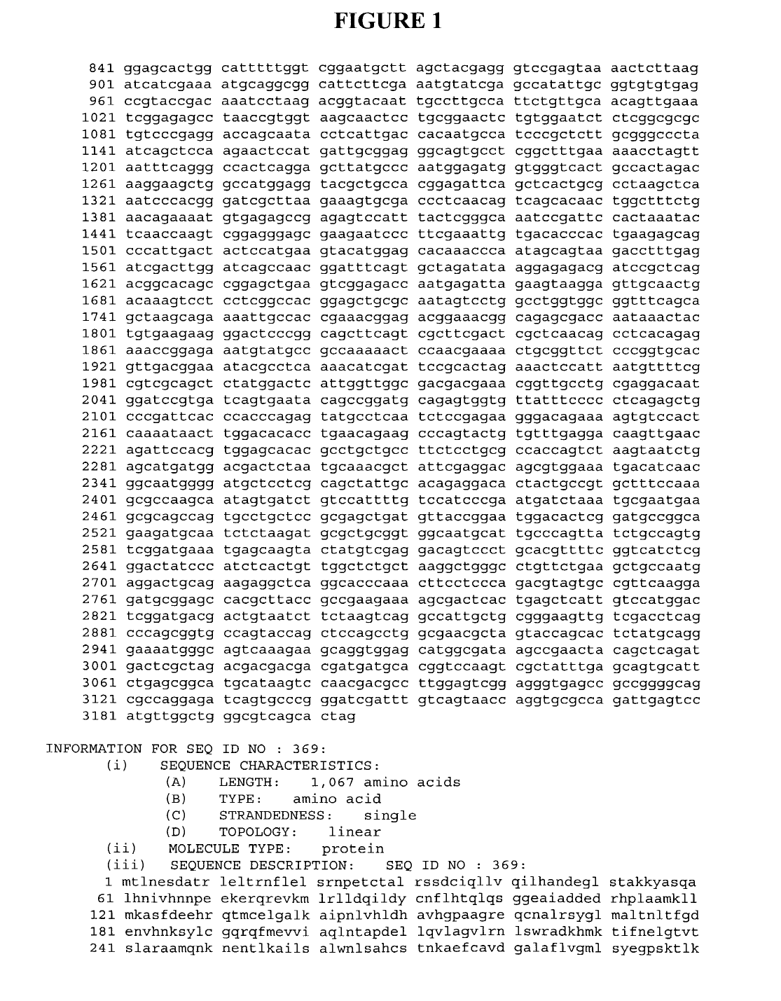

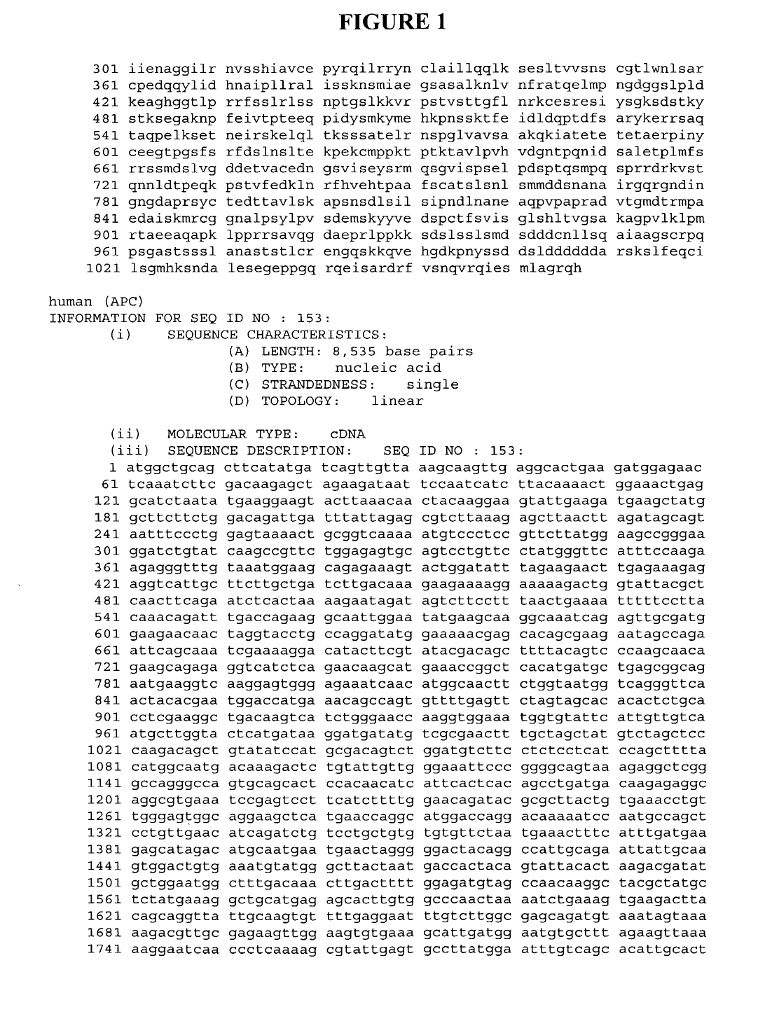

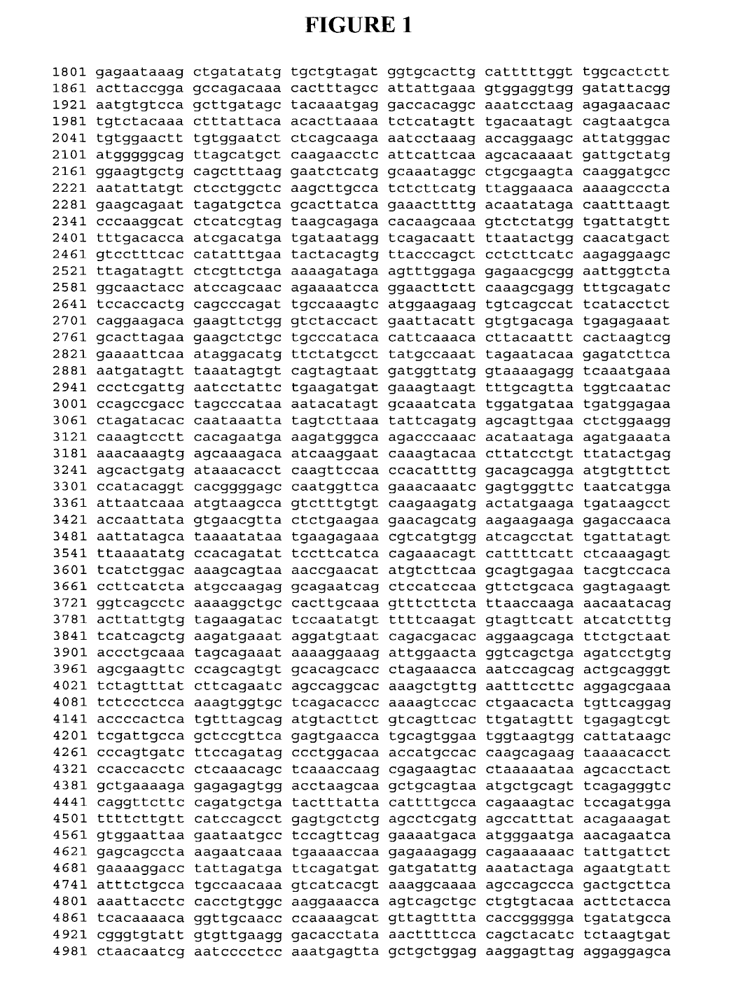

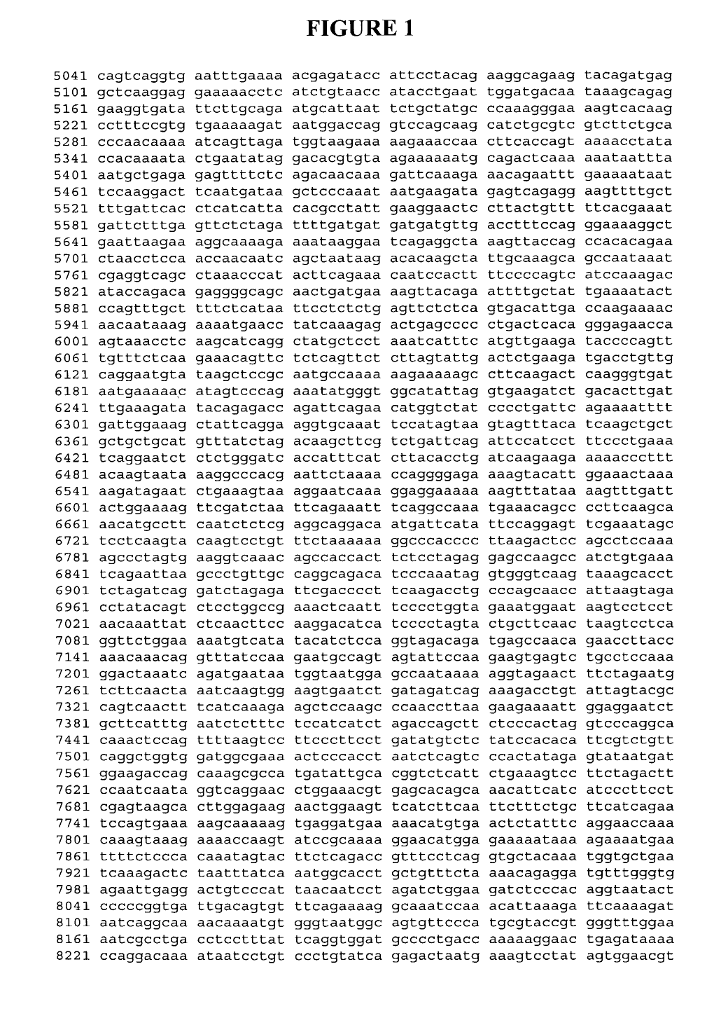

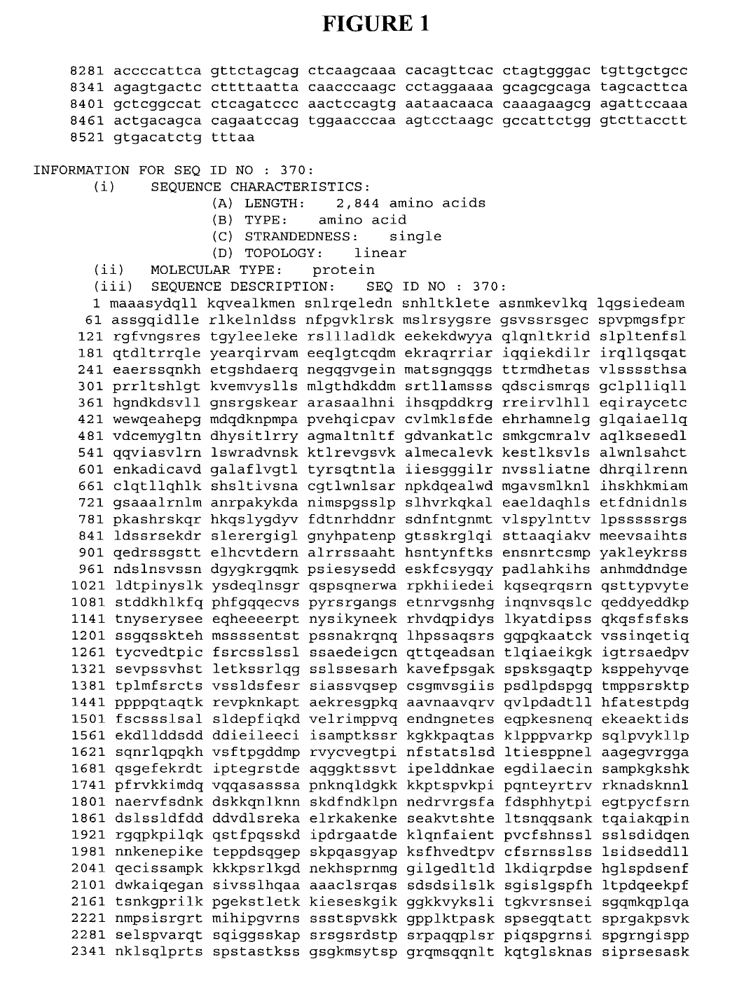

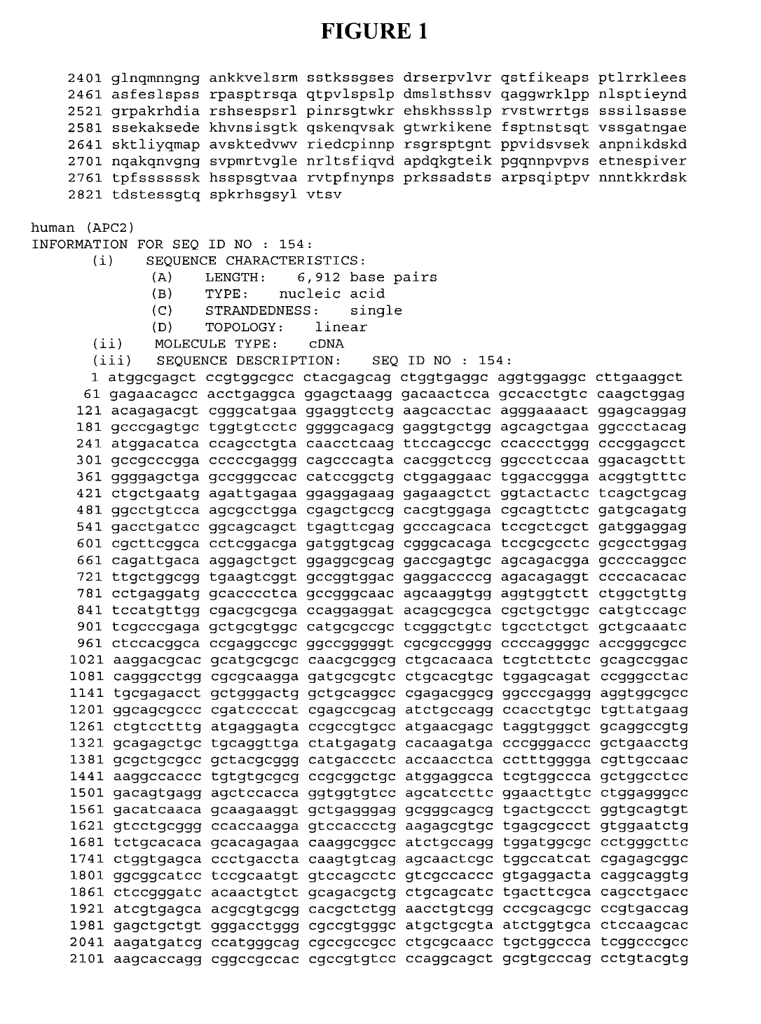

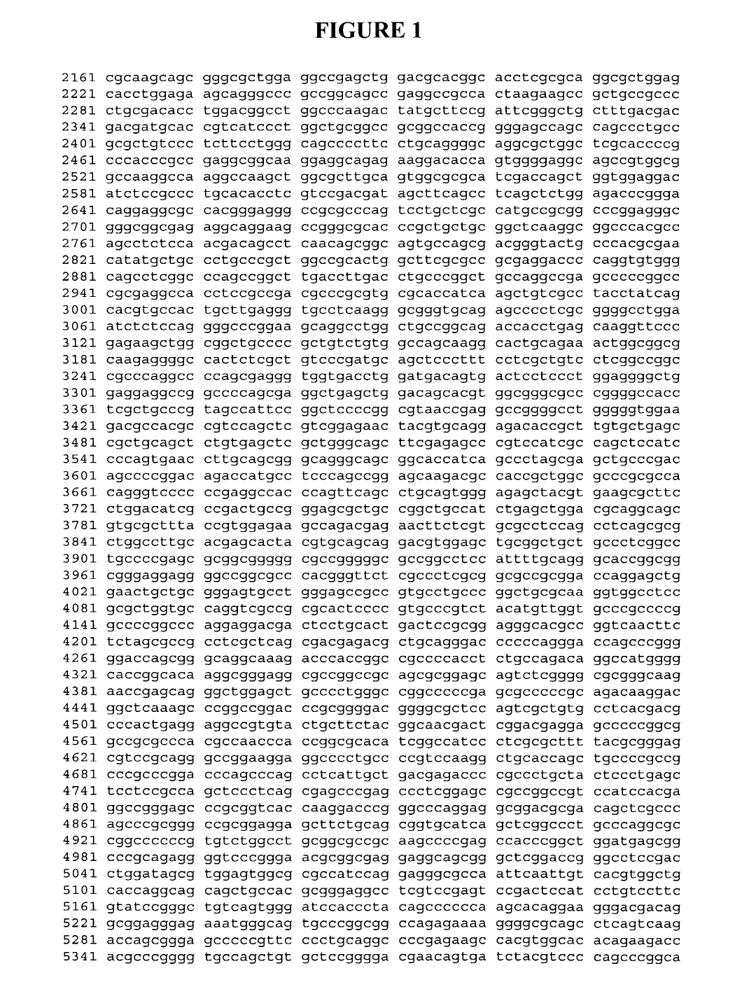

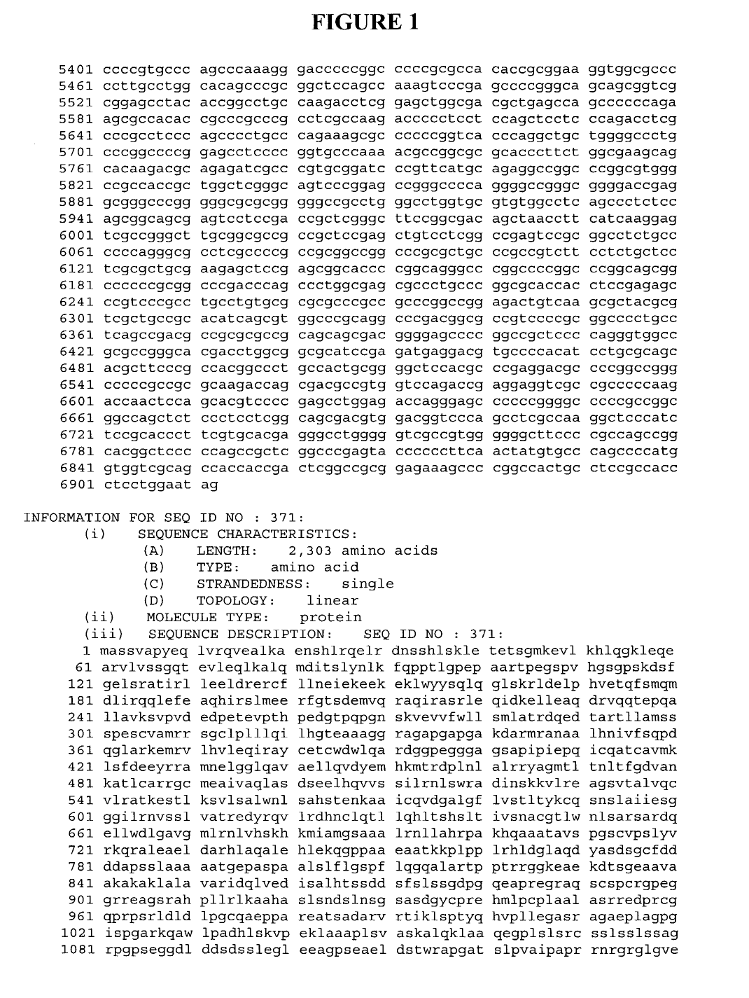

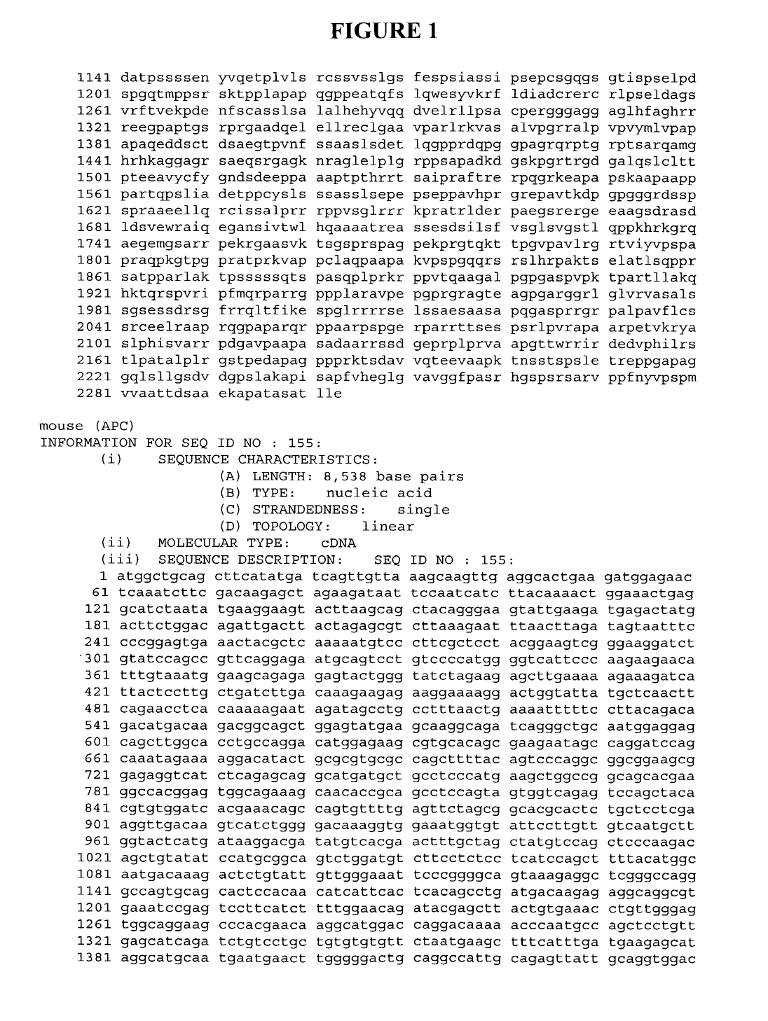

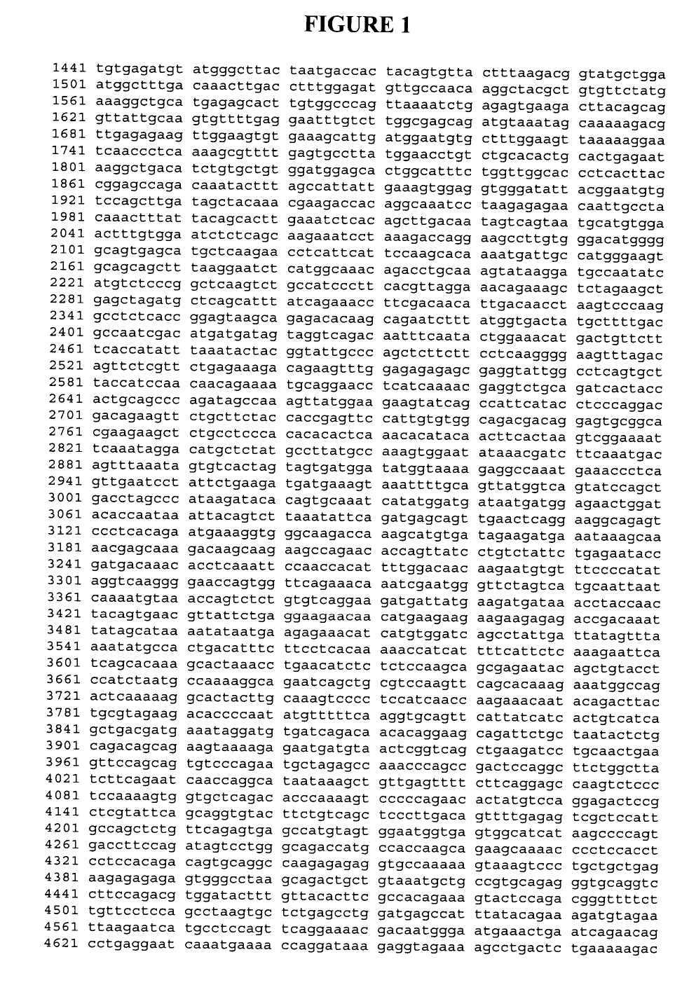

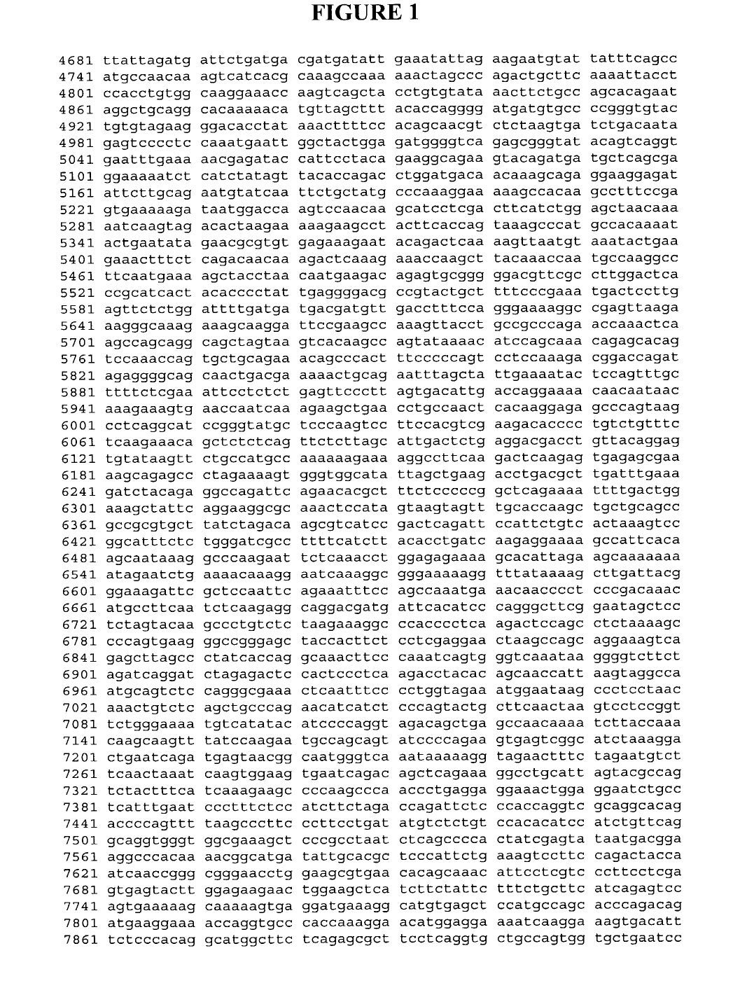

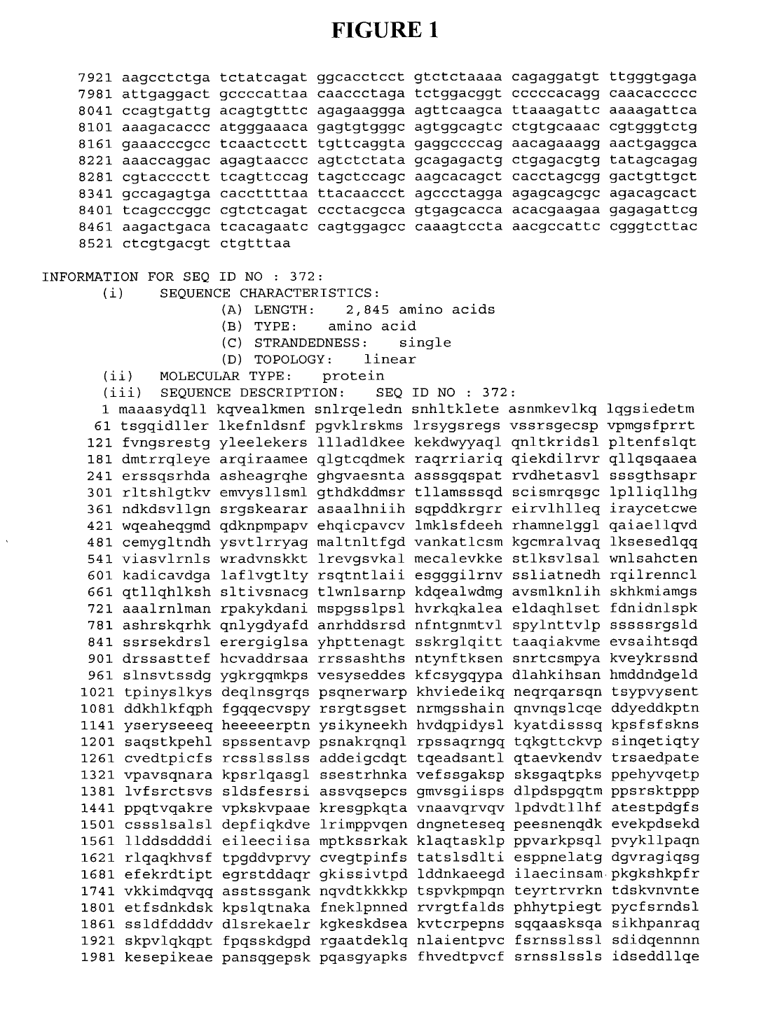

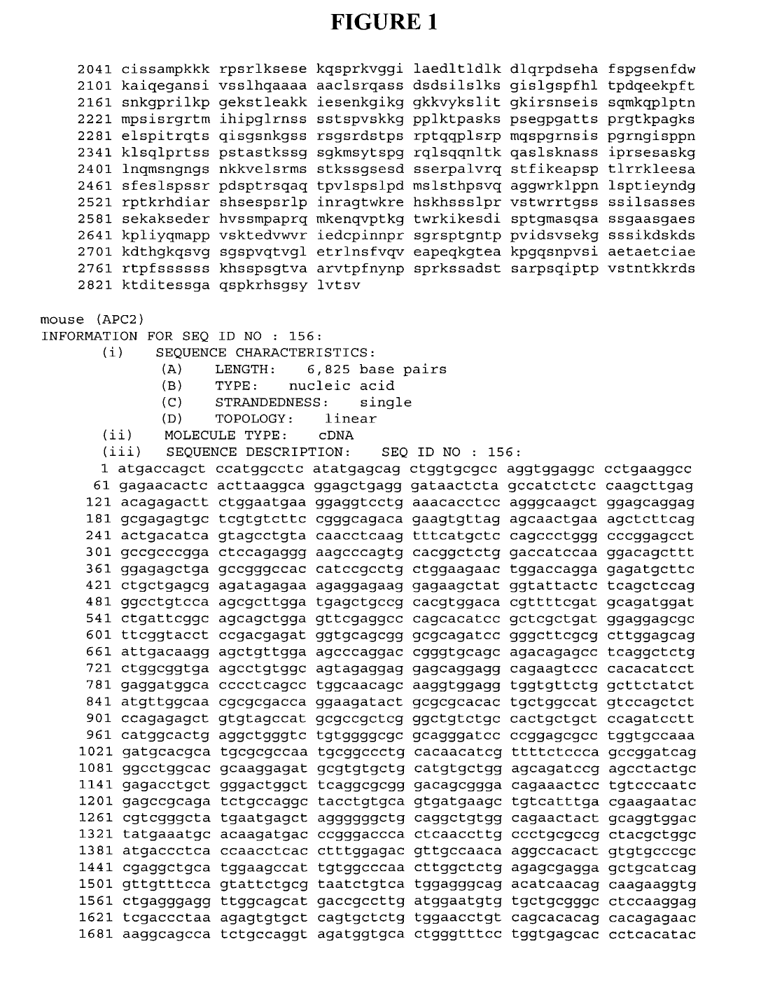

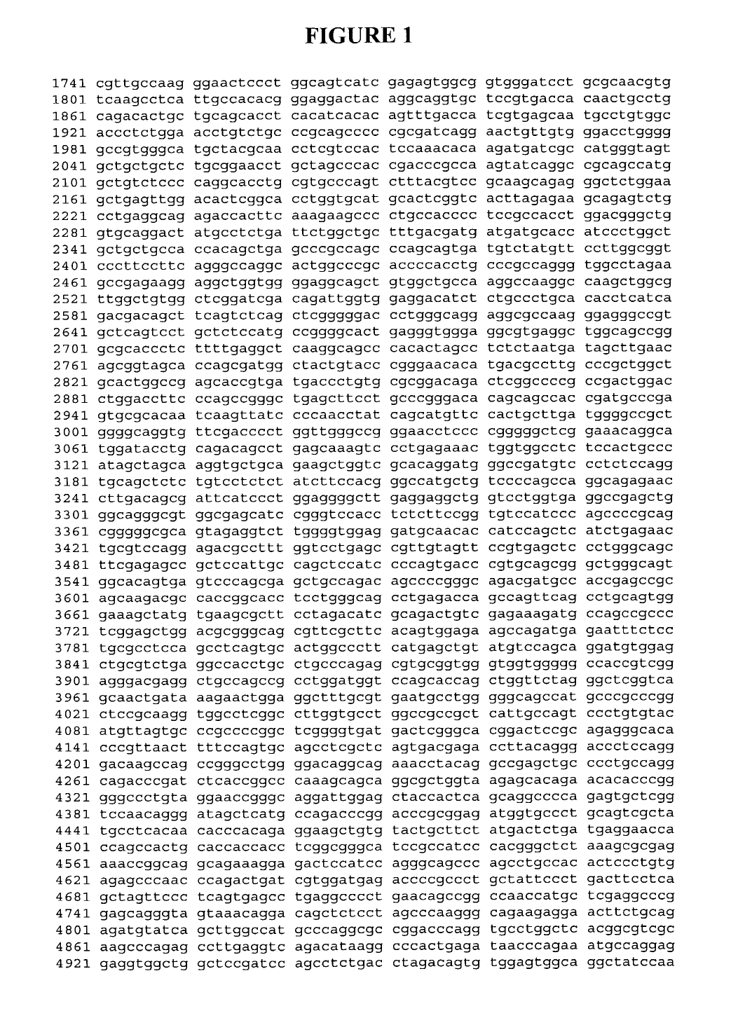

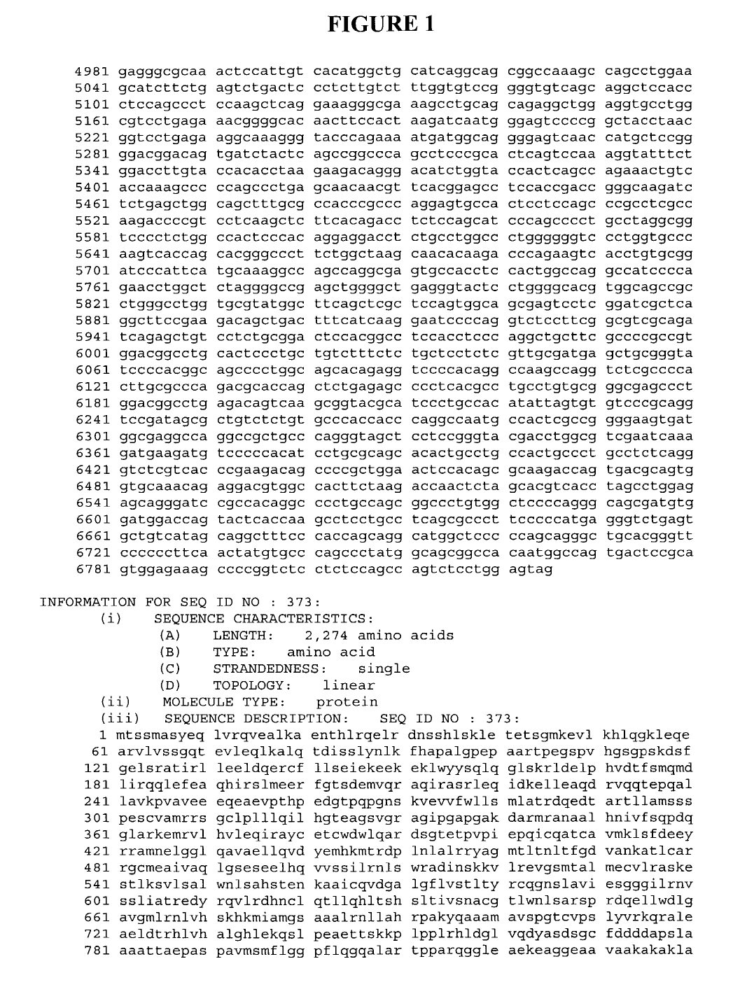

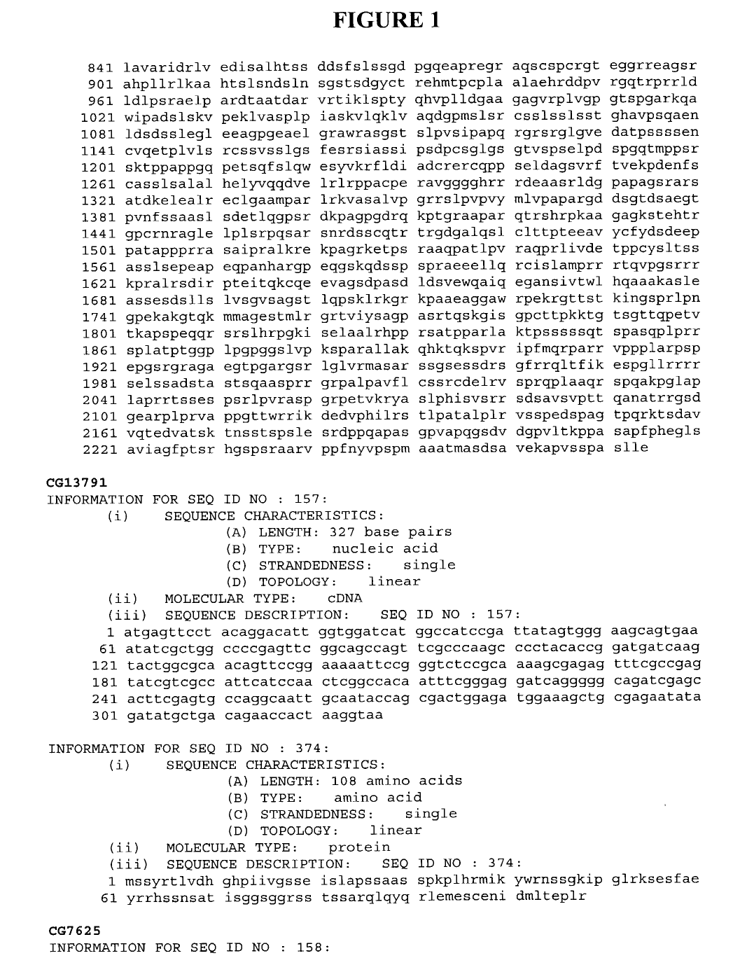

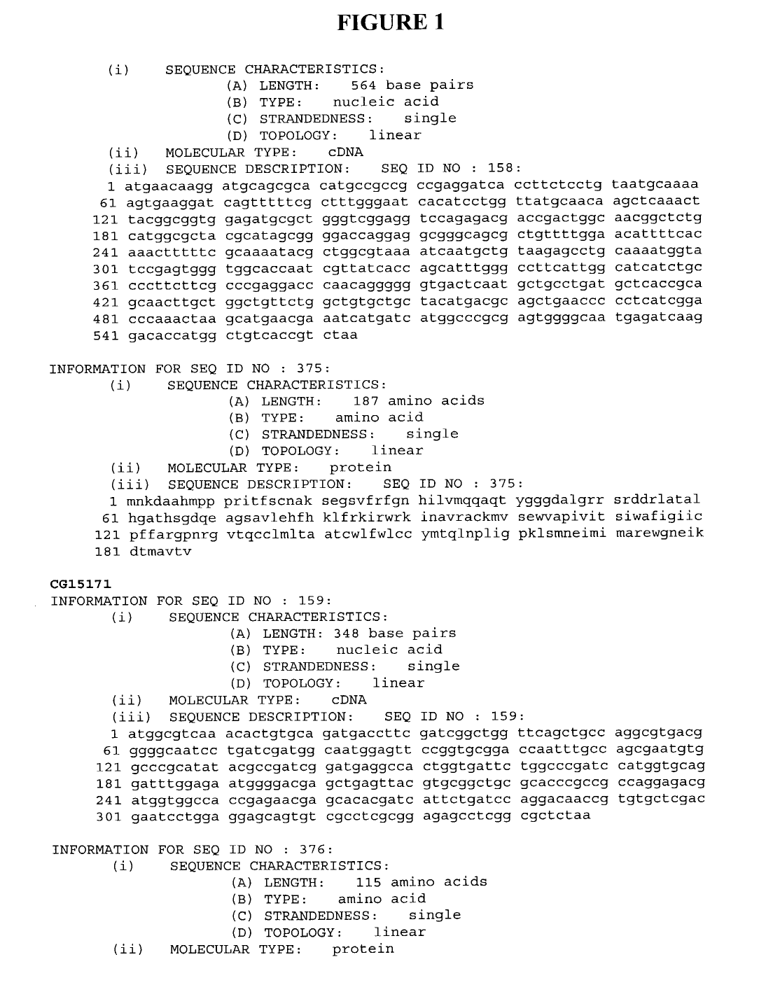

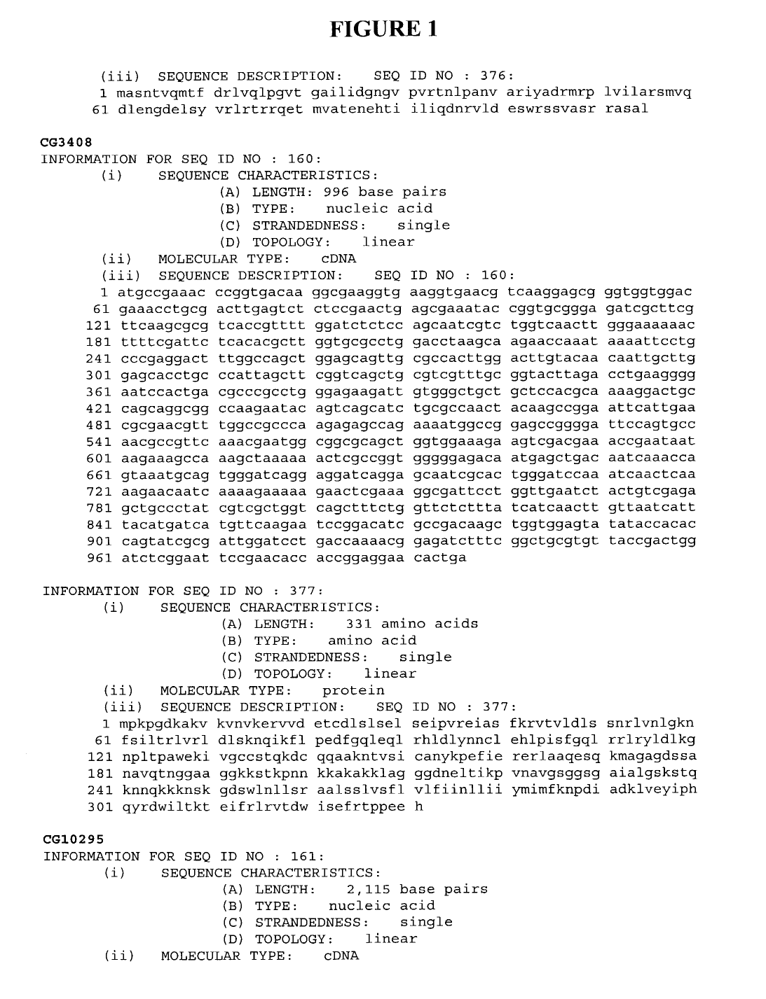

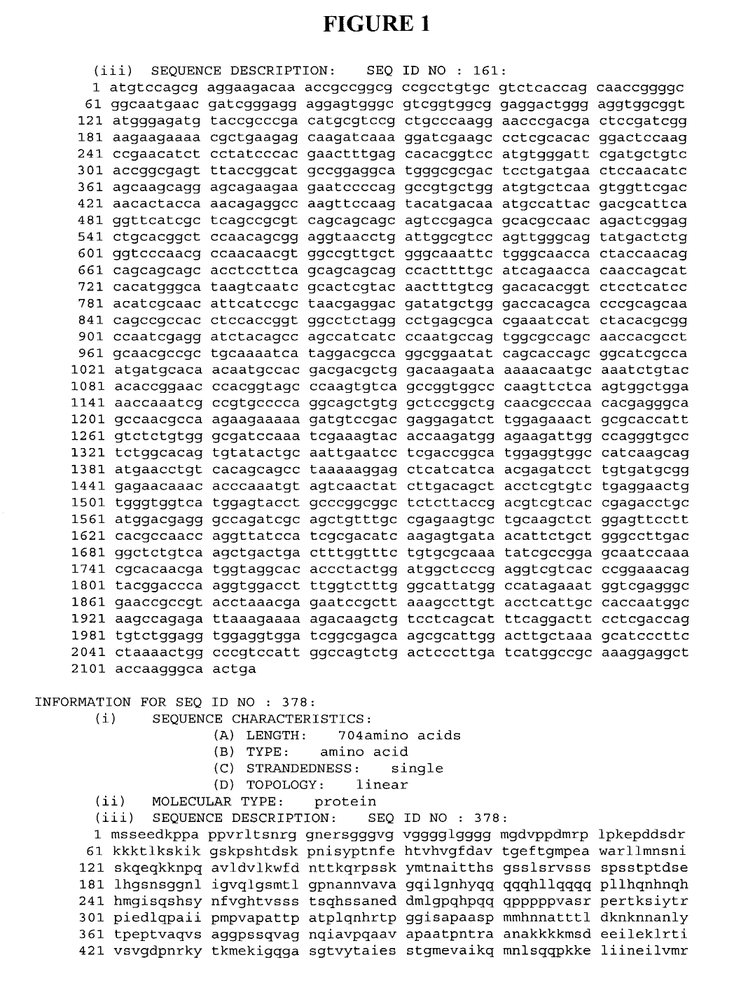

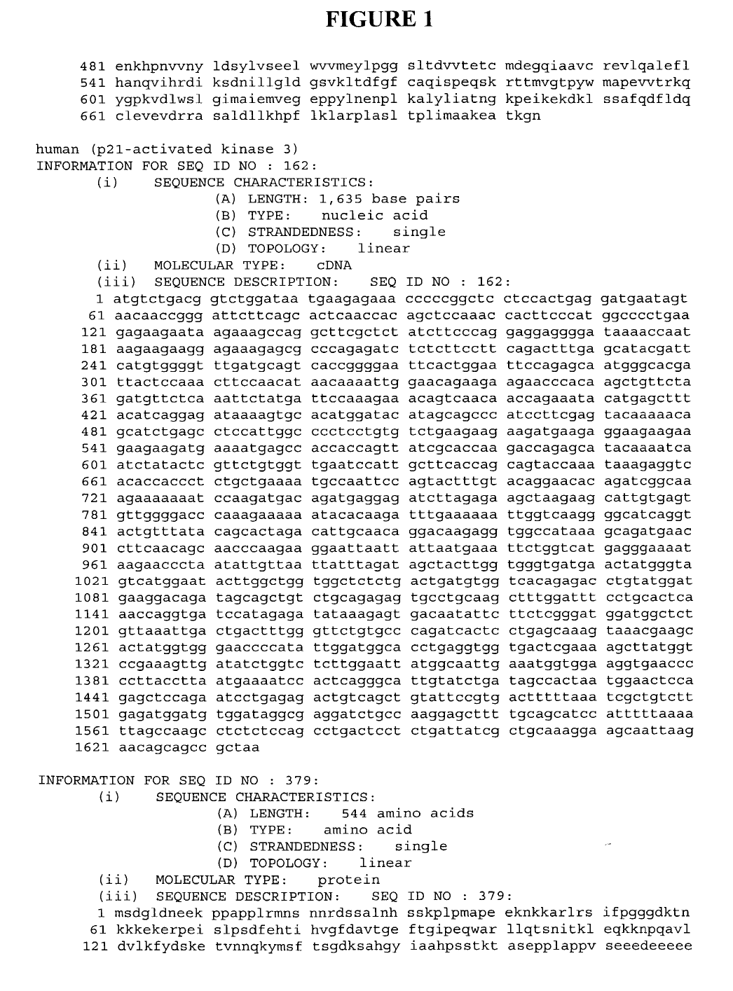

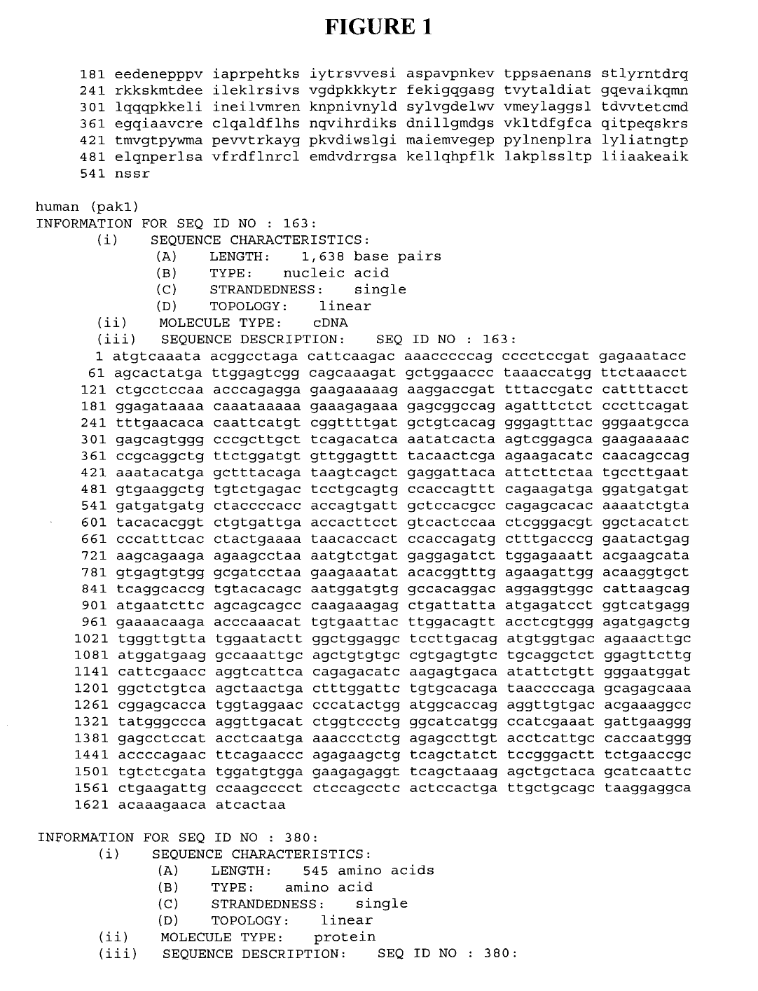

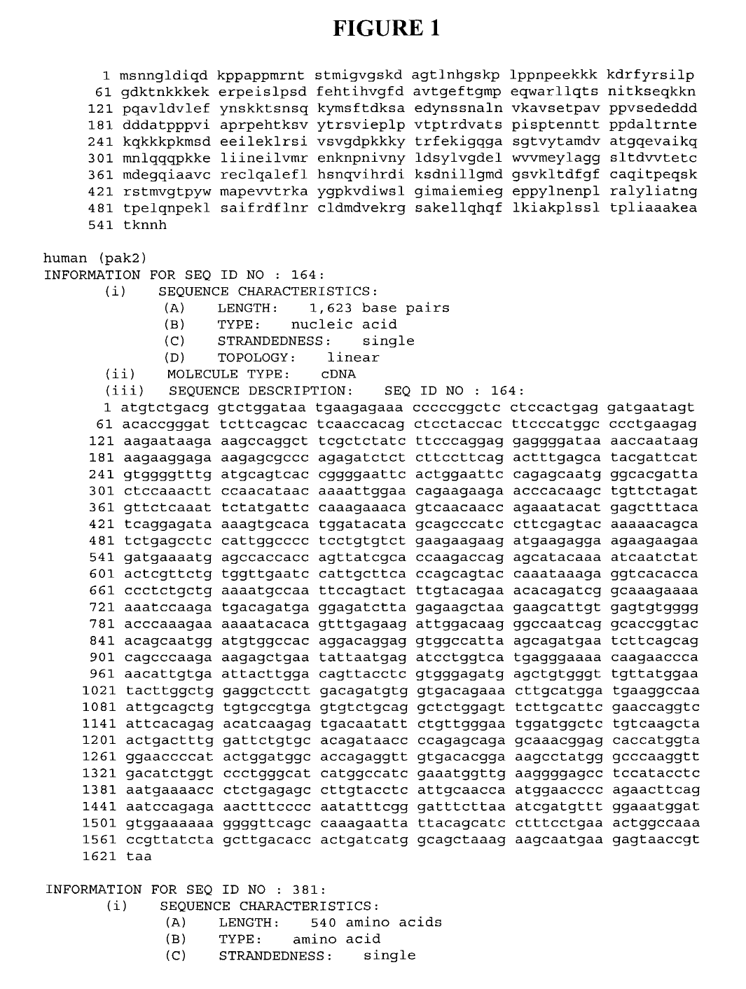

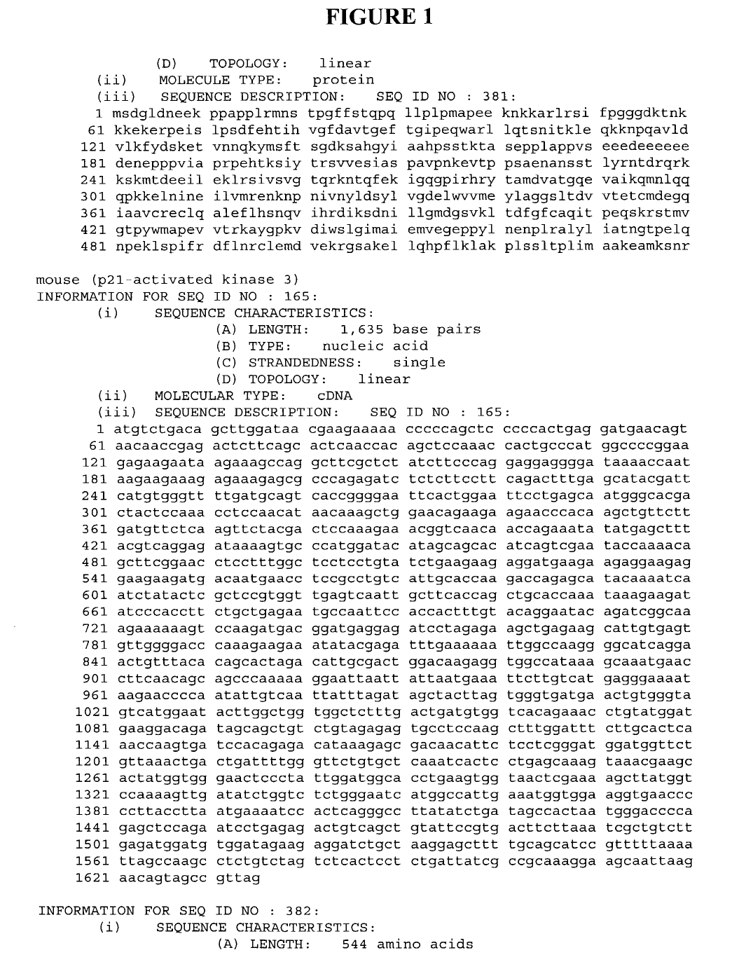

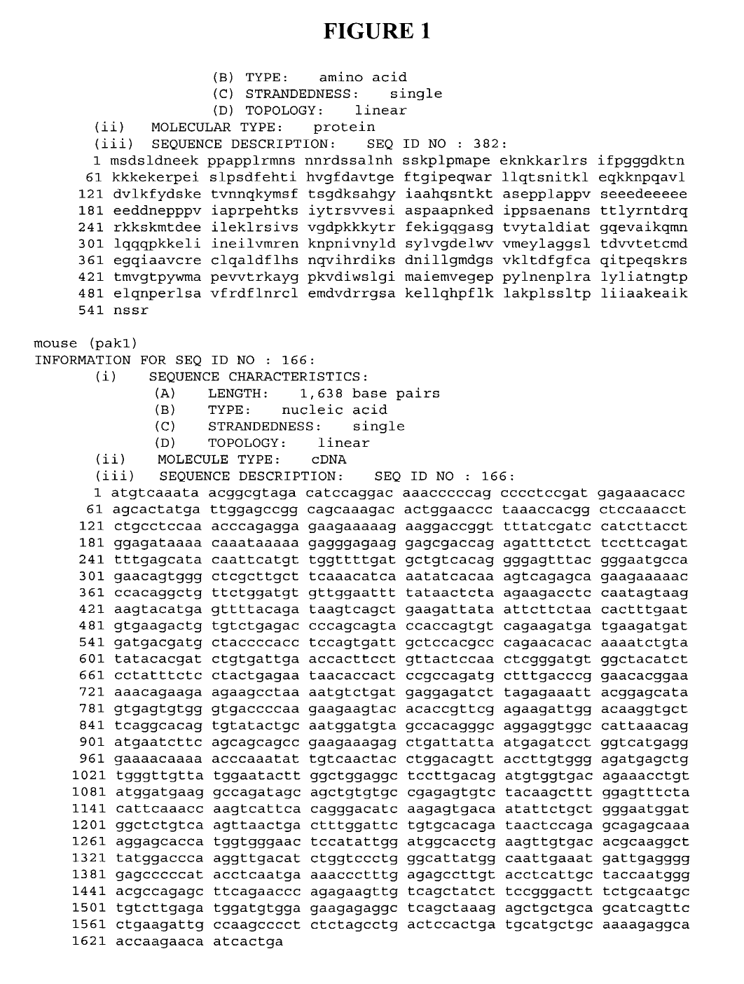

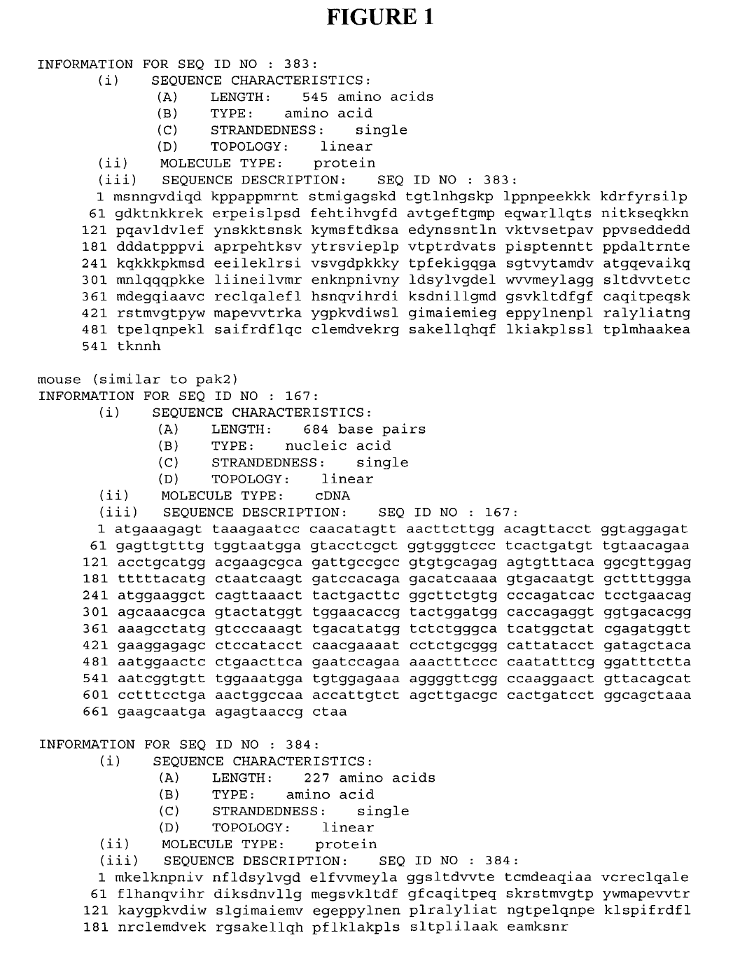

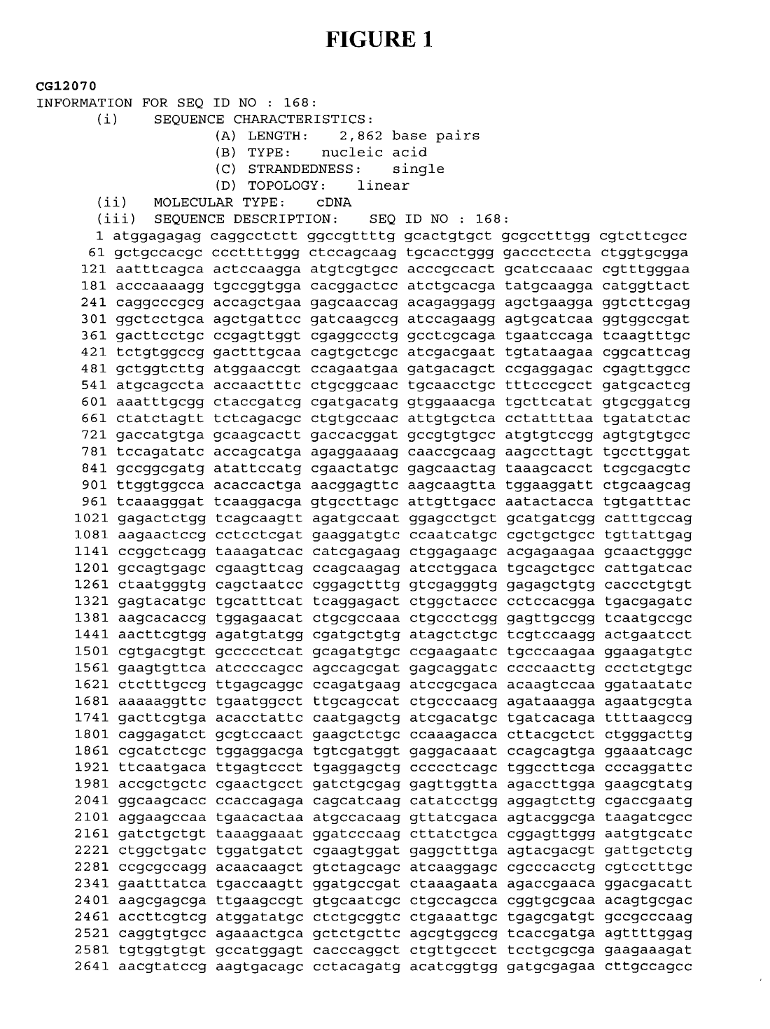

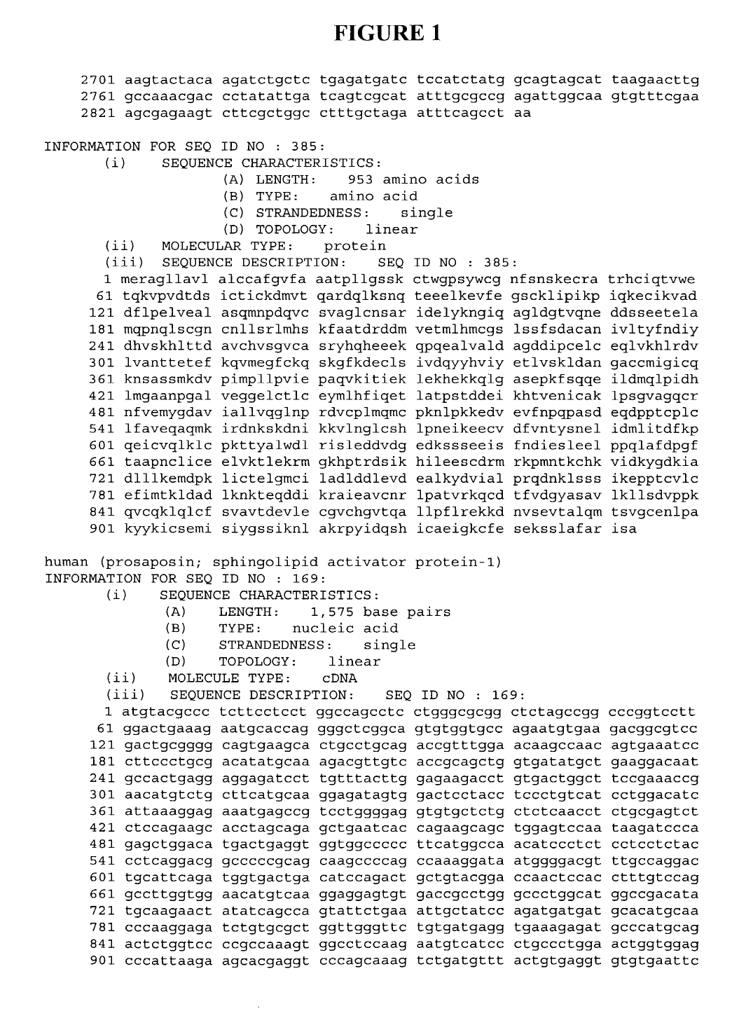

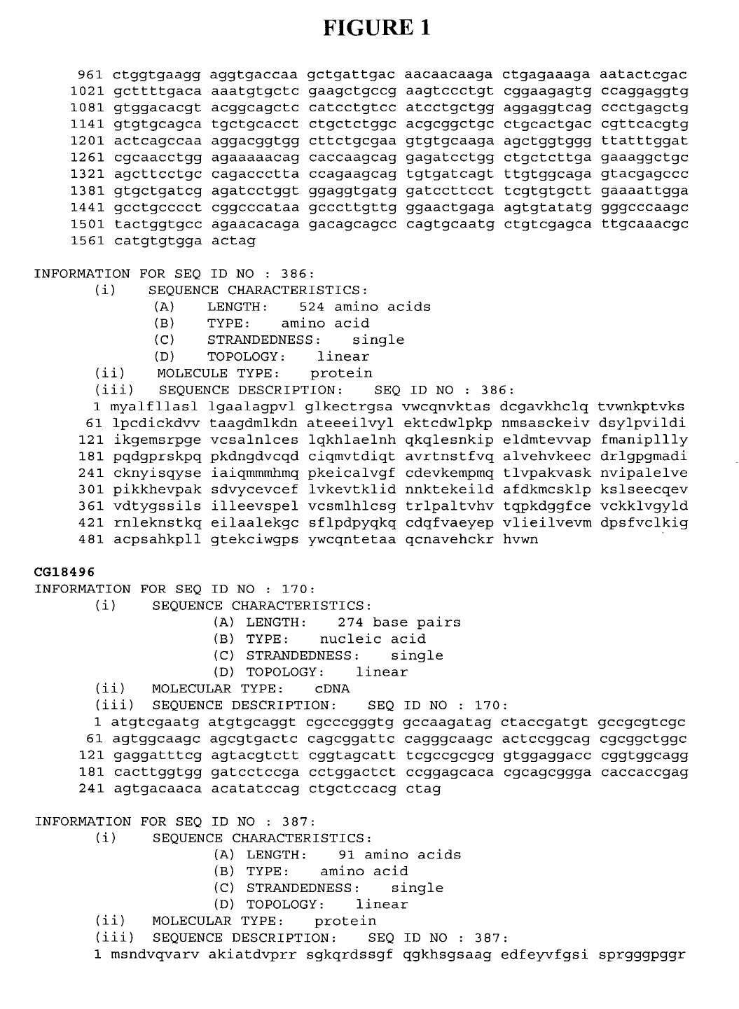

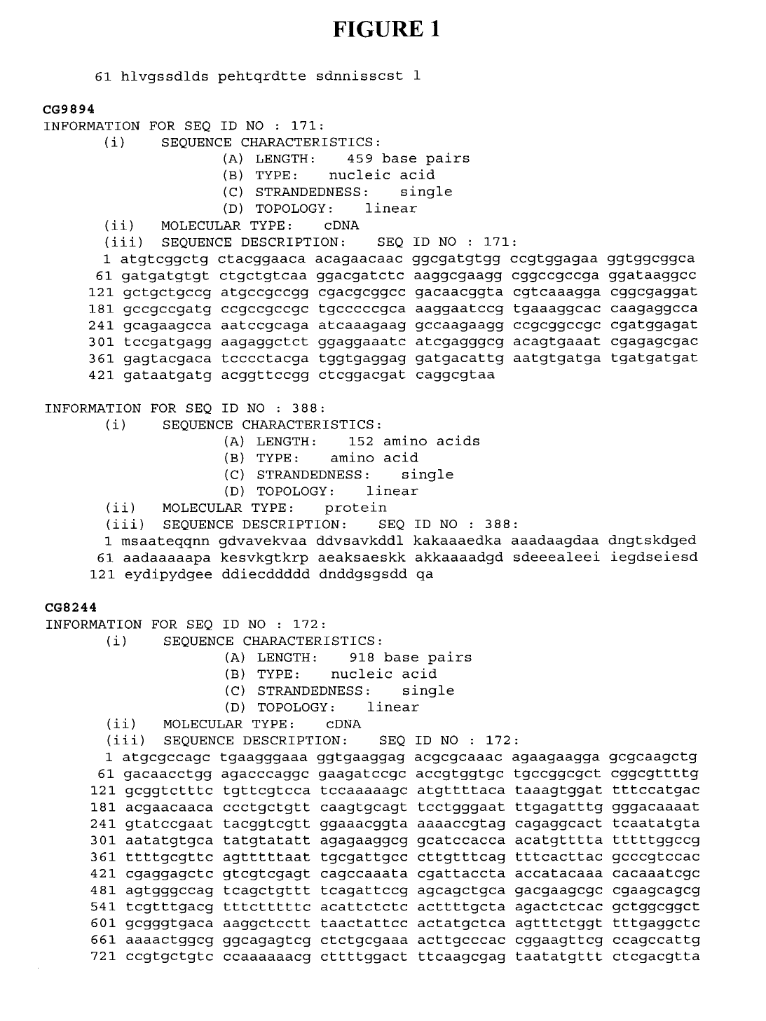

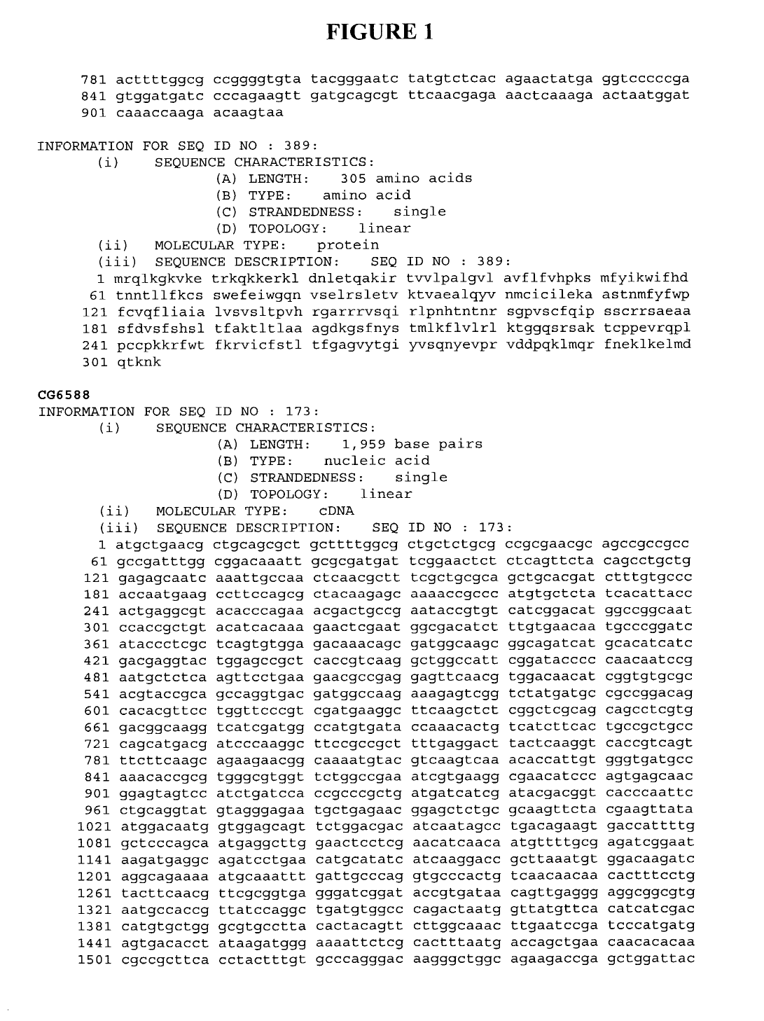

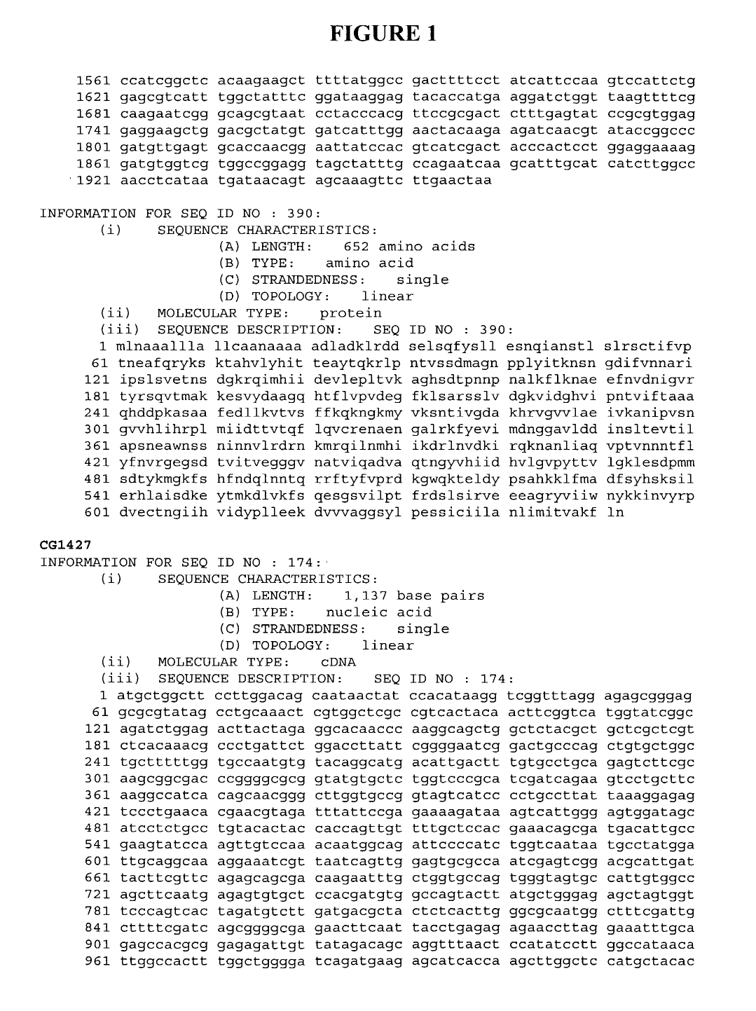

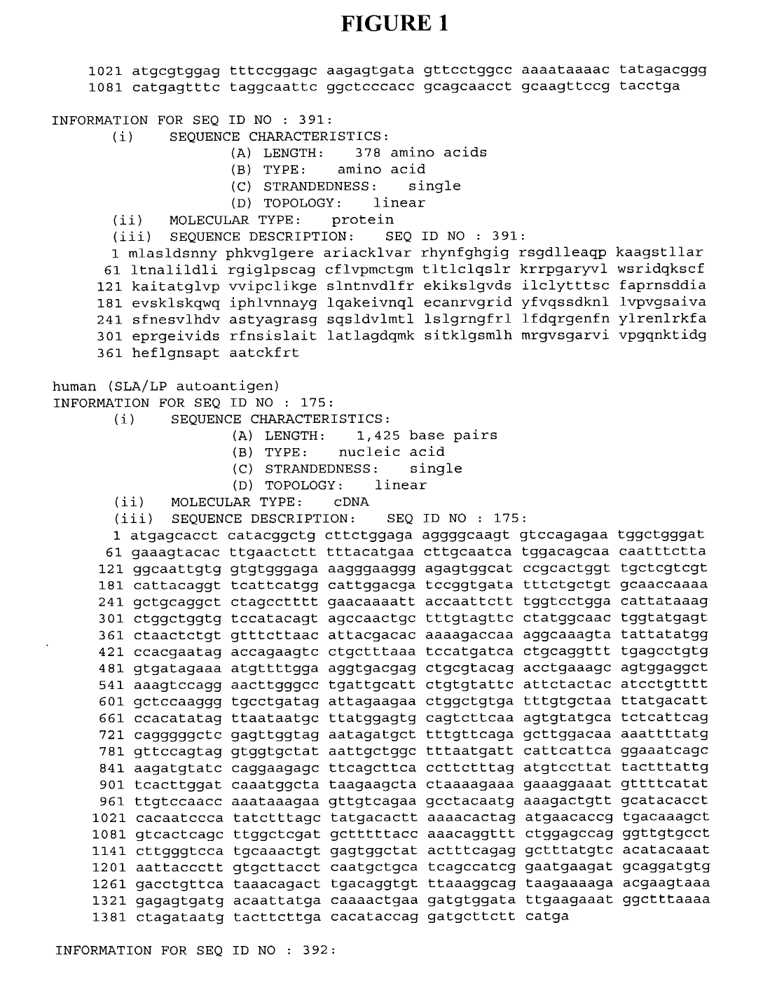

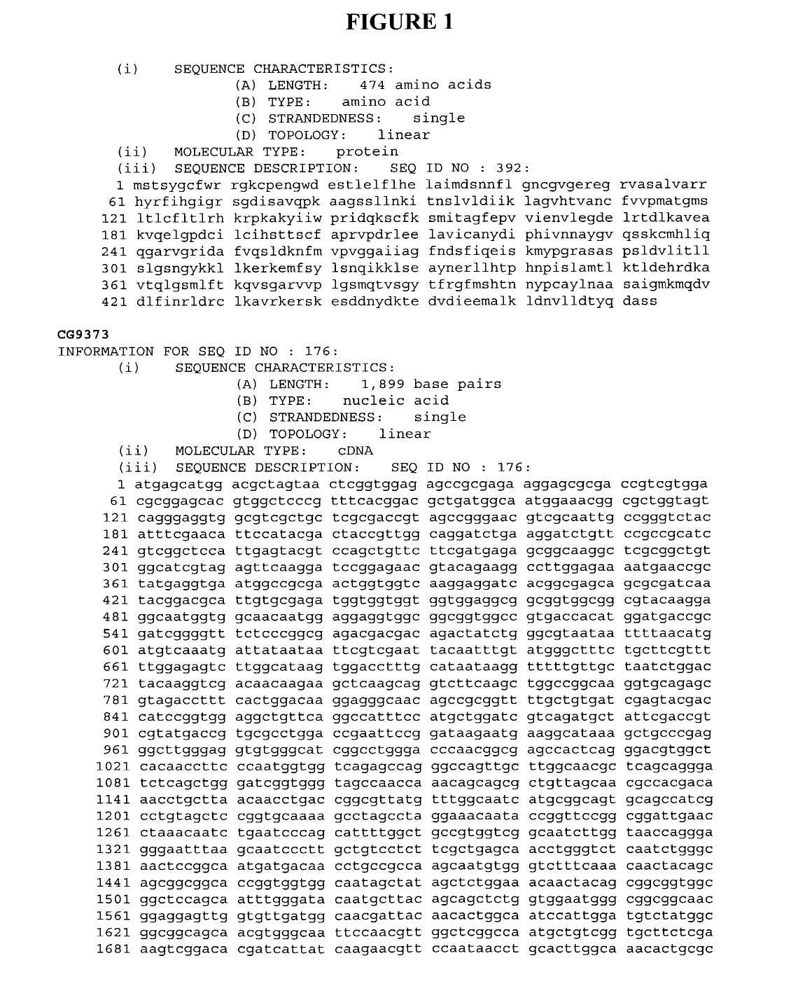

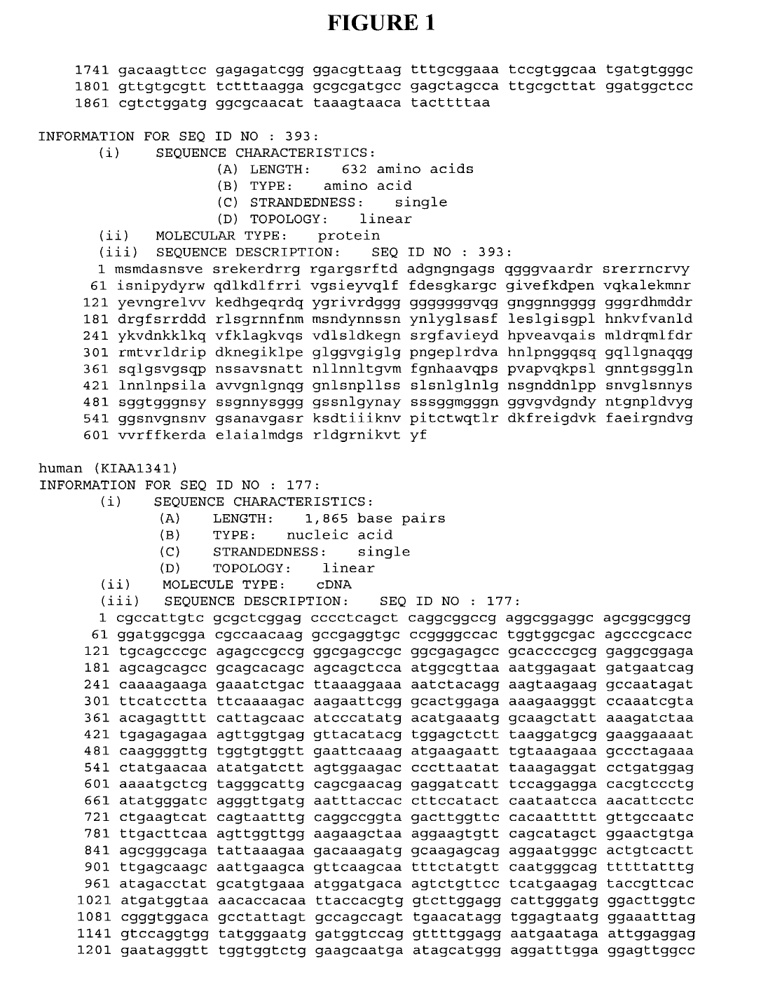

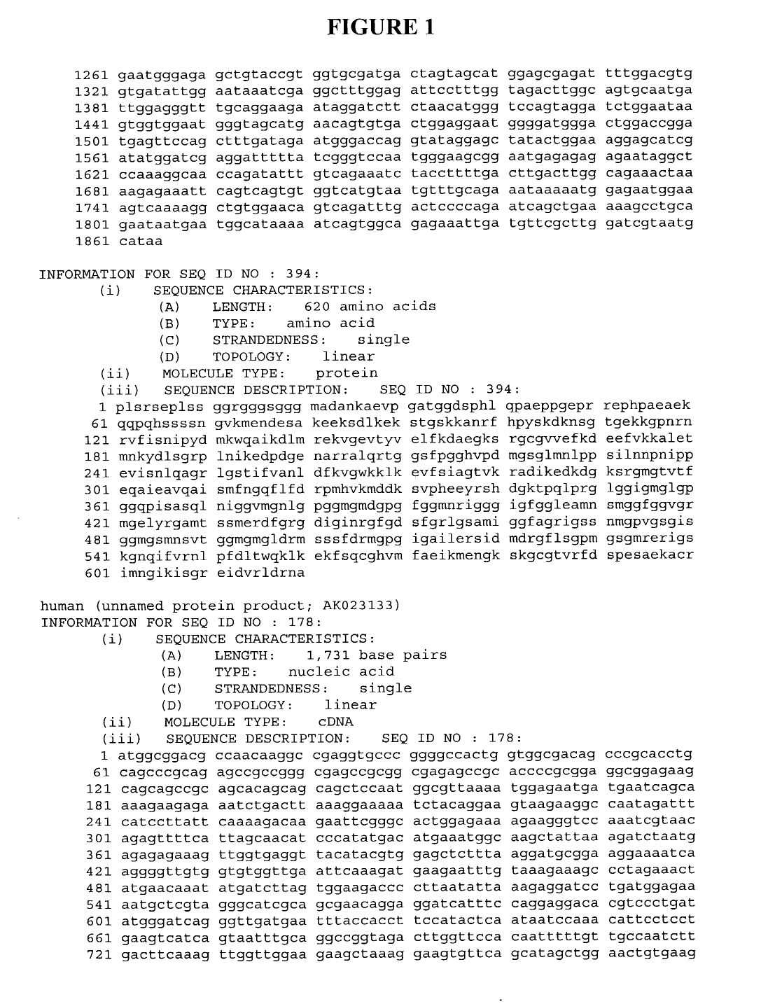

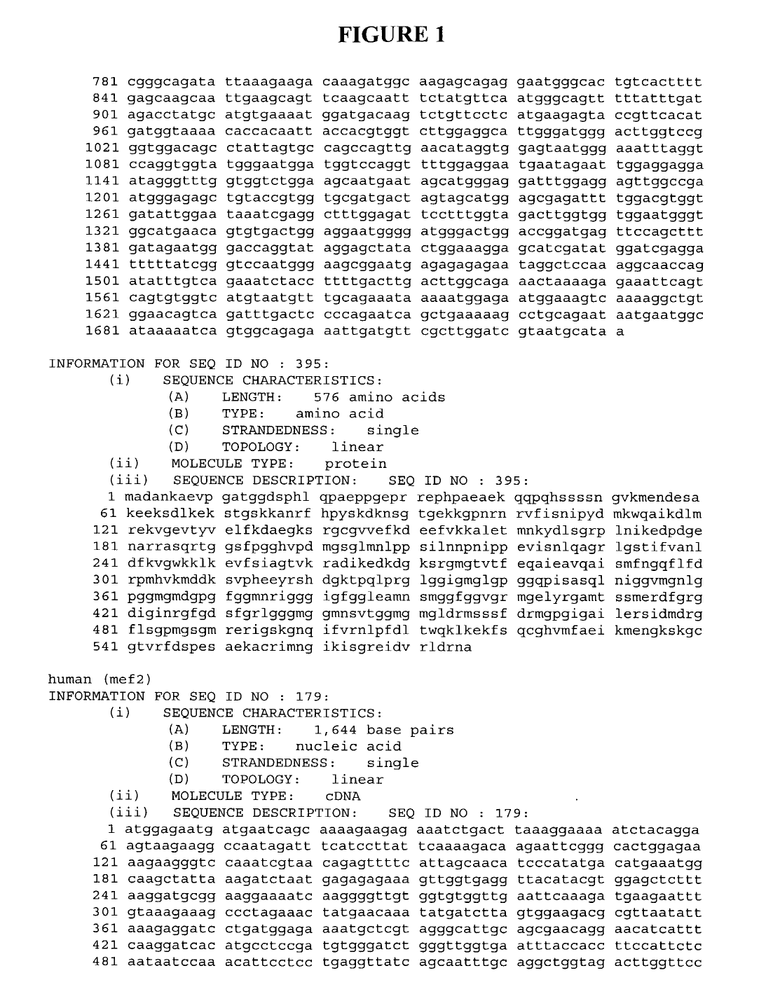

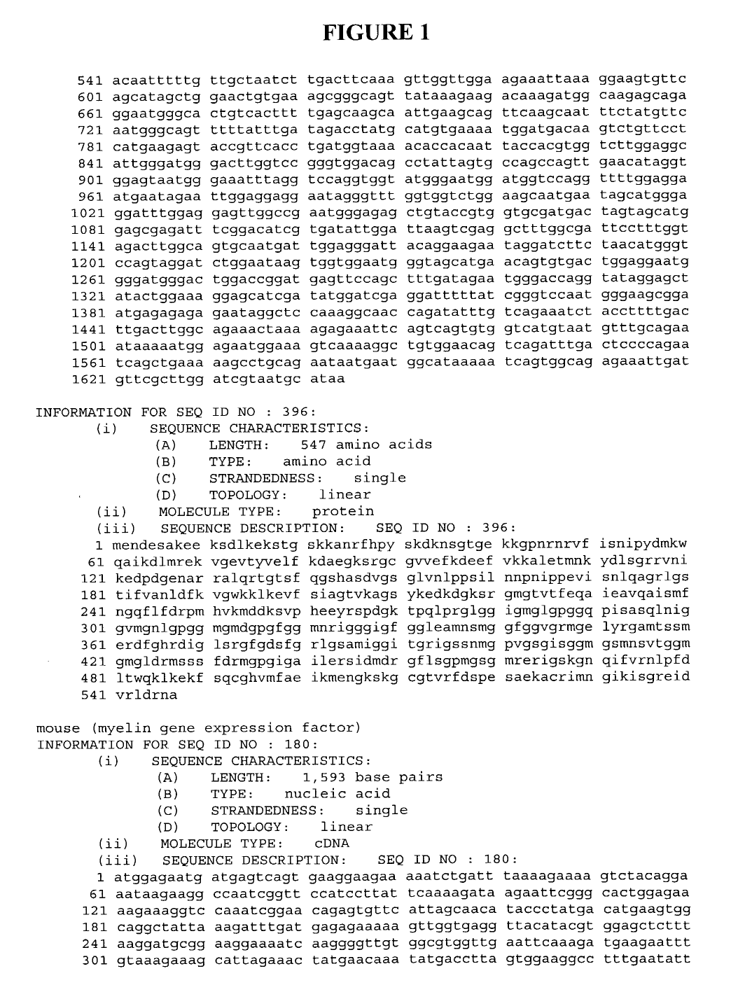

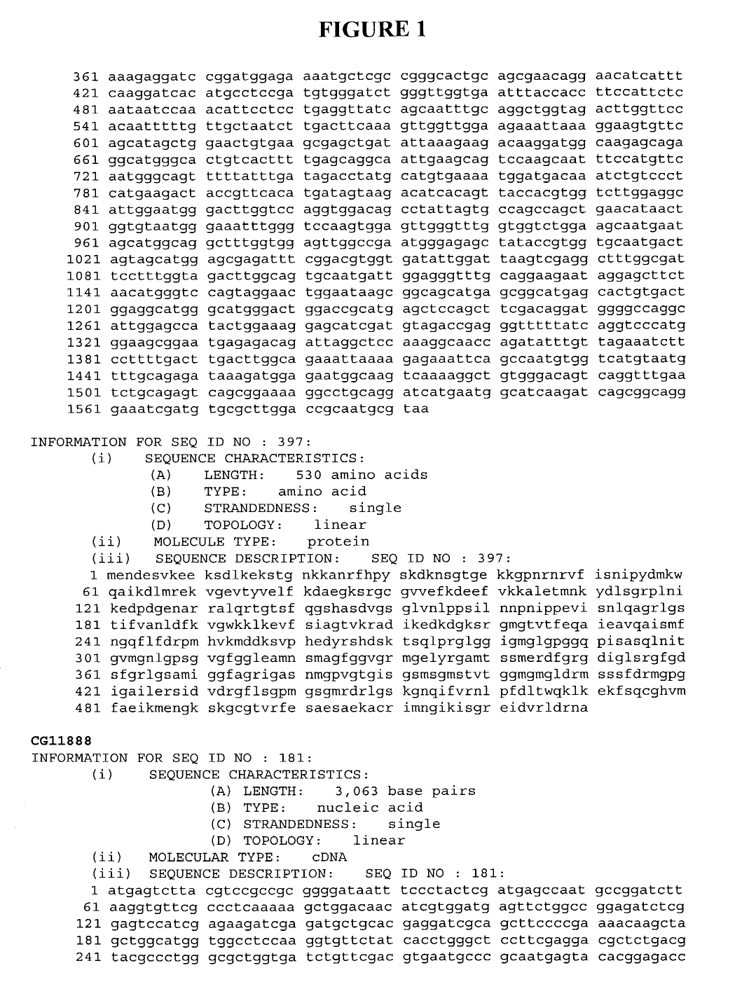

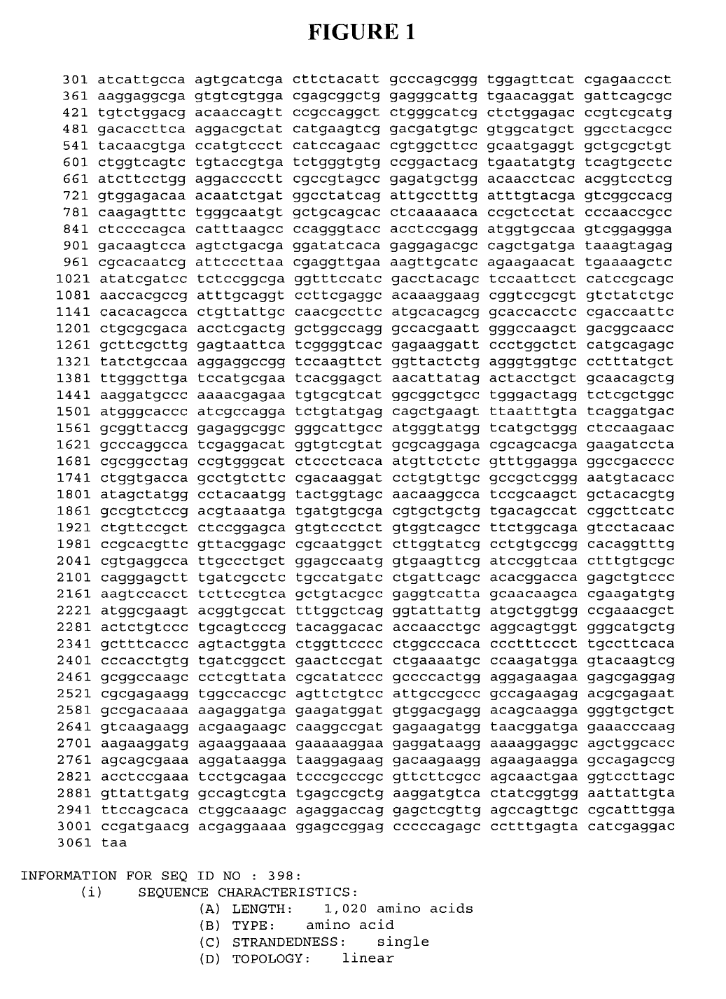

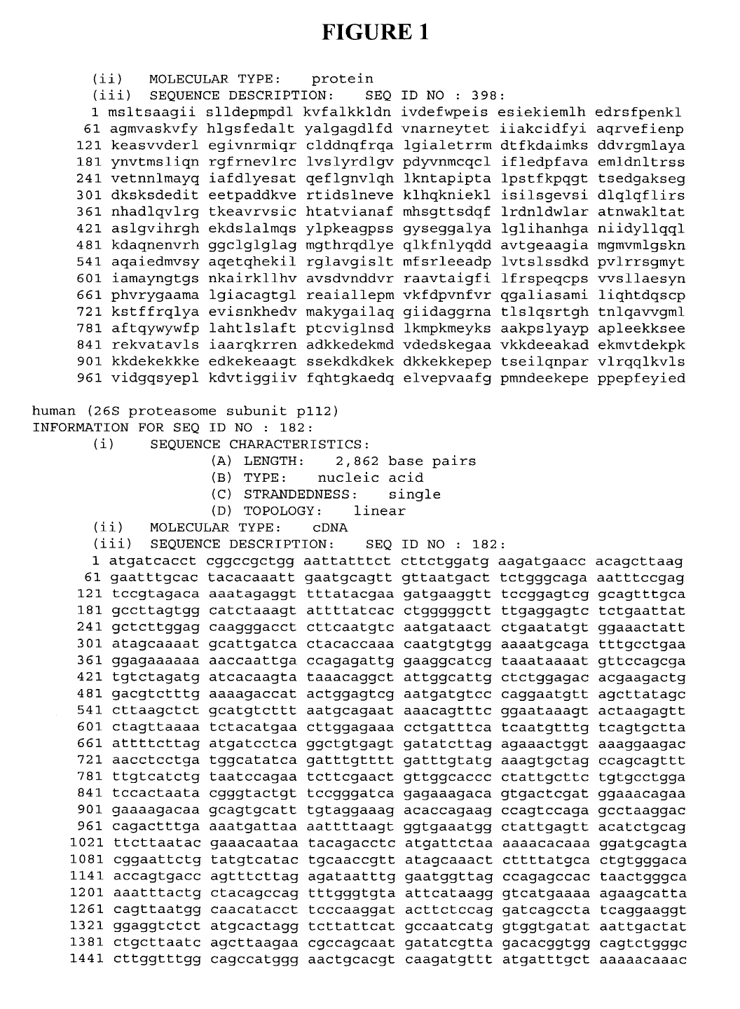

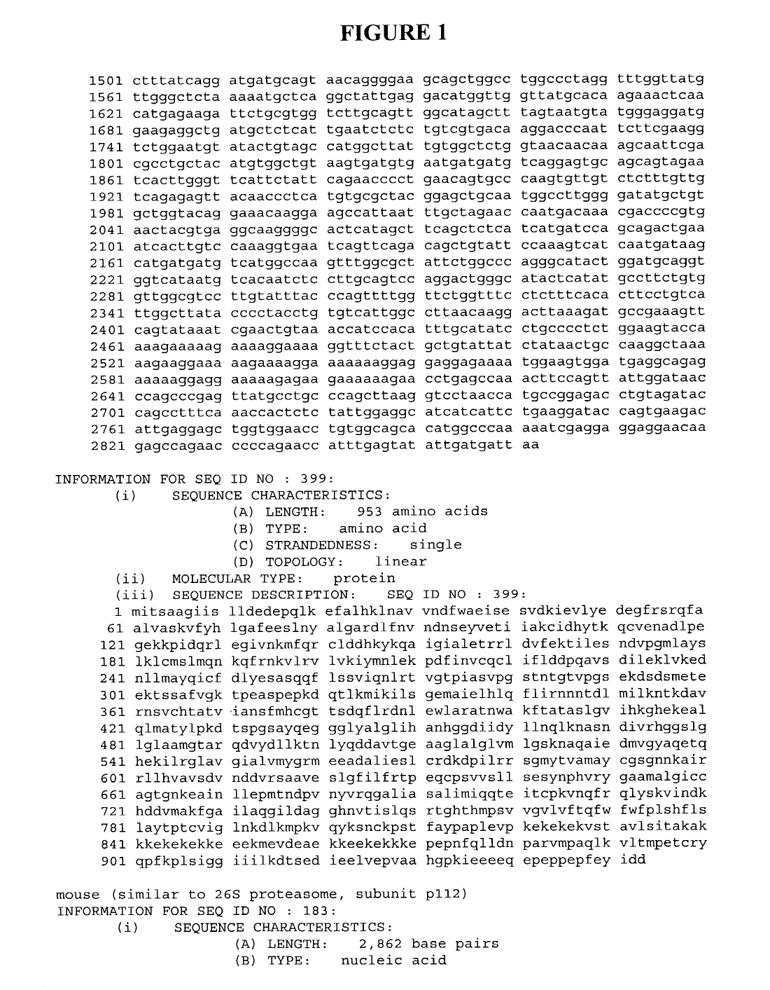

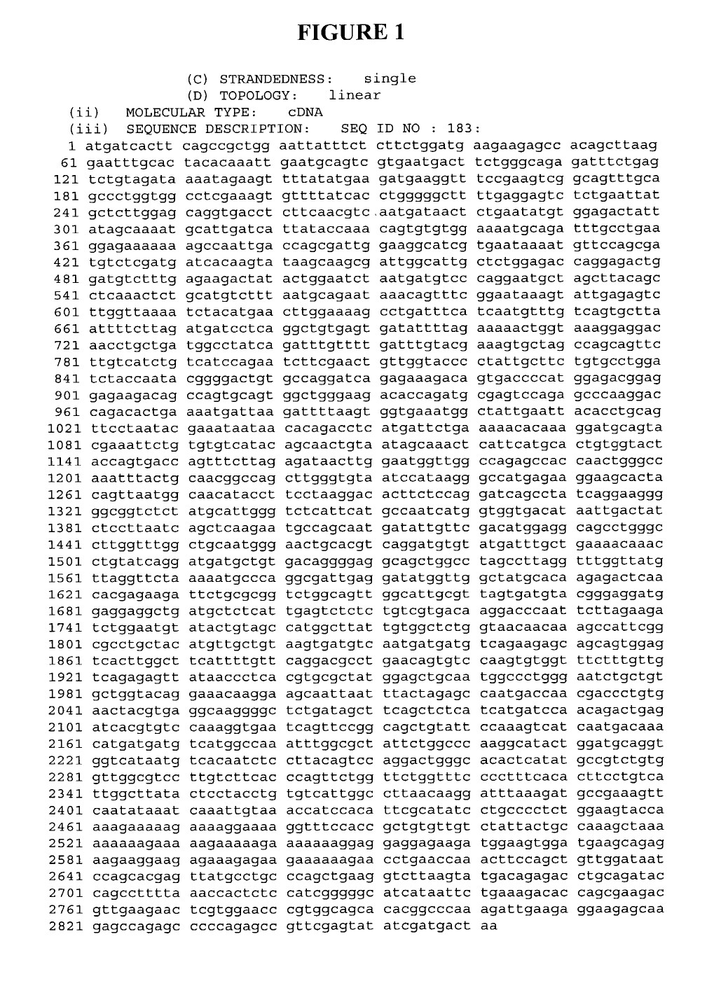

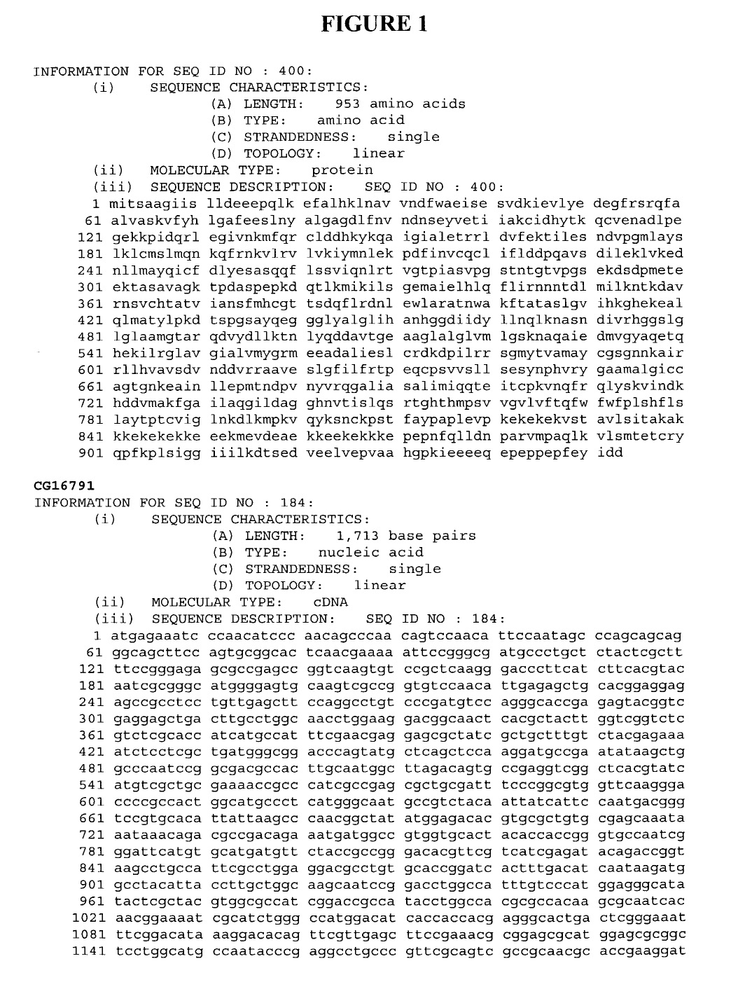

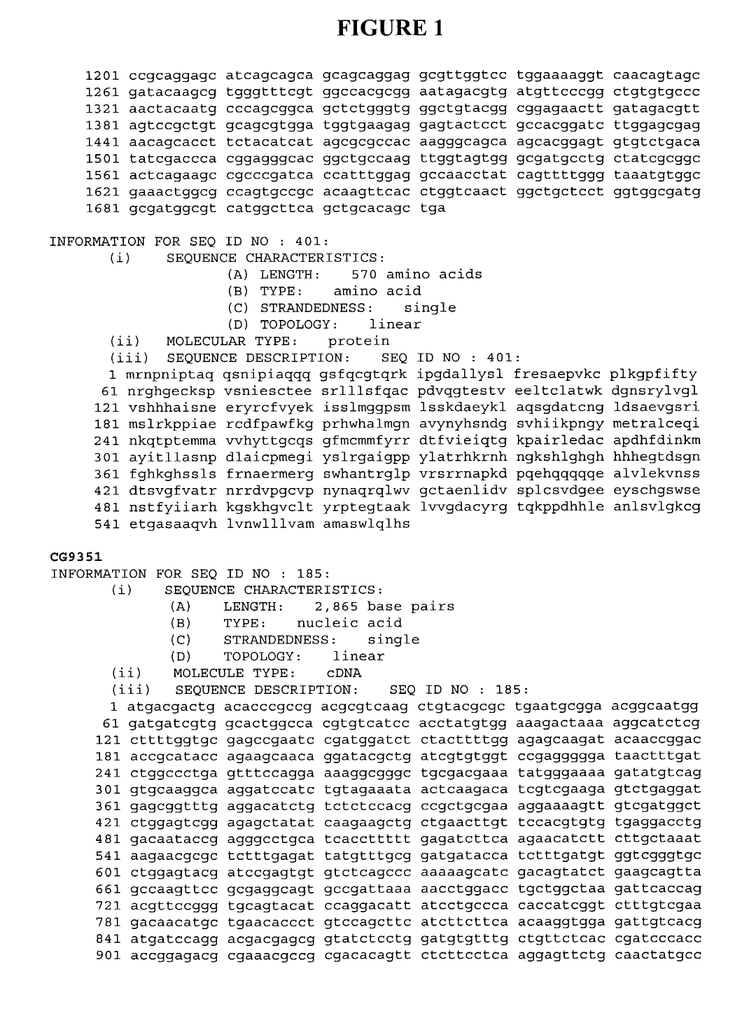

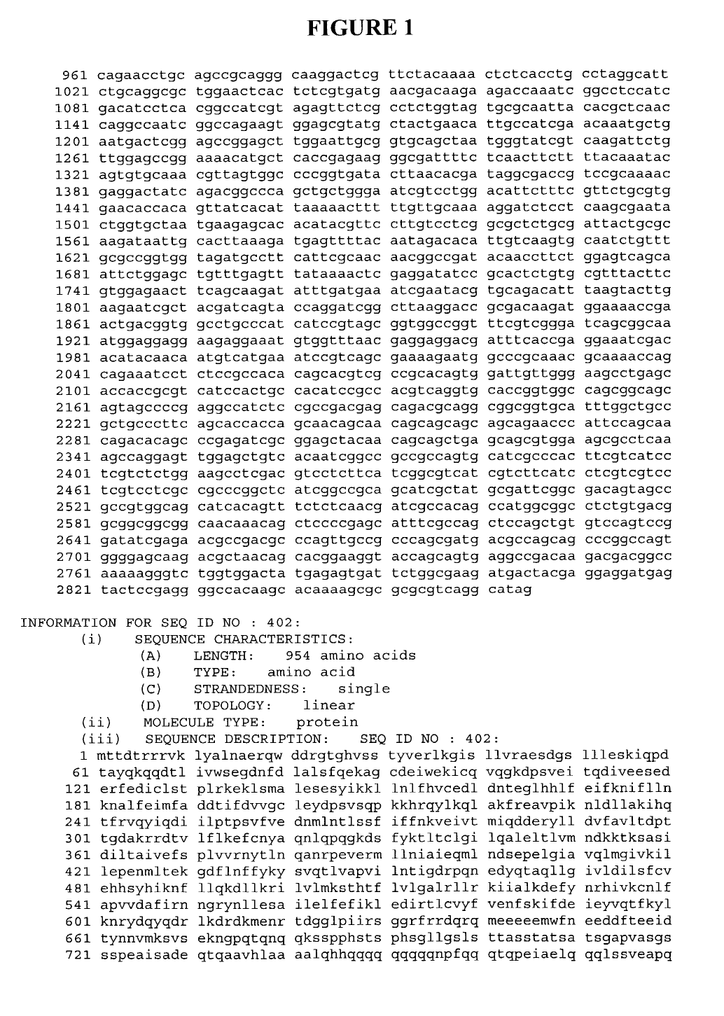

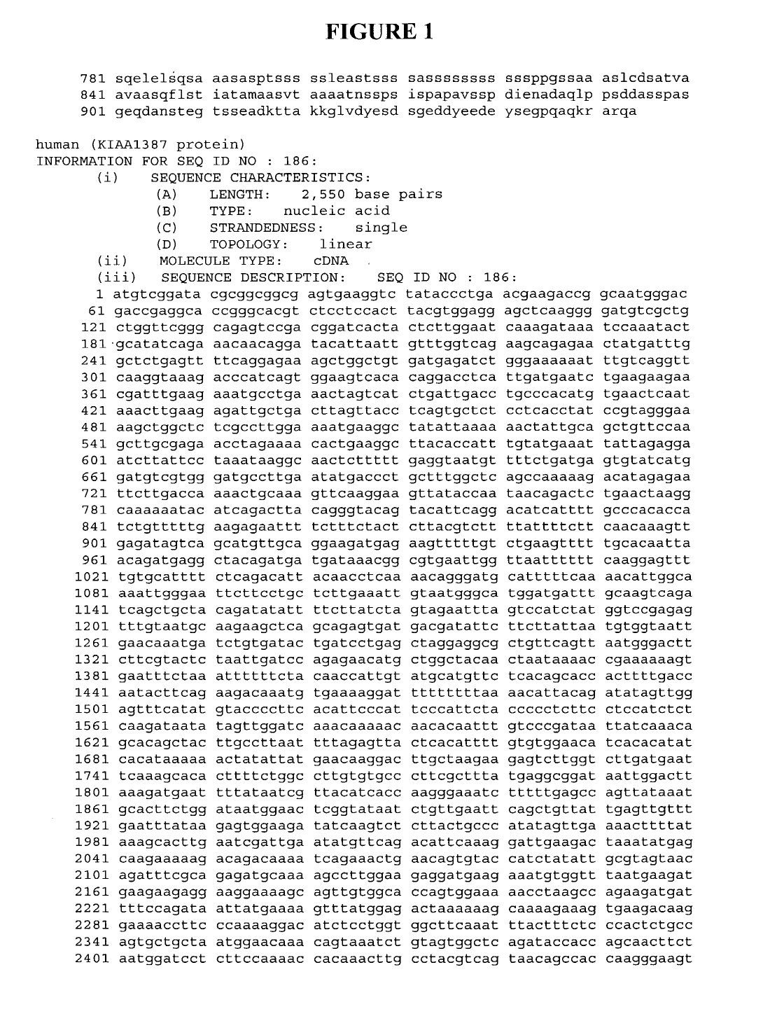

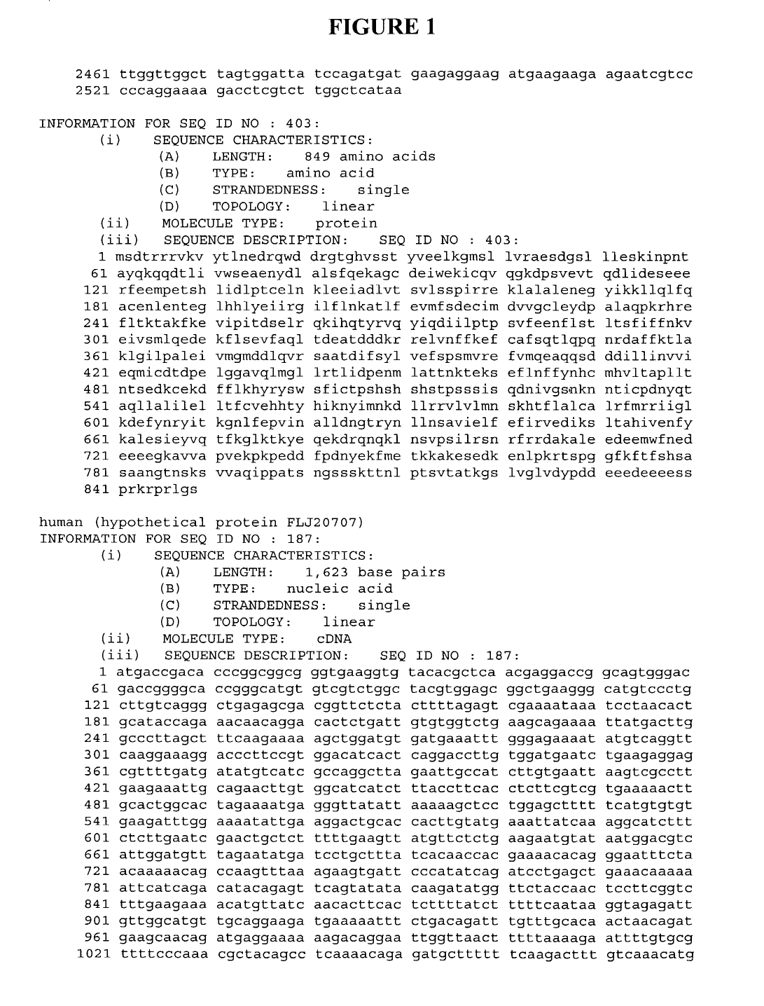

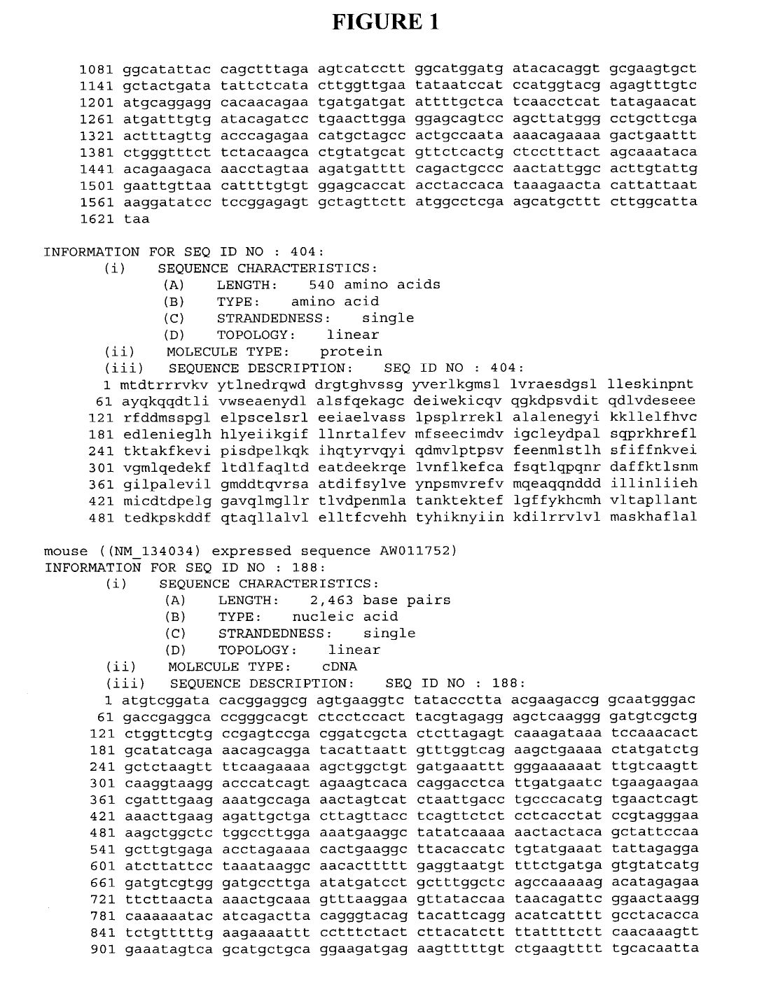

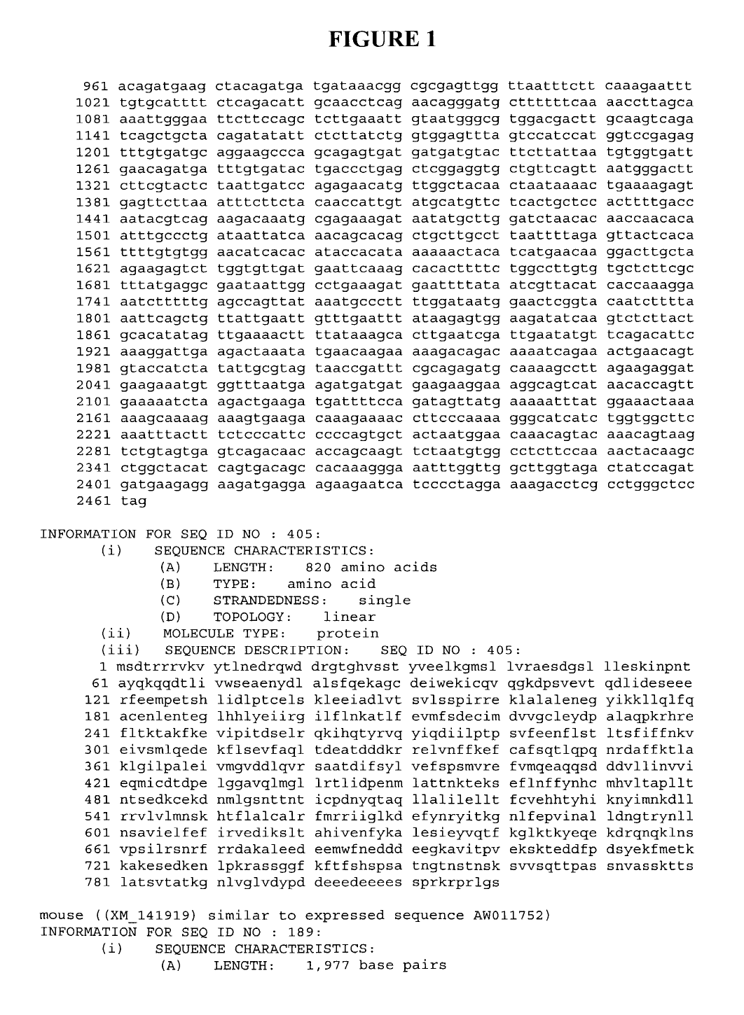

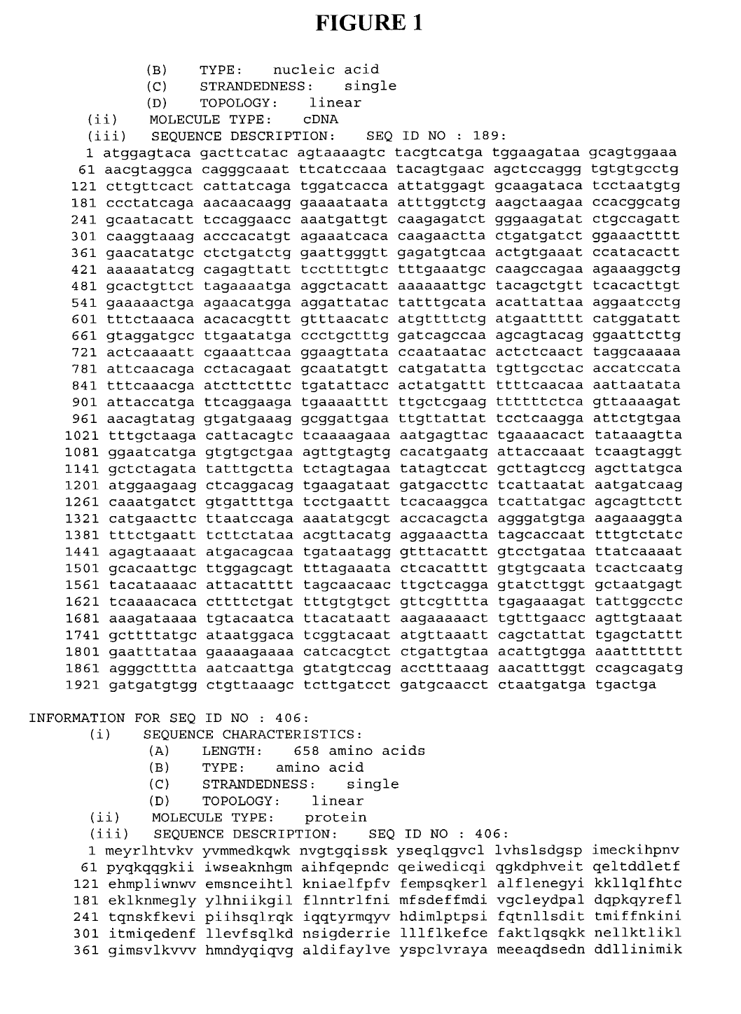

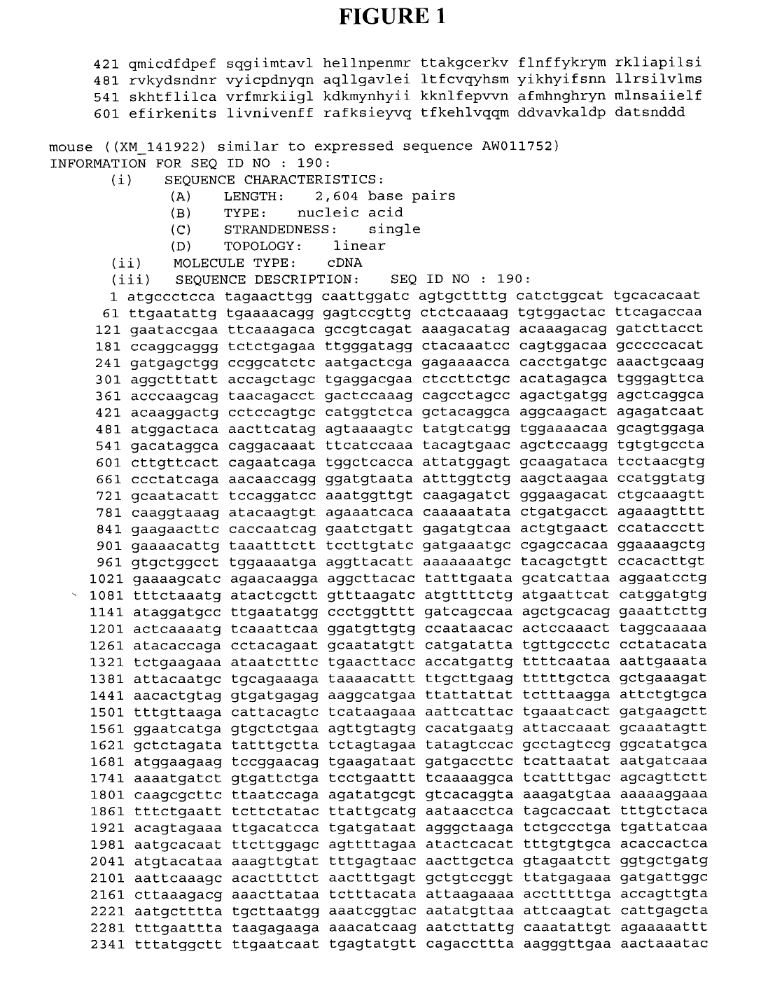

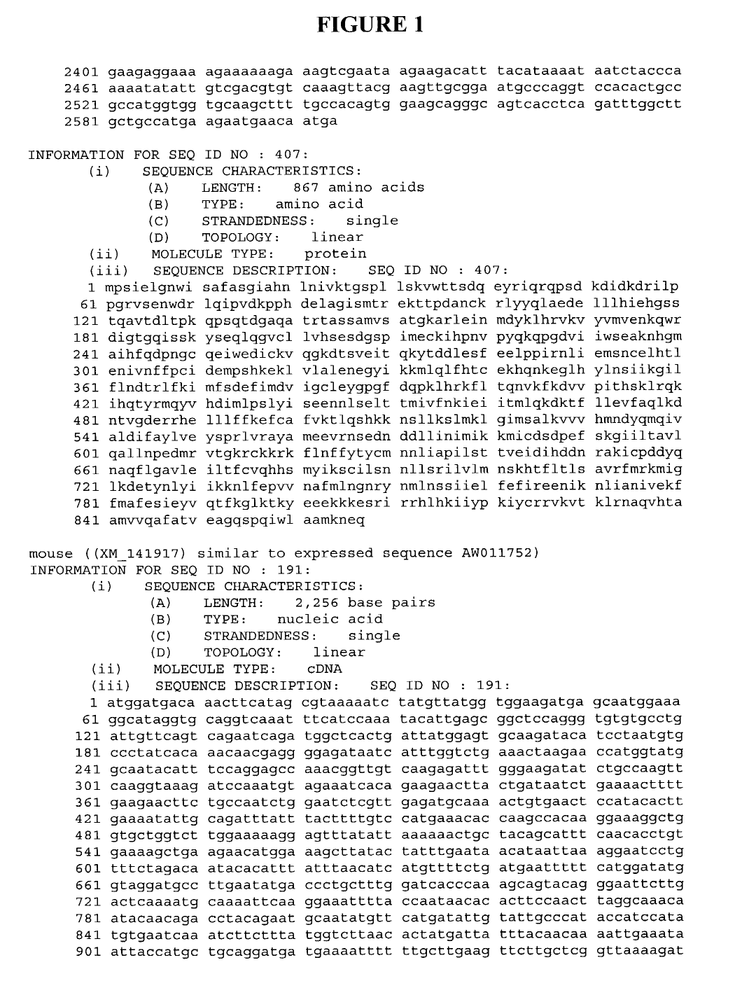

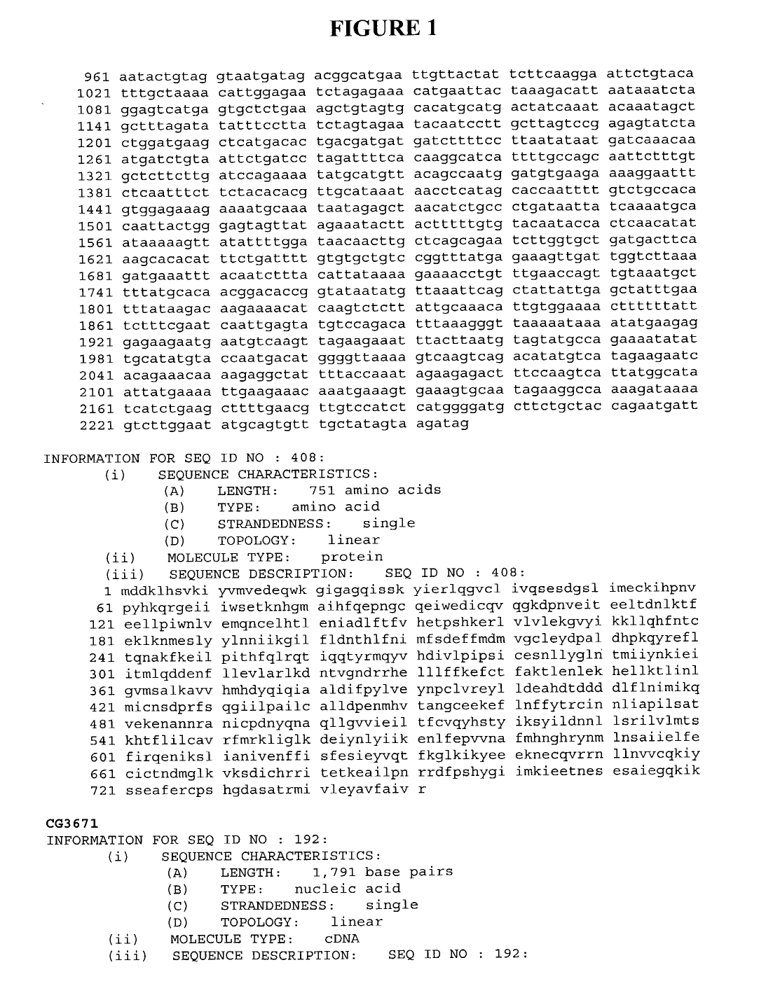

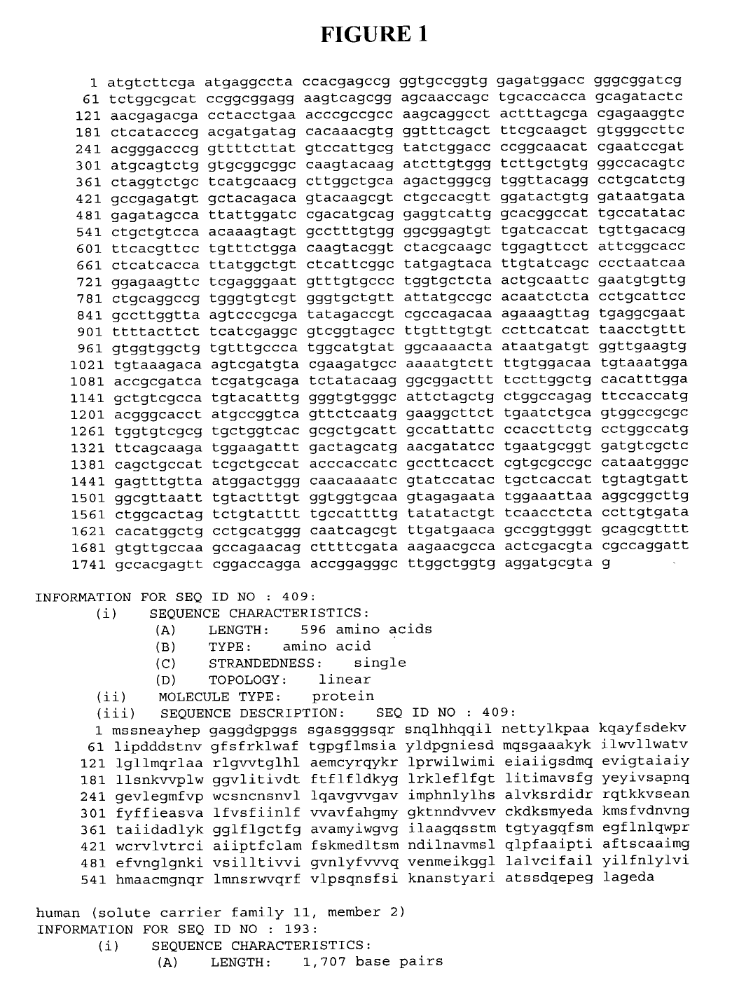

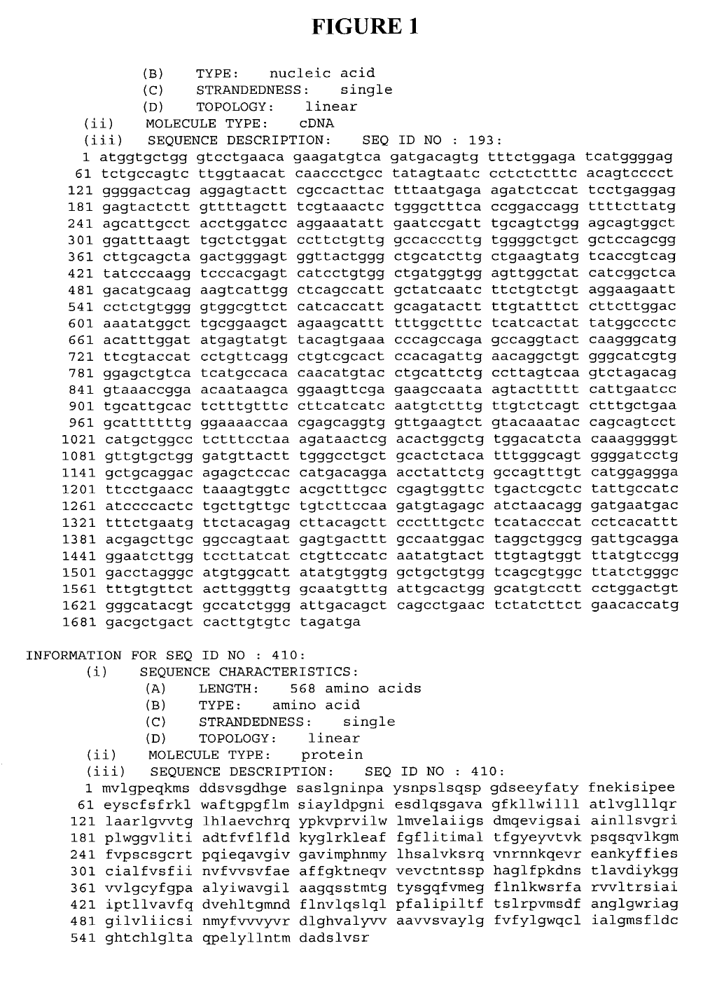

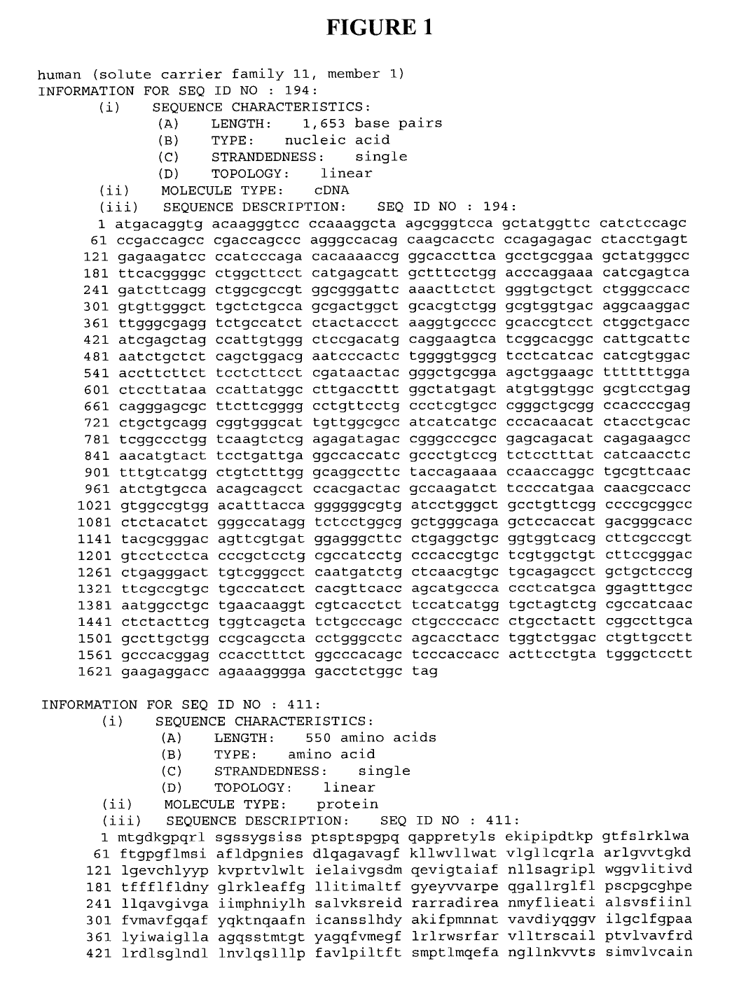

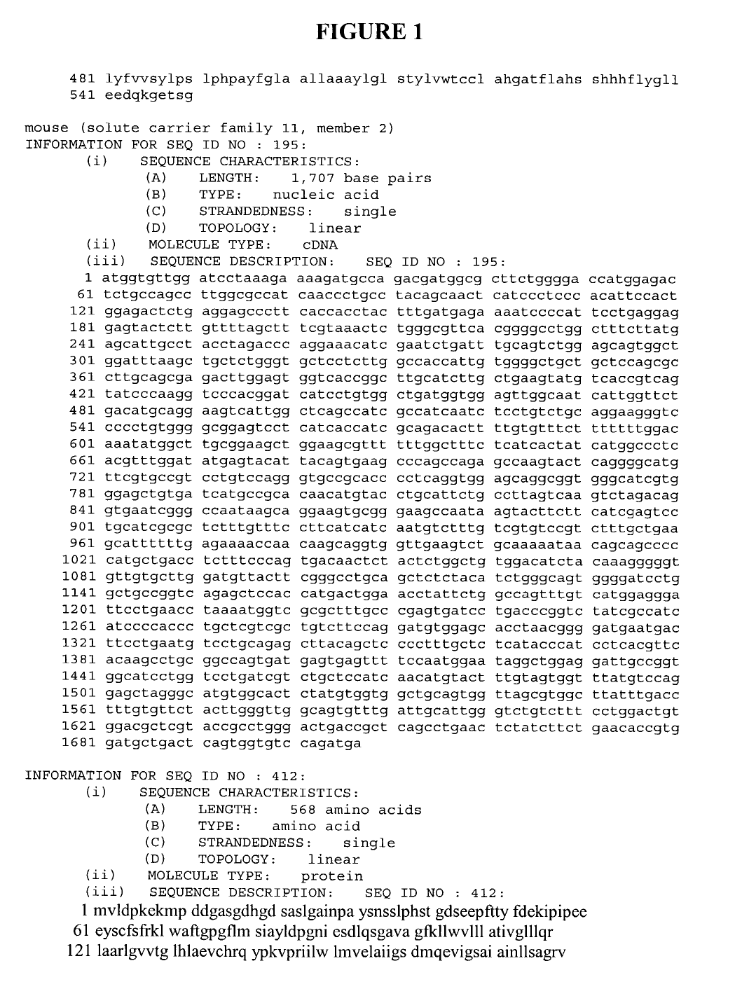

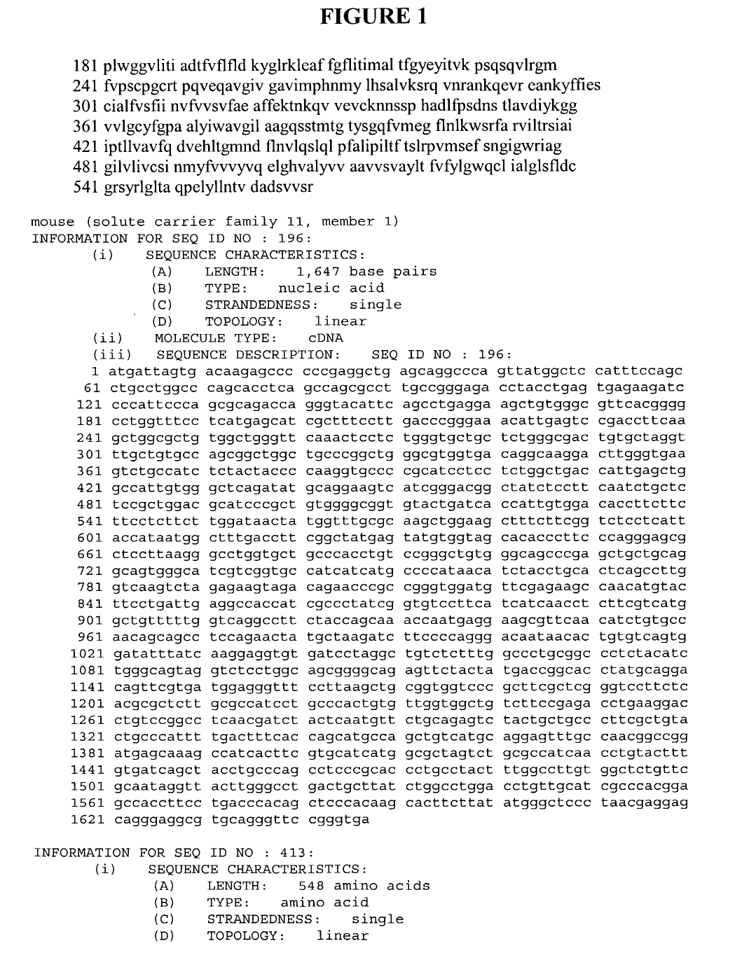

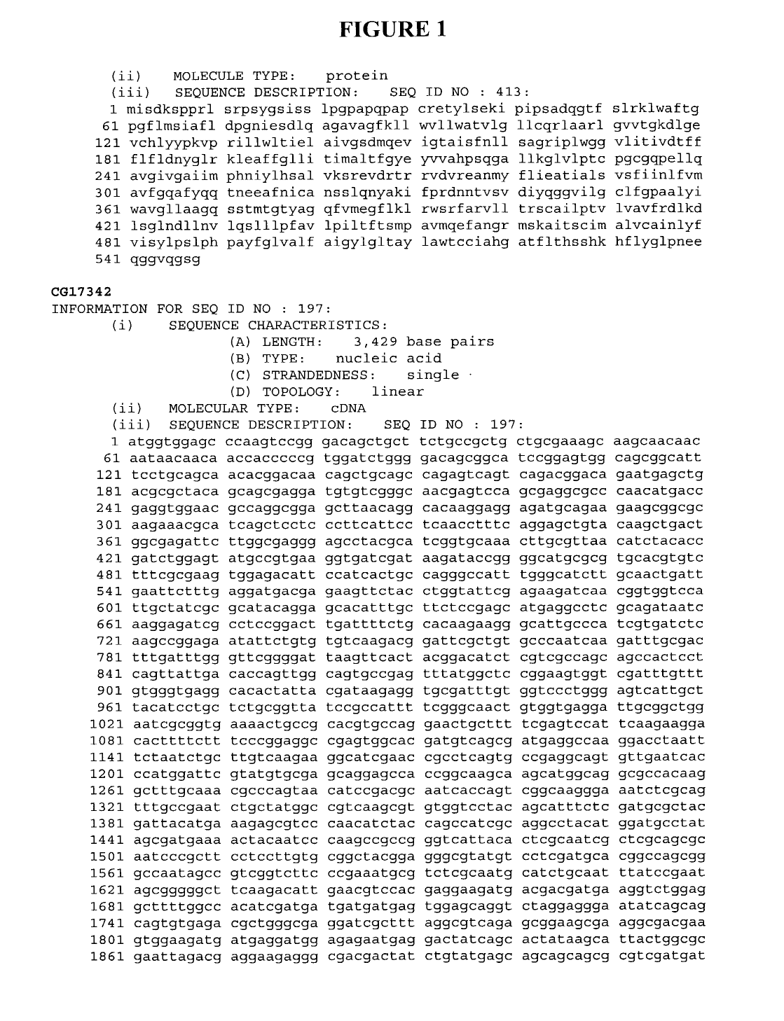

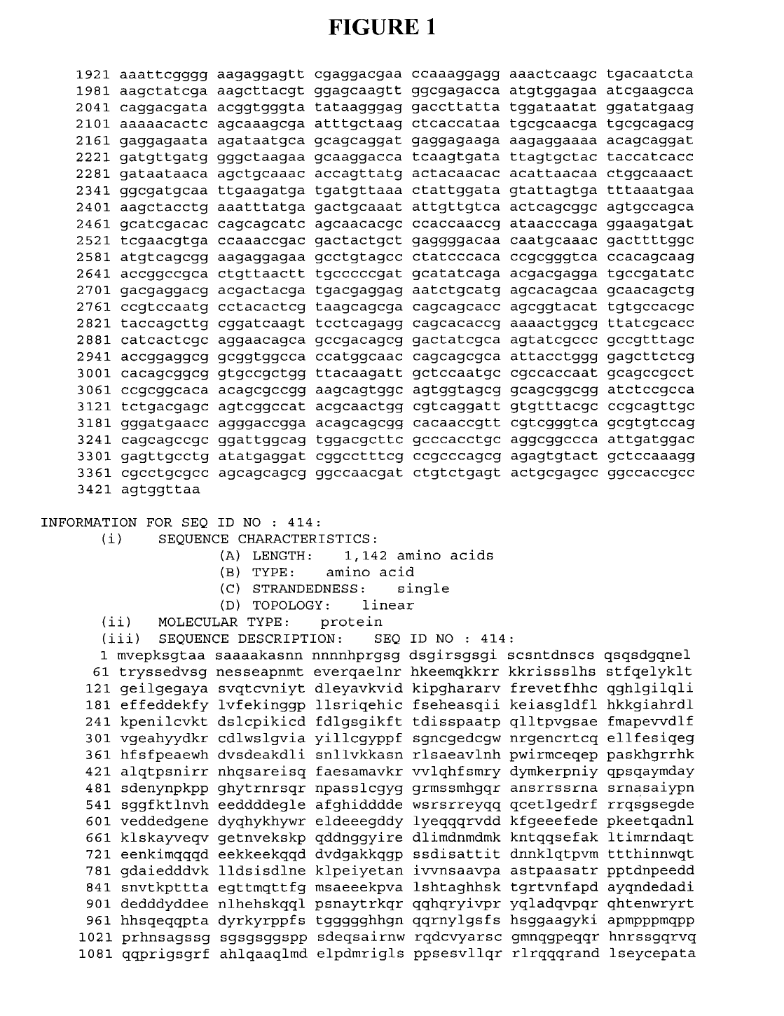

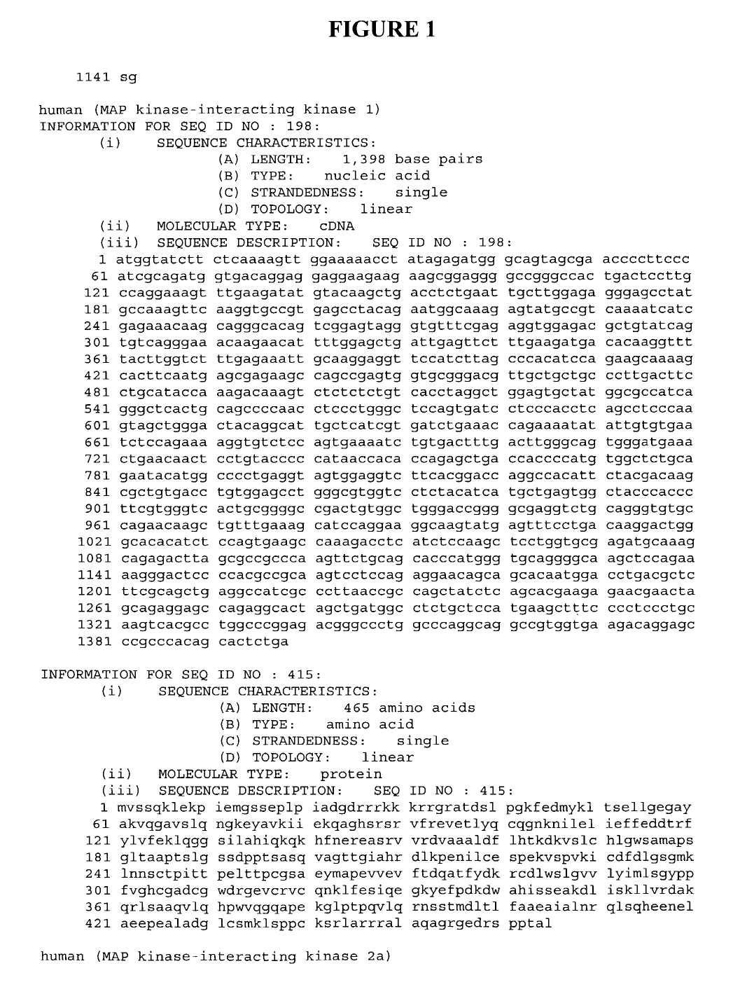

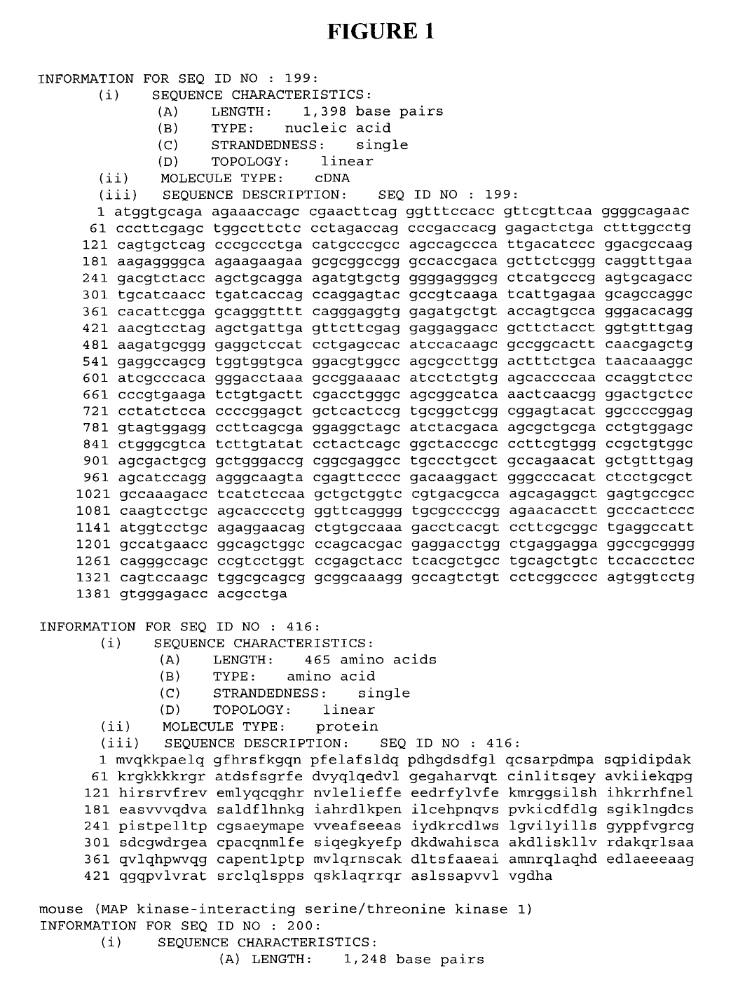

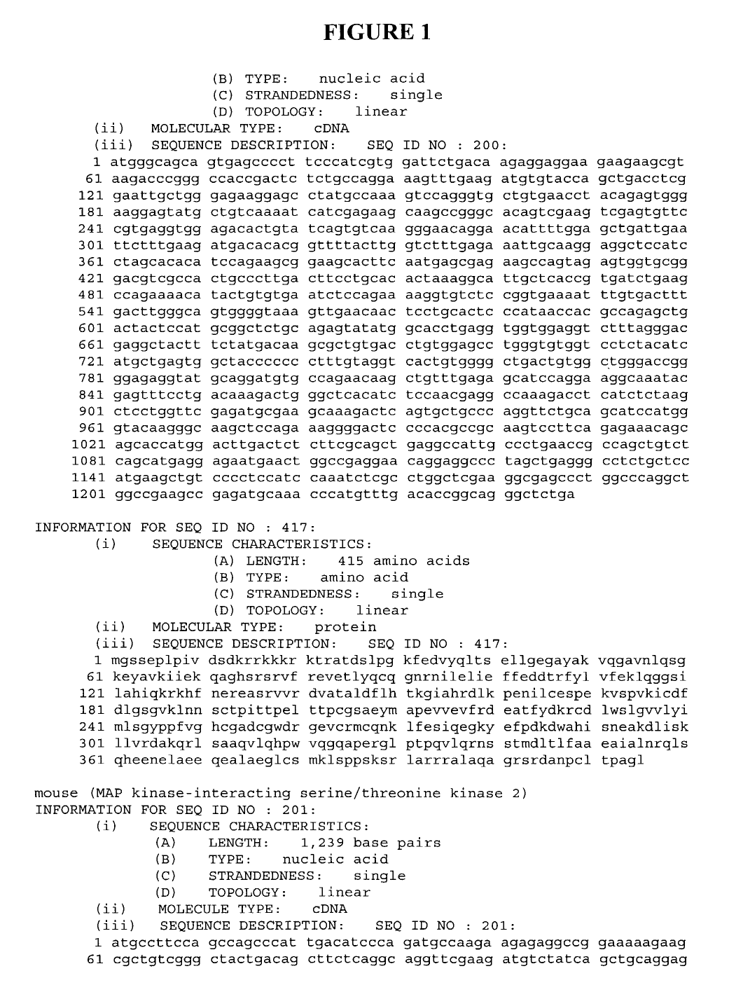

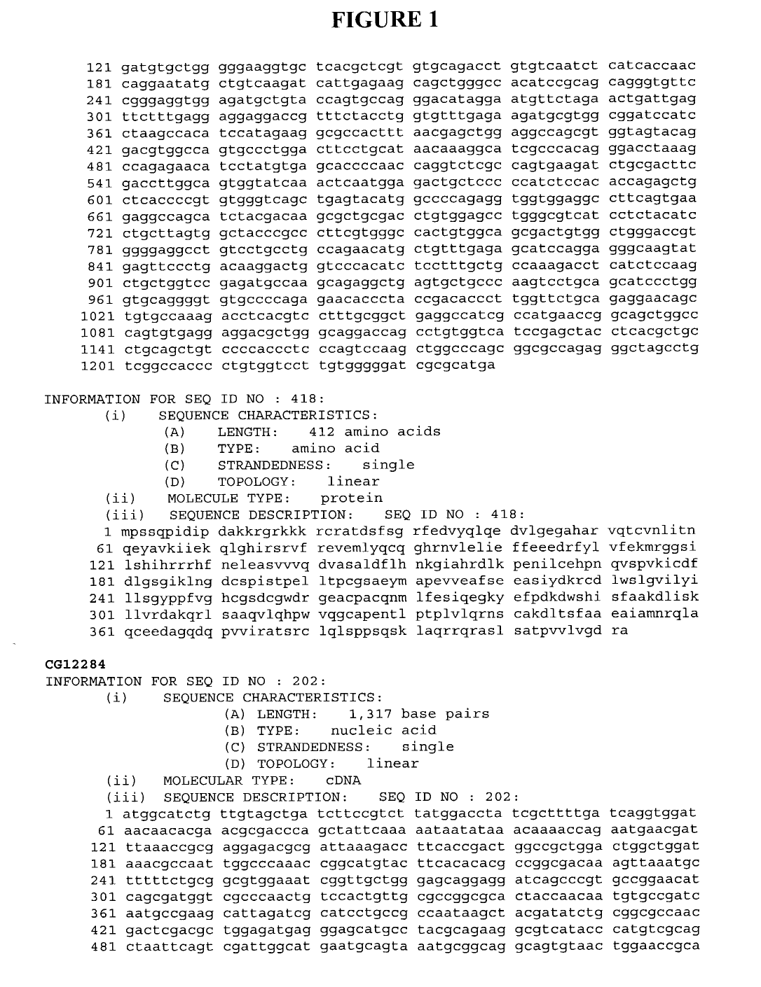

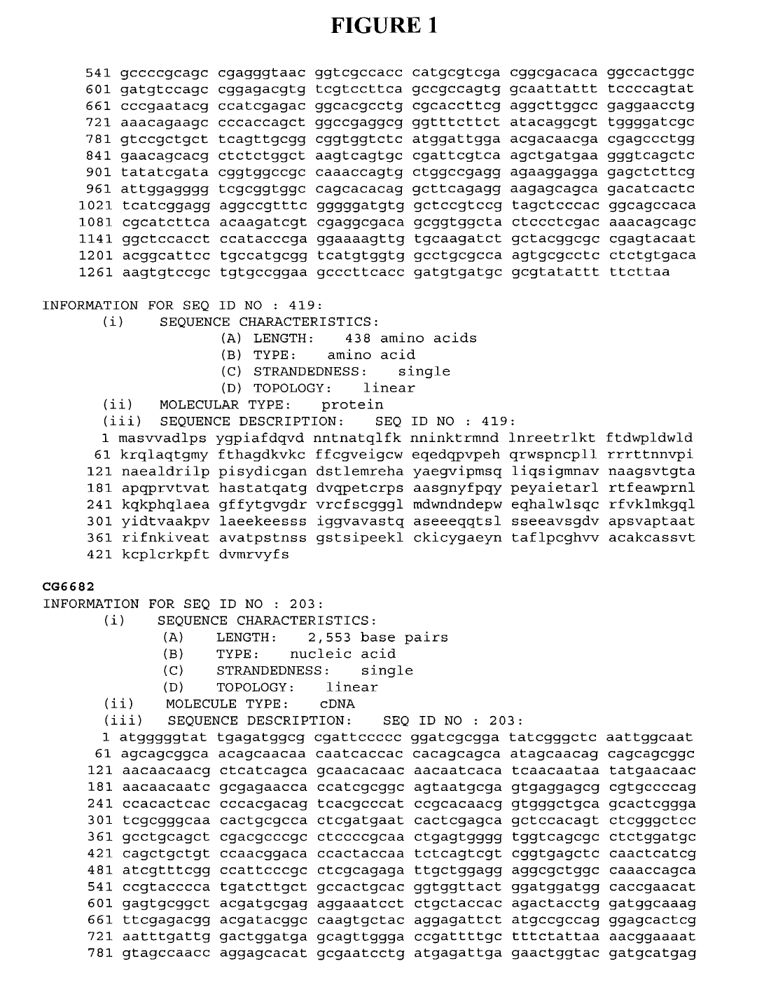

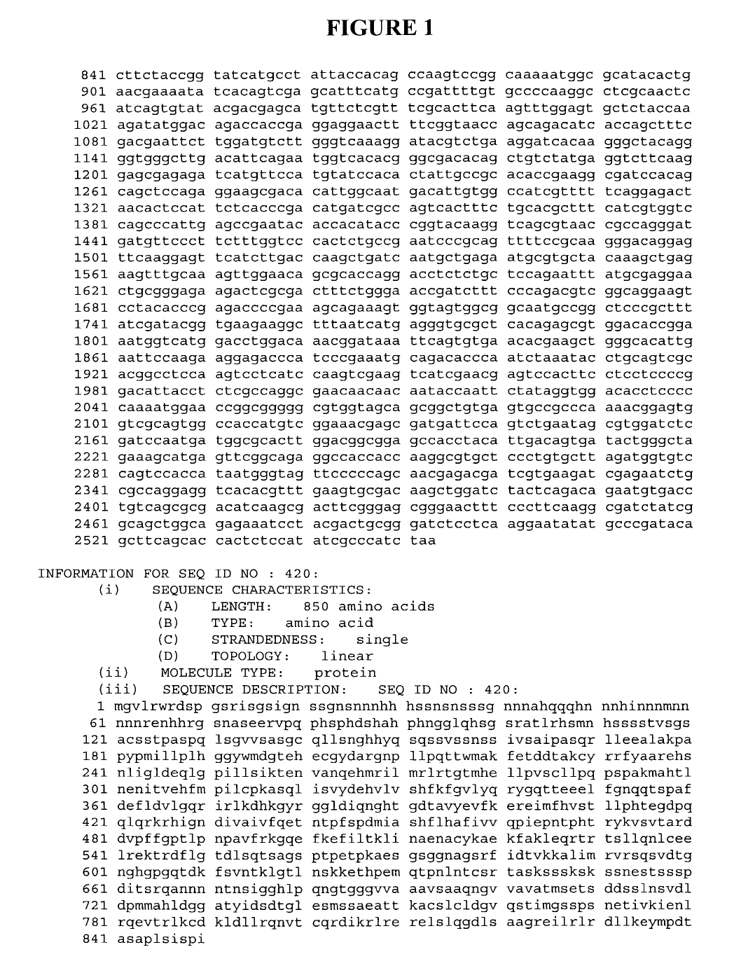

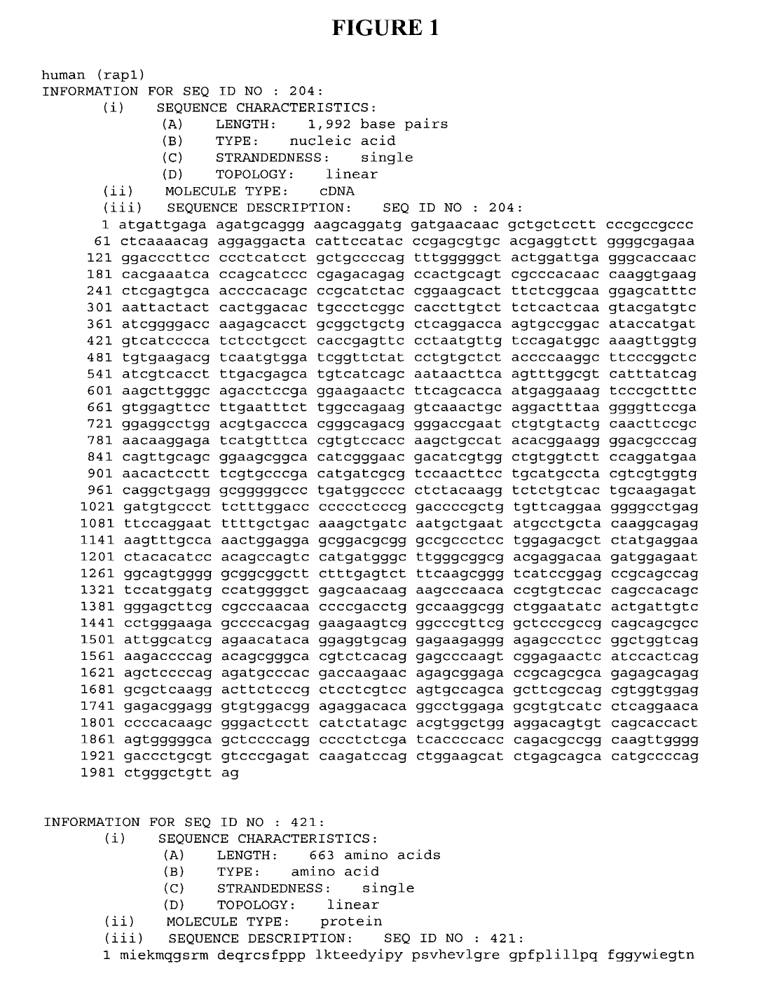

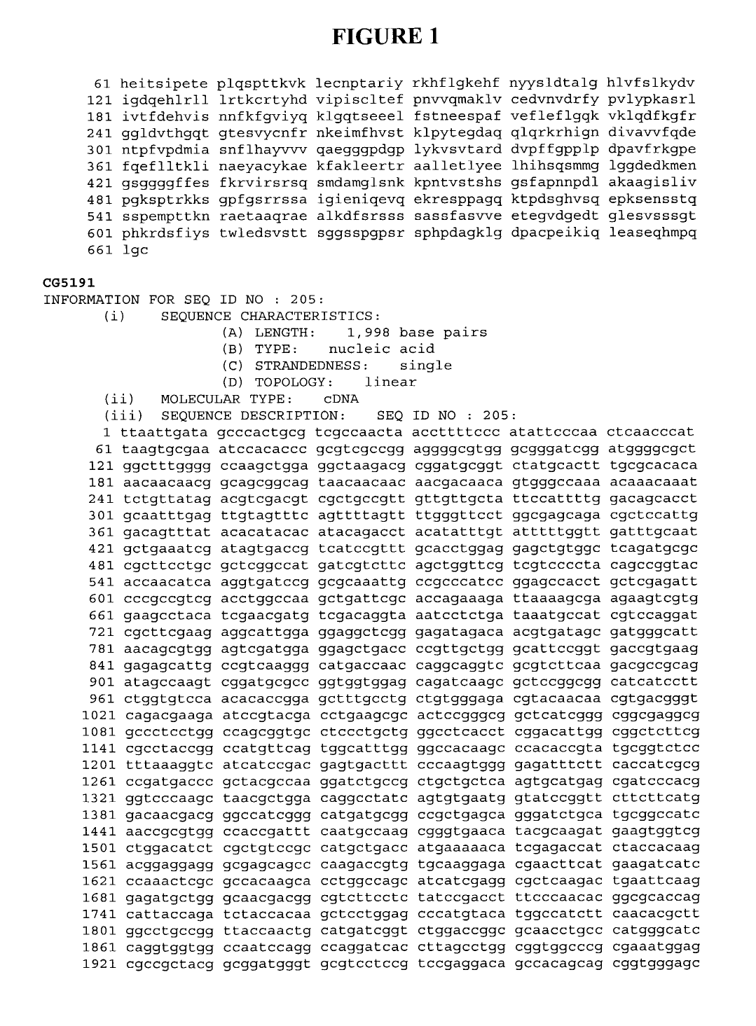

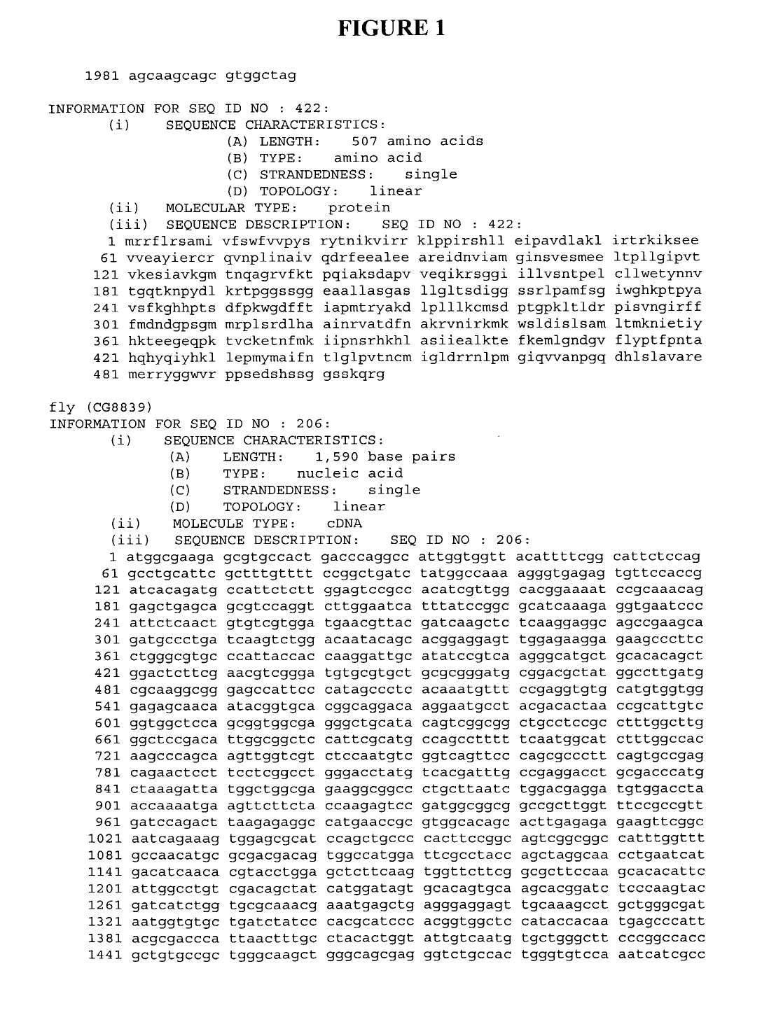

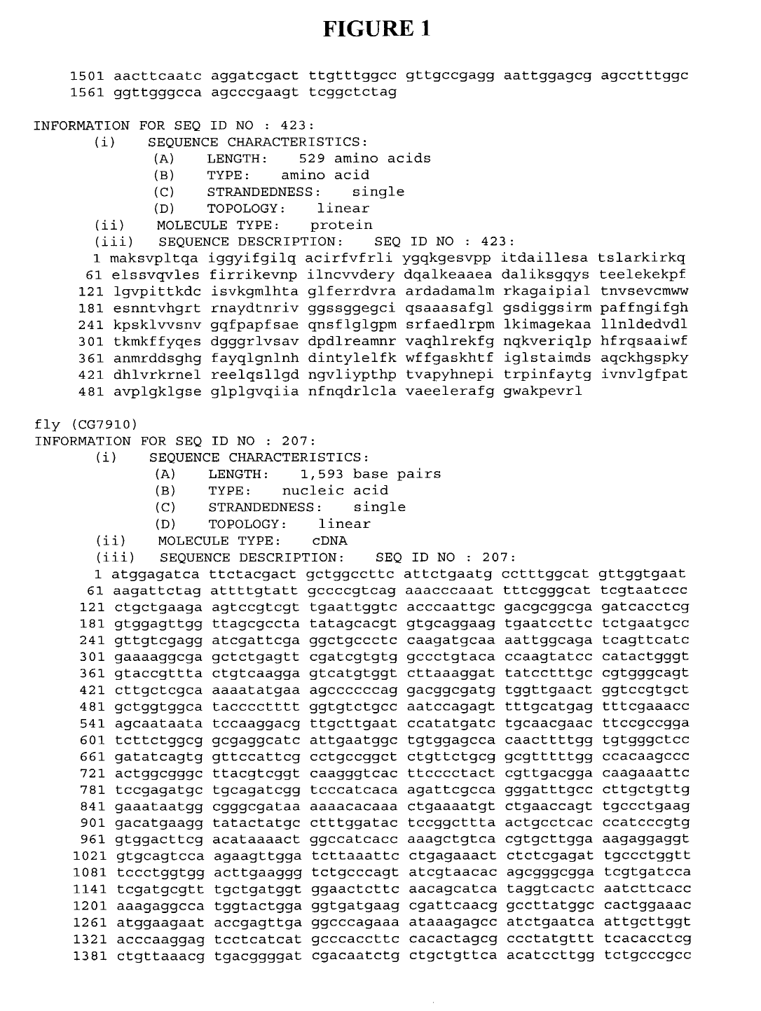

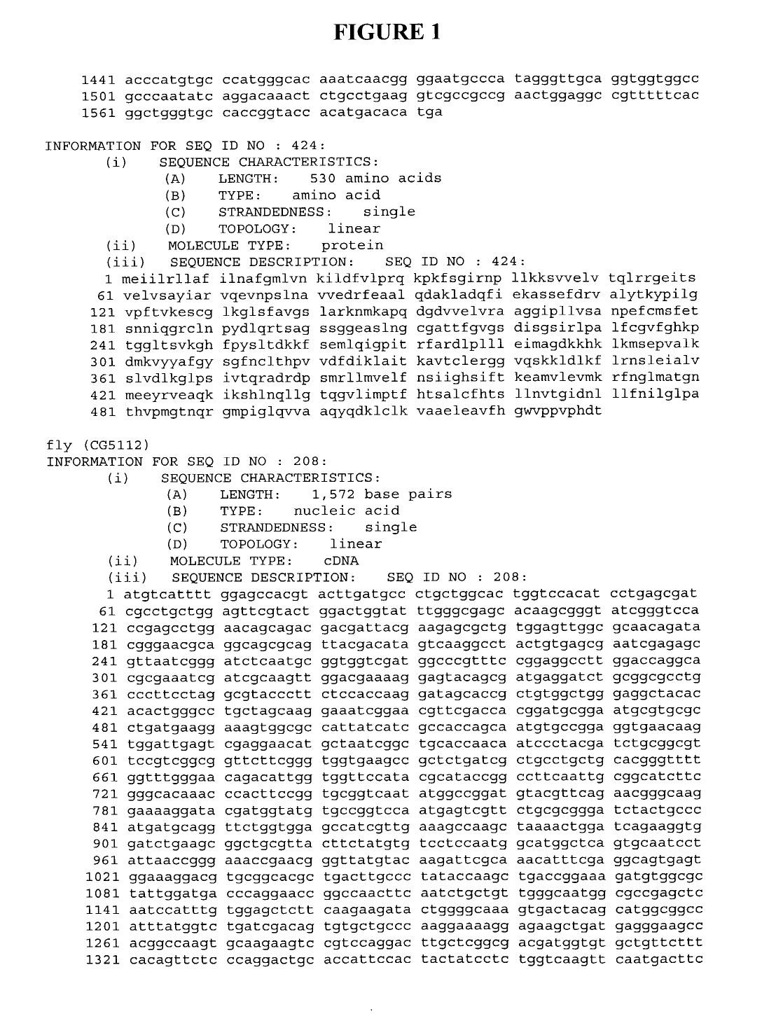

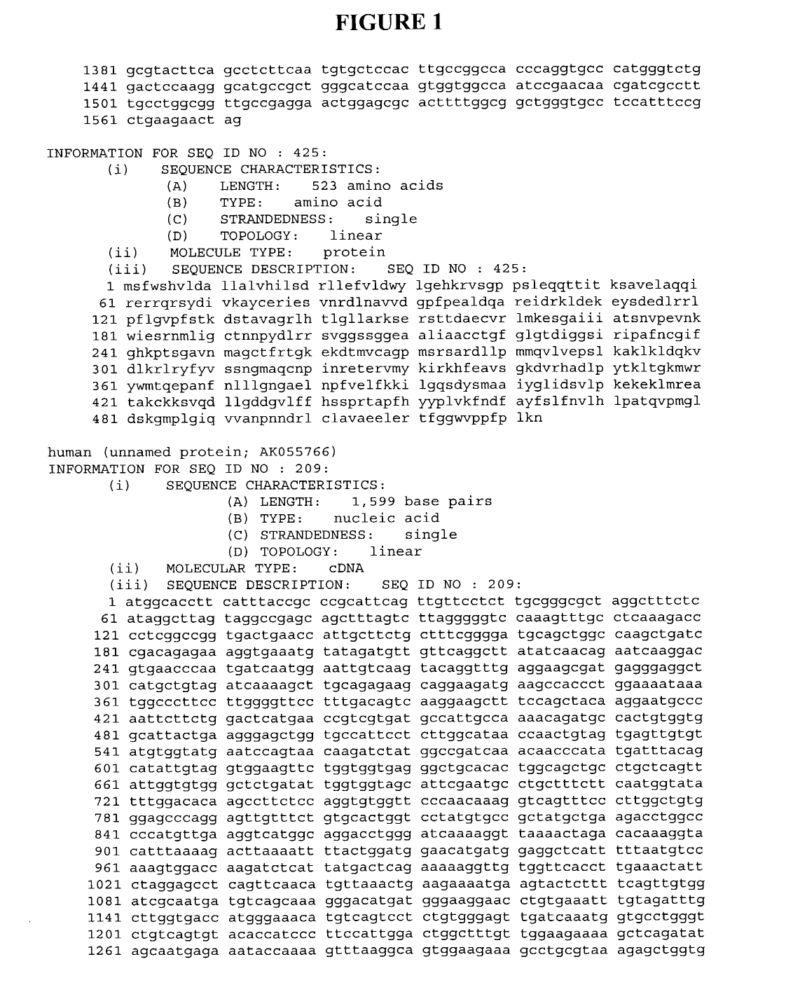

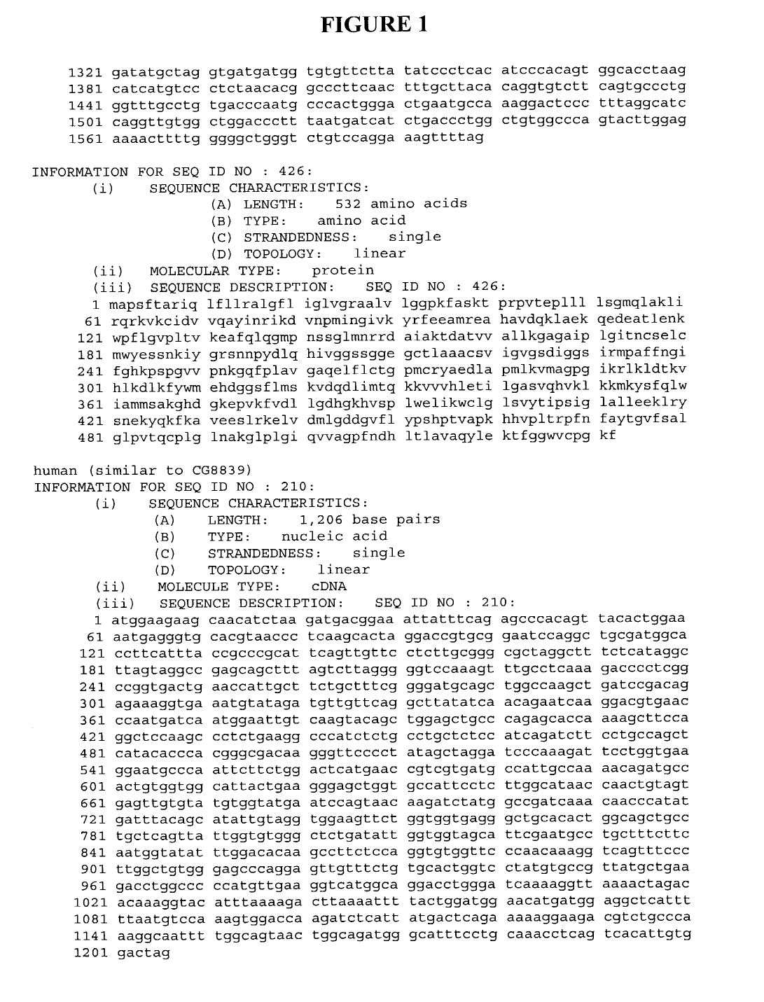

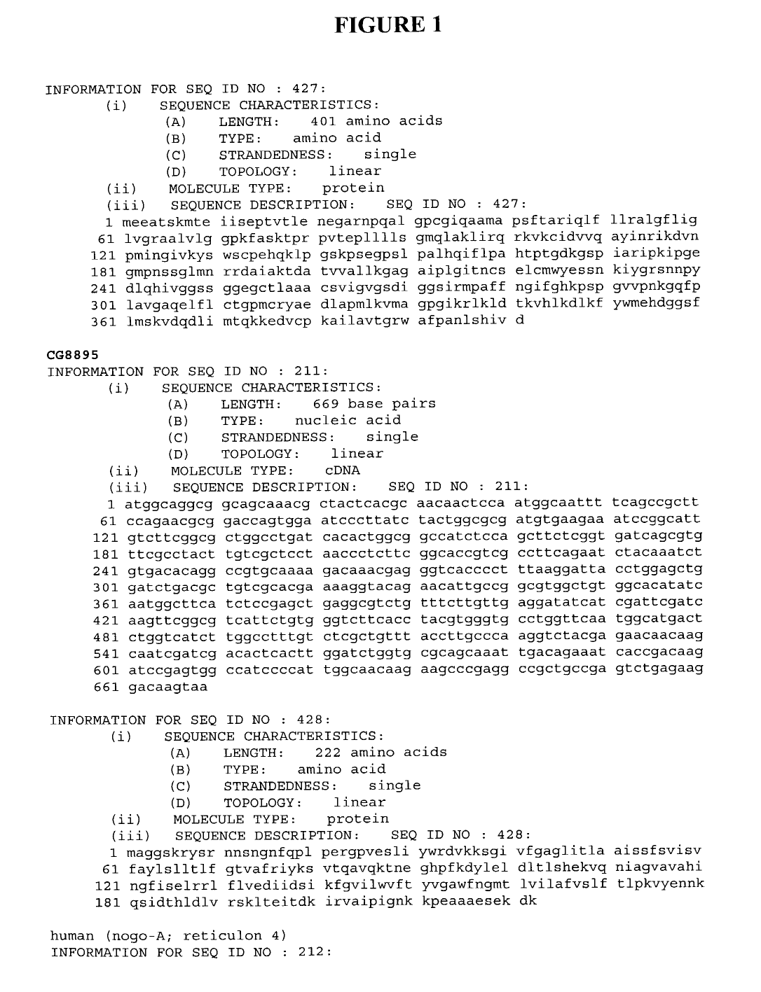

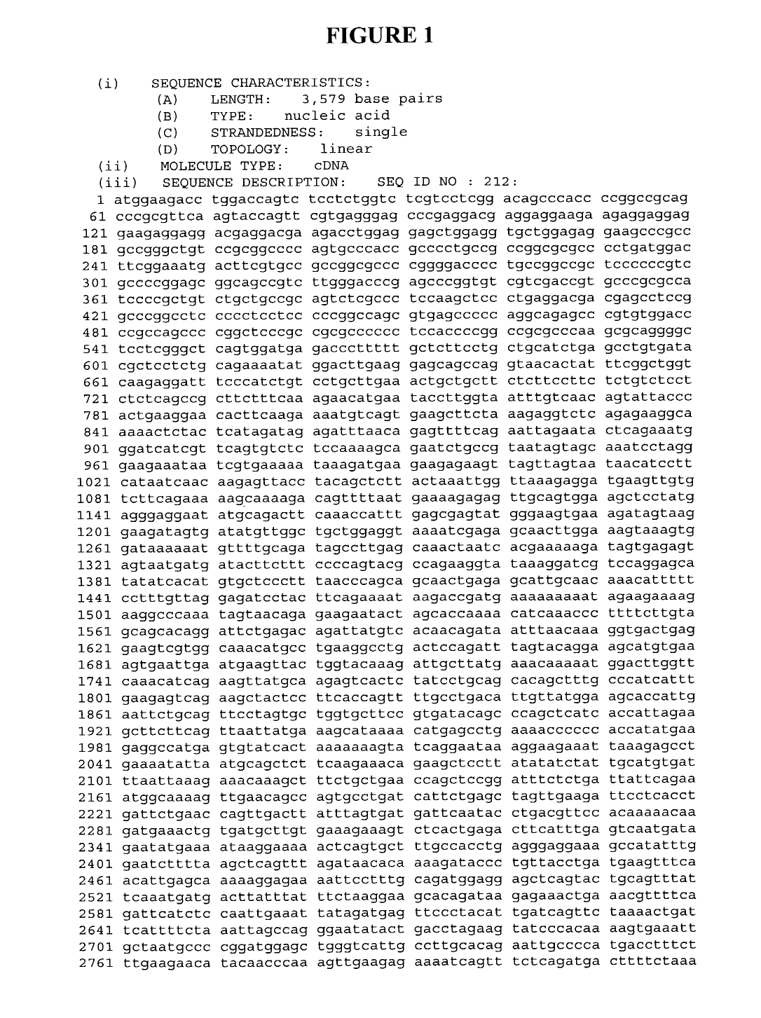

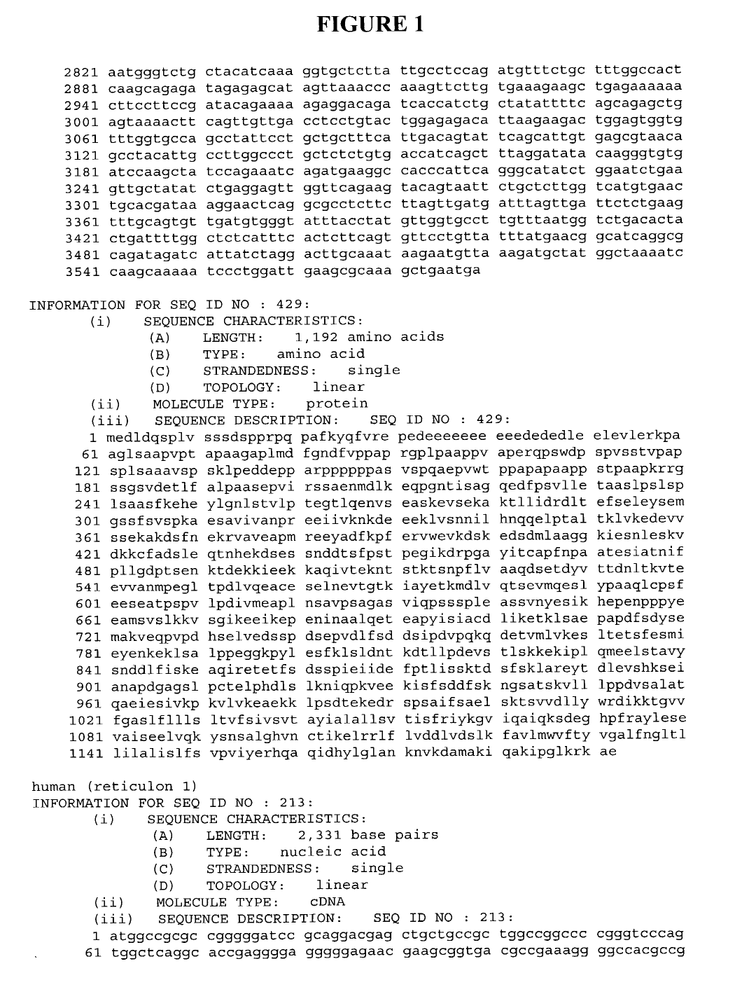

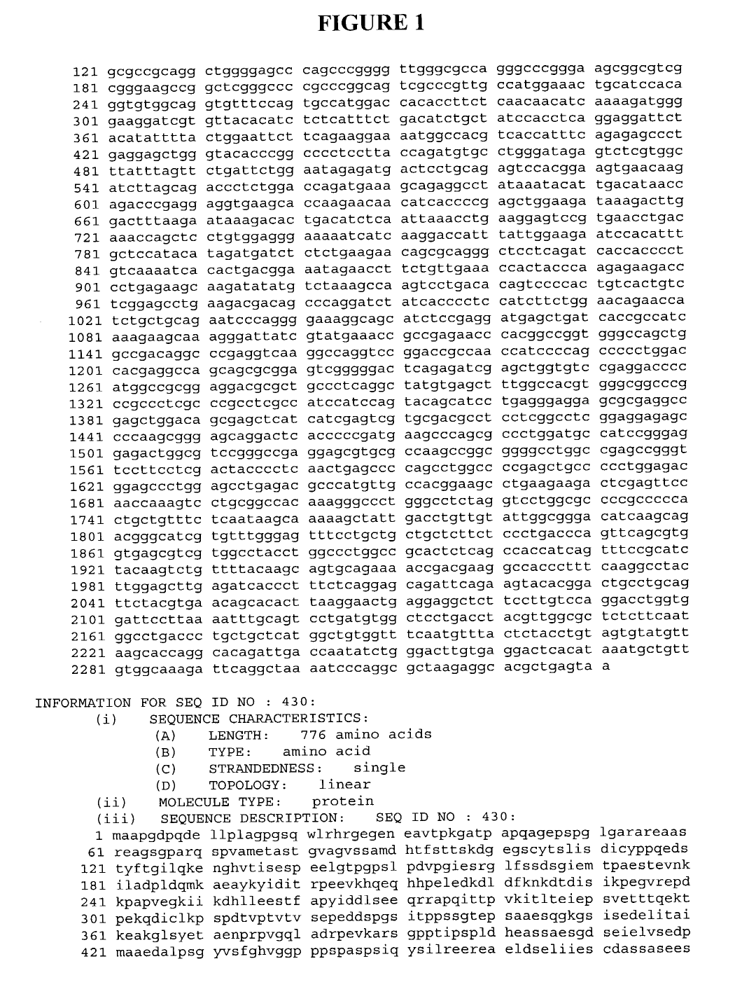

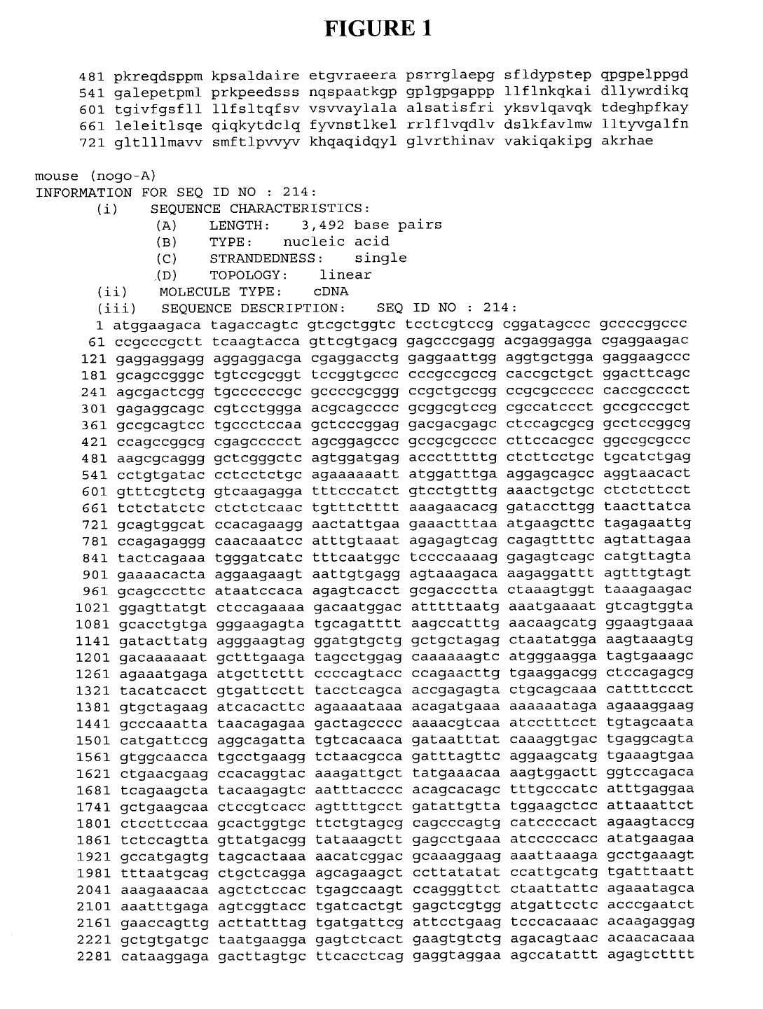

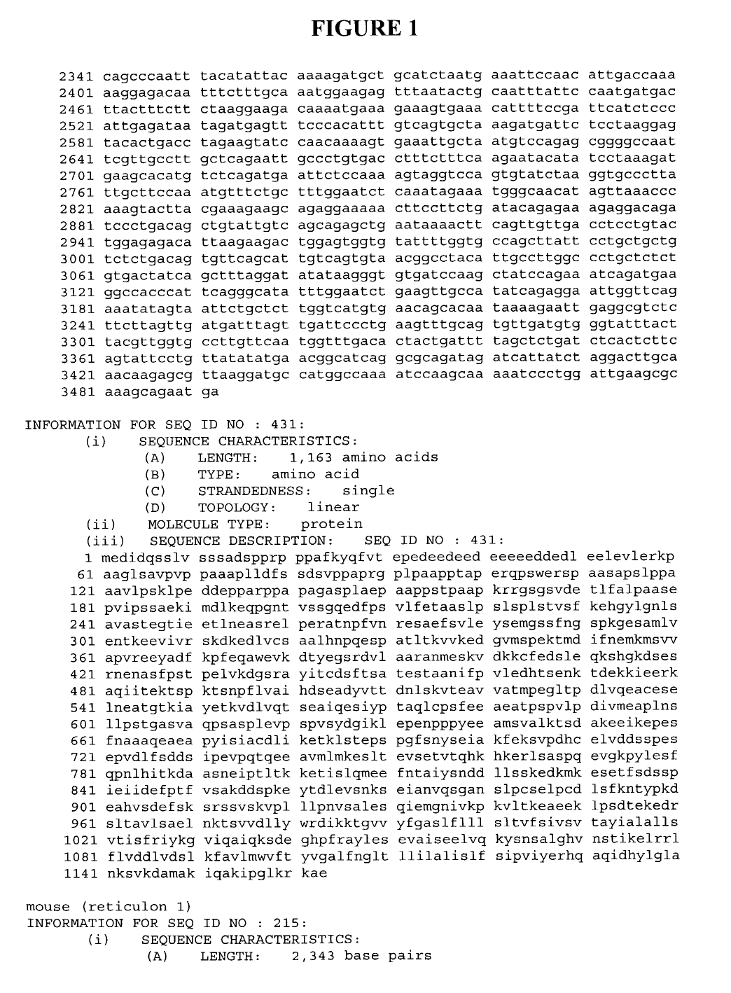

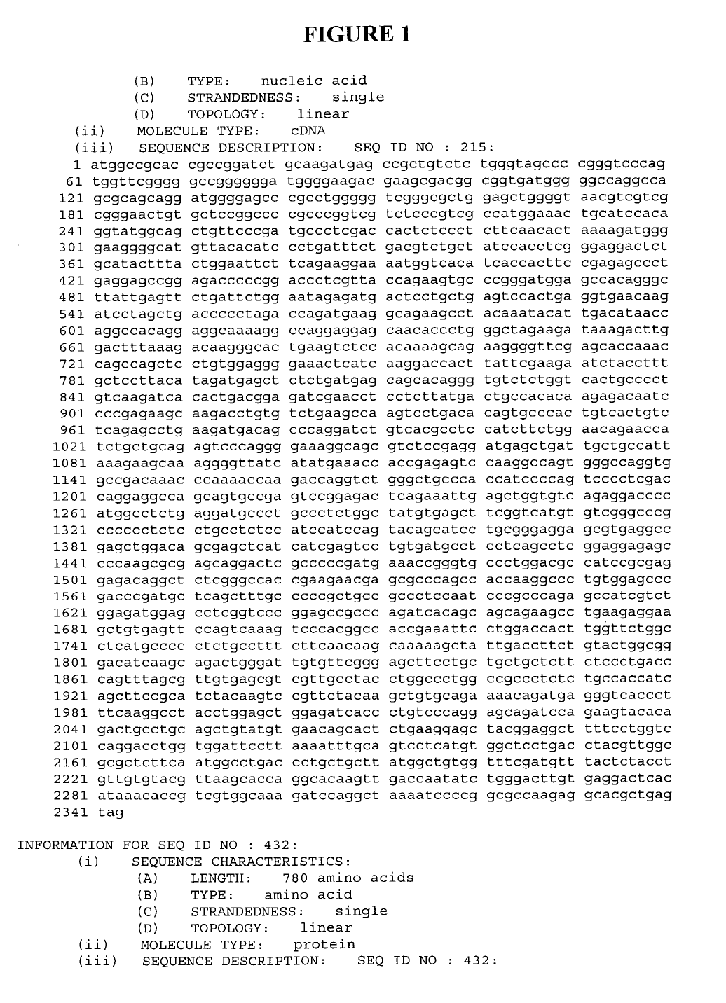

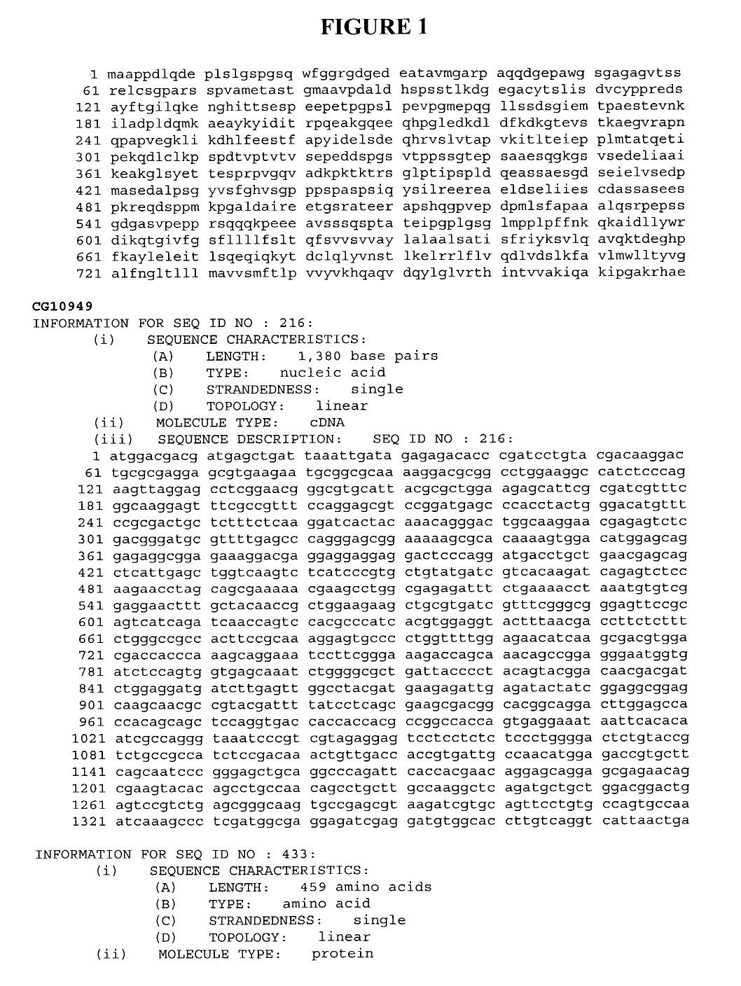

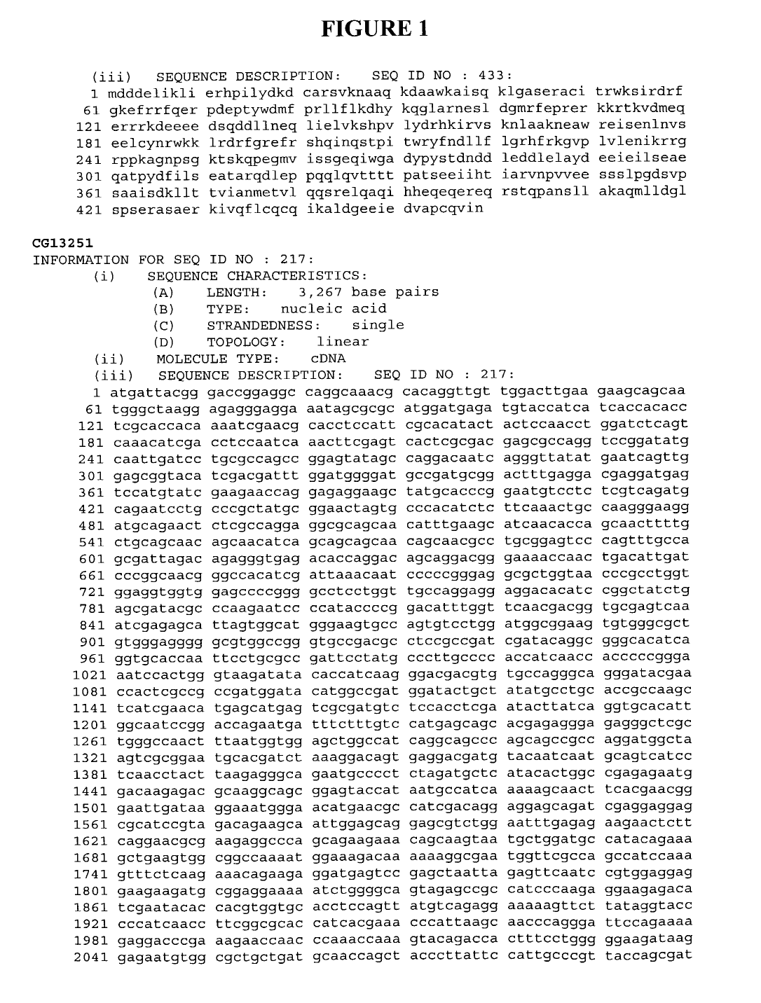

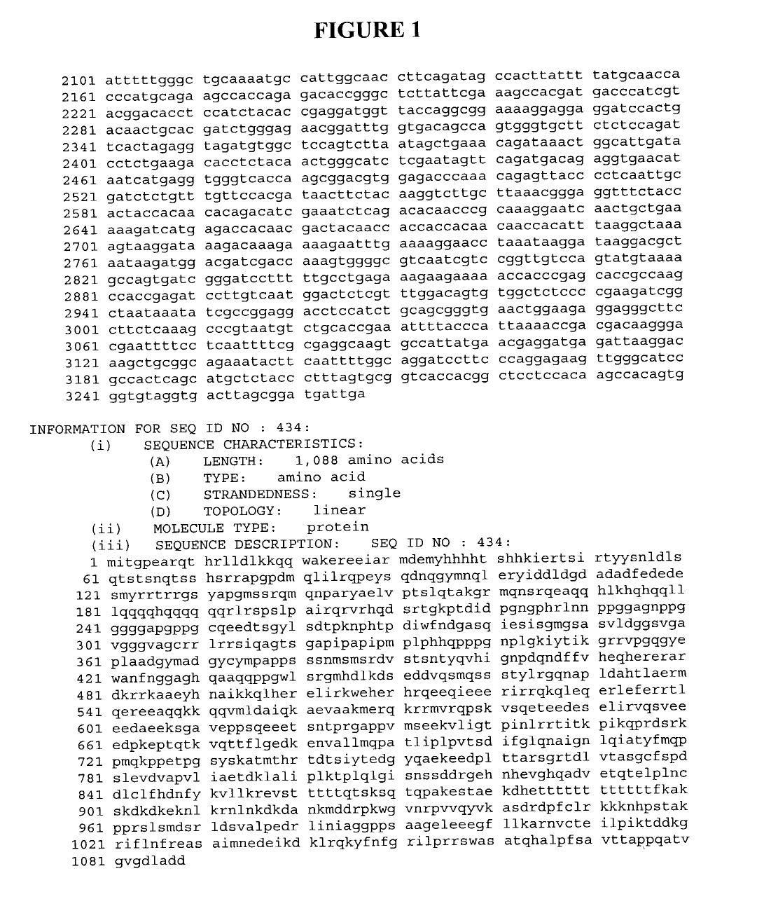

### GENERAL DESCRIPTION OF THE INVENTION

The present invention relates to newly identified nucleic acids, their encoded proteins, and to the use of such nucleic acids and proteins. The invention also relates the correlation between the expression of genes and fat cell size and number. The invention also relates to modifying the activity of a protein that affects the number and/or size of fat cells by regulating the expression of the nucleic acids, homologs, or active variants or their encoded proteins.

Thus, the present invention provides genes and proteins whose expression or activity may be utilized in drug screening (e.g., to identify drugs that alter the expression or activity) and therapeutics contexts.

In experiments conducted during the development of the present invention, the approaches of using Drosophila melanogaster (hereafter referred to as Drosophila) as a system in which to study human disease-associated networks and of genetic screens were combined to identify modifiers of the number and/or size of fat cells.

One of the most profound and surprising biological discoveries in the last two decades is that most animals across the animal kingdom, including humans, possess many of the same genes that function in similar ways in cells, tissues and organs. In fact, only 94 of an estimated 1,278 human gene families are vertebrate-specific. Furthermore, at least 77% of known human disease genes have at least one counterpart within the genome of Drosophila, a model organism and workhorse in the study of genetics (Reiter et al., Gen. Res. 111:1114 (2001); Table 1). Many genes implicated in human diseases, including signaling pathways and effectors of tissue- and cell-specification, were originally identified and characterized in the fruit fly. Thus, genes within most human disease-associated networks are present in the fruit fly genome and have comparable roles in fly biology.

TABLE 1
Drosophila shares many important aspects of biology and disease
pathways with humans

| Genes shared between humans & Drosophila | Human disease relevance |
|---|---|
| Signaling pathway | |
| Notch, presenilin, APP | Alzheimer's disease; |
| | leukemia |
| Hedgehog, ptc | Basal cell carcinoma; |
| | medulloblastoma |
| Insulins, InR, P13K, PDK | Diabetes |
| TGF-beta, Wnt | Colon cancer |
| G-protein coupled receptors | Obesity/diabetes; |
| | hypertension |
| Tissue formation | |
| SREBP, PPAR-gamma | Obesity/diabetes |
| MyoD, Mef | Muscular dystrophy; |
| | cardiomyopathy |
| Pax-6 | Aniridia |
| Cell structural/biological components | |
| p53, Akt, Rb, Ab1, EGF-R | Transformation & |
| | malignancy |
| KCNQ1, KCNH2, SCN5A | Long-QT syndrome |
| KCNQ3, BFNC2, EBN1, KCNQ2 | Neonatal epilepsy |
| PKD1 | Polycystic kidney disease |
| Alpha-syn, parkin, UCHL-1 | Parkinson's disease |

These striking parallels in biological processes among animals are reflected in commercial applications for methods of treatment for human conditions. For example, the protein products of the Transforming Growth Factor-beta (TGF-beta) gene family act as signaling molecules and regulate diverse biological activities (Hogan, Curr. Op. Gen. Dev., 6:432 (1996)). One subset of this family, the Bone Morphogenetic Proteins (BMPs), is characterized by its ability to induce bone formation, both when added to or expressed in cultured cells and when implanted in animals (Sampath et al., Proc. Natl. Acad. Sci. USA 90:6004 (1993)). This has been exploited in a medical device (FDA ref. no. H010002) in which the BMP protein OP-1 is indicated for use as an alternative to autograft in recalcitrant long bone nonunions. The Drosophila counterpart of the OP-1 protein, called 60A, exhibits a very similar biological activity in rats and is sufficient to induce bone formation within dose ranges that have been reported for OP-1 (Sampath et al., 1993). It is therefore expected that Drosophila nucleic acids, their encoded proteins, and the networks within which they interact will have biological activities almost identical to their human homologs. Hence, it is advantageous to use the strengths of Drosophila as an experimental system to study human disease gene-associated networks in genetic modifier screens.

Genetic screens are used to discover genes that carry out various biological activities (St. Johnston, Nat. Rev. Gen. 3:176 (2002)). A change in the activity of a gene in an organism, either through its loss of function or over-expression, can cause a detectable phenotype by disrupting normal biological processes. Genes for which changes in their activity can affect the number and/or size of fat cells are indicated as being part of fat cell proliferation, differentiation, or the metabolism of fats or sugars. As described in detail below, the present invention provides a number of genes whose expression is correlated to the regulation of fat cell size and/or number.

The screens used during the development of the present invention, as described in the examples below, provide unparalleled capability for identifying genes and proteins with desired in vivo activities. In particular, the present invention employs a collection of genetically modified organisms that statistically represent at least one organism that overexpresses each gene of the genome. These animals can be crossed against a disease model (e.g., an animal expressing a human gene associated with disease) to determine which of the genes of the genome alter the phenotype of the model. Thus, a comprehensive survey of the in vivo activity of each gene in the genome is provided. Unlike existing high-throughput in vitro methods (microarrays, proteomic methods, twin-hybrid systems), the methods used in the present invention provide high-throughput in vivo results. While in vitro approaches have successfully generated dense arrays of data, they lack a critical ability to discriminate among the hundreds or thousands of identified targets because they do not assess their functional relevance.

Genetic modifier screens, such as those of the present invention, offer a superior alternative to other systems because they identify only genes of biological relevance. Genetic modifier screens are used to test interactions among genes that act together within networks to carry out various biological activities. A change in the activity of a gene in an organism, either through its loss of function or mis-expression, can cause a detectable phenotype by disrupting normal biological processes. A change in the activity of another gene that acts together within a gene network with the first will often detectably modify this phenotype by either enhancing or suppressing it. Changes in the activity of genes that do not genetically interact with the first gene will not specifically modify the phenotype. Genetic modifier screens hold a crucial advantage over yeast two-hybrid or proteomics systems in their ability to detect interactions among genes whose products may not physically interact. Furthermore, they are far better than DNA microarray technologies because genetic modifier screens identify interactions of biological importance, not just associations between gene expression patterns and different cell- or tissue-types.

Definitions

To facilitate understanding of the invention, a number of terms are defined below.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where amino acid sequence is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “competes for binding” is used in reference to a first polypeptide with an activity which binds to the same substrate as does a second polypeptide with an activity, where the second polypeptide is a variant of the first polypeptide or a related or dissimilar polypeptide. The efficiency (e.g., kinetics or thermodynamics) of binding by the first polypeptide may be the same as or greater than or less than the efficiency substrate binding by the second polypeptide. For example, the equilibrium binding constant (K_{D}) for binding to the substrate may be different for the two polypeptides. The term “Kₘ” as used herein refers to the Michaelis-Menton constant for an enzyme and is defined as the concentration of the specific substrate at which a given enzyme yields one-half its maximum velocity in an enzyme catalyzed reaction.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent (50×Denhardt's contains per 500 ml: 5 g Ficoll (Type 400, Pharamcia), 5 g BSA (Fraction V; Sigma)) and 100 g/ml denatured salmon sperm DNA followed by washing in a solution comprising 5×SSPE, 0.1% SDS at 42 C when a probe of about 500 nucleotides in length is employed.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math. 2: 482 (1981)) by the homology alignment algorithm of Needleman and Wunsch (Needleman and Wunsch, J. Mol. Biol. 48:443 (1970)), by the search for similarity method of Pearson and Lipman (Pearson and Lipman, Proc. Natl. Acad. Sci. USA 85:2444 (1988)), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions with its various ligands and/or substrates.

The term “polymorphic locus” is a locus present in a population that shows variation between members of the population (i.e., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

As used herein, the term “polymorphism information” refers to the presence or absence of one or more polymorphisms (e.g., mutations) in a gene.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q replicase, MDV-1 RNA is the specific template for the replicase (D. L. Kacian et al., Proc. Natl. Acad. Sci. USA 69:3038 (1972)). Other nucleic acid will not be replicated by this amplification enzyme. Similarly, in the case of T7 RNA polymerase, this amplification enzyme has a stringent specificity for its own promoters (Chamberlin et al., Nature 228:227 (1970)). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 (1989)). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989)).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., mRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand. Regions of a nucleic acid sequences that are accessible to antisense molecules can be determined using available computer analysis methods.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a gene includes, by way of example, such nucleic acid in cells ordinarily expressing the gene where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets which specify stop codons (i.e., TAA, TAG, TGA).

As used herein, the term “purified” or “to purify” refers to the removal of contaminants from a sample. For example, antibodies are purified by removal of contaminating non-immunoglobulin proteins; they are also purified by the removal of immunoglobulin that does not bind a target of interest. The removal of non-immunoglobulin proteins and/or the removal of immunoglobulins that do not bind the target results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 (1989)).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 (1989)).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “transgene” as used herein refers to a foreign gene that is placed into an organism by introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term host cell refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Example 10, for a protocol for performing Northern blot analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 (1973)), has been modified by several groups to optimize conditions for particular types of cells. The art is well aware of these numerous modifications.

The term “test compound” or “agent” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

### DETAILED DESCRIPTION OF THE INVENTION

The present invention relates to newly identified nucleic acids, their encoded proteins, and to the use of such nucleic acids and proteins. The invention also relates the correlation between the expression of genes and fat cell size and number. The invention also relates to modifying the activity of a protein that affects the number and/or size of fat cells by regulating the expression of the nucleic acids, homologs, or active variants or their encoded proteins. The present invention also encompasses methods for screening for agents that inhibit or potentiate action of a target gene or protein. The present invention also relates to methods for screening for susceptibility to obesity or obesity or wasting-related diseases and conditions.

Experiments conducted during the development of the present invention have identified a number of genes whose expression was shown to correlate to changes in fat cell size and/or number. The genes are provided in Table 2, below (NA SEQ=nucleic acid SEQ ID NO.; AA=amino acid SEQ ID NO.; a+=more fat cells; a−=less fat cells; s+=larger fat cells; s−=smaller fat cells; red=overall reduced animal growth).

TABLE 2

| Gene name | Phenotypic | | | NA | AA |
|---|---|---|---|---|---|
| Fly | Effect | Homologue | Common Name | SEQ | SEQ |
| CG5555 | red. | | | 1 | 218 |
| | | Human | BRCA1-associated protein 2 | 2 | 219 |
| | | Mouse | BRAP2 variant 1 | 3 | 220 |
| CG11630 | a+ | | | 4 | 221 |
| CG1973 | red. | | | 5 | 222 |
| | | Human | kinase-like protein | 6 | 223 |
| | | Mouse | N-terminal kinase-like | 7 | 224 |
| CG12521 | s+ | | | 8 | 225 |
| CG13988 | a+ | | | 9 | 226 |
| CG10364 | a+ | | | 10 | 227 |
| CG10120 | red. | | | 11 | 228 |
| | | Fly | Malic enzyme | 12 | 229 |
| | | Fly | CG30097 | 13 | 230 |
| | | Fly | CG7848 | 14 | 231 |
| | | Human | malic enzyme 1 | 15 | 232 |
| | | Human | malic enzyme 3 | 16 | 233 |
| | | Human | malic enzyme | 17 | 234 |
| | | Mouse | malic enzyme 1 | 18 | 235 |
| | | Mouse | malic enzyme 2 | 19 | 236 |
| | | Mouse | similar to malic enzyme 3 | 20 | 237 |
| CG10480 | s+ | | | 21 | 238 |
| | | Human | regulator of chromosome condensation 1 | 22 | 239 |
| | | Mouse | regulator of chromosome condensation 1 | 23 | 240 |
| CG10198 | a+ | | | 24 | 241 |
| | | Human | nucleoporin 98 | 25 | 242 |
| | | rat | | 26 | 243 |
| CG7664 | a+ | | | 27 | 244 |
| CG9761 | a+ | | neprilysin 2 | 28 | 245 |
| | | Fly | neprilysin 1 | 29 | 246 |
| | | Fly | CG4058 | 30 | 247 |
| | | Fly | neprilysin 3 | 31 | 248 |
| | | Fly | CG14527 | 32 | 249 |
| | | Fly | CG5527 | 33 | 250 |
| | | Human | neprilysin-like metallopeptidase 2 | 34 | 251 |
| | | Human | endothelin converting enzyme 1 | 35 | 252 |
| | | Human | endothelin converting enyzme 2 | 36 | 253 |
| | | Human | neprilysin | 37 | 254 |
| | | Human | endothelin converting enzyme-like 1 | 38 | 255 |
| | | Human | phosphate regulating neutral | 39 | 256 |
| | | | endopeptidase | | |
| | | Mouse | neprilysin-like peptidase 1 | 40 | 257 |
| | | Mouse | membrane metallo endopeptidase | 41 | 258 |
| | | Mouse | endothelin converting enyzme 2 | 42 | 259 |
| | | Mouse | endothelin-converting enzyme-like 1 | 43 | 260 |
| | | Mouse | phosphate regulating neutral | 44 | 261 |
| | | | endopeptidase | | |
| | | Mouse | similar to endothelin-converting enzyme 1 | 45 | 262 |
| CG11993 | s+, a+ | | | 46 | 263 |
| CG8121 | s+, a+ | | | 47 | 264 |
| CG9021 | a+ | | | 48 | 265 |
| CG4974 | a+ | | | 49 | 266 |
| | | Fly | Dally-like protein | 50 | 267 |
| | | Human | glypican 5 | 51 | 268 |
| | | Human | glypican 1 | 52 | 269 |
| | | Human | glypican 4 | 53 | 270 |
| | | Human | glypican 6 | 54 | 271 |
| | | Human | glypican 3 | 55 | 272 |
| | | Mouse | glypican 1 | 56 | 273 |
| | | Mouse | glypican 6 | 57 | 274 |
| | | Mouse | glypican 4 | 58 | 275 |
| | | Mouse | glypican 3 | 59 | 276 |
| CG11706 | a+ | | | 60 | 277 |
| CG13299 | red. | | | 61 | 278 |
| CG9894 | a+ | | | 62 | 279 |
| CG12078 | red. | | | 63 | 280 |
| CG11711 | a+ | | | 64 | 281 |
| | | Fly | CG13852 | 65 | 282 |
| | | Human | mob1 | 66 | 283 |
| | | Human | similar to MOB-LAK | 67 | 284 |
| | | Mouse | mob1 | 68 | 285 |
| | | Mouse | ovary-specific MOB-like protein | 69 | 286 |
| | | Mouse | similar to MOB-LAK | 70 | 287 |
| CG4422 | a+ | | | 71 | 288 |
| | | Fly | GDP dissociation inhibitor | 72 | 289 |
| | | Human | GDI1 | 73 | 290 |
| | | Human | GDI2 | 74 | 291 |
| | | Mouse | GDI1 | 75 | 292 |
| | | Mouse | GDI2 | 76 | 293 |
| | | Mouse | GDI3 | 77 | 294 |
| CG15259 | a+ | | | 78 | 295 |
| CG9674 | a+ | | | 79 | 296 |
| CG10544 | a+ | | | 80 | 297 |
| CG17646 | a+ | | | 81 | 298 |
| | | Fly | C3164 | 82 | 299 |
| | | Fly | CG9663 | 83 | 300 |
| | | Fly | CG3164 | 84 | 301 |
| | | Fly | CG31689; CG9892 | 85 | 302 |
| | | Fly | CG2969 | 86 | 303 |
| | | Fly | CG5853; CG4619 | 87 | 304 |
| | | Fly | CG4822 | 88 | 305 |
| | | Fly | CG7346 | 89 | 306 |
| | | Human | ATP-binding cassette, subfamily G, | 90 | 307 |
| | | | member 1 | | |
| | | Human | ATP-binding cassette, sub-family G, | 91 | 308 |
| | | | member 4 | | |
| | | Mouse | ATP-binding cassette, subfamily G, | 92 | 309 |
| | | | member 1 | | |
| | | Mouse | ATP-binding cassette, sub-family G, | 93 | 310 |
| | | | member 4 | | |
| CG5643 | a+ | | | 94 | 311 |
| | | Fly | CG7901; PP2A-B′ | 95 | 312 |
| | | Human | protein phosphatase 2, regulatory subunit | 96 | 313 |
| | | | B | | |
| | | Human | PPP2R5D | 97 | 314 |
| | | Mouse | Protein phosphatase 2A regulatory B | 98 | 315 |
| | | | subunit | | |
| | | Mouse | protein phosphatase 2A B56delta | 99 | 316 |
| | | | regulatory subunit | | |
| CG7051 | s+ | | | 100 | 317 |
| | | Fly | CG13930 | 101 | 318 |
| | | Human | axonemal dynein intermediate chain 1 | 102 | 319 |
| | | Mouse | similar to hypothetical protein FLJ23129 | 103 | 320 |
| CG9366 | red. | | | 104 | 321 |
| | | Fly | Rac1 | 105 | 322 |
| | | Fly | Rac2 | 106 | 323 |
| | | Fly | Mig-2-like GTPase Mt1 | 107 | 324 |
| | | Fly | CDC42 | 108 | 325 |
| | | Human | Rac3 | 109 | 326 |
| | | Human | Rac1 | 110 | 327 |
| | | Human | Rac2 | 111 | 328 |
| | | Mouse | Rac1 | 112 | 329 |
| | | Mouse | Rac3 | 113 | 330 |
| | | Mouse | Rac2 | 114 | 331 |
| CG11914 | a+ | | | 115 | 332 |
| CG5841 | a+ | | | 116 | 333 |
| | | Fly | CG17492 | 117 | 334 |
| | | Human | skeletrophin | 118 | 335 |
| | | Human | unnamed protein; AK097106 | 119 | 336 |
| | | Human | unnamed protein; AK096295 | 120 | 337 |
| | | Human | KIAA1323 | 121 | 338 |
| | | Mouse | skeletrophin | 122 | 339 |
| | | Mouse | similar to CG17492 | 123 | 340 |
| CG17360 | a− | | | 124 | 341 |
| CG10336 | s+ | | | 125 | 342 |
| CG17024 | s+ | | | 126 | 343 |
| | | Human | phosphoribosylaminoimidazole | 127 | 344 |
| | | | carboxylase | | |
| | | Mouse | phosphoribosylaminoimidazole | 128 | 345 |
| | | | carboxylase | | |
| CG7664 | a+ | | | 129 | 346 |
| | | Human | AP-4 | 130 | 347 |
| | | Mouse | AP-4 | 131 | 348 |
| CG1658 | a+ | | | 132 | 349 |
| | | Human | Similar to CDC-like kinase 2 | 133 | 350 |
| | | Human | clk-3 | 134 | 351 |
| | | Human | clk-4 | 135 | 352 |
| | | Human | clk-1 | 136 | 353 |
| | | Mouse | clk-3 | 137 | 354 |
| | | Mouse | clk-4 | 138 | 355 |
| | | Mouse | clk-1 | 139 | 356 |
| | | Mouse | similar to clk-4 | 140 | 357 |
| CG6783 | s+ | | | 141 | 358 |
| | | Human | fatty acid binding protein | 142 | 359 |
| | | Human | mammary-derived growth inhibitor | 143 | 360 |
| | | Mouse | fatty acid binding protein 7, brain | 144 | 361 |
| CG3779 | a+ | | | 145 | 362 |
| | | Human | h-numb | 146 | 363 |
| | | Human | numb-related | 147 | 364 |
| | | Mouse | m-numb | 148 | 365 |
| | | Mouse | numb-like protein | 149 | 366 |
| CG12086 | a+ | | | 150 | 367 |
| CG1451 | s+ | | | 151 | 368 |
| | | Fly | APC2 | 152 | 369 |
| | | Human | APC | 153 | 370 |
| | | Human | APC2 | 154 | 371 |
| | | Mouse | APC | 155 | 372 |
| | | Mouse | APC2 | 156 | 373 |
| CG13791 | a− | | | 157 | 374 |
| CG7625 | s+, a+ | | | 158 | 375 |
| CG15171 | a+ | | | 159 | 376 |
| CG3408 | s+ | | | 160 | 377 |
| CG10295 | s+ | | | 161 | 378 |
| | | Human | p21-activated kinase 3 | 162 | 379 |
| | | Human | pak1 | 163 | 380 |
| | | Human | pak2 | 164 | 381 |
| | | Mouse | p21-activated kinase 3 | 165 | 382 |
| | | Mouse | pak1 | 166 | 383 |
| | | Mouse | similar to pak2 | 167 | 384 |
| CG12070 | s+ | | | 168 | 385 |
| | | Human | prosaposin; sphingolipid activator protein- | 169 | 386 |
| CG18496 | a+ | | | 170 | 387 |
| CG9894 | s+ | | | 171 | 388 |
| CG8244 | red. | | | 172 | 389 |
| CG6588 | red. | | | 173 | 390 |
| CG1427 | red. | | | 174 | 391 |
| | | Human | SLA/LP autoantigen | 175 | 392 |
| CG9373 | red. | | | 176 | 393 |
| | | Human | KIAA1341 | 177 | 394 |
| | | Human | unnamed protein product; AK023133 | 178 | 395 |
| | | Human | mef2 | 179 | 396 |
| | | Mouse | myelin gene expression factor | 180 | 397 |
| CG11888 | s+ | | | 181 | 398 |
| | | Human | 26S proteasome subunit p112 | 182 | 399 |
| | | Mouse | similar to 26S proteasome, subunit p112 | 183 | 400 |
| CG16791 | red. | | | 184 | 401 |
| CG9351 | a+ | | | 185 | 402 |
| | | Human | KIAA1387 protein | 186 | 403 |
| | | Human | hypothetical protein FLJ20707 | 187 | 404 |
| | | Mouse | NM_134034 expressed sequence | 188 | 405 |
| | | | AW011752 | | |
| | | Mouse | XM_141919 similar to expressed | 189 | 406 |
| | | | sequence AW011752 | | |
| | | Mouse | XM_141922 similar to expressed | 190 | 407 |
| | | | sequence AW011752 | | |
| | | Mouse | XM_141917 similar to expressed | 191 | 408 |
| | | | sequence AW011752 | | |
| CG3671 | red. | | | 192 | 409 |
| | | Human | solute carrier family 11, member 2 | 193 | 410 |
| | | Human | solute carrier family 11, member 1 | 194 | 411 |
| | | Mouse | solute carrier family 11, member 2 | 195 | 412 |
| | | Mouse | solute carrier family 11, member 1 | 196 | 413 |
| CG17342 | s+, a− | | | 197 | 414 |
| | | Human | MAP kinase-interacting kinase 1 | 198 | 415 |
| | | Human | MAP kinase-interacting kinase 2a | 199 | 416 |
| | | Mouse | MAP kinase-interacting serine/threonine | 200 | 417 |
| | | | kinase 1 | | |
| | | Mouse | MAP kinase-interacting serine/threonine | 201 | 418 |
| | | | kinase 2 | | |
| CG12284 | a− | | | 202 | 419 |
| CG6682 | s+ | | | 203 | 420 |
| | | Human | rap1 | 204 | 421 |
| CG5191 | a+ | | | 205 | 422 |
| | | Fly | CG8839 | 206 | 423 |
| | | Fly | CG7910 | 207 | 424 |
| | | Fly | CG5112 | 208 | 425 |
| | | Human | unnamed protein; AK055766 | 209 | 426 |
| | | Human | similar to CG8839 | 210 | 427 |
| CG8895 | s+ | | | 211 | 428 |
| | | Human | nogo-A; reticulon 4 | 212 | 429 |
| | | Human | reticulon 1 | 213 | 430 |
| | | Mouse | nogo-A | 214 | 431 |
| | | Mouse | reticulon 1 | 215 | 432 |
| CG10949 | red. | | | 216 | 433 |
| CG13251 | s+, a− | | | 217 | 434 |

Each of the genes and proteins in Table 2 provide useful targets. In some embodiments, the expression of a gene or genes in Table 2 is detected in a biological sample to assess the status of a cell, tissue, or organism (e.g., to determine a metabolic or differentiation state of a cell, tissue, or organism). In other embodiments, the expression of a gene or genes in Table 2 is monitored in the presence or absence of an agent to determine whether the agent affects the expression of the gene and/or affects the size or number of fat cells in a tissue or organism. In yet other embodiments, the expression of a gene or genes in Table 2 is altered in vitro or in vivo to affect a change in the number and/or size of fat cells. Compositions and methods for practicing these aspects of the invention are provided in detail below.

I. Polynucleotides

The present invention provides nucleic acids encoding genes functionally correlated to fat cell size and or number, as well as, homologs, variants, and mutants of such genes. In some embodiments, the present invention provide polynucleotide sequences that are capable of hybridizing to the sequences in Table 2 under conditions of low to high stringency as long as the polynucleotide sequence capable of hybridizing encodes a protein that retains a biological activity of the naturally occurring (e.g., expression of the gene alters fat cell size or number). In some embodiments, the protein that retains a biological activity of naturally occurring gene is 70% homologous to wild-type gene, preferably 80% homologous to wild-type gene, more preferably 90% homologous to wild-type gene, and most preferably 95% homologous to wild-type gene. In preferred embodiments, hybridization conditions are based on the melting temperature (Tₘ) of the nucleic acid binding complex and confer a defined “stringency” as explained above (See e.g., Wahl, et al., Meth. Enzymol., 152:399-407 (1987), incorporated herein by reference).

In other embodiments of the present invention, alleles of gene are provided. In preferred embodiments, alleles result from a mutation, (i.e., a change in the nucleic acid sequence) and generally produce altered mRNAs or polypeptides whose structure or function may or may not be altered. Any given gene may have none, one, or many allelic forms. Common mutational changes that give rise to alleles are generally ascribed to deletions, additions or substitutions of nucleic acids. Each of these types of changes may occur alone, or in combination with the others, and at the rate of one or more times in a given sequence.

In still other embodiments of the present invention, the nucleotide sequences of the present invention may be engineered in order to alter a gene coding sequence for a variety of reasons, including but not limited to, alterations which modify the cloning, processing and/or expression of the gene product. For example, mutations may be introduced using techniques that are well known in the art (e.g., site-directed mutagenesis to insert new restriction sites, to alter glycosylation patterns, to change codon preference, etc.).

In some embodiments of the present invention, the polynucleotide sequence of the gene may be extended utilizing the nucleotide sequences in various methods known in the art to detect upstream sequences such as promoters and regulatory elements. For example, it is contemplated that restriction-site polymerase chain reaction (PCR) finds use in the present invention. This is a direct method that uses universal primers to retrieve unknown sequence adjacent to a known locus (Gobinda et al., PCR Methods Applic., 2:318-22 (1993)). First, genomic DNA is amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

In another embodiment, inverse PCR is used to amplify or extend sequences using divergent primers based on a known region (Triglia et al., Nucleic Acids Res., 16:8186 (1988)). The primers may be designed using Oligo 4.0 (National Biosciences Inc, Plymouth Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template. In still other embodiments, walking PCR is utilized. Walking PCR is a method for targeted gene walking that permits retrieval of unknown sequence (Parker et al., Nucleic Acids Res., 19:3055-60 (1991)). The PROMOTERFINDER kit (Clontech) uses PCR, nested primers and special libraries to “walk in” genomic DNA. This process avoids the need to screen libraries and is useful in finding intron/exon junctions.

Preferred libraries for screening for full-length cDNAs include mammalian libraries that have been size-selected to include larger cDNAs. Also, random primed libraries are preferred, in that they will contain more sequences that contain the 5′ and upstream gene regions. A randomly primed library may be particularly useful in case where an oligo d(T) library does not yield full-length cDNA. Genomic mammalian libraries are useful for obtaining introns and extending 5′ sequence.

A modified peptide can be produced in which the nucleotide sequence encoding the polypeptide has been altered, such as by substitution, deletion, or addition. In particularly preferred embodiments, these modifications do not significantly reduce the activity of the modified gene product. In other words, construct “X” can be evaluated in order to determine whether it is a member of the genus of modified or variant genes of the present invention as defined functionally, rather than structurally. In preferred embodiments, the activity of the variant or mutant gene product is evaluated by carrying out a functional screen (See e.g., Example section below, introducing the altered gene into a fly model to determine size and/or number of fat cells).

Moreover, as described above, variant forms of gene are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present invention provide variants of the genes disclosed herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur -containing (cysteine and methionine) (e.g., Stryer ed., Biochemistry, pg. 17-21, 2nd ed, WH Freeman and Co., 1981). Whether a change in the amino acid sequence of a peptide results in a functional homolog can be readily determined by assessing the ability of the variant peptide to function in a fashion similar to the wild-type protein. Peptides having more than one replacement can readily be tested in the same manner.

More rarely, a variant includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

As described in more detail below, variants may be produced by methods such as directed evolution or other techniques for producing combinatorial libraries of variants, described in more detail below. In still other embodiments of the present invention, the nucleotide sequences of the present invention may be engineered in order to alter a coding sequence including, but not limited to, alterations that modify the cloning, processing, localization, secretion, and/or expression of the gene product. For example, mutations may be introduced using techniques that are well known in the art (e.g., site-directed mutagenesis to insert new restriction sites, alter glycosylation patterns, or change codon preference, etc.).

II. Polypeptides

In other embodiments, the present invention provides polypeptide sequences as shown in Table 2 that are correlated to altered fat cell size or number. Other embodiments of the present invention provide fragments, fusion proteins or functional equivalents of these proteins. In still other embodiment of the present invention, nucleic acid sequences corresponding to these various gene homologs and mutants may be used to generate recombinant DNA molecules that direct the expression of the protein homologs and mutants in appropriate host cells. In some embodiments of the present invention, the polypeptide may be a naturally purified product, in other embodiments it may be a product of chemical synthetic procedures, and in still other embodiments it may be produced by recombinant techniques using a prokaryotic or eukaryotic host (e.g., by bacterial, yeast, higher plant, insect and mammalian cells in culture). In some embodiments, depending upon the host employed in a recombinant production procedure, the polypeptide of the present invention may be glycosylated or may be non-glycosylated. In other embodiments, the polypeptides of the invention may also include an initial methionine amino acid residue.

In one embodiment of the present invention, due to the inherent degeneracy of the genetic code, DNA sequences other than the polynucleotide sequences of Table 2 which encode substantially the same or a functionally equivalent amino acid sequence, may be used to clone and express the gene. In general, such polynucleotide sequences hybridize to the gene sequences under conditions of high to medium stringency as described above. As will be understood by those of skill in the art, it may be advantageous to produce nucleotide sequences possessing non-naturally occurring codons. Therefore, in some preferred embodiments, codons preferred by a particular prokaryotic or eukaryotic host (Murray et al., Nucl. Acids Res., 17 (1989)) are selected, for example, to increase the rate of gene expression or to produce recombinant RNA transcripts having desirable properties, such as a longer half-life, than transcripts produced from naturally occurring sequence.

### 1. Vectors for Production of Polypeptides

The polynucleotides of the present invention may be employed for producing polypeptides by recombinant techniques. Thus, for example, the polynucleotide may be included in any one of a variety of expression vectors for expressing a polypeptide. In some embodiments of the present invention, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In particular, some embodiments of the present invention provide recombinant constructs comprising one or more of the sequences as broadly described above. In some embodiments of the present invention, the constructs comprise a vector, such as a plasmid or viral vector, into which a sequence of the invention has been inserted, in a forward or reverse orientation. In still other embodiments, the heterologous structural sequence is assembled in appropriate phase with translation initiation and termination sequences. In preferred embodiments of the present invention, the appropriate DNA sequence is inserted into the vector using any of a variety of procedures. In general, the DNA sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial—pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia); and 2) Eukaryotic—pWLNEO, pSV2CAT, pOG44, PXT1, pSG (Stratagene) pSVK3, pBPV, pMSG, pSVL (Pharmacia). Any other plasmid or vector may be used as long as they are replicable and viable in the host. In some preferred embodiments of the present invention, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements.

In certain embodiments of the present invention, the DNA sequence in the expression vector is operatively linked to an appropriate expression control sequence(s) (promoter) to direct mRNA synthesis. Promoters useful in the present invention include, but are not limited to, the LTR or SV40 promoter, the E. coli lac or trp, the phage lambda P_{L} and P_{R}, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (e.g., dihydrofolate reductase or neomycin resistance for eukaryotic cell culture, or tetracycline or ampicillin resistance in E. coli).

In some embodiments of the present invention, transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin bp 100 to 270, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

### 2. Host Cells for Production of Polypeptides

In a further embodiment, the present invention provides host cells containing the above-described constructs. In some embodiments of the present invention, the host cell is a higher eukaryotic cell (e.g., a mammalian or insect cell). In other embodiments of the present invention, the host cell is a lower eukaryotic cell (e.g., a yeast cell). In still other embodiments of the present invention, the host cell can be a prokaryotic cell (e.g., a bacterial cell). Specific examples of host cells include, but are not limited to, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, (Gluzman, Cell 23:175 (1981)), C127, 3T3, 293, 293T, HeLa and BHK cell lines.

The constructs in host cells can be used in a conventional manner to produce the gene product encoded by the recombinant sequence. In some embodiments, introduction of the construct into the host cell can be accomplished by calcium phosphate transfection, DEAE-Dextran mediated transfection, or electroporation (See e.g., Davis et al., Basic Methods in Molecular Biology, (1986)). Alternatively, in some embodiments of the present invention, the polypeptides of the invention can be synthetically produced by conventional peptide synthesizers.

Proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989).

In some embodiments of the present invention, following transformation of a suitable host strain and growth of the host strain to an appropriate cell density, the selected promoter is induced by appropriate means (e.g., temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

### 3. Purification of Polypeptides

The present invention also provides methods for recovering and purifying polypeptides from recombinant cell cultures including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. In other embodiments of the present invention, protein refolding steps can be used as necessary, in completing configuration of the mature protein. In still other embodiments of the present invention, high performance liquid chromatography (HPLC) can be employed for final purification steps.

The present invention further provides polynucleotides having the coding sequence fused in frame to a marker sequence which allows for purification of the polypeptide of the present invention. A non-limiting example of a marker sequence is a hexahistidine tag which may be supplied by a vector, preferably a pQE-9 vector, which provides for purification of the polypeptide fused to the marker in the case of a bacterial host, or, for example, the marker sequence may be a hemagglutinin (HA) tag when a mammalian host (e.g., COS-7 cells) is used. The HA tag corresponds to an epitope derived from the influenza hemagglutinin protein (Wilson et al., Cell, 37:767 (1984)).

### 4. Truncation Mutants of Polypeptides

In addition, the present invention provides fragments of the polypeptides (i.e., truncation mutants). In some embodiments of the present invention, when expression of a portion of the protein is desired, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al., J. Bacteriol., 169:751 (1987)) and Salmonella typhimurium and its in vitro activity has been demonstrated on recombinant proteins (Miller et al., Proc. Natl. Acad. Sci. USA 84:2718 (1990)). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP.

### 5. Fusion Proteins Containing Polypeptides

The present invention also provides fusion proteins incorporating all or part of the polypeptides of Table 2. Accordingly, in some embodiments of the present invention, the coding sequences for the polypeptide can be incorporated as a part of a fusion gene including a nucleotide sequence encoding a different polypeptide. It is contemplated that this type of expression system will find use under conditions where it is desirable to produce an immunogenic fragment of a protein. In some embodiments of the present invention, the VP6 capsid protein of rotavirus is used as an immunologic carrier protein for portions of the polypeptide, either in the monomeric form or in the form of a viral particle. In other embodiments of the present invention, the nucleic acid sequences corresponding to the portion of protein against which antibodies are to be raised can be incorporated into a fusion gene construct which includes coding sequences for a late vaccinia virus structural protein to produce a set of recombinant viruses expressing fusion proteins comprising a portion of the polypeptide as part of the virion. It has been demonstrated with the use of immunogenic fusion proteins utilizing the hepatitis B surface antigen fusion proteins that recombinant hepatitis B virions can be utilized in this role as well. Similarly, in other embodiments of the present invention, chimeric constructs coding for fusion proteins containing a portion of polypeptide and the poliovirus capsid protein are created to enhance immunogenicity of the set of polypeptide antigens (See e.g., EP Publication No. 025949; and Evans et al., Nature 339:385 (1989); Huang et al., J. Virol., 62:3855 (1988); and Schlienger et al, J. Virol., 66:2 (1992)).

In still other embodiments of the present invention, the multiple antigen peptide system for peptide-based immunization can be utilized. In this system, a desired portion of polypeptide is obtained directly from organo-chemical synthesis of the peptide onto an oligomeric branching lysine core (see e.g., Posnett et al., J. Biol. Chem., 263:1719 (1988); and Nardelli et al., J. Immunol., 148:914 (1992)). In other embodiments of the present invention, antigenic determinants of the proteins can also be expressed and presented by bacterial cells.

In addition to utilizing fusion proteins to enhance immunogenicity, it is widely appreciated that fusion proteins can also facilitate the expression of proteins, such as the proteins of the present invention. Accordingly, in some embodiments of the present invention, polypeptides can be generated as a glutathione-S-transferase (i.e., GST fusion protein). It is contemplated that such GST fusion proteins will enable easy purification of the polypeptide, such as by the use of glutathione-derivatized matrices (See e.g, Ausabel et al. (eds.), Current Protocols in Molecular Biology, John Wiley & Sons, NY (1991)). In another embodiment of the present invention, a fusion gene coding for a purification leader sequence, such as a poly-(His)/enterokinase cleavage site sequence at the N-terminus of the desired portion of the polypeptide, can allow purification of the expressed fusion protein by affinity chromatography using a Ni²⁺ metal resin. In still another embodiment of the present invention, the purification leader sequence can then be subsequently removed by treatment with enterokinase (See e.g., Hochuli et al., J. Chromatogr., 411:177 (1987); and Janknecht et al., Proc. Natl. Acad. Sci. USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment of the present invention, the fusion gene can be synthesized by conventional techniques including automated DNA synthesizers. Alternatively, in other embodiments of the present invention, PCR amplification of gene fragments can be carried out using anchor primers which give rise to complementary overhangs between two consecutive gene fragments which can subsequently be annealed to generate a chimeric gene sequence (See e.g., Current Protocols in Molecular Biology, supra).

### 6. Variants of Polypeptides

Still other embodiments of the present invention provides mutant or variant forms of the polypeptides (i.e., muteins). It is possible to modify the structure of a peptide having an activity of the original polypeptide for such purposes as enhancing therapeutic or prophylactic efficacy, or stability (e.g., ex vivo shelf life, and/or resistance to proteolytic degradation in vivo). Such modified peptides are considered functional equivalents of peptides having an activity of the subject proteins as defined herein. A modified peptide can be produced in which the amino acid sequence has been altered, such as by amino acid substitution, deletion, or addition.

Moreover, as described above, variant forms (e.g., mutants) of the subject proteins are also contemplated as being equivalent to those peptides and DNA molecules that are set forth in more detail. For example, as described above, the present invention encompasses mutant and variant proteins that contain conservative or non-conservative amino acid substitutions.

This invention further contemplates a method of generating sets of combinatorial mutants of the present proteins, as well as truncation mutants, and is especially useful for identifying potential variant sequences (i.e., homologs) that are functional (e.g., whose presence modifies the number or size of fat cells). The purpose of screening such combinatorial libraries is to generate, for example, novel homologs that can act as either agonists or antagonists, or alternatively, possess novel activities all together.

Therefore, in some embodiments of the present invention, homologs are engineered by the present method to provide more efficient modification of fat cell size or number. In other embodiments of the present invention, combinatorially-derived homologs are generated which have a selective potency relative to a naturally occurring polypeptides. Such proteins, when expressed from recombinant DNA constructs, can be used in gene therapy protocols.

Still other embodiments of the present invention provide homologs that have intracellular half-lives dramatically different than the corresponding wild-type protein. For example, the altered protein can be rendered either more stable or less stable to proteolytic degradation or other cellular process that result in destruction of, or otherwise inactivate the polypeptide. Such homologs, and the genes which encode them, can be utilized to alter the location of polypeptide expression by modulating the half-life of the protein. For instance, a short half-life can give rise to more transient biological effects and, when part of an inducible expression system, can allow tighter control of protein levels within the cell. As above, such proteins, and particularly their recombinant nucleic acid constructs, can be used in gene therapy protocols.

In still other embodiments of the present invention, homologs are generated by the combinatorial approach to act as antagonists, in that they are able to interfere with the ability of the corresponding wild-type protein to regulate cell function.

In some embodiments of the combinatorial mutagenesis approach of the present invention, the amino acid sequences for a population of homologs or other related proteins are aligned, preferably to promote the highest homology possible. Such a population of variants can include, for example, homologs from one or more species, or homologs from the same species but which differ due to mutation. Amino acids that appear at each position of the aligned sequences are selected to create a degenerate set of combinatorial sequences.

In a preferred embodiment of the present invention, the combinatorial library is produced by way of a degenerate library of genes encoding a library of polypeptides which each include at least a portion of potential protein sequences. For example, a mixture of synthetic oligonucleotides can be enzymatically ligated into gene sequences such that the degenerate set of potential sequences are expressible as individual polypeptides, or alternatively, as a set of larger fusion proteins (e.g., for phage display) containing the set of sequences therein.

There are many ways by which the library of potential homologs can be generated from a degenerate oligonucleotide sequence. In some embodiments, chemical synthesis of a degenerate gene sequence is carried out in an automatic DNA synthesizer, and the synthetic genes are ligated into an appropriate gene for expression. The purpose of a degenerate set of genes is to provide, in one mixture, all of the sequences encoding the desired set of potential sequences. The synthesis of degenerate oligonucleotides is well known in the art (See e.g., Narang, Tetrahedron Lett., 39:39 (1983); Itakura et al., Recombinant DNA, in Walton (ed.), Proceedings of the 3rd Cleveland Symposium on Macromolecules, Elsevier, Amsterdam, pp 273-289 (1981); Itakura et al., Annu. Rev. Biochem., 53:323 (1984); Itakura et al., Science 198:1056 (1984); Ike et al., Nucl. Acid Res., 11:477 (1983)). Such techniques have been employed in the directed evolution of other proteins (See e.g., Scott et al., Science 249:386 (1980); Roberts et al., Proc. Natl. Acad. Sci. USA 89:2429 (1992); Devlin et al., Science 249: 404 (1990); Cwirla et al., Proc. Natl. Acad. Sci. USA 87: 6378 (1990); as well as U.S. Pat. Nos. 5,223,409, 5,198,346, and 5,096,815, each of which is incorporated herein by reference).

It is contemplated that the nucleic acids, and fragments and variants thereof, can be utilized as starting nucleic acids for directed evolution. These techniques can be utilized to develop variants having desirable properties such as increased or decreased ability to alter fat cell size or number.

In some embodiments, artificial evolution is performed by random mutagenesis (e.g., by utilizing error-prone PCR to introduce random mutations into a given coding sequence). This method requires that the frequency of mutation be finely tuned. As a general rule, beneficial mutations are rare, while deleterious mutations are common. This is because the combination of a deleterious mutation and a beneficial mutation often results in an inactive enzyme. The ideal number of base substitutions for targeted gene is usually between 1.5 and 5 (Moore and Arnold, Nat. Biotech., 14, 458 (1996); Leung et al., Technique, 1:11 (1989); Eckert and Kunkel, PCR Methods Appl., 1:17-24 (1991); Caldwell and Joyce, PCR Methods Appl., 2:28 (1992); and Zhao and Arnold, Nuc. Acids. Res., 25:1307 (1997)). After mutagenesis, the resulting clones are selected for desirable activity (e.g., screened for ability to alter fat cell size or number). Successive rounds of mutagenesis and selection are often necessary to develop enzymes with desirable properties. It should be noted that only the useful mutations are carried over to the next round of mutagenesis.

In other embodiments of the present invention, the polynucleotides of the present invention are used in gene shuffling or sexual PCR procedures (e.g., Smith, Nature, 370:324 (1994); U.S. Pat. Nos. 5,837,458; 5,830,721; 5,811,238; 5,733,731; all of which are herein incorporated by reference). Gene shuffling involves random fragmentation of several mutant DNAs followed by their reassembly by PCR into full-length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DNaseI and subjected to multiple rounds of PCR with no added primer. The lengths of random fragments approach that of the uncleaved segment as the PCR cycles proceed, resulting in mutations in present in different clones becoming mixed and accumulating in some of the resulting sequences. Multiple cycles of selection and shuffling have led to the functional enhancement of several enzymes (Stemmer, Nature, 370:398 (1994); Stemmer, Proc. Natl. Acad. Sci. USA, 91:10747 (1994); Crameri et al., Nat. Biotech., 14:315 (1996); Zhang et al., Proc. Natl. Acad. Sci. USA, 94:4504 (1997); and Crameri et al., Nat. Biotech., 15:436 (1997)). Variants produced by directed evolution can be screened for activity by the methods described in Examples.

A wide range of techniques are known in the art for screening gene products of combinatorial libraries made by point mutations, and for screening cDNA libraries for gene products having a certain property. Such techniques will be generally adaptable for rapid screening of the gene libraries generated by the combinatorial mutagenesis or recombination of homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

### 7. Chemical Synthesis of Polypeptides

In an alternate embodiment of the invention, the coding sequence of the gene encoding the polypeptide is synthesized, whole or in part, using chemical methods well known in the art (See e.g., Caruthers et al., Nucl. Acids Res. Symp. Ser., 7:215 (1980); Crea and Horn, Nucl. Acids Res., 9:2331 (1980); Matteucci and Caruthers, Tetrahedron Lett., 21:719 (1980); and Chow and Kempe, Nucl. Acids Res., 9:2807 (1981)). In other embodiments of the present invention, the protein itself is produced using chemical methods to synthesize either an entire amino acid sequence or a portion thereof. For example, peptides can be synthesized by solid phase techniques, cleaved from the resin, and purified by preparative high performance liquid chromatography (See e.g., Creighton, Proteins Structures And Molecular Principles, W H Freeman and Co, New York N.Y. (1983)). In other embodiments of the present invention, the composition of the synthetic peptides is confirmed by amino acid analysis or sequencing (See e.g., Creighton, supra).

Direct peptide synthesis can be performed using various solid-phase techniques (Roberge et al., Science 269:202 (1995)) and automated synthesis may be achieved, for example, using ABI 431A Peptide Synthesizer (Perkin Elmer) in accordance with the instructions provided by the manufacturer. Additionally, the amino acid sequence of polypeptides, or any part thereof, may be altered during direct synthesis and/or combined using chemical methods with other sequences to produce a variant polypeptide.

III. Detection of Alleles and/or Expression

A. Alleles

In some embodiments, the present invention includes alleles of the genes in Table 2 that increase or decrease a subject's susceptibility to obesity or wasting diseases. Known polymorphisms in genes can be obtained from any number of public databases, including, but not limited to the OMIM and dbSNP databases.

B. Detection of Alleles and/or Expression

Accordingly, the present invention provides methods for determining whether a subject has an increased susceptibility to conditions involving fat cell size or number (e.g., obesity, diabetes, wasting diseases, cancer, etc.) by detecting expression levels of a gene or genes in Table 2 or by detecting particular alleles of a gene or genes in Table 2. In other embodiments, the present invention provides methods for providing a prognosis of increased risk for such diseases and conditions to an individual based on the presence or absence of one or more alleles or the expression of a particular gene.

A number of methods are available for analysis of gene expression and allele identification. Protocols and commercially available kits or services for performing multiple variations of these assays are available. In some embodiments, assays are performed in combination or in hybrid (e.g., different reagents or technologies from several assays are combined to yield one assay). The following exemplary assays are useful in the present invention: direct sequencing assays, PCR assays, hybridization assays (including microarray assays), enzymatic detection of sequences (see e.g., TAQMAN assay, Applera Corporation, INVADER assay, Third Wave Technologies), mass spectroscopy assays, and differential antibody binding assays.

C. Kits

The present invention also provides kits for determining whether an individual expresses a gene or genes of Table 2 or has a particular allele of said gene or genes. In some preferred embodiments, a plurality of genes are analyzed to provide, for example, an expression profile that indicates a biological status of cells, tissues, or organisms. In some embodiments, the kits are useful determining whether the subject is at risk of developing a disease or condition. The diagnostic kits are produced in a variety of ways. In some embodiments, the kits contain at least one reagent for specifically detecting a gene allele or gene expression. In other preferred embodiments, the reagents are primers for amplifying the region of DNA containing the region of interest. In still other embodiments, the reagents are antibodies that preferentially bind proteins of interest. In some embodiments, the kit contains instructions for determining whether the subject is at risk for developing a disease or condition. In some embodiments, the kits include ancillary reagents such as buffering agents, nucleic acid stabilizing reagents, protein stabilizing reagents, and signal producing systems (e.g., florescence generating systems). The test kit may be packaged in any suitable manner, typically with the elements in a single container or various containers as necessary along with a sheet of instructions for carrying out the test. In some embodiments, the kits also preferably include a positive control sample.

D. Bioinformatics

In some embodiments, the analysis of data is obtained from any of the above methods automated. For example, in some embodiments, the present invention provides a bioinformatics research system comprising a plurality of computers running a multi-platform object oriented programming language (See e.g., U.S. Pat. No. 6,125,383; herein incorporated by reference). In some embodiments, one of the computers stores data. In some embodiments, data or an analysis of the data is made available (e.g., over an electronic communications network) to researchers, physicians, or other individuals.

IV. Generation of Antibodies

Antibodies can be generated to allow for the detection of protein or to alter the bioavailability of activity of protein. The antibodies may be prepared using various immunogens. In one embodiment, the immunogen is a peptide to generate antibodies that recognize a polypeptide of Table 2. Such antibodies include, but are not limited to polyclonal, monoclonal, chimeric, single chain, Fab fragments, and Fab expression libraries.

Various procedures known in the art may be used for the production of polyclonal antibodies directed against the polypeptide. For the production of antibody, various host animals can be immunized by injection with the peptide corresponding to the epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies directed toward the polypeptide, it is contemplated that any technique that provides for the production of antibody molecules by continuous cell lines in culture will find use with the present invention (See e.g., Harlow and Lane, Antibodies: A Laboratory Manual, Cold Spring Harbor Laboratory Press, Cold Spring Harbor, N.Y.). These include but are not limited to the hybridoma technique originally developed by Köhler and Milstein (Köhler and Milstein, Nature 256:495-497 (1975)), as well as the trioma technique, the human B-cell hybridoma technique (See e.g., Kozbor et al., Immunol. Tod., 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

In an additional embodiment of the invention, monoclonal antibodies are produced in germ-free animals utilizing technology such as that described in PCT/US90/02545). Furthermore, it is contemplated that human antibodies will be generated by human hybridomas (Cote et al., Proc. Natl. Acad. Sci. USA 80:2026-2030 (1983)) or by transforming human B cells with EBV virus in vitro (Cole et al., in Monoclonal Antibodies and Cancer Therapy, Alan R. Liss, pp. 77-96 (1985)).

In addition, it is contemplated that techniques described for the production of single chain antibodies (U.S. Pat. No. 4,946,778; herein incorporated by reference) will find use in producing specific single chain antibodies. An additional embodiment of the invention utilizes the techniques described for the construction of Fab expression libraries (Huse et al., Science 246:1275-1281 (1989)) to allow rapid and easy identification of monoclonal Fab fragments with the desired specificity for the polypeptide.

It is contemplated that any technique suitable for producing antibody fragments will find use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody will be accomplished by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g.,gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. (As is well known in the art, the immunogenic peptide should be provided free of the carrier molecule used in any immunization protocol. For example, if the peptide was conjugated to KLH, it may be conjugated to BSA, or used directly, in a screening assay.)

The foregoing antibodies can be used in methods known in the art relating to the localization and structure of the polypeptide (e.g., for Western blotting), measuring levels thereof in appropriate biological samples, etc. The antibodies can be used to detect the polypeptide in a biological sample from an individual. The biological sample can be a biological fluid, such as, but not limited to, blood, serum, plasma, interstitial fluid, urine, cerebrospinal fluid, and the like, containing cells.

The biological samples can then be tested directly for the presence of the polypeptide using an appropriate strategy (e.g., ELISA or radioimmunoassay) and format (e.g., microwells, dipstick (e.g., as described in International Patent Publication WO 93/03367), etc. Alternatively, proteins in the sample can be size separated (e.g., by polyacrylamide gel electrophoresis (PAGE), in the presence or not of sodium dodecyl sulfate (SDS), and the presence of GENE detected by immunoblotting (Western blotting). Immunoblotting techniques are generally more effective with antibodies generated against a peptide corresponding to an epitope of a protein, and hence, are particularly suited to the present invention.

V. Modulation of Expression and/or Activity

The expression of the genes in Table 2 may be modified in cells, tissues, or organisms, in vitro or in vivo using any number of technologies. In some embodiments, antibodies are used to block the activity of the proteins in Table 2. In other embodiments, antisense oligonucleotides (antisense RNAs, transcription factor decoys, siRNAs, etc.) are used to reduce or prevent the generation of mRNA or conversion of mRNA to protein. Available software programs and/or biological methods may be use to design suitable antisense oligonucleotides. For example, in some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in U.S. Patent WO0198537A2, herein incorporated by reference.

In yet other embodiments, compounds the directly or indirectly regulate expression may be used (e.g., small molecule drugs, hormones, signal transduction pathway regulators, etc.). As discussed in detail below, cells, tissue, or organisms may also be transgenically altered to change or regulate the expression of the genes (e.g., adding an inducible promoter, knocking out a gene, adding a gene or regulatory sequence).

Modulation of expression has many uses. For example, modulation of expression finds use in assays to identify biological consequences of the change in expression in one or more cell or tissue types. Cells with modulated expression also find use in screening assays to identify agents or stimuli that counteract or increase the change in expression (e.g., and thereby reverse or potentiate an effect on change in fat cell size or number). Modulation of expression can also be use to generate animal models. Modulation of expression also finds use in therapeutic applications, where the change in expression increases or decreases fat cell size or number, as desired.

The present invention provides methods and compositions suitable for gene therapy to alter gene expression, production, or function. In some embodiments, it is contemplated that the gene therapy is performed by providing a subject with a wild-type allele of the gene.

Viral vectors commonly used for in vivo or ex vivo targeting and therapy procedures are DNA-based vectors and retroviral vectors. Methods for constructing and using viral vectors are known in the art (See e.g., Miller and Rosman, BioTech., 7:980-990 (1992)). Preferably, the viral vectors are replication defective, that is, they are unable to replicate autonomously in the target cell. In general, the genome of the replication defective viral vectors that are used within the scope of the present invention lack at least one region that is necessary for the replication of the virus in the infected cell. These regions can either be eliminated (in whole or in part), or be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (i.e., on the isolated DNA) or in situ, using the techniques of genetic manipulation or by treatment with mutagenic agents.

Preferably, the replication defective virus retains the sequences of its genome that are necessary for encapsidating the viral particles. DNA viral vectors include an attenuated or defective DNA viruses, including, but not limited to, herpes simplex virus (HSV), papillomavirus, Epstein Barr virus (EBV), adenovirus, adeno-associated virus (AAV), and the like. Defective viruses, that entirely or almost entirely lack viral genes, are preferred, as defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Mol. Cell. Neurosci., 2:320-330 (1991)), defective herpes virus vector lacking a glycoprotein L gene (See e.g., Patent Publication RD 371005 A), or other defective herpes virus vectors (See e.g., WO 94/21807; and WO 92/05263); an attenuated adenovirus vector, such as the vector described by Stratford-Perricaudet et al. (J. Clin. Invest., 90:626-630 (1992); See also, La Salle et al., Science 259:988-990 (1993)); and a defective adeno-associated virus vector (Samulski et al., J. Virol., 61:3096-3101 (1987); Samulski et al., J. Virol., 63:3822-3828 (1989); and Lebkowski et al., Mol. Cell. Biol., 8:3988-3996 (1988)).

Preferably, for in vivo administration, an appropriate immunosuppressive treatment is employed in conjunction with the viral vector (e.g., adenovirus vector), to avoid immuno-deactivation of the viral vector and transfected cells. For example, immunosuppressive cytokines, such as interleukin-12 (IL-12), interferon-gamma (IFN-), or anti-CD4 antibody, can be administered to block humoral or cellular immune responses to the viral vectors. In addition, it is advantageous to employ a viral vector that is engineered to express a minimal number of antigens.

In a preferred embodiment, the vector is an adenovirus vector. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to type 2 or type 5 human adenoviruses (Ad 2 or Ad 5), or adenoviruses of animal origin (See e.g., WO94/26914). Those adenoviruses of animal origin that can be used within the scope of the present invention include adenoviruses of canine, bovine, murine (e.g., Mav1, Beard et al., Virol., 75-81 (1990)), ovine, porcine, avian, and simian (e.g., SAV) origin. Preferably, the adenovirus of animal origin is a canine adenovirus, more preferably a CAV2 adenovirus (e.g. Manhattan or A26/61 strain (ATCC VR-800)).

Preferably, the replication defective adenoviral vectors of the invention comprise the ITRs, an encapsidation sequence and the nucleic acid of interest. Still more preferably, at least the E1 region of the adenoviral vector is non-functional. The deletion in the E1 region preferably extends from nucleotides 455 to 3329 in the sequence of the Ad5 adenovirus (PvuII-BglII fragment) or 382 to 3446 (HinfII-Sau3A fragment). Other regions may also be modified, in particular the E3 region (e.g., WO95/02697), the E2 region (e.g., WO94/28938), the E4 region (e.g., WO94/28152, WO94/12649 and WO95/02697), or in any of the late genes L1-L5.

In a preferred embodiment, the adenoviral vector has a deletion in the E1 region (Ad 1.0). Examples of E1-deleted adenoviruses are disclosed in EP 185,573, the contents of which are incorporated herein by reference. In another preferred embodiment, the adenoviral vector has a deletion in the E1 and E4 regions (Ad 3.0). Examples of E1/E4-deleted adenoviruses are disclosed in WO95/02697 and WO96/22378. In still another preferred embodiment, the adenoviral vector has a deletion in the E1 region into which the E4 region and the nucleic acid sequence are inserted.

The replication defective recombinant adenoviruses according to the invention can be prepared by any technique known to the person skilled in the art (See e.g., Levrero et al., Gene 101:195 (1991); EP 185 573; and Graham, EMBO J., 3:2917 (1984)). In particular, they can be prepared by homologous recombination between an adenovirus and a plasmid which carries, inter alia, the DNA sequence of interest. The homologous recombination is accomplished following co-transfection of the adenovirus and plasmid into an appropriate cell line. The cell line that is employed should preferably (i) be transformable by the elements to be used, and (ii) contain the sequences that are able to complement the part of the genome of the replication defective adenovirus, preferably in integrated form in order to avoid the risks of recombination. Examples of cell lines that may be used are the human embryonic kidney cell line 293 (Graham et al., J. Gen. Virol., 36:59 (1977)), which contains the left-hand portion of the genome of an Ad5 adenovirus (12%) integrated into its genome, and cell lines that are able to complement the E1 and E4 functions, as described in applications WO94/26914 and WO95/02697. Recombinant adenoviruses are recovered and purified using standard molecular biological techniques, that are well known to one of ordinary skill in the art.

The adeno-associated viruses (AAV) are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The use of vectors derived from the AAVs for transferring genes in vitro and in vivo has been described (See e.g., WO 91/18088; WO 93/09239; U.S. Pat. No. 4,797,368; U.S. Pat. No. 5,139,941; and EP 488 528, all of which are herein incorporated by reference). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In another embodiment, the gene can be introduced in a retroviral vector (e.g., as described in U.S. Pat. Nos. 5,399,346, 4,650,764, 4,980,289 and 5,124,263; all of which are herein incorporated by reference; Mann et al., Cell 33:153 (1983); Markowitz et al., J. Virol., 62:1120 (1988); PCT/US95/14575; EP 453242; EP178220; Bernstein et al. Genet. Eng., 7:235 (1985); McCormick, BioTechnol., 3:689 (1985); WO 95/07358; and Kuo et al., Blood 82:845 (1993)). The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Defective retroviral vectors are also disclosed in WO95/02697.

In general, in order to construct recombinant retroviruses containing a nucleic acid sequence, a plasmid is constructed that contains the LTRs, the encapsidation sequence and the coding sequence. This construct is used to transfect a packaging cell line, which cell line is able to supply in trans the retroviral functions that are deficient in the plasmid. In general, the packaging cell lines are thus able to express the gag, pol and env genes. Such packaging cell lines have been described in the prior art, in particular the cell line PA317 (U.S. Pat. No. 4,861,719, herein incorporated by reference), the PsiCRIP cell line (See, WO90/02806), and the GP+envAm-12 cell line (See, WO89/07150). In addition, the recombinant retroviral vectors can contain modifications within the LTRs for suppressing transcriptional activity as well as extensive encapsidation sequences that may include a part of the gag gene (Bender et al., J. Virol., 61:1639 (1987)). Recombinant retroviral vectors are purified by standard techniques known to those having ordinary skill in the art.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner et. al., Proc. Natl. Acad. Sci. USA 84:7413-7417 (1987); See also, Mackey, et al., Proc. Natl. Acad. Sci. USA 85:8027-8031 (1988); Ulmer et al., Science 259:1745-1748 (1993)). The use of cationic lipids may promote encapsulation of negatively charged nucleic acids, and also promote fusion with negatively charged cell membranes (Felgner and Ringold, Science 337:387-388 (1989)). Particularly useful lipid compounds and compositions for transfer of nucleic acids are described in WO95/18863 and WO96/17823, and in U.S. Pat. No. 5,459,127, herein incorporated by reference.

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce the vector in vivo as a naked DNA plasmid. Methods for formulating and administering naked DNA to mammalian muscle tissue are disclosed in U.S. Pat. Nos. 5,580,859 and 5,589,466, both of which are herein incorporated by reference.

DNA vectors for gene therapy can be introduced into the desired host cells by methods known in the art, including but not limited to transfection, electroporation, microinjection, transduction, cell fusion, DEAE dextran, calcium phosphate precipitation, use of a gene gun, or use of a DNA vector transporter (See e.g., Wu et al., J. Biol. Chem., 267:963 (1992); Wu and Wu, J. Biol. Chem., 263:14621 (1988); and Williams et al., Proc. Natl. Acad. Sci. USA 88:2726 (1991)). Receptor-mediated DNA delivery approaches can also be used (Curiel et al., Hum. Gene Ther., 3:147 (1992); and Wu and Wu, J. Biol. Chem., 262:4429 (1987)).

VI. Transgenic Animals Expressing Exogenous Genes and Homologs, Mutants, and Variants Thereof

The present invention contemplates the generation of transgenic animals comprising an exogenous gene of Table 2 or homologs, mutants, or variants thereof. In preferred embodiments, the transgenic animal displays an altered phenotype as compared to wild-type animals (e.g., has increased or decreased fat cell size or number). In some embodiments, the altered phenotype is the overexpression of mRNA for a gene as compared to wild-type levels of expression. In other embodiments, the altered phenotype is the decreased expression of mRNA for an endogenous gene as compared to wild-type levels of endogenous expression. Methods for analyzing the presence or absence of such phenotypes include Northern blotting, mRNA protection assays, and RT-PCR. In other embodiments, the transgenic animals have a knock out mutation of the gene.

The transgenic animals of the present invention find use in dietary and drug screens. In some embodiments, the transgenic animals are fed test or control diets and the response of the animals to the diets is evaluated. In other embodiments, test compounds (e.g., a drug that is suspected of being useful to treat obesity) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter which allows reproducible injection of 1-2 picoliters (p1) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 (1985)). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 (1976)). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. (1986)). The viral vector system used to introduce the transgene is typically a replication-defective retrovirus carrying the transgene (Jahner et al., Proc. Natl. Acad Sci. USA 82:6927 (1985)). Transfection is easily and efficiently obtained by culturing the blastomeres on a monolayer of virus-producing cells (Van der Putten, supra; Stewart, et al., EMBO J., 6:383 (1987)). Alternatively, infection can be performed at a later stage. Virus or virus-producing cells can be injected into the blastocoele (Jahner et al., Nature 298:623 (1982)). Most of the founders will be mosaic for the transgene since incorporation occurs only in a subset of cells which form the transgenic animal. Further, the founder may contain various retroviral insertions of the transgene at different positions in the genome which generally will segregate in the offspring. In addition, it is also possible to introduce transgenes into the germline, albeit with low efficiency, by intrauterine retroviral infection of the midgestation embryo (Jahner et al., supra (1982)). Additional means of using retroviruses or retroviral vectors to create transgenic animals known to the art involves the micro-injection of retroviral particles or mitomycin C-treated cells producing retrovirus into the perivitelline space of fertilized eggs or early embryos (PCT International Application WO 90/08832 (1990), and Haskell and Bowen, Mol. Reprod. Dev., 40:386 (1995)).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 (1981); Bradley et al, Nature 309:255 (1984); Gossler et al., Proc. Acad. Sci. USA 83:9065 (1986); and Robertson et al., Nature 322:445 (1986)). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 (1988)). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells which have integrated the transgene. This technique obviates the need for growth of the transfected ES cells under appropriate selective conditions prior to transfer into the blastocoel.

In still other embodiments, homologous recombination is utilized knock-out gene function or create deletion mutants. Methods for homologous recombination are described in U.S. Pat. No. 5,614,396, incorporated herein by reference.

VII. Drug Screening

The present invention provides methods and compositions for using the genes and gene products of Table 2 as targets for screening drugs, other agents, or stimuli that alter fat cell number or size or otherwise modulates a desired phenotype (e.g., disease phenotype).

A technique for drug screening provides high throughput screening for compounds having suitable binding affinity to peptides and is described in detail in WO 84/03564, incorporated herein by reference. Briefly, large numbers of different small peptide test compounds are synthesized on a solid substrate, such as plastic pins or some other surface. The peptide test compounds are then reacted with peptides of Table 2 and washed. Bound peptides are then detected by methods well known in the art.

Another technique uses antibodies directed against the peptides of Table 2, generated as discussed above. Such antibodies capable of specifically binding to target peptides compete with a test compound for binding to the target peptide. In this manner, the antibodies can be used to detect the presence of any target compound that shares one or more antigenic determinants of the peptide.

The present invention contemplates many other means of screening compounds. The examples provided above are presented merely to illustrate a range of techniques available. One of ordinary skill in the art will appreciate that many other screening methods can be used.

In particular, the present invention contemplates the use of cell lines transfected with or expressing the target gene and variants or mutants thereof for screening compounds for activity, and in particular to high throughput screening of compounds from combinatorial libraries (e.g., libraries containing greater than 10⁴ compounds). The cell lines of the present invention can be used in a variety of screening methods. In some embodiments, the cells can be used in second messenger assays that monitor signal transduction following activation of cell-surface receptors. In other embodiments, the cells can be used in reporter gene assays that monitor cellular responses at the transcription/translation level. In still further embodiments, the cells can be used in cell proliferation assays to monitor the overall growth/no growth response of cells to external stimuli. Cell numbers can be counted using flow cytometry or other conventional methods. Fat cell content in cells can be measured by any suitable method, including but not limited to staining with Oil Red O.

In second messenger assays, the host cells are preferably transfected as described above with vectors encoding a polypeptide of interest or variants or mutants thereof. The host cells are then treated with a compound or plurality of compounds (e.g., from a combinatorial library) and assayed for the presence or absence of a response. It is contemplated that at least some of the compounds in the combinatorial library can serve as agonists, antagonists, activators, or inhibitors of the protein or proteins encoded by the vectors. It is also contemplated that at least some of the compounds in the combinatorial library can serve as agonists, antagonists, activators, or inhibitors of protein acting upstream or downstream of the protein encoded by the vector in a signal transduction pathway.

In some embodiments, the second messenger assays measure fluorescent signals from reporter molecules that respond to intracellular changes (e.g., Ca²⁺ concentration, membrane potential, pH, IP₃, cAMP, arachidonic acid release) due to stimulation of membrane receptors and ion channels (e.g., ligand gated ion channels; see Denyer et al., Drug Discov. Today 3:323 (1998); and Gonzales et al., Drug. Discov. Today 4:431-39 (1999)). Examples of reporter molecules include, but are not limited to, FRET (florescence resonance energy transfer) systems (e.g., Cuo-lipids and oxonols, EDAN/DABCYL), calcium sensitive indicators (e.g., Fluo-3, FURA 2, INDO 1, and FLUO3/AM, BAPTA AM), chloride-sensitive indicators (e.g., SPQ, SPA), potassium-sensitive indicators (e.g., PBFI), sodium-sensitive indicators (e.g., SBFI), and pH sensitive indicators (e.g., BCECF).

In general, the host cells are loaded with the indicator prior to exposure to the compound. Responses of the host cells to treatment with the compounds can be detected by methods known in the art, including, but not limited to, fluorescence microscopy, confocal microscopy (e.g., FCS systems), flow cytometry, microfluidic devices, FLIPR systems (See, e.g., Schroeder and Neagle, J. Biomol. Screening 1:75 (1996)), and plate-reading systems. In some preferred embodiments, the response (e.g., increase in fluorescent intensity) caused by compound of unknown activity is compared to the response generated by a known agonist and expressed as a percentage of the maximal response of the known agonist. The maximum response caused by a known agonist is defined as a 100% response. Likewise, the maximal response recorded after addition of an agonist to a sample containing a known or test antagonist is detectably lower than the 100% response.

The cells are also useful in reporter gene assays. Reporter gene assays involve the use of host cells transfected with vectors encoding a nucleic acid comprising transcriptional control elements of a target gene (e.g., a gene that controls the biological expression and function of a disease target) spliced to a coding sequence for a reporter gene. Therefore, activation of the target gene results in activation of the reporter gene product.

VIII. Pharmaceutical Compositions Containing Nucleic Acid, Peptides, and Analogs

The present invention further provides pharmaceutical compositions which may comprise all or portions of polynucleotide sequences (e.g., of Table 2), polypeptides, inhibitors or antagonists of bioactivity, including antibodies, alone or in combination with at least one other agent, such as a stabilizing compound, and may be administered in any sterile, biocompatible pharmaceutical carrier, including, but not limited to, saline, buffered saline, dextrose, and water.

The methods of the present invention find use in treating diseases or altering physiological states. Peptides can be administered to the patient intravenously in a pharmaceutically acceptable carrier such as physiological saline. Standard methods for intracellular delivery of peptides can be used (e.g., delivery via liposome). Such methods are well known to those of ordinary skill in the art. The formulations of this invention are useful for parenteral administration, such as intravenous, subcutaneous, intramuscular, and intraperitoneal. Therapeutic administration of a polypeptide intracellularly can also be accomplished using gene therapy as described above.

As is well known in the medical arts, dosages for any one patient depends upon many factors, including the patient's size, body surface area, age, the particular compound to be administered, sex, time and route of administration, general health, and interaction with other drugs being concurrently administered.

Accordingly, in some embodiments of the present invention, nucleotide and amino acid sequences can be administered to a patient alone, or in combination with other nucleotide sequences, drugs or hormones or in pharmaceutical compositions where it is mixed with excipient(s) or other pharmaceutically acceptable carriers. In one embodiment of the present invention, the pharmaceutically acceptable carrier is pharmaceutically inert. In another embodiment of the present invention, polynucleotide sequences or amino acid sequences may be administered alone to individuals subject to or suffering from a disease or condition (e.g., obesity, diabetes, etc.).

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. For example, an effective amount of the pharmaceutical agent may be that amount that regulates fat cell size or number. Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (i.e., dosage).

Pharmaceutical preparations which can be used orally include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising a compound of the invention formulated in a pharmaceutical acceptable carrier may be prepared, placed in an appropriate container, and labeled for treatment of an indicated condition. For polynucleotide or amino acid sequences, conditions indicated on the label may include treatment of condition related to apoptosis.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

For any compound used in the method of the invention, the therapeutically effective dose can be estimated initially from cell culture assays. Then, preferably, dosage can be formulated in animal models (particularly murine models) to achieve a desirable circulating concentration range.

A therapeutically effective dose refers to that amount of which ameliorates symptoms of the disease state or condition. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds which exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; or 5,225,212, all of which are herein incorporated by reference). Those skilled in the art will employ different formulations for the polypeptide or nucleic acid (e.g., of Table 2) than for the inhibitors of polypeptide or nucleic acid expression.

### EXPERIMENTAL

The following examples are provided in order to demonstrate and further illustrate certain preferred embodiments and aspects of the present invention and are not to be construed as limiting the scope thereof.

### EXAMPLE 1

### Identification of Genes that Modify the Number and/or Size of Fat Cells

The ability to ectopically express nucleic acids in a conditional, tissue-specific manner is an important tool in Drosophila biology. One method, used widely by those skilled in the art, is the Gal4-UAS system (Brand and Perrimon, Development 118:401 (1993)). Briefly, this system comprises genetically crossing two different transgenic Drosophila lines that carry transposable elements, called P-elements (U.S. Pat. No. 4,670,388), within their genomes. A P-element insertion in one line contains a nucleic acid fragment encoding the yeast Gal4 transcriptional activator protein. Gal4 protein can be expressed either by an endogenous promoter in the Drosophila genome upstream of the insertion site of a P-element carrying the Gal4 open reading frame and a minimal promoter, or by a regulatory element that is engineered into the P-element upstream of the Gal4 open reading frame and a minimal promoter. In the former case Gal4 protein is expressed in the pattern of the endogenous enhancer, while in the latter, Gal4 protein is expressed in the pattern of the regulatory element placed upstream of it. The second transgenic Drosophila line carries a P-element containing a tandem array of fourteen upstream activating sequence (UAS) sites upstream of a nucleic acid encoding a gene of interest. One embodiment of this latter type of P-element that is commonly used by those skilled in the art has the UAS sites upstream of an hsp70 promoter and a multiple cloning site to facilitate the insertion of a gene of interest and is referred to as pUAST (Brand and Perrimon, 1993). A genetic cross of the two Drosophila lines results in the inheritance of both kinds of P-elements in a fraction of their progeny. In these progeny, the Gal4 protein is locally mis-expressed in some tissues, in which it binds to the UAS sites in the second P-element, resulting in expression of the gene of interest. This system exhibits a tremendous amount of flexibility in both the patterns of Gal4 expression lines available, and in being able to engineer pUAST constructs containing any gene of interest.

To screen for modifiers of the number and/or size of fat cells, a genetic screen was employed. Genetic screens, as described earlier, can be used to discover other genes that execute biological processes. One method known to those skilled in the art employs a library of EP fly lines (Rorth et al., Development 125:1049 (1998)). EP lines each contain a genomic insertion of a P-element that contains fourteen tandem copies of the upstream activator sequences (UAS) from yeast immediately upstream of a basal promoter. This sequence is bound with high affinity by the Gal4 transcriptional activator protein. The insertion of an EP element thus places a Gal4-inducible promoter either (1) near the 5′ (upstream) end of a gene or; (2) places an inducible promoter within a gene. Wherever Gal4 protein is present within the fly, genes into which an EP element has inserted will either be (1) activated or; (2) inactivated via expression of an anti-sense RNA.

To identify genes that modify the number and/or size of fat cells, flies carrying a heat shock-inducible Gal4 driver (hsp70-Gal4), well-known to those skilled in the art and available from the Bloomington Stock Center (Indiana, U.S.A.), were crossed to each of approximately 24,400 independent EP lines. The eggs produced in these crosses were collected for 24 hours and subsequently raised at 25° C. for 48 hours. Developing flies were then reared at 29° C. for two days to induce the over- or under-expression of genes tagged by EP insertions in each different cross. Following heat shock, fly larvae were dissected by tearing them in half and inverting them to reveal their fat bodies in a manner frequently used by those skilled in the art. Fat cells in the fat bodies were observed under a microscope (Stemi 2000/KL1500 model, ZEISS, Germany). EP lines were considered to have insertions into genes that modify the number and/or size of fat cells if there was a detectable increase or decrease in either or both of these traits in flies containing both hsp70-Gal4 and an EP insertion compared to their wild-type siblings (containing either hsp70-Gal4 or an EP element alone).

188 EP lines met the criteria as modifiers of fat cell number and/or size. Each of these has an insertion into a gene that affects the number and/or size of fat cells.

To determine the genomic regions into which the EP elements had inserted in each of the fat cell number and/or size modifying EP lines and thereby identify the causative open reading frames, DNA sequences flanking each EP element were recovered using inverse PCR on genomic DNA from each EP line according to the method by J. Rehm at the Berkeley Drosophila Genome Project website, which is very commonly utilized both those skilled in the art.

Briefly, for each EP fly line 15-20 frozen flies were completely macerated in 500 μL of grinding buffer (350 mM NaCl, 7M Urea, 100 mM Tris, 10 mM EDTA and 2% SDS), and proteins were removed by a phenol/chloroform extraction procedure. Genomic DNA was then precipitated with 100% ethanol, air-dried, and resuspended in 100 μL of TE buffer (10 mM Tris-Cl, 1 mM EDTA). Genomic DNA was then digested with HinPI endonuclease (MBI Fermentas,) for 2 hrs at 37° C. and the residual enzyme activity subsequently heat inactivated. The digested DNA was then treated with T4 DNA ligase (MBI Fermentas) overnight at 4° C. The ligated DNA was recovered precipitated with isopropanol and sodium acetate (pH 5.2), and solubilized in 100 μL of TE (10 mM Tris-Cl, 1 mM EDTA).

10 μL of the ligated DNA was then amplified via PCR using primers 5R3 (5′-GCGAATCATTAAAGTGGGTATC-3′ SEQ ID NO: 435) and 5F3 (5′-GAGATGCATCTACACAAGGAAC-3′ SEQ ID NO: 436) that are targeted to the 5′ region of EP element sequences. PCR products were purified via elution through a PCR purification column (Qiagen) with 50μL of TE.

Secondary PCR reactions were then performed under cycling and buffer conditions described above using the 5R2 (5′-AATAGCACACTTCGGCAC-3′ SEQ ID NO: 437) and 5F1 (5′-AATGAACCACTCGGAACC-3′ SEQ ID NO: 438) primers. As previously, the PCR products were then purified over a column and stored at −20° C.

The secondary PCR products from each of the EP lines were sequenced using the Sp1 primer (5′-ACA CAA CCT TTC CTC TCA ACA A-3′ SEQ ID NO: 439) and the ABI BigDye Terminator Cycle Sequencing Ready Reaction Kit (Perkin Elmer), and run on an ABI 377XL Automated Sequencer (Perkin Elmer) in polymerized Long Ranger Gel Solution (FMC Bioproducts).

If at least 20 base pairs of flanking sequence were recovered from the cloned inverse PCR product from an EP insertion, the sequence was searched against all Drosophila sequences using the BLAST search tool, familiar to those skilled in art, at the Berkeley Drosophila Genome Project website, and matches were validated by their e-values, which establish the statistically significant threshold for reporting database sequence matches (Altschul, J. Mol. Evol. 36:290 (1993)). The scaffold of Drosophila genomic sequence that showed the highest sequence identity with each flanking DNA sequence was selected, and the exact position of the EP insertion relative to the flanking sequence was determined. Expressed sequence tags, complementary DNA's, and predicted open reading frames closest to the inserted EP element were searched for, and the direction of genes relative to the EP transcription unit (dictated by the direction of the EP promoter and UAS sites) was analyzed using databases in websites hosted by the National Center for Biotechnology Information and Berkeley Drosophila Genome Project. Candidate genes associated with an EP insertion and an open reading frame or an EST were identified as genes that modify the number and/or size of fat cells.

Drosophila, human, mouse, and rat homologs of the Drosophila fat cell size and/or number modifying genes were identified by inputting the amino acid sequences encoded by open reading frame sequences or ESTs associated with the Drosophila fat cell size and/or number modifying genes in blastp searches using the BLAST search tool of the National Center for Biotechnology Information. Drosophila, human, mouse, and rat proteins whose sequences matched the Drosophila fat cell size and/or number modifier protein sequences with sufficiently-low e-values, and their encoding nucleic acids are contemplated to modify fat cell size and/or number.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A method for identifying compounds that influence fat cell number or size, comprising:
a. providing:
i. a cell that expresses a gene selected from the group consisting of SEQ ID Nos: 1-217; and
ii. an agent;
b. exposing said cell to said agent; and
c. identifying fat cell number or size relative to cells not exposed to said agent.

2. The method of claim 1, wherein said cell is in vitro.

3. The method of claim 1, wherein said cell is in vivo.

4. The method of claim 1, wherein said cell is contained in a tissue.

5. The method of claim 1, wherein said agent comprises an antibody.

6. The method of claim 1, further comprising the step of conducting a safety study with said agent.

7. The method of claim 1, further comprising the step of conducting an efficacy study with said agent.

8. The method of claim 1, further comprising the step of selling said agent with a label indicating use of the agent for treating a disease or condition associated with fat cell size or number.

9. The method of claim 1, further comprising the step of marketing said agent for sale to patients having a disease or condition associated with fat cell size or number.

10. A method for identifying compounds that influence fat cell number or size, comprising:
a. providing:
i. an expression vector comprising a gene selected from the group consisting of SEQ ID Nos: 1-217; and
ii. an agent;
b. exposing said expression vector to said agent;
c. detecting a change in expression of said gene relative to the expression of said gene in an expression vector not exposed to said agent;
d. treating a subject with said agent; and
e. identifying fat cell number or size in said subject.

11. The method of claim 10, wherein said agent comprises an antisense oligonucleotide.

12. The method of claim 10, wherein said subject comprises a mammal.

13. The method of claim 10, wherein said subject comprises a human.

14. A method for identifying compounds that influence fat cell number or size, comprising:
a. providing:
i. polypeptide selected from the group consisting of SEQ ID Nos: 218-434; and
ii. an agent;
b. exposing said polypeptide to said agent;
c. detecting binding of said agent to said polypeptide or a change in an activity of said polypeptide;
d. treating a subject with said agent;
e. identifying fat cell number or size in said subject.

15. The method of claim 14, wherein said agent comprises an antibody.

16. The method of claim 14, wherein said subject comprises a mammal.

17. The method of claim 14, wherein said subject comprises a human.

18. A method for regulating fat cell size or number, comprising:
a. providing:
i. a subject containing fat cells; and
ii. an agent that changes the expression of a gene selected from the group consisting of SEQ ID NOs: 1-217; and
b. treating said subject with said agent under conditions such that fat cell size or number in said subject is altered.

19. The method of claim 18, wherein said agent comprises an expression vector that expresses said gene.

20. A method for regulating fat cell size or number, comprising:
a. providing:
i. a subject containing fat cells; and
ii. an agent that changes the activity of a polypeptide selected from the group consisting of SEQ ID NOs: 218-434; and
b. treating said subject with said agent under conditions such that fat cell size or number in said subject is altered.

21. The method of claim 20, wherein said agent comprises said polypeptide.

22. The method of claim 21, wherein said agent comprises an antibody that binds to said polypeptide.

23. A composition comprising a nucleic acid that encodes a polypeptide selected from the group consisting of SEQ ID NOs: 218-434.

24. A composition comprising an expression vector comprising said nucleic acid of claim 23.

25. The composition of claim 24, wherein said expression vector further comprises a neuronal-specific promoter.

26. A composition comprising a host cell, said host cell comprising the expression vector of claim 24.

27. A composition comprising an antisense oligonucleotide that hybridizes under stringent conditions to said nucleic acid of claim 23.

28. A composition comprising a polypeptide selected from the group consisting of SEQ ID NOs: 218-434.

29. A composition comprising an antibody that specifically binds to said polypeptide of claim 28.
